SEQUENCE LISTING (221 sequences)

SEQ ID NO: 1 - protein, 7 aa, SV40 virus
PKKKRKV

SEQ ID NO: 2 - protein, 16 aa, unidentified
  REGION 1..16: note = nucleoplasmin bipartite NLS
KRPAATKKAGQAKKKK

SEQ ID NO: 3 - protein, 9 aa, unidentified
  REGION 1..9: note = c-myc NLS
PAAKRVKLD

SEQ ID NO: 4 - protein, 11 aa, unidentified
  REGION 1..11: note = c-myc NLS
RQRRNELKRSP

SEQ ID NO: 5 - protein, 38 aa, unidentified
  REGION 1..38: note = hRNPA1 M9 NLS
NQSSNFGPMKGGNFGGRSSGPYGGGGQYFAKPRNQGGY

SEQ ID NO: 6 - protein, 42 aa, unidentified
  REGION 1..42: note = IBB domain from importin-alpha
RMRIZFKNKGKDTAELRRRRVEVSVELRKAKKDEQILKRRNV

SEQ ID NO: 7 - protein, 8 aa, unidentified
  REGION 1..8: note = myoma T protein
VSRKRPRP

SEQ ID NO: 8 - protein, 8 aa, unidentified
  REGION 1..8: note = myoma T protein
PPKKARED

SEQ ID NO: 9 - protein, 8 aa, Homo sapiens
PQPKKKPL

SEQ ID NO: 10 - protein, 12 aa, Mus musculus
SALIKKKKKMAP

SEQ ID NO: 11 - protein, 5 aa, Influenza virus
DRLRR

SEQ ID NO: 12 - protein, 10 aa, Hepatitus virus
RKLKKKIKKL

SEQ ID NO: 13 - protein, 10 aa, Mus musculus
REKKKFLKRR

SEQ ID NO: 14 - protein, 20 aa, Homo sapiens
KRKGDEVDGVDEVAKKKSKK

SEQ ID NO: 15 - protein, 17 aa, Homo sapiens
RKCLQAGMNLEARKTKK

SEQ ID NO: 16 - protein, 7 aa, Influenza virus
PKQKKRK

SEQ ID NO: 17 - protein, 30 aa, synthetic construct
  REGION 1..30: note = synthetic sequence
MDSLLMNRRKFLYQFKNVRWAKGRRETYLC

SEQ ID NO: 18 - protein, 5 aa, synthetic construct
  REGION 1..5: note = immunoglobulin hinge region
DKTHT

SEQ ID NO: 19 - protein, 4 aa, synthetic construct
  REGION 1..4: note = immunoglobulin hinge region
CPPC

SEQ ID NO: 20 - protein, 15 aa, synthetic construct
  REGION 1..15: note = immunoglobulin hinge region
CPEPKSCDTPPPCPR

SEQ ID NO: 21 - protein, 12 aa, synthetic construct
  REGION 1..12: note = immunoglobulin hinge region
ELKTPLGDTTHT

SEQ ID NO: 22 - protein, 10 aa, synthetic construct
  REGION 1..10: note = immunoglobulin hinge region
KSCDKTHTCP

SEQ ID NO: 23 - protein, 7 aa, synthetic construct
  REGION 1..7: note = immunoglobulin hinge region
KCCVDCP

SEQ ID NO: 24 - protein, 7 aa, synthetic construct
  REGION 1..7: note = immunoglobulin hinge region
KYGPPCP

SEQ ID NO: 25 - protein, 15 aa, Homo sapiens
EPKSCDKTHTCPPCP

SEQ ID NO: 26 - protein, 12 aa, Homo sapiens
ERKCCVECPPCP

SEQ ID NO: 27 - protein, 17 aa, Homo sapiens
ELKTPLGDTTHTCPRCP

SEQ ID NO: 28 - protein, 12 aa, Homo sapiens
SPNMVPHAHHAQ

SEQ ID NO: 29 - protein, 45 aa, Homo sapiens
TTTPAPRPPTPAPTIASQPLSLRPEACRPAAGGAVHTRGLDFACD

SEQ ID NO: 30 - protein, 24 aa, unidentified
  REGION 1..24: note = transmembrane sequence
IYIWAPLAGTCGVLLLSLVITLYC

SEQ ID NO: 31 - protein, 23 aa, unidentified
  REGION 1..23: note = CD8 beta derived transmembrane sequence
LGLLVAGVLVLLVSLGVAIHLCC

SEQ ID NO: 32 - protein, 25 aa, unidentified
  REGION 1..25: note = CD4 derived transmembranse sequence
ALIVLGGVAGLLLFIGLGIFFCVRC

SEQ ID NO: 33 - protein, 23 aa, unidentified
  REGION 1..23: note = CD3 zeta derived transmembrane sequence
LCYLLDGILFIYGVILTALFLRV

SEQ ID NO: 34 - protein, 26 aa, unidentified
  REGION 1..26: note = CD28 derived transmembrane sequence
WVLVVVGGVLACYSLLVTVAFIIFWV

SEQ ID NO: 35 - protein, 26 aa, unidentified
  REGION 1..26: note = CD134 (OX40) derived transmembrane sequence
VAAILGLGLVLGLLGPLAILLALYLL

SEQ ID NO: 36 - protein, 24 aa, unidentified
  REGION 1..24: note = CD7 derived transmembrane sequence
ALPAALAVISFLLGLGLGVACVLA

SEQ ID NO: 37 - protein, 42 aa, unidentified
  REGION 1..42: note = 4-1BB polypeptide
KRGRKKLLYIFKQPFMRPVQTTQEEDGCSCRFPEEEEGGCEL

SEQ ID NO: 38 - protein, 44 aa, unidentified
  REGION 1..44: note = CD28 polypeptide
FWVRSKRSRLLHSDYMNMTPRRPGPTRKHYQPYAPPRDFAAYRS

SEQ ID NO: 39 - protein, 35 aa, unidentified
  REGION 1..35: note = ICOS polypeptide
TKKKYSSSVHDPNGEYMFMRAVNTAKKSRLTDVTL

SEQ ID NO: 40 - protein, 37 aa, unidentified
  REGION 1..37: note = OX40 polypeptide
RRDQRLPPDAHKPPGGGSFRTPIQEEQADAHSTLAKI

SEQ ID NO: 41 - protein, 114 aa, unidentified
  REGION 1..114: note = BTLA polypeptide
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 42 - protein, 49 aa, unidentified
  REGION 1..49: note = CD27 polypeptide
HQRRKYRSNKGESPVEPAEPCRYSCPREEEGSTIPIQEDYRKPEPACSP

SEQ ID NO: 43 - protein, 187 aa, unidentified
  REGION 1..187: note = CD30 polypeptide
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 44 - protein, 54 aa, unidentified
  REGION 1..54: note = GITR polypeptide
HIWQLRSQCMWPRETQLLLEVPPSTEDARSCQFPEEERGERSAEEKGRLGDLWV

SEQ ID NO: 45 - protein, 60 aa, unidentified
  REGION 1..60: note = HVEM polypeptide
CVKRRKPRGDVVKVIVSVQRKRQEAEGEATVIEALQAPPDVTTVAVEETIPSFTGRSPNH

SEQ ID NO: 46-47 - skipped (2 entries)

SEQ ID NO: 48 - protein, 512 aa, Vesicular stomatitis virus
IMKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 49 - protein, 563 aa, Baboon endogenous retrovirus
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 50 - protein, 566 aa, Influenza virus
  VARIANT 239..240: note = Xaa can be any naturally occurring amino acid
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRXXEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 51 - protein, 566 aa, Influenza virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNNSFFSRLNWLTHLKFKYPALNVTMPNNEKFDKLYIWGVHHPGTDNDQISLYAQASGRITVSTKRSQQTVIPSIGSRPRIRDVPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 52 - protein, 568 aa, Influenza virus
MEKIVLLLAIVSLVKSDQICIGYHANNSTEQVDTIMEKNVTVTHAQDILEKTHNGKLCDLNGVKPLILRDCSVAGWLLGNPMCDEFINVPEWSYIVEKASPANDLCYPGDFNDYEELKHLLSRTNHFEKIQIIPKSSWSNHDASSGVSSACPYHGRSSFFRNVVWLIKKNSAYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIIDKMNTQFEAVGREFNNLERRIENLNKQMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILSIYSTVASSLALAIMVAGLSLWMCSNGSLQCRICI

SEQ ID NO: 53 - protein, 560 aa, Influenza virus
MNTQILVFALIAIIPTNADKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 54 - protein, 226 aa, Hepatitis B Virus
MENTTSGFLGPLLVLQAGFFLLTRNLTIPQSLDSWWTSLNFLGGAPTCPGQNSQSPTSNHSPTSCPPICPGYRWMCLRRFIIFLFILLLCLIFLLVLLDYQGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 55 - protein, 281 aa, Hepatitis B Virus
MQWNSTAFHQALQDPKVRGLYFPAGGSSSGTVNPAPNIASHISSISARTGDPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 56 - protein, 399 aa, Hepatitis B Virus
MGLSWTVPLEWGKNHSTTNPLGFFPDHQLDPAFRANTRNPDWDHNPNKDHWTEANKVGVGAFGPGFTPPHGGLLGWSPQAQGMLKTLPADPPPASTNRQSGRQPTPITPPLRDTHPQAMQWNSTTFHQALQDPKVSALYLPAGGSSSGTVNPVPTTASLISSIFSRIGDPAPNMESITSGFLGPLLVLQAGFFLLTKILTIPQSLDSWWTSLNFLGGAPVCLGQNSQSPTSSHSPTSCPPICPGYRWMCLRRFIIFLFILLLCLIFLLVLLDYQGMLPVCPLIPGSSTTSTGPCRTCTTLAQGTSMFPSCCCSKPSDGNCTCIPIPSSWAFGKFLWEWASARFSWLSLLVPFVQWFAGLSPTVWLSVIWMMWYWGPSLYNILSPFIPLLPIFFCLWVYI

SEQ ID NO: 57 - protein, 226 aa, Hepatitis B Virus
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWAPNLHNILSPFLPLLPIFLCLWVYI

SEQ ID NO: 58 - protein, 400 aa, Hepatitis B Virus
MGGWSSKPRKGMGTNLAVPNPLGFFPDHQLDPAFKANSDNPDWDLNTHKDYWPDAWKVGVGAFGPGFTPPHGGLLGWSPQAQGLLTTVPAAPPPASTNRQSGRQPTPLSPPLRDTHPQAMKWNSTTFHQTLQDPRVRALYLPAGGSSSGTVSPAQNTVSAISSILSKTGDPVPNMESIASGLLGPLLVLQAGFFLLTKILTIPQSLDSWWTSLNFLGGTPVCLGQNSQSQISSHSPTCCPPTCPGYRWMCLRRFIIFLCILLLCLIFLLVLLDYQGMLPVCPLIPGSSTTSTGPCKTCTAPAQGTSMFPSCCCTKPTDGNCTCIPIPSSWAFAKYLWEWASVRFSWLSLLVPFVQWFVGLSPTVWLSVIWMMWFWGPSLYNILSPFIPLLPIFFCLWVYI

SEQ ID NO: 59 - protein, 281 aa, Hepatitis B Virus
MQWNSTTFHQTLQDPRVRGLYFPAGGSSSGTVNPVPTTVSHISSIFSRIGDPALNMENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLSVCPLIPGSTTTSTGPCKTCTTPAQGTSIHPSCCCTKPSDGNCTWIPIPSSWAFGKFLWEWASARFSWLSLLVPFVQWFVGLSPTVWLSVIWIMWYWGPSLYSILSPFLPLLPIFFCLWVYI

SEQ ID NO: 60 - protein, 524 aa, Rabies virus
MVPQALLFVPLLVFPLCFGKFPIYTIPDKLGPWSPIDIHHLSCPNNLVVEDEGCTNLSGFSYMELKVGYISAIKVNGFTCTGVVTEAETYTNFVGYVTTTFKRKHFRPTPDACRSAYNWKMAGDPRYEESLHNPYPDYHWLRTVKTTKESLVIISPSVADLDPYDKSLHSRVFPSGKCSGITVSSTYCSTNHDYTIWMPENLRLGTSCDIFINSRGKRASKGSQTCGFIDERGLYKSLKGACKLKLCGVLGLRLMDGTWVAMQTSDETKWCPPDQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPEGHVLIPEMQSSLLQQHMELLESSVIPLMHPLADPSTVFKEGDEAEDFVEVHLPDVHKQVSGVNLGLPNWGKYVLLSAGALIALMLIIFLLTCCRRVNRPESTQHSLGGKRRKVSITSQSGKIISSWESYKSGGETRL

SEQ ID NO: 61 - protein, 522 aa, Mokola virus
MNIPCFVVILSLATTHSLGEFPLYTIPEKIEKWTPIDMIHLSCPNNLLSEEEGCNAESSFTYFELKSGYLAHQKVPGFTCTGVVNEAETYTNFVGYVTTTFKRKHFRPTVAACRDAYNWKVSGDPRYEESLHTPYPDSSWLRTVTTTKESLLIISPSIVEMDIYGRTLHSPMFPSGVCSNVYPSVPSCETNHDYTLWLPEDPSLSLVCDIFTSSNGKKAMNGSRICGFKDERGFYRSLKGACKLTLCGRPGIRLFDGTWVSFTKPDVHVWCTPNQLINIHNDRLDEIEHLIVEDIIKKREECLDTLETILMSQSVSFRRLSHFRKLVPGYGKAYTILNGSLMETNVYYKRVDKWADILPSKGCLKVGQQCMEPVKGVLFNGIIKGPDGQILIPEMQSEQLKQHMDLLKAAVFPLRHPLISREAVFKKDGDADDFVDLHMPDVHKSVSDVDLGLPHWGFWMLIGATIVAFVVLVCLLRVCCKRVRRRRSGRATQEIPLSFPSAPVPRAKVVSSWESYKGLPGT

SEQ ID NO: 62 - protein, 498 aa, Lymphocytic choriomeningitis virus
MGQIVTMFEALPHIIDEVINIVIIVLIIITSIKAVYNFATCGILALISFLLLAGRSCGLYGLDGPDIYKGIYQFKSVEFDMSHLNLTMPNACSANNSHHYISMGNSGLELTFTNDSIISHNFCNLTSAFNKKTFDHTLMSIVSSLHLSIRGNSNYKAVSCDFNSGITIQYNLSFSDAQSALSQCKTFRGRVLDMFRTAFGGKYMRSGWGWTGSDGKTTWCSQTSYQYLIIQNRTWENHCRYAGPFGMARILFAQEKTKFLTRRLAGTFTWTLSDSSGVDNPGGYCLTRWMILAADLKCFGNTAVAKCNMNHDEEFCDMLRLIDYNKAALSKFKEDVESALHLFKVTVNSLVSDQLLMRNHLRDLMGVPYCNYSRFWYLEHTKTGETSVPKCWLVTNGSYLNETHFSDQIEQEADNMITDMLRKDYIKRQGSTPLALMDLLMFSTSAYLVSVFLHLVKIPTHRHIKGGSCPKPHRLTNKGICSCGAFKVPGVKTVWKRR

SEQ ID NO: 63 - protein, 498 aa, Lymphocytic choriomeningitis virus
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 64 - protein, 498 aa, Lymphocytic choriomeningitis virus
MGQIVTMFEALPHIIDEVINIVIIVLIVITGIKAVYNFATCGIFALISFLLLAGRSCGMYGLKGPDIYKGVYQFKSVEFDMSHLNLTMPNACSANNSHHYISMGTSGLELTFTNDSIISHNFCNLTSAFNKKTFDHTLMSIVSSLHLSIRGNSNYKAVSCDFNNGITIQYNLTFSDAQSAQSQCRTFRGRVLDMFRTAFGGKYMRSGWGWTGSDGKTTWCSQTSYQYLIIQNRTWENHCTYAGPFGMSRILLSQEKTKFFTRRLAGTFTWTLSDSSGVENPGGYCLTKWMILAAELKCFGNTAVAKCNVNHDAEFCDMLRLIDYNKAALSKFKEDVESALHLFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLVSIFLHLVKIPTHRHIKGGSCPKPHRLTNKGICSCGAFKVPGVKTVWKRR

SEQ ID NO: 65 - protein, 207 aa, Lymphocytic choriomeningitis virus
MYGLKGPDIYKGVYQFKSVEFDMSHLNLTMPNACSANNSHHYISMGTSGLELTFTNDSIISHNFCNLTSAFNKKTFDHTLMSIVSSLHLSIRGNSNYKAVSCDFNNGITIQYNLTFSDAQSAQSQCRTFRGRVLDMFRTAFGGKYMRSGWGWTGSDGKTTWCSQTSYQYLIIQNRTWENHCTYAGPFGMSRILLSQEKTKFFTRRLA

SEQ ID NO: 66 - protein, 233 aa, Lymphocytic choriomeningitis virus
GTFTWTLSDSSGVENPGGYCLTKWMILAAELKCFGNTAVAKCNVNHDAEFCDMLRLIDYNKAALSKFKEDVESALHLFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLVSIFLHLVKIPTHRHIKGGSCPKPHRLTNKGICSCGAFKVPGVKTVWKRR

SEQ ID NO: 67 - protein, 438 aa, Ross River virus
  VARIANT 23: note = Xaa can be any naturally occurring amino acid
  VARIANT 35..36: note = Xaa can be any naturally occurring amino acid
  VARIANT 314: note = Xaa can be any naturally occurring amino acid
  VARIANT 358..359: note = Xaa can be any naturally occurring amino acid
YEHTATIPNVVGFPYKAHIERNXFSPMTLQLEVVXXSLEPTLNLEYITCEYKTVVPSPFIKCCGTSECSSKEQPDYQCKVYTGVYPFMWGGAYCFCDSENTQLSEAYVDRSDVCKHDHALAYKAHTASLKATIRISYGTINQTTEAFVNGEHAVNVGGSKFIFGPISTAWSPFDNKIVVYKDDVYNQDFPPYGSGQPGRFGDIQSRTVESKDLYANTALKLSRPSPGVVHVPYTQTPSGFKYWLKEKGSSLNTKAPFGCKIKTNPVRAMDCAVGSIPVSMDIPDSAFTRVVDAPAVTDLSCQVAVCTHSSDFGXVATLSYKTDKPGKCAVHSHSNVATLQEATVDVKEDGKVTVHFSXXSASPAFKVSVCDAKTTCTAACEPPKDHIVPYGASHNNQVFPDMSGTAMTWVQRMASGLGGLALIAVVVLVLVTCITMRR

SEQ ID NO: 68 - protein, 422 aa, Ross River virus
  VARIANT 18: note = Xaa can be any naturally occurring amino acid
  VARIANT 113: note = Xaa can be any naturally occurring amino acid
  VARIANT 236: note = Xaa can be any naturally occurring amino acid
  VARIANT 293: note = Xaa can be any naturally occurring amino acid
  VARIANT 415: note = Xaa can be any naturally occurring amino acid
SVIEHFNVYKATRPYLAXCADCGDGYFCYSPVAIEKIRDEASDGMLKIQVSAQIGLDKAGTHAHTKMRYMAGHDVQESKRDSLRVYTSAACSIHGTMGHFIVAHCPPGDYLKXSFEDANSHVKACKVQYKHDPLPVGREKFVVRPHFGVELPCTSYQLTTAPTDEEIDMHTPPDIPDRTLLSQTAGNVKITAGGRTIRYNCTCGRDNVGTTSTDKTINTCKIDQCHAAVTSHDKWXFTSPFVPRADQTARKGKVHVPFPLTNVTCRVPLARAPDVTYGKKEVTLRLHPDHPTXFSYRSLGAVPHPYEEWVDKFSERIIPVTEEGIEYQWGNNPPVRLWAQLTTEGKPHGWPHEIIQYYYGLYPAATIAAVSGASLMALLTLAATCCMLATARRKCLTPYALTPGAVVPLTLGLLXCAPRANA

SEQ ID NO: 69 - protein, 438 aa, Semliki Forest virus
YEHSTVMPNVVGFPYKAHIERPGYSPLTLQMQVVETSLEPTLNLEYITCEYKTVVPSPYVKCCGASECSTKEKPDYQCKVYTGVYPFMWGGAYCFCDSENTQLSEAYVDRSDVCRHDHASAYKAHTASLKAKVRVMYGNVNQTVDVYVNGDHAVTIGGTQFIFGPLSSAWTPFDNKIVVYKDEVFNQDFPPYGSGQPGRFGDIQSRTVESNDLYANTALKLARPSPGMVHVPYTQTPSGFKYWLKEKGTALNTKAPFGCQIKTNPVRAMNCAVGNIPVSMNLPDSAFTRIVEAPTIIDLTCTVATCTHSSDFGGVLTLTYKTNKNGDCSVHSHSNVATLQEATAKVKTAGKVTLHFSTASASPSFVVSLCSARATCSASCEPPKDHIVPYAASHSNVVFPDMSGTALSWVQKISGGLGAFAIGAILVLVVVTCIGLRR

SEQ ID NO: 70 - protein, 439 aa, Sindbis virus
YEHATTVPNVPQIPYKALVERAGYAPLNLEITVMSSEVLPSTNQEYITCKFTTVVPSPKIKCCGSLECQPAAHADYTCKVFGGVYPFMWGGAQCFCDSENSQMSEAYVELSADCASDHAQAIKVHTAAMKVGLRIVYGNTTSFLDVYVNGVTPGTSKDLKVIAGPISASFTPFDHKVVIHRGLVYNYDFPEYGAMKPGAFGDIQATSLTSKDLIASTDIRLLKPSAKNVHVPYTQASSGFEMWKNNSGRPLQETAPFGCKIAVNPLRAVDCSYGNIPISIDIPNAAFIRTSDAPLVSTVKCEVSECTYSADFGGMATLQYVSDREGQCPVHSHSSTATLQESTVHVLEKGAVTVHFSTASPQANFIVSLCGKKTTCNAECKPPADHIVSTPHKNDQEFQAAISKTSWSWLFALFGGASSLLIIGLMIFACSMMLTSTRR

SEQ ID NO: 71 - protein, 423 aa, Sindbis virus
SVIDDFTLTSPYLGTCSYCHHTVPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLKETTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANA

SEQ ID NO: 72 - protein, 676 aa, Ebola Zaire virus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 73 - protein, 644 aa, Ebola Zaire virus
IPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 74 - protein, 677 aa, Ebola Reston virus
MGSGYQLLQLPRERFRKTSFLVWVIILFQRAISMPLGIVTNSTLKATEIDQLVCRDKLSSTSQLKSVGLNLEGNGIATDVPSATKRWGFRSGVPPKVVSYEAGEWAENCYNLEIKKSDGSECLPLPPDGVRGFPRCRYVHKVQGTGPCPGDLAFHKNGAFFLYDRLASTVIYRGTTFAEGVVAFLILSEPKKHFWKATPAHEPVNTTDDSTSYYMTLTLSYEMSNFGGNESNTLFKVDNHTYVQLDRPHTPQFLVQLNETLRRNNRLSNSTGRLTWTLDPKIEPDVGEWAFWETKKNFSQQLHGENLHFQIPSTHTNNSSDQSPAGTVQGKISYHPPANNSELVPTDSPPVVSVLTAGRTEEMSTQGLTNGETITGFTANPMTTTIAPSPTMTSEVDNNVPSEQPNNTASIEDSPPSASNETIYHSEMDPIQGSNNSAQSPQTKTTPAPTTSPMTQDPQETANSSKPGTSPGSAAGPSQPGLTINTVSKVADSLSPTRKQKRSVRQNTANKCNPDLYYWTAVDEGAAVGLAWIPYFGPAAEGIYIEGVMHNQNGLICGLRQLANETTQALQLFLRATTELRTYSLLNRKAIDFLLQRWGGTCRILGPSCCIEPHDWTKNITDEINQIKHDFIDNPLPDHGDDLNLWTGWRQWIPAGIGIIGVIIAIIALLCICKILC

SEQ ID NO: 75 - protein, 681 aa, Marburg virus
MKTTCFLISLILIQGTKNLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSDWGVLTNLGILLLLSIAVLIALSCICRIFTKYIG

SEQ ID NO: 76 - protein, 669 aa, Murine leukemia virus
MESTTLSKPFKNQVNPWGPLIVLLILGGVNPVALGNSPHQVFNLTWEVTNGDRETVWAIAGNHPLWTWWPDLTPDLCMLALHGPSYWGLEYRAPFSPPPGPPCCSGSSDSTPGCSRDCEEPLTSYTPRCNTAWNRLKLSKVTHAHNEGFYVCPGPHRPRWARSCGGPESFYCASWGCETTGRASWKPSSSWDYITVSNNLTSDQATPVCKGNEWCNSLTIRFTSFGKQATSWVTGHWWGLRLYVSGHDPGLIFGIRLKITDSGPRVPIGPNPVLSDRRPPSRPRPTRSPPPSNSTPTETPLTLPEPPPAGVENRLLNLVKGAYQALNLTSPDKTQECWLCLVSGPPYYEGVAVLGTYSNHTSAPANCSVASQHKLTLSEVTGQGLCIGAVPKTHQVLCNTTQKTSDGSYYLAAPTGTTWACSTGLTPCISTTILDLTTDYCVLVELWPRVTYHSPSYVYHQFEGRAKYKREPVSLTLALLLGGLTMGGIAAGVGTGTTALVATQQFQQLQAAMHDDLKEVEKSITNLEKSLTSLSEVVLQNRRGLDLLFLKEGGLCAALKEECCFYADHTGLVRDSMAKLRERLSQRQKLFESQQGWFEGLFNKSPWFTTLISTIMGPLIILLLILLFGPCILNRLVQFIKDRISVVQALVLTQQYHQLKTIRDCKSRE

SEQ ID NO: 77 - protein, 669 aa, Murine leukemia virus
MESTTLSKPFKNQVNPWGPLIVLLILRGVNPVTLGNSPHQVFNLTWEVTNGDRETVWAITGNHPLWTWWPDLTPDLCMLALHGPSYWGLEYRAPFSPPPGPPCCSGSSDSTPGCSRDCEEPLTSYTPRCNTAWNRLKLSKVTHAHNGGFYVCPGPHRPRWARSCGGPESFYCASWGCETTGRASWKPSSSWDYITVSNNLTSDQATPVCKGNKWCNSLTIRFTSFGKQATSWVTGHWWGLRLYVSGHDPGLIFGIRLKITDSGPRVPIGPNPVLSDRRPPSRPRPTRSPPPSNSTPTETPLTLPEPPPAGVENRLLNLVKGAYQALNLTSPDKTQECWLCLVSGPPYYEGVAVLGTYSNHTSAPANCSVASQHKLTLSEVTGQGLCIGAVPKTHQVLCNTTQKTSDGSYYLAAPTGTTWACSTGLTPCISTTILDLTTDYCVLVELWPRVTYHSPSYVYHQFERRAKYKREPVSLTLALLLGGLTMGGIAAGVGTGTTALVATQQFQQLQAAMHDDLKEVEKSITNLEKSLTSLSEVVLQNRRGLDLLFLKEGGLCAALKEECCFYADHTGLVRDSMAKLRERLSQRQKLFESQQGWFEGLFNKSPWFTTLISTIMGPLIILLLILLFGPCILNRLVQFIKDRISVVQALVLTQQYHQLKIIEDCKSRE

SEQ ID NO: 78 - protein, 654 aa, Murine leukemia virus
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 79 - protein, 645 aa, Murine leukemia virus
MEGPAFSKPLKDKINPWKSLMVMGVYLRVGMAESPHQVFNVTWRVTNLMTGRTANATSLLGTVQDAFPRLYFDLCDLVGEEWDPSDQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYQALNLTNPDKTQECWLCLVSGPPYYEGVAVVGTYTNHSTAPASCTATSQHKLTLSEVTGQGLCMGALPKTHQALCNTTQSAGSGSYYLAAPAGTMWACSTGLTPCLSTTMLNLTTDYCVLVELWPRIIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGQFNRSPWFTTLISTIMGPLIVLLLILLFGPCILNRLVQFVKDRISVVQALVLTQQYHQLKPIEYEP

SEQ ID NO: 80 - protein, 644 aa, Polytropic mink cell focus-forming virus
MEGSAFSKPLKDKINPWGPLIVMGILVRAGASVQRDSPHQIFNVTWRVTNLMTGQTANATSLLGTMTDTFPKLYFDLCDLVGDYWDDPEPDIGDGCRTPGGRRRTRLYDFYVCPGHTVPIGCGGPGEGYCGKWGCETTGQAYWKPSSSWDLISLKRGNTPKDQGPCYDSSVSSGVQGATPGGRCNPLVLEFTDAGRKASWDAPKVWGLRLYRSTGADPVTRFSLTRQVLNVGPRVPIGPNPVITDQLPPSQPVQIMLPRPPHPPPSGTVSMVPGAPPPSQQPGTGDRLLNLVEGAYQALNLTSPDKTQECWLCLVSGPPYYEGVAVLGTYSNHTSAPANCSVASQHKLTLSEVTGQGLCVGAVPKTHQALCNTTQKTSDGSYYLAAPAGTIWACNTGLTPCLSTTVLNLTTDYCVLVELWPKVTYHSPDYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFTTLISTIMGPLIVLLLILLLGPCILNRLVQFVKDRISVVQALILTQQYHQLKSIEPEEVESRE

SEQ ID NO: 81 - protein, 408 aa, Polytropic mink cell focus-forming virus
VQHDSPHQVFNVTWRVTNLMTGQTANATSLLGTMTDAFPKLYFDLCDLIGDDWDETGLGCRTPGGRKRARTFDFYVCPGHTVPTGCGGPREGYCGKWGCETTGQAYWKPSSLWDLISLKRGNTPQNQGPCYDSSAVSSDIKGATPGGRCNPLVLEFTDAGKKASWDGPKVWGLRLYRSTGTDPVTRFSLTRRVLNIGPRVPIGPNPVIIDQLPPSRPVQIMLPRPPQPPPPGAASIVPETAPPSNQPGTGDRLLNLVDGAYQALNLTSPDKTQECWLCLVAEPPYYEGVAVLGTYSNHTSAPANCSVASQHKLTLSEVTGRGLCIGTVPKTHQALCNTTLKTNKGSYYLVAPAGTTWACNTGLTPCLSATVLNRTTDYCVLVELWPRVTYHPPSYVYSQFEKSYRHKR

SEQ ID NO: 82 - protein, 685 aa, Gibbon ape leukemia virus
MVLLPGSMLLTSNLHHLRHQMSPGSWKRLIILLSCVFGGGGTSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKILVLRQKYQALENEGNL

SEQ ID NO: 83 - protein, 644 aa, Gibbon ape leukemia virus
TSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKILVLRQKYQALENEGNL

SEQ ID NO: 84 - protein, 448 aa, Gibbon ape leukemia virus
TSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKR

SEQ ID NO: 85 - protein, 181 aa, Gibbon ape leukemia virus
EAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKIL

SEQ ID NO: 86 - protein, 565 aa, RD114 retrovirus
MKLPTGMVILCSLIIVRAGFDDPRKAIALVQKQHGKPCECSGGQVSEAPPNSIQQVTCPGKTAYLMTNQKWKCRVTPKNLTPSGGELQNCPCNTFQDSMHSSCYTEYRQCRANNKTYYTATLLKIRSGSLNEVQILQNPNQLLQSPCRGSINQPVCWSATAPIHISDGGGPLDTKRVWTVQKRLEQIHKAMHPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTGLCVQASLLPDIDIIPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFSRLMAFINDRLNVVHAMVLAQQYQALKAEEEAQD

SEQ ID NO: 87 - protein, 565 aa, Sendai virus
MTAYIQRSQCISTSLLVVLTTLVSCQIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIKTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPIDISLNLADATNFLQDSKAELEKARKILSEVGRWYNSRETVITIIVVMVVILVVIIVIIIVLYRLRRSMLMGNPDDRIPRDTYTLEPKIRHMYTNGGFDAMAKER

SEQ ID NO: 88 - protein, 540 aa, Sendai virus
QIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIKTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPIDISLNLADATNFLQDSKAELEKARKILSEVGRWYNSRETVITIIVVMVVILVVIIVIIIVLYRLRRSMLMGNPDDRIPRDTYTLEPKIRHMYTNGGFDAMAEKR

SEQ ID NO: 89 - protein, 91 aa, Sendai virus
QIPRDRLSNIGVIVDEGKSLKIAGSHESRYIVLSLVPGVDFENGCGTAQVIQYKSLLNRLLIPLRDALDLQEALITVTNDTTQNAGAPQSR

SEQ ID NO: 90 - protein, 449 aa, Sendai virus
FFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIKTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPIDISLNLADATNFLQDSKAELEKARKILSEVGRWYNSRETVITIIVVMVVILVVIIVIIIVLYRLRRSMLMGNPDDRIPRDTYTLEPKIRHMYTNGGFDAMAKER

SEQ ID NO: 91 - protein, 575 aa, Sendai virus
MDGDRSKRDSYWSTSPGGSTTKLVSDSERSGKVDTWLLILAFTQWALSIATVIICIVIAARQGYSMERYSMTVEALNTSNKEVKESLTSLIRQEVITRAANIQSSVQTGIPVLLNKNSRDVIRLIEKSCNRQELTQLCDSTIAVHHAEGIAPLEPHSFWRCPAGEPYLSSDPEVSLLPGPSLLSGSTTISGCVRLPSLSIGEAIYAYSSNLITQGCADIGKSYQVLQLGYISLNSDMFPDLNPVVSHTYDINDNRKSCSVVATGTRGYQLCSMPIVDERTDYSSDGIEDLVLDILDLKGRTKSHRYSNSEIDLDHPFSALYPSVGSGIATEGSLIFLGYGGLTTPLQGDTKCRIQGCQQVSQDTCNEALKITWLGGKQVVSVLIQVNDYLSERPRIRVTTIPITQNYLGAEGRLLKLGDQVYIYTRSSGWHSQLQIGVLDVSHPLTISWTPHEALSRPGNEDCNWYNTCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES

SEQ ID NO: 92 - protein, 611 aa, Jaagsiekte sheep retrovirus
MPKRRAGFRKGWYARQRNSLTHQMQRMTLSEPTSELPTQRQIEALMRYAWNEAHVQPPVTPTNILIMLLLLLQRIQNGAAATFWAYIPDPPMLQSLGWDKETVPVYVNDTSLLGGKSDIHISPQQANISFYGLTTQYPMCFSYQSQHPHCIQVSADISYPRVTISGIDEKTGMRSYRDGTGPLDIPFCDKHLSIGIGIDTPWTLCRARIASVYNINNANTTLLWDWAPGGTPDFPEYRGQHPPISSVNTAPIYQTELWKLLAAFGHGNSLYLQPNISGSKYGDVGVTGFLYPRACVPYPFMVIQGHMEITPSLNIYYLNCSNCILTNCIRGVAKGEQVIIVKQPAFVMLPVEITEEWYDETALELLQRINTALSRPKRGLSLIILGIVSLITLIATAVTASVSLAQSIQVAHTVDSLSSNVTKVMGTQENIDKKIEDRLPALYDVVRVLGEQVQSINFRMKIQCHANYKWICVTKKPYNTSDFPWDKVKKHLQGIWFNTTVSLDLLQLHNEILDIENSPKATLNIADTVDNFLQNLFSNFPSLHSLWRSIIAMGAVLTFVLIIICLAPCLIRSIVKEFLHMRVLIHKNMLQHQHLMELLNNKERGAAGDDP

SEQ ID NO: 93 - protein, 505 aa, Baculovirus
MFHLLTLLLLLFINMNLYLAGEHCNVQMKNGPYRIKNLAITPPRETLKKDVTVTIVETDYEENVLIGYKGYYQAYGYNGGSLDANTRLEETMESLPLTKEDLLTWTYRQECEVGEELIDRWGSDSDDCYRNKDGRGVWVKTKELVKRQNNNHFAHHTCNRSWRCGFSTAKMYSKLVCDDETNDCKVFILDNTGKPINITTNEVLYRDGVNMMLKSKPTFTRREEKVACLLVKDELNPDKTREHCLIDSDIYDLSNNNWFCMFNKCIKRNVDSVVKKRPNKWMHNLAPKYSEGATATKGDMMHIQEELMYENDLLKMNIELVHAHMNKLNNIIHDLIVSIAKVDERLIGNLMNISVSSVFLSDDTFLLMPCTNPPQHTSNCYNNSIYREGRWVFNEDTSECIDFNNYRELSIDDDIEFWIPTIGNTTYHDSWKDASGWSFVAQQKSNLIMTMENTKFGGVGTSLSDITSMSEGELTAKLTTFVFSHIVTFILIIILIILCICLLKK

SEQ ID NO: 94 - protein, 509 aa, Baculovirus
MLRITLLILFLVRFVSGAEHCNAQMKSGPWRIKNLPIAPPKETLQKDVDVEIVETDLDENVIIGYKGYYQAYAYNGGSLDPNTSVDETTQTLNIDKDDLITWGDRRKCEVGEELIDQWGSDSDSCFKDKLGRGVWVAGKELVKRKNNNHFAHHTCNRSWRCGVSTAKMYTRLECDNETDDCKVTILDINGTVINVTENEVLHRDGVSMILKQKSTFTRRTEKVACLLIKDDKSDPYSITREHCLIDNDIFDLSKNTWNCKFNRCIKRRSENVVKKRPPTWRHNEPPKHSEGTTATKGDLMHIQEELMYENDLLRMNLELLHAHINKLNNMMHDLIVSVAKVDERLIGNLMNNSVSSTFLSDDTFLLMPCTNPPPHTSNCYNNSIYKEGRWVANTDSSQCIDFRNYKELAIDDDIEFWIPTIGNTSYHESWKDASGWSFIAQQKSNLISTMENTKFGGHTTSLSDIGDMAKGELNATLYSFMLGHGFSFFLIIGVIVFLICMVRSRVRAF

SEQ ID NO: 95 - protein, 530 aa, Chandipura virus
MTSSVTISVILLISFIAPSYSSLSIAFPENTKLDWKPVTKNTRYCPMGGEWFLEPGLQEESFLSSTPIGATPSKSDGFLCHAAKWVTTCDFRWYGPKYITHSIHNIKPTRSDCDTALASYKSGTLVSPGFPPESCGYASVTDSEFLVIMITPHHVGVDDYRGHWVDPLFVGGECDQSYCDTIHNSSVWIPADQTKKNICGQSFTPLTVTVAYDKTKEIAAGAIVFKSKYHSHMEGARTCRLSYCGRNGIKFPNGEWVSLDVKTKIQEKPLLPLFKECPAGTEVRSTLQSDGAQVLTSEIQRILDYSLCQNTWDKVERKEPLSPLDLSYLASKSPGKGLAYTVINGTLSFAHTRYVRMWIDGPVLKEMKGKRESPSGISSDIWTQWFKYGDMEIGPNGLLKTAGGYKFPWHLIGMGIVDNELHELSEANPLDHPQLPHAQSIADDSEEIFFGDTGVSKNPVELVTGWFTSWKESLAAGVVLILVVVLIYGVLRCFPVLCTTCRKPKWKKGVERSDSFEMRIFKPNNMRARV

SEQ ID NO: 96 - protein, 1251 aa, Venezuelan equine encephalitis virus
MFPFQPMYPMQPMPYRNPFAAPRRPWFPRTDPFLAMQVQELTRSMANLTFKQRRDAPPEGPSAKKPKKEASQKQKGGGQGKKKKNQGKKKAKTGPPNPKAQNGNKKKTNKKPGKRQRMVMKLESDKTFPIMLEGKINGYACVVGGKLFRPMHVEGKIDNDVLAALKTKKASKYDLEYADVPQNMRADTFKYTHEKPQGYYSWHHGAVQYENGRFTVPKGVGAKGDSGRPILDNQGRVVAIVLGGVNEGSRTALSVVMWNEKGVTVKYTPENCEQWSLVTTMCLLANVTFPCAQPPICYDRKPAETLAMLSVNVDNPGYDELLEAAVKCPGSTEELFKEYKLTRPYMARCIRCAVGSCHSPIAIEAVKSDGHDGYVRLQTSSQYGLDSSGNLKGRTMRYDMHGTIKEIPLHQVSLHTSRPCHIVDGHGYFLLARCPAGDSITMEFKKDSVTHSCSVPYEVKFNPVGRELYTHPPEHGVEQACQVYAHDAQNRGAYVEMHLPGSEVDSSLVSLSGSSVTVTPPVGTSALVECECGGTKISETINKTKQFSQCTKKEQCRAYRLQNDKWVYISDKLPKAAGATLKGKLHVPFLLADGKCTVPLAPEPMITFGFRSVSLKLHPKNPTYLTTRQLADEPHYTHELISEPAVRNFTVTGKGWEFVWGNHPPKRFWAQETAPGNPHGLPHEVITHYYHRYPMSTILGLSICAAIATVSVAASTWLFCRSRVACLTPYRLTPNARIPFCLAVLCCARTARAETTWESLDHLWNNNQQMFWIQLLIPLAALIVVTRLLRCVCCVVPFLVMAGAAGAGAYEHATTMPSQAGISYNTIVNRAGYAPLPISITPTKIKLIPTVNLEYVTCHYKTGMDSPAIKCCGSQECTPTYRPDEQCKVFTGVYPFMWGGAYCFCDTENTQVSKAYVMKSDDCLADHAEAYKAHTASVQAFLNITVGEHSIVTTVYVNGETPVNFNGVKLTAGPLSTAWTPFDRKIVQYAGEIYNYDFPEYGAGQPGAFGDIQSRTVSSSDLYANTNLVLQRPKAGAIHVPYTQAPSGFEQWKKDKAPSLKSTAPFGCEIYTNPIRAENCAVGSIPLAFDIPDALFTRVSETPTLSAAECTLNECVYSSDFGGIATVKYSASKSGKCAVHVPSGTATLKEAAVELTEQGSATIHFSTANIHPEFRLQICTSYVTCKGDCHPPKDHIVTHPQYHAQTFTAAVSKTAWTWLTSLLGGSAVIIIIGLVLATIVAMYVLTNQKHN

SEQ ID NO: 97 - protein, 423 aa, Venezuelan equine encephalitis virus
STEELFKEYKLTRPYMARCIRCAVGSCHSPIAIEAVKSDGHDGYVRLQTSSQYGLDSSGNLKGRTMRYDMHGTIKEIPLHQVSLHTSRPCHIVDGHGYFLLARCPAGDSITMEFKKDSVTHSCSVPYEVKFNPVGRELYTHPPEHGVEQACQVYAHDAQNRGAYVEMHLPGSEVDSSLVSLSGSSVTVTPPVGTSALVECECGGTKISETINKTKQFSQCTKKEQCRAYRLQNDKWVYISDKLPKAAGATLKGKLHVPFLLADGKCTVPLAPEPMITFGFRSVSLKLHPKNPTYLTTRQLADEPHYTHELISEPAVRNFTVTGKGWEFVWGNHPPKRFWAQETAPGNPHGLPHEVITHYYHRYPMSTILGLSICAAIATVSVAASTWLFCRSRVACLTPYRLTPNARIPFCLAVLCCARTARA

SEQ ID NO: 98 - protein, 442 aa, Venezuelan equine encephalitis virus
YEHATTMPSQAGISYNTIVNRAGYAPLPISITPTKIKLIPTVNLEYVTCHYKTGMDSPAIKCCGSQECTPTYRPDEQCKVFTGVYPFMWGGAYCFCDTENTQVSKAYVMKSDDCLADHAEAYKAHTASVQAFLNITVGEHSIVTTVYVNGETPVNFNGVKLTAGPLSTAWTPFDRKIVQYAGEIYNYDFPEYGAGQPGAFGDIQSRTVSSSDLYANTNLVLQRPKAGAIHVPYTQAPSGFEQWKKDKAPSLKSTAPFGCEIYTNPIRAENCAVGSIPLAFDIPDALFTRVSETPTLSAAECTLNECVYSSDFGGIATVKYSASKSGKCAVHVPSGTATLKEAAVELTEQGSATIHFSTANIHPEFRLQICTSYVTCKGDCHPPKDHIVTHPQYHAQTFTAAVSKTAWTWLTSLLGGSAVIIIIGLVLATIVAMYVLTNQKHN

SEQ ID NO: 99 - protein, 491 aa, Lassa virus
MGQIVTFFQEVPHVIEEVMNIVLIALSVLAVLKGLYNFATCGLVGLVTFLLLCGRSCTTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLFVFSTSFYLISIFLHLVKIPTHRHIVGKSCPKPHRLNHMGICSCGLYKQPGVPVKWKR

SEQ ID NO: 100 - protein, 607 aa, Avian leukosis virus
MEAVIKMRRALFLQAFLTGRPGKASKKDPKKNPLATSKKDPEKTPLLPTRVNYILIIGVLVLCEVTGVRADVHLLEQPGNLWITWANRTGQTDFCLSTQSATSPFQTCLIGIPSPISEGDFKGYVSDNCTTLGTDRLVSSASITGGPDNSTTLTYRKVSCLLLKLNVSMWNEPPELQLLGSQSLPNITDITQISGVAGGCVGFRPKGVPWYLGWSQGEATRFLLRHPSFSNLTGPFTVVTADRHNLFMGSEYCGAYGYRFWEIYNCSQEGQQYRCGKARRPRPQSPETQCTRQGGIWVNRSKEINETEPFSFTVNCTASNLGNASGCCGKAGTILPGIWVDSTQGNFTKPKALPPAIFLICGDRAWQGIPSRPVGGPCYLGKLTMLAPNHTDILKILANSSRTGIRRRRSVSHLDDTCSDEVQLWGPTARIFASILAPGVAAAQALREIERLACWSVKQANLTTSLLGDLLDDVTSIRHAVLQNRAAIDFLLLAHGHGCEDIAGMCCFNLSDHSESIQKKFQLMKEHVNKIGVDSDPIGSWLRGLFGGIGGWAVHLLKGLLLGLVVILLLVVCLPCFLQFVSSSIRKMINNSVSYHTEYRKMQGGAV

SEQ ID NO: 101 - protein, 614 aa, Avian leukosis virus
MEAVIKMRRALFLQAFLTGHPGKVSKKDSKKKPPATGKRDPEKTPLLPTRVNYILIIGVLVLCEVTGVRADVHLLEQPGNLWITWANRTGQTDFCLSTQSATSPFQTCLIGIPSPISEGDFKGYVSGNCTALGTHRLVSSGIHGGPDNSTTLTYRKVSCLLLKLNVSLLDEPSELQLLGSQSLPNITNITQIPSVAGGCIGFTPYGSPAGVYGWDRRQVTHILLTDPGSNPFFNKASNSSKPFTVVTADRHNLFMGSEYCGAYGYRFWEMYNCSQMRQNWSICMDVWGRGLPESWCTSTGGIWVNQSKEINETEPFSFTANCTGSNLGNVSGCCGESITILPPGAWVDSTQGSFTKPKALPPGIFLICGDRAWQGIPSRPVGGPCYLGKLTMLAPNHTDILKILANSSQTGVRHKRSVTHLDDTCSDEVQLWGPTARIFASILAPGVAAAQALREIERLACWSVKQANLTTSLLGDLLDDVTSIRHAVLQNRAAIDFLLLAHGHGCEDIAGMCCFNLSDHSESIQKKFQLMKEHVNKIGVDSDPIGSWLRGLFGGIGEWAVHLLKGLLLGLVVILLLVVCLPCFLQFVSSSIRKMINNSISYHTEYRKMQGGAV

SEQ ID NO: 102 - protein, 569 aa, Avian leukosis virus
MEAVIKAFLTGHPGKVSKKDSKKKPPATSKKDPEKTPLLPSRGYFFFPTILVCVVIISVVPGVGGVHLLRQPGNVWVTWANKTGRTDFCLSLQSATSPFRTCLIGIPQYPLNTFKGYVTNVTACDNDADLASQTACLIKALNTTLPWDPQELDILGSQMIKNGTTRTCVTFGSVCYKENNRSRVCHNFDGNFNGTGGAEAELRDFIAKWKSDDLLIRPYVNQSWTMVSPINVESFSISRRYCGFTSNETRYYRGDLSNWCGSKRGKWSAGYSNRTKCSSNTTGCGGNCTTEWNYYAYGFTFGKQPEVLWNNGTAKALPPGIFLICGDRAWQGIPRNALGGPCYLGQLTMLSPNFTTWITYGPNITGHRRSRRAIRGLSPDCSDEVQLWSATARIFASFFAPGVAAAQALKEIERLACWSVKQANLTSLILNAMLEDMNSIRHAVLQNRAAIDFLLLAQGHGCQDVEGMCCFNLSDHSESIHKALQAMKEHTEKIQVEDDPIGDWFTRTFGDLGRWLAKGVKTLLFALLVIVCLLAIIPCIIKCFQDCLSRTMNQFMDERIRYHRIREQL

SEQ ID NO: 103 - protein, 488 aa, Human T-lymphotropic virus 1
MGKFLATLILFFQFCPLILGDYSPSCCTLTVGVSSYHSKPCNPAQPVCSWTLDLLALSADQALQPPCPNLVSYSSYHATYSLYLFPHWIKKPNRNGGGYYSASYSDPCSLKCPYLGCQSWTCPYTGAVSSPYWKFQQDVNFTQEVSHLNINLHFSKCGFPFSLLVDAPGYDPIWFLNTEPSQLPPTAPPLLSHSNLDHILEPSIPWKSKLLTLVQLTLQSTNYTCIVCIDRASLSTWHVLYSPNVSVPSLSSTPLLYPSLALPAPHLTLPFNWTHCFDPQIQAIVSSPCHNSLILPPFSLSPVPTLGSRSRRAVPVAVWLVSALAMGAGVAGGITGSMSLASGKSLLHEVDKDISQLTQAIVKNHKNLLKIAQYAAQNRRGLDLLFWEQGGLCKALQEQCCFLNITNSHVSILQERPPLENRVLTGWGLNWDLGLSQWAREALQTGITLVALLLLVILAGPCILRQLRHLPSRVRYPHYSLINPESSL

SEQ ID NO: 104 - protein, 989 aa, Human foamy virus
MAPPMTLQQWIIWKKMNKAHEALQNTTTVTEQQKEQIILDIQNEEVQPTRRDKFRYLLYTCCATSSRVLAWMFLVCILLIIVLVSCFVTISRIQWNKDIQVLGPVIDWNVTQRAVYQPLQTRRIARSLRMQHPVPKYVEVNMTSIPQGVYYEPHPEPIVVKERVLGLSQILMINSENIANNANLTQEVKKLLTEMVNEEMQSLSDVMIDFEIPLGDPRDQEQYIHRKCYQEFANCYLVKYKEPKPWPKEGLIADQCPLPGYHAGLTYNRQSIWDYYIKVESIRPANWTTKSKYGQARLGSFYIPSSLRQINVSHVLFCSDQLYSKWYNIENTIEQNERFLLNKLNNLTSGTSVLKKRALPKDWSSQGKNALFREINVLDICSKPESVILLNTSYYSFSLWEGDCNFTKDMISQLVPECDGFYNNSKWMHMHPYACRFWRSKKNEKEETKCRDGETKRCLYYPLWDSPESTYDFGYLAYQKNFPSPICIEQQKIRDQDYEVYSLYQERKIASKAYGIDTVLFSLKNFLNYTGTPVNEMPNARAFVGLIDPKFPPSYPNVTREHYTSCNNRKRRSVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIFKIVSWIPTKKKNQ

SEQ ID NO: 105 - protein, 446 aa, Human foamy virus
SLRMQHPVPKYVEVNMTSIPQGVYYEPHPEPIVVKERVLGLSQILMINSENIANNANLTQEVKKLLTEMVNEEMQSLSDVMIDFEIPLGDPRDQEQYIHRKCYQEFANCYLVKYKEPKPWPKEGLIADQCPLPGYHAGLTYNRQSIWDYYIKVESIRPANWTTKSKYGQARLGSFYIPSSLRQINVSHVLFCSDQLYSKWYNIENTIEQNERFLLNKLNNLTSGTSVLKKRALPKDWSSQGKNALFREINVLDICSKPESVILLNTSYYSFSLWEGDCNFTKDMISQLVPECDGFYNNSKWMHMHPYACRFWRSKKNEKEETKCRDGETKRCLYYPLWDSPESTYDFGYLAYQKNFPSPICIEQQKIRDQDYEVYSLYQERKIASKAYGIDTVLFSLKNFLNYTGTPVNEMPNARAFVGLIDPKFPPSYPNVTREHYTSCNNRKRR

SEQ ID NO: 106 - protein, 417 aa, Human foamy virus
SVDNNYAKLRSMGYALTGAVQTLSQISDINDENLQQGIYLLRDHVITLMEATLHDISVMEGMFAVQHLHTHLNHLKTMLLERRIDWTYMSSTWLQQQLQKSDDEMKVIKRIARSLVYYVKQTHSSPTATAWEIGLYYELVIPKHIYLNNWNVVNIGHLVKSAGQLTHVTIAHPYEIINKECVETIYLHLEDCTRQDYVICDVVKIVQPCGNSSDTSDCPVWAEAVKEPFVQVNPLKNGSYLVLASSTDCQIPPYVPSIVTVNETTSCFGLDFKRPLVAEERLSFEPRLPNLQLRLPHLVGIIAKIKGIKIEVTSSGESIKEQIERAKAELLRLDIHEGDTPAWIQQLAAATKDVWPAAASALQGIGNFLSGTAQGIFGTAFSLLGYLKPILIGVGVILLVILIFKIVSWIPTKKKNQ

SEQ ID NO: 107 - protein, 983 aa, Visna-maedi virus
MASKESKPSRTTRRGMEPPLRETWNQVLQELVKRQQQEEEEQQGLVSGKKKSWVSIDLLGTEGKDIKKVNIWEPCEKWFAQVVWGVLWVLQIVLWGCLMWEVRKGNQCQAEEVIALVSDPGGFQRVQHVETVPVTCVTKNFTQWGCQPEGAYPDPELEYRNISREILEEVYKQDWPWNTYHWPLWQMENMRQWMKENEKEYKERTNKTKEDIDDLVAGRIRGRFCVPYPYALLRCEEWCWYPESINQETGHAEKIKINCTKAKAVSCTEKMSLAAVQRVYWEKEDEESMKFLNIKACNISLRCQDEGKSPGGCVQGYPIPKGAEIIPEAMKYLRGKKSRYGGIKDKNGELKLPLSVRVWVRMANLSGWVNGTPPYWSARINGSTGINGTRWYGIGTLHHLGCNISSNPERGICNFTGELWIGGDKFPYYYTPSWNCSQNWTGHPVWHVFRYLDMTEHMTSRCIQRPKRHNITVGNGTITGNCSVTNWDGCNCTRSGNHLYNSTSGGLLVIICRQNSTITGIMGTNTNWTTMWNIYQNCSRCNNSSLDRTGSGTLGTVNNLKCSLPHRNESNKWTCKSQRDSYIAGRDFWGKVKAKYSCESNLGGLDSMMHQQMLLQRYQVIRVRAYTYGVVEMPQSYMEAQGENKRSRRNLQRKKRGIGLVIVLAIMAIIAAAGAGLGVANAVQQSYTRTAVQSLANATAAQQEVLEASYAMVQHIAKGIRILEARVARVEALVDRMMVYQELDCWHYQHYCVTSTRSEVANYVNWTRFKDNCTWQQWEEEIEQHEGNLSLLLREAALQVHIAQRDARRIPDAWKAIQEAFNWSSWFSWLKYIPWIIMGIVGLMCFRILMCVISMCLQAYKQVKQIRYTQVTVVIEAPVELEEKQKRNGDGTNGCASLERERRTSHRSFIQIWRATWWAWKTSPWRHNWRTMPYITLLPILVIWQWMEENGWNGENQHKKKKERVDCQDREQMPTLENDYVEL

SEQ ID NO: 108 - protein, 877 aa, Visna-maedi virus
QCQAEEVIALVSDPGGFQRVQHVETVPVTCVTKNFTQWGCQPEGAYPDPELEYRNISREILEEVYKQDWPWNTYHWPLWQMENMRQWMKENEKEYKERTNKTKEDIDDLVAGRIRGRFCVPYPYALLRCEEWCWYPESINQETGHAEKIKINCTKAKAVSCTEKMSLAAVQRVYWEKEDEESMKFLNIKACNISLRCQDEGKSPGGCVQGYPIPKGAEIIPEAMKYLRGKKSRYGGIKDKNGELKLPLSVRVWVRMANLSGWVNGTPPYWSARINGSTGINGTRWYGIGTLHHLGCNISSNPERGICNFTGELWIGGDKFPYYYTPSWNCSQNWTGHPVWHVFRYLDMTEHMTSRCIQRPKRHNITVGNGTITGNCSVTNWDGCNCTRSGNHLYNSTSGGLLVIICRQNSTITGIMGTNTNWTTMWNIYQNCSRCNNSSLDRTGSGTLGTVNNLKCSLPHRNESNKWTCKSQRDSYIAGRDFWGKVKAKYSCESNLGGLDSMMHQQMLLQRYQVIRVRAYTYGVVEMPQSYMEAQGENKRSRRNLQRKKRGIGLVIVLAIMAIIAAAGAGLGVANAVQQSYTRTAVQSLANATAAQQEVLEASYAMVQHIAKGIRILEARVARVEALVDRMMVYQELDCWHYQHYCVTSTRSEVANYVNWTRFKDNCTWQQWEEEIEQHEGNLSLLLREAALQVHIAQRDARRIPDAWKAIQEAFNWSSWFSWLKYIPWIIMGIVGLMCFRILMCVISMCLQAYKQVKQIRYTQVTVVIEAPVELEEKQKRNGDGTNGCASLERERRTSHRSFIQIWRATWWAWKTSPWRHNWRTMPYITLLPILVIWQWMEENGWNGENQHKKKKERVDCQDREQMPTLENDYVEL

SEQ ID NO: 109 - protein, 550 aa, Visna-maedi virus
QCQAEEVIALVSDPGGFQRVQHVETVPVTCVTKNFTQWGCQPEGAYPDPELEYRNISREILEEVYKQDWPWNTYHWPLWQMENMRQWMKENEKEYKERTNKTKEDIDDLVAGRIRGRFCVPYPYALLRCEEWCWYPESINQETGHAEKIKINCTKAKAVSCTEKMSLAAVQRVYWEKEDEESMKFLNIKACNISLRCQDEGKSPGGCVQGYPIPKGAEIIPEAMKYLRGKKSRYGGIKDKNGELKLPLSVRVWVRMANLSGWVNGTPPYWSARINGSTGINGTRWYGIGTLHHLGCNISSNPERGICNFTGELWIGGDKFPYYYTPSWNCSQNWTGHPVWHVFRYLDMTEHMTSRCIQRPKRHNITVGNGTITGNCSVTNWDGCNCTRSGNHLYNSTSGGLLVIICRQNSTITGIMGTNTNWTTMWNIYQNCSRCNNSSLDRTGSGTLGTVNNLKCSLPHRNESNKWTCKSQRDSYIAGRDFWGKVKAKYSCESNLGGLDSMMHQQMLLQRYQVIRVRAYTYGVVEMPQSYMEAQGENKRSRRNLQRKKR

SEQ ID NO: 110 - protein, 327 aa, Visna-maedi virus
GIGLVIVLAIMAIIAAAGAGLGVANAVQQSYTRTAVQSLANATAAQQEVLEASYAMVQHIAKGIRILEARVARVEALVDRMMVYQELDCWHYQHYCVTSTRSEVANYVNWTRFKDNCTWQQWEEEIEQHEGNLSLLLREAALQVHIAQRDARRIPDAWKAIQEAFNWSSWFSWLKYIPWIIMGIVGLMCFRILMCVISMCLQAYKQVKQIRYTQVTVVIEAPVELEEKQKRNGDGTNGCASLERERRTSHRSFIQIWRATWWAWKTSPWRHNWRTMPYITLLPILVIWQWMEENGWNGENQHKKKKERVDCQDREQMPTLENDYVEL

SEQ ID NO: 111 - protein, 1255 aa, Severe acute respiratory syndrome-associated coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 112 - protein, 605 aa, Severe acute respiratory syndrome-associated coronavirus
CDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIVIVMVTILLCCMTSCCSCLKGACSCGSCCKFDEDDSEPVLKGVKL

SEQ ID NO: 113 - protein, 253 aa, Severe acute respiratory syndrome-associated coronavirus
PNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSE

SEQ ID NO: 114 - protein, 298 aa, Respiratory syncytial virus
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 115 - protein, 574 aa, Respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 116 - protein, 549 aa, Respiratory syncytial virus
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 117 - protein, 111 aa, Respiratory syncytial virus
QNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRR

SEQ ID NO: 118 - protein, 438 aa, Respiratory syncytial virus
FLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 119 - protein, 572 aa, Human parainfluenza virus type 3
MEYWKHTNHGKDAGNELETSMATHGNKLTNKITYILWTIILVLLSIVFIIVLINSIKSEKAHESLLQNINNEFMEITEKIQMASDNTNDLIQSGVNTRLLTIQSHVQNYIPISLTQQMSDLRKFISEITIRNDNQEVLPQRITHDVGIKPLNPDDFWRCTSGLPSLMKTPKIRLMPGPGLLAMPTTVDGCIRTPSLVINDLIYAYTSNLITRGCQDIGKSYQVLQIGIITVNSDLVPDLNPRISHTFNINDNRKSCSLALLNTDVYQLCSTPKVDERSDYASPGIEDIVLDIVNYDGSISTTRFKNNNISFDQPYAALYPSVGPGIYYKGKIIFLGYGGLEHPINENVICNTTGCPGKTQRDCNQASHSPWFSDRRMVNSIIVVDKGLNSIPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAILNRTLSAGYTTTSCITHYNKGYCFHIVEINHKSLNTLQPMLFKTEIPKSCS

SEQ ID NO: 120 - protein, 539 aa, Human parainfluenza virus type 3
MPISILLIITTMIMASHCQIDITKLQHVGVLVNSPKGMKISQNFETRYLILSLIPKIDDSNSCGDQQIKQYKRLLDRLIIPLYDGLRLQKDVIVANQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPADITLNNSVALDPIDISIELNKAKSDLEESKEWIRRSNQKLDSIGSWHQSSTTIIVILIMMIILFIINITIITIAIKYYRIQKRNRVDQNDKPYVLTNK

SEQ ID NO: 121 - protein, 192 aa, Hepatitis C virus
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDA

SEQ ID NO: 122 - protein, 363 aa, Hepatitis C virus
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 123 - protein, 563 aa, Autographa californica nuclear polyhedrosis virus
MNTQILVFALVAVIPTNADKICLGHHAVSNGTKVNTLTERGVEVVNATETVERTNIPKICSKGKRTTDLGQCGLLGTITGPPQCDQFLEFSADLIIERREGNDVCYPGKFVNEEALRQILRGSGGIDKETMGFTYSGIRTNGTTSACRRSGSSFYAEMEWLLSNTDNASFPQMTKSYKNTRRESALIVWGIHHSGSTTEQTKLYGSGNKLITVGSSKYHQSFVPSPGTRPQINGQSGRIDFHWLILDPNDTVTFSFNGAFIAPNRASFLRGKSMGIQSDVQVDANCEGECYHSGGTITSRLPFQNINSRAVGKCPRYVKQESLLLATGMKNVPEPSKKREKRGLFGAIAGFIENGWEGLVDGWYGFRHQNAQGEGTAADYKSTQSAIDQITGKLNRLIEKTNQQFELIDNEFTEVEKQIGNLINWTKDFITEVWSYNAELLVAMENQHTIDLADSEMNKLYERVRKQLRENAEEDGTGCFEIFHKCDDDCMASIRNNTYDHSKYREEAMQNRIQIDPVKLSSGYKDVILWFSFGASCFLLLAIAVGLVFICVKNGNMRCTICI

SEQ ID NO: 124 - protein, 492 aa, Autographa californica nuclear polyhedrosis virus
AEHCNAQMKTGPYKIKNLDITPPKETLQKDVEITIVETDYNENVIIGYKGYYQAYAYNGGSLDPNTRVEETMKTLNVGKEDLLMWSIRQQCEVGEELIDRWGSDSDDCFRDNEGRGQWVKGKELVKRQNNNHFAHHTCNKSWRCGISTSKMYSRLECQDDTDECQVYILDAEGNPINVTVDTVLHRDGVSMILKQKSTFTTRQIKAACLLIKDDKNNPESVTREHCLIDNDIYDLSKNTWNCKFNRCIKRKVEHRVKKRPPTWRHNVRAKYTEGDTATKGDLMHIQEELMYENDLLKMNIELMHAHINKLNNMLHDLIVSVAKVDERLIGNLMNNSVSSTFLSDDTFLLMPCTNPPAHTSNCYNNSIYKEGRWVANTDSSQCIDFSNYKELAIDDDVEFWIPTIGNTTYHDSWKDASGWSFIAQQKSNLITTMENTKFGGVGTSLSDITSMAEGELAAKLTSFMFGHVVNFVIILIVILFLYCMIRNRNRQY

SEQ ID NO: 125 - protein, 617 aa, Measles virus
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLSRGYNVSSIVTMTSQGMYGGTYLVEKPNLSSKGSELSQLSMYRVFEVGVIRNPGLGAPVFHMTNYFEQPVSNDLSNCMVALGELKLAALCHGGDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPSRSFSYFYPFRLPIKGIPIELQVECFTWDQKLWCRHFCVLADSESGGHITHSGMVGMGVSCTVTREDGTNSR

SEQ ID NO: 126 - protein, 553 aa, Measles virus
MSIMGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIETIRQAGQEMILAVQGVQDYINNELIPSMNQLSCDLIGQKLGLKLLRYYTEILSLFGPSLRDPISAEISIQALSYALGGDINKVLEKLGYSGGDLLGILESGGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGYTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGRCNKKGEQVGMSRPGLKPDLTGTSKSYVRSL

SEQ ID NO: 127 - protein, 7 aa, synthetic construct
  REGION 1..7: note = synthetic sequence
ENLYTQS

SEQ ID NO: 128 - protein, 5 aa, synthetic construct
  REGION 1..5: note = synthetic sequence
DDDDK

SEQ ID NO: 129 - protein, 4 aa, synthetic construct
  REGION 1..4: note = synthetic sequence
LVPR

SEQ ID NO: 130 - protein, 8 aa, synthetic construct
  REGION 1..8: note = synthetic sequence
LEVLFQGP

SEQ ID NO: 131 - protein, 10 aa, synthetic construct
  REGION 1..10: note = synthetic sequence
CGLVPAGSGP

SEQ ID NO: 132 - protein, 12 aa, synthetic construct
  REGION 1..12: note = synthetic sequence
SLLKSRMVPNFN

SEQ ID NO: 133 - protein, 12 aa, synthetic construct
  REGION 1..12: note = synthetic sequence
SLLIARRMPNFN

SEQ ID NO: 134 - protein, 12 aa, synthetic construct
  REGION 1..12: note = synthetic sequence
SKLVQASASGVN

SEQ ID NO: 135 - protein, 12 aa, synthetic construct
  REGION 1..12: note = synthetic sequence
SSYLKASDAPDN

SEQ ID NO: 136 - protein, 12 aa, synthetic construct
  REGION 1..12: note = synthetic sequence
RPKPQQFFGLMN

SEQ ID NO: 137 - protein, 12 aa, synthetic construct
  REGION 1..12: note = synthetic sequence
SLRPLALWRSFN

SEQ ID NO: 138 - protein, 12 aa, synthetic construct
  REGION 1..12: note = synthetic sequence
SPQGIAGQRNFN

SEQ ID NO: 139 - protein, 14 aa, synthetic construct
  REGION 1..14: note = synthetic sequence
DVDERDVRGFASFL

SEQ ID NO: 140 - protein, 12 aa, synthetic construct
  REGION 1..12: note = synthetic sequence
SLPLGLWAPNFN

SEQ ID NO: 141 - protein, 12 aa, synthetic construct
  REGION 1..12: note = synthetic sequence
SLLIFRSWANFN

SEQ ID NO: 142 - protein, 12 aa, synthetic construct
  REGION 1..12: note = synthetic sequence
SGVVIATVIVIT

SEQ ID NO: 143 - protein, 12 aa, synthetic construct
  REGION 1..12: note = synthetic sequence
SLGPQGIWGQFN

SEQ ID NO: 144 - protein, 12 aa, synthetic construct
  REGION 1..12: note = synthetic sequence
KKSPGRVVGGSV

SEQ ID NO: 145 - protein, 12 aa, synthetic construct
  REGION 1..12: note = synthetic sequence
PQGLLGAPGILG

SEQ ID NO: 146 - protein, 31 aa, synthetic construct
  REGION 1..31: note = synthetic sequence
HGPEGLRVGFYESDVMGRGHARLVHVEEPHT

SEQ ID NO: 147 - protein, 12 aa, synthetic construct
  REGION 1..12: note = synthetic sequence
GPQGLAGQRGIV

SEQ ID NO: 148 - protein, 12 aa, synthetic construct
  REGION 1..12: note = synthetic sequence
GGSGQRGRKALE

SEQ ID NO: 149 - protein, 12 aa, synthetic construct
  REGION 1..12: note = synthetic sequence
SLSALLSSDIFN

SEQ ID NO: 150 - protein, 12 aa, synthetic construct
  REGION 1..12: note = synthetic sequence
SLPRFKIIGGFN

SEQ ID NO: 151 - protein, 12 aa, synthetic construct
  REGION 1..12: note = synthetic sequence
SLLGIAVPGNFN

SEQ ID NO: 152 - protein, 12 aa, synthetic construct
  REGION 1..12: note = synthetic sequence
FFKNIVTPRTPP

SEQ ID NO: 153 - protein, 7 aa, synthetic construct
  REGION 1..7: note = synthetic sequence
ENLYFQS

SEQ ID NO: 154 - protein, 7 aa, synthetic construct
  REGION 1..7: note = synthetic sequence
ENLYFQP

SEQ ID NO: 155 - protein, 8 aa, synthetic construct
  REGION 1..8: note = synthetic sequence
SQNYPIVQ

SEQ ID NO: 156 - protein, 7 aa, synthetic construct
  REGION 1..7: note = synthetic sequence
ENAYFQS

SEQ ID NO: 157 - protein, 7 aa, synthetic construct
  REGION 1..7: note = synthetic sequence
ENLRFQS

SEQ ID NO: 158 - protein, 7 aa, synthetic construct
  REGION 1..7: note = synthetic sequence
ENLFFQS

SEQ ID NO: 159 - protein, 7 aa, synthetic construct
  REGION 1..7: note = synthetic sequence
ETVRFQS

SEQ ID NO: 160 - protein, 7 aa, synthetic construct
  REGION 1..7: note = synthetic sequence
ETLRFQS

SEQ ID NO: 161 - protein, 7 aa, synthetic construct
  REGION 1..7: note = synthetic sequence
ETARFQS

SEQ ID NO: 162 - protein, 7 aa, synthetic construct
  REGION 1..7: note = synthetic sequence
ETVYFQS

SEQ ID NO: 163 - protein, 7 aa, synthetic construct
  REGION 1..7: note = synthetic sequence
ENVYFQS

SEQ ID NO: 164 - DNA, 25 bp, synthetic construct
  misc_feature 1..25: note = synthetic sequence
  source 1..25: mol_type = other DNA
catcgatcttaagtcgcgactcgag

SEQ ID NO: 165 - DNA, 33 bp, synthetic construct
  misc_feature 1..33: note = synthetic sequence
  source 1..33: mol_type = other DNA
aattctcgagtcgcgacttaagatcgatggtac

SEQ ID NO: 166 - DNA, 20 bp, synthetic construct
  misc_feature 1..20: note = synthetic sequence
  source 1..20: mol_type = other DNA
gagtagcgcgagcacagcta

SEQ ID NO: 167 - DNA, 20 bp, synthetic construct
  misc_feature 1..20: note = synthetic sequence
  source 1..20: mol_type = other DNA
agagtctctcagctggtaca

SEQ ID NO: 168 - DNA, 20 bp, synthetic construct
  misc_feature 1..20: note = synthetic sequence
  source 1..20: mol_type = other DNA
gctgaagcactgcacgccat

SEQ ID NO: 169 - DNA, 20 bp, synthetic construct
  misc_feature 1..20: note = synthetic sequence
  source 1..20: mol_type = other DNA
aagtaaaacctctacaaatg

SEQ ID NO: 170 - DNA, 20 bp, synthetic construct
  misc_feature 1..20: note = synthetic sequence
  source 1..20: mol_type = other DNA
gtattactgatattggtggg

SEQ ID NO: 171 - DNA, 20 bp, synthetic construct
  misc_feature 1..20: note = synthetic sequence
  source 1..20: mol_type = other DNA
tcacccagtctagtgcatgc

SEQ ID NO: 172 - DNA, 20 bp, synthetic construct
  misc_feature 1..20: note = synthetic sequence
  source 1..20: mol_type = other DNA
gacgcttatcgacgccctaa

SEQ ID NO: 173 - DNA, 22 bp, synthetic construct
  misc_feature 1..22: note = synthetic sequence
  source 1..22: mol_type = other DNA
catcactggcatctggactcca

SEQ ID NO: 174 - DNA, 24 bp, synthetic construct
  misc_feature 1..24: note = synthetic sequence
  source 1..24: mol_type = other DNA
tgctcttgaagtccatagacctca

SEQ ID NO: 175 - DNA, 33 bp, synthetic construct
  misc_feature 1..33: note = synthetic sequence
  source 1..33: mol_type = other DNA
gctcttccgatcttgcgggccttgtcctgattg

SEQ ID NO: 176 - DNA, 33 bp, synthetic construct
  misc_feature 1..33: note = synthetic sequence
  source 1..33: mol_type = other DNA
gctcttccgatctagatccagccctggactagc

SEQ ID NO: 177 - DNA, 32 bp, synthetic construct
  misc_feature 1..32: note = synthetic sequence
  source 1..32: mol_type = other DNA
gctcttccgatctaagctgacagcattcgggc

SEQ ID NO: 178 - DNA, 33 bp, synthetic construct
  misc_feature 1..33: note = synthetic sequence
  source 1..33: mol_type = other DNA
gctcttccgatctgaagtcacggagcgagagag

SEQ ID NO: 179 - DNA, 20 bp, synthetic construct
  misc_feature 1..20: note = synthetic sequence
  source 1..20: mol_type = other DNA
gttcgggcgccactgctaga

SEQ ID NO: 180 - DNA, 22 bp, synthetic construct
  misc_feature 1..22: note = synthetic sequence
  source 1..22: mol_type = other DNA
ttaagcctcaataaagcttgcc

SEQ ID NO: 181 - DNA, 21 bp, synthetic construct
  misc_feature 1..21: note = synthetic sequence
  source 1..21: mol_type = other DNA
tactgacgctctcgcacccat

SEQ ID NO: 182 - DNA, 33 bp, synthetic construct
  misc_feature 1..33: note = synthetic sequence
  source 1..33: mol_type = other DNA
gctcttccgatctaggtccgagcagttaactgg

SEQ ID NO: 183 - DNA, 31 bp, synthetic construct
  misc_feature 1..31: note = synthetic sequence
  source 1..31: mol_type = other DNA
gctcttccgatctacttagcgggcgcctaga

SEQ ID NO: 184 - DNA, 127 bp, synthetic construct
  misc_feature 1..127: note = synthetic sequence
  source 1..127: mol_type = other DNA
gccacctacggcaagctgaccctgaagttcatctgcaccaccggcaagctgcccgtgccctggcccaccctcgtgaccaccctgacgtacggcgtgcagtgcttcagccgctaccccgaccacatga

SEQ ID NO: 185 - DNA, 330 bp, synthetic construct
  misc_feature 1..330: note = synthetic sequence
  source 1..330: mol_type = other DNA
aagctgacagcattcgggccgagatgtctcgctccgtggccttagtggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctc

SEQ ID NO: 186 - DNA, 317 bp, synthetic construct
  misc_feature 1..317: note = synthetic sequence
  source 1..317: mol_type = other DNA
aagctgacagcattcgggccgagatgtctcgctccaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctc

SEQ ID NO: 187 - DNA, 279 bp, synthetic construct
  misc_feature 1..279: note = synthetic sequence
  source 1..279: mol_type = other DNA
aagctgacagcattcgggccgagatgtctcgctccgtggccttagctgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctc

SEQ ID NO: 188 - DNA, 319 bp, synthetic construct
  misc_feature 1..319: note = synthetic sequence
  source 1..319: mol_type = other DNA
aagctgacagcattcgggccgagatgtctcgctccgtggccttaaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctc

SEQ ID NO: 189 - DNA, 332 bp, synthetic construct
  misc_feature 1..332: note = synthetic sequence
  source 1..332: mol_type = other DNA
aagctgacagcattcgggccgagatgtctcgctccgtggccttagactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctc

SEQ ID NO: 190 - DNA, 420 bp, synthetic construct
  misc_feature 1..420: note = synthetic sequence
  source 1..420: mol_type = other DNA
gagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttccactgtgctcgcgctactctctctttctggcctggaggctatccagcgtgagtctctcctaccctcccgctctggtccttcctctcccgctctgcaccctctgtggccctcgctgtgctctctcgctccgtgacttc

SEQ ID NO: 191 - DNA, 421 bp, synthetic construct
  misc_feature 1..421: note = synthetic sequence
  source 1..421: mol_type = other DNA
gagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttccagctgtgctcgcgctactctctctttctggcctggaggctatccagcgtgagtctctcctaccctcccgctctggtccttcctctcccgctctgcaccctctgtggccctcgctgtgctctctcgctccgtgacttc

SEQ ID NO: 192 - DNA, 425 bp, synthetic construct
  misc_feature 1..425: note = synthetic sequence
  source 1..425: mol_type = other DNA
gagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttccagtttgctctgctcgcgctactctctctttctggcctggaggctatccagcgtgagtctctcctaccctcccgctctggtccttcctctcccgctctgcaccctctgtggccctcgctgtgctctctcgctccgtgacttc

SEQ ID NO: 193 - DNA, 422 bp, synthetic construct
  misc_feature 1..422: note = synthetic sequence
  source 1..422: mol_type = other DNA
gagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttccagtctgtgctcgcgctactctctctttctggcctggaggctatccagcgtgagtctctcctaccctcccgctctggtccttcctctcccgctctgcaccctctgtggccctcgctgtgctctctcgctccgtgacttc

SEQ ID NO: 194 - DNA, 421 bp, synthetic construct
  misc_feature 1..421: note = synthetic sequence
  source 1..421: mol_type = other DNA
gagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttccagtagtgctcgcgctactctctctttctggcctggaggctatccagcgtgagtctctcctaccctcccgctctggtccttcctctcccgctctgcaccctctgtggccctcgctgtgctctctcgctccgtgacttc

SEQ ID NO: 195 - DNA, 418 bp, synthetic construct
  misc_feature 1..418: note = synthetic sequence
  source 1..418: mol_type = other DNA
gagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttccagtgctcgcgctactctctctttctggcctggaggctatccagcgtgagtctctcctaccctcccgctctggtccttcctctcccgctctgcaccctctgtggccctcgctgtgctctctcgctccgtgacttc

SEQ ID NO: 196 - DNA, 430 bp, synthetic construct
  misc_feature 1..430: note = synthetic sequence
  source 1..430: mol_type = other DNA
gagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttccagttggcgtccctgtgctcgcgctactctctctttctggcctggaggctatccagcgtgagtctctcctaccctcccgctctggtccttcctctcccgctctgcaccctctgtggccctcgctgtgctctctcgctccgtgacttc

SEQ ID NO: 197 - DNA, 436 bp, synthetic construct
  misc_feature 1..436: note = synthetic sequence
  source 1..436: mol_type = other DNA
gagagctcctctggtttccctttcgctttcaagtccctgttcgggcgccactgctagagattttccacactgactaaaagggtctgagggatctctagttaccagagtcacacaacagacgggcacatactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttccagtgctccgtggccttccagtgctcgcgctactctctctttctggcctggaggctatccagcgtgagtctctcctaccctcccgctctggtccttcctctcccgctctgcaccctctgtggccctcgctgtgctctctcgctccgtgacttc

SEQ ID NO: 198 - DNA, 448 bp, synthetic construct
  misc_feature 1..448: note = synthetic sequence
  source 1..448: mol_type = other DNA
gagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagccggctagagccggctgaggcggcgctgccggaagtagtagctcgcgctactctctctttctggcctggaggctatccagcgtgagtctctcctaccctcccgctctggtccttcctctcccgctctgcaccctctgtggccctcgctgtgctctctcgctccgtgacttc

SEQ ID NO: 199 - DNA, 436 bp, synthetic construct
  misc_feature 1..436: note = synthetic sequence
  source 1..436: mol_type = other DNA
gagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaagcaaaaagcagatcttgtcttcgttgggagtgaattagcccttccagtgctccgtggccttccagtgctcgcgctactctctctttctggcctggaggctatccagcgtgagtctctcctaccctcccgctctggtccttcctctcccgctctgcaccctctgtggccctcgctgtgctctctcgctccgtgacttc

SEQ ID NO: 200 - protein, 1368 aa, Streptococcus pyogenes [sequence omitted]

SEQ ID NO: 201 - protein, 1368 aa, synthetic construct
  REGION 1..1368: note = synthetic sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 202 - protein, 1368 aa, synthetic construct
  REGION 1..1368: note = synthetic sequence
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 203 - protein, 1368 aa, synthetic construct
  REGION 1..1368: note = synthetic sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 204 - protein, 1368 aa, synthetic construct
  REGION 1..1368: note = synthetic sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 205 - protein, 1367 aa, synthetic construct
  REGION 1..1367: note = synthetic sequence
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 206 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 207 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 208 - protein, 1306 aa, Acidaminococcus sp.
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 209 - protein, 1306 aa, synthetic construct
  REGION 1..1306: note = synthetic sequence
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMANSNAATGEDYINSPVRDLNGVCFDSRFQNPEWPMDADANGAYHIALKGQLLLNHLKESKDLKLQNGISNQDWLAYIQELRN

SEQ ID NO: 210 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 211 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 212 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 213 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 214 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 215 - protein, 1285 aa, Herbinix hemicellulosilytica
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 216 - protein, 422 aa, Semliki Forest virus
SVSQHFNVYKATRPYIAYCADCGAGHSCHSPVAIEAVRSEATDGMLKIQFSAQIGIDKSDNHDYTKIRYADGHAIENAVRSSLKVATSGDCFVHGTMGHFILAKCPPGEFLQVSIQDTRNAVRACRIQYHHDPQPVGREKFTIRPHYGKEIPCTTYQQTTAETVEEIDMHMPPDTPDRTLLSQQSGNVKITVGGKKVKYNCTCGTGNVGTTNSDMTINTCLIEQCHVSVTDHKKWQFNSPFVPRADEPARKGKVHIPFPLDNITCRVPMAREPTVIHGKREVTLHLHPDHPTLFSYRTLGEDPQYHEEWVTAAVERTIPVPVDGMEYHWGNNDPVRLWSQLTTEGKPHGWPHQIVQYYYGLYPAATVSAVVGMSLLALISIFASCYMLVAARSKCLTPYALTPGAAVPWTLGILCCAPRAHA

SEQ ID NO: 217 - protein, 512 aa, Autographa californica nuclear polyhedrosis virus
MVSAIVLYVLLAAAAHSAFAAEHCNAQMKTGPYKIKNLDITPPKETLQKDVEITIVETDYNENVIIGYKGYYQAYAYNGGSLDPNTRVEETMKTLNVGKEDLLMWSIRQQCEVGEELIDRWGSDSDDCFRDNEGRGQWVKGKELVKRQNNNHFAHHTCNKSWRCGISTSKMYSRLECQDDTDECQVYILDAEGNPINVTVDTVLHRDGVSMILKQKSTFTTRQIKAACLLIKDDKNNPESVTREHCLIDNDIYDLSKNTWNCKFNRCIKRKVEHRVKKRPPTWRHNVRAKYTEGDTATKGDLMHIQEELMYENDLLKMNIELMHAHINKLNNMLHDLIVSVAKVDERLIGNLMNNSVSSTFLSDDTFLLMPCTNPPAHTSNCYNNSIYKEGRWVANTDSSQCIDFSNYKELAIDDDVEFWIPTIGNTTYHDSWKDASGWSFIAQQKSNLITTMENTKFGGVGTSLSDITSMAEGELAAKLTSFMFGHVVNFVIILIVILFLYCMIRNRNRQY

SEQ ID NO: 218-219 - skipped (2 entries)

SEQ ID NO: 220 - protein, 6 aa, synthetic construct
  REGION 1..6: note = synthetic sequence
  VARIANT 2: note = The amino acid at position 2 is Leu or Gln
PXGMTS

SEQ ID NO: 221 - protein, 5 aa, synthetic construct
  REGION 1..5: note = synthetic sequence
  VARIANT 2: note = The amino acid at position 2 is Leu or Gln
PXGMT

(19) United States
(12) Patent Application Publication
(10) Pub. No.: US 2026/0146247 A1
(43) Pub. Date: May 28, 2026

(54) Compositions and Methods for Targeted Delivery of CRISPR-CAS Effector Polypeptides and Transgenes

(71) Applicant: The Regents of the University of California, Oakland, CA (US)

(72) Inventors: Jennifer A. Doudna, Berkeley, CA (US); Jennifer Hamilton, Berkeley, CA (US); Connor Tsuchida, Berkeley, CA (US)

(21) Appl. No.: 19/461,166

(22) Filed: Jan. 27, 2026

Related U.S. Application Data
(62) Division of application No. 17/824,626, filed on May 25, 2022, now Pat. No. 12,565,647.
(60) Provisional application No. 63/194,679, filed on May 28, 2021.

(51) Int. Cl. C12N 15/11; C07K 14/16; C12N 9/22; C12N 15/86

(52) U.S. Cl. CPC C12N 15/111; C07K 14/161; C12N 9/22; C12N 15/86; C12N 2310/20; C12N 2740/16023; C12N 2740/16043

(57) ABSTRACT

The present disclosure provides virus-like particles (VLPs) comprising: i) a CRISPR-Cas effector polypeptide; ii) a recombinant lentivirus comprising a nucleotide sequence encoding a therapeutic polypeptide having a length of from about 250 amino acids to about 3,000 amino acids, where the VLP comprises a pseudotyping viral glycoprotein and/or a polypeptide that provides for binding to a target cell. The present disclosure provides systems for producing a VLP. The present disclosure provides methods of delivering a therapeutic protein, using a VLP of the present disclosure.

### CROSS-REFERENCE

This application is a divisional of U.S. patent application Ser. No. 17/824,626, filed May 25, 2022, which claims the benefit of U.S. Provisional Patent Application No. 63/194,679, filed May 28, 2021, which applications are incorporated herein by reference in their entirety.

### INCORPORATION BY REFERENCE OF SEQUENCE LISTING PROVIDED AS A TEXT FILE

A Sequence Listing is provided herewith as a Sequence Listing XML, “BERK-449.xml” created on Jan. 23, 2026 and having a size of 306,538 bytes. The contents of the Sequence Listing XML are incorporated by reference herein in their entirety.

### INTRODUCTION

Engineering target specificity into immune cells enables the antigen-specific elimination of cells expressing cancer-associated epitopes. Currently approved cell therapies require isolation of patient T cells, viral introduction of a chimeric antigen receptor (CAR) to redirect cytotoxic activity towards target cells, and subsequent reintroduction into the body.

RNA-mediated adaptive immune systems in bacteria and archaea rely on Clustered Regularly Interspaced Short Palindromic Repeat (CRISPR) genomic loci and CRISPR-associated (Cas) proteins that function together to provide protection from invading viruses and plasmids. Genome editing can be carried out using a CRISPR-Cas system comprising a CRISPR-Cas effector polypeptide and a guide RNA. CRISPR-Cas systems are revolutionizing the field of gene editing and genome engineering. Efficient methods for delivering CRISPR-Cas genome editing components into target cells are needed, for both ex vivo and in vivo applications. Current delivery strategies have drawbacks. For example, delivery of a recombinant virus encoding a CRISPR-Cas effector polypeptide leads to prolonged CRISPR-Cas effector polypeptide expression in target cells, thus increasing the likelihood for off-target gene editing events. Others have used a ribonucleoprotein (RNP) comprising a CRISPR-Cas effector polypeptide and guide RNA (gRNA) to deliver the genome editing components into a cell.

There is a need in the art for strategies for modifying immune cells.

### SUMMARY

The present disclosure provides virus-like particles (VLPs) comprising: i) a CRISPR-Cas effector polypeptide; ii) a recombinant lentivirus comprising a nucleotide sequence encoding a therapeutic polypeptide having a length of from about 250 amino acids to about 3,000 amino acids, where the VLP comprises a pseudotyping viral glycoprotein and/or a polypeptide that provides for binding to a target cell. The present disclosure provides systems for producing a VLP. The present disclosure provides methods of delivering a therapeutic protein, using a VLP of the present disclosure.

### BRIEF DESCRIPTION OF THE DRAWINGS

FIG. 1A-1G depict production and characterization of Cas9-VLPs.

FIG. 2A-2F depict efficient genome editing by Cas9 VLPs.

FIG. 3A-3F depict generation of highly engineered CAR-expressing primary human T cells using Cas9-VLPs.

FIG. 4A-4C depict targeting Cas9-VLP genome editing to CD4+ T cells by HIV-1 Envelope pseudotyping.

FIG. 5A-5E depict Cas9-VLP-mediated homology-directed repair (HDR).

FIG. 6A-6F depict genome editing using various Cas9-VLP formulations.

FIG. 7A-7F depict data showing that traceless Cas9-VLPs mediate genome editing without viral transgene insertion and hybrid Cas9-VLPs do not require a lentiviral-encoded guide RNA expression cassette.

FIG. 8A-8D depict targeted integration of the lentiviral genome into the Cas9 RNP target site. FIG. 8C presents, from top to bottom SEQ ID NOs:185-189. FIG. 8D presents, from top to bottom, SEQ ID NOs:190-199.

FIG. 9A-9B depict representative flow cytometry gating strategy for quantifying genome editing in primary human T cells.

FIG. 10A-10D depict optimization of CAR-Cas9-VLP production & representative flow cytometry gating strategy for Cas9-VLP-mediated multiplexed genome engineering of primary human CAR-T cells.

FIG. 11A-11B depict Cas9-VLP genome editing as a function of multiplicity of infection (MOI) and quantity of CA.

FIG. 12 depicts functional cytokine production and surface receptor expression in Cas9-VLP generated CAR-T cells.

FIG. 13A-13G depict characterization of bald and HIV-1 Env pseudotyped Cas9-VLPs.

FIG. 14 provides Table 1, which includes protospacer sequences (from top to bottom SEQ ID NOs:166-170).

FIG. 15 provides Table 2, which provides genomic amplification and sequencing primers (from top to bottom SEQ ID NOs:171-178, 175, 179, 180, 176, 175, 180, 179, 176, 175, 176, 175, 181, 181, 176, 177, 182, 182, 178, 171, 172, 182, 183).

FIG. 16 provides Table 3 (SEQ ID NO: 184).

FIG. 17 provides Table 4.

FIG. 18A-18P provide amino acid sequences of CRISPR-Cas effector polypeptides.

### DEFINITIONS

“Heterologous,” as used herein, means a nucleotide or polypeptide sequence that is not found in the native nucleic acid or protein, respectively. For example, in the context of a retroviral gag polyprotein, a “heterologous” protease cleavage site is a protease cleavage site that is not found naturally in a retroviral gag polyprotein. Similarly, in the context of a retrovirus (e.g., a lentivirus), a “heterologous” protease is a protease that is not normally encoded by the retrovirus. As another example, relative to a CRISPR-Cas effector polypeptide, a heterologous polypeptide comprises an amino acid sequence from a protein other than the CRISPR-Cas effector polypeptide. As another example, a CRISPR-Cas effector protein (e.g., a dead CRISPR-Cas effector protein) can be fused to an active domain from a non-CRISPR-Cas effector protein (e.g., a cytidine deaminase), and the sequence of the active domain could be considered a heterologous polypeptide (it is heterologous to the CRISPR-Cas effector protein).

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “naturally-occurring” as used herein as applied to a nucleic acid, a protein, a cell, or an organism, refers to a nucleic acid, cell, protein, or organism that is found in nature.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

“Heterologous,” as used herein, refers to a nucleotide or amino acid sequence that is not found in the native nucleic acid or protein, respectively. For example, relative to a Cas9 polypeptide, a heterologous polypeptide comprises an amino acid sequence from a protein other than the Cas9 polypeptide. Thus, for example, a polymerase polypeptide is heterologous to a Cas9 polypeptide.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, nucleotide sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant nucleotide sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below).

Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such artificial combination can be carried out to join together nucleic acid segments of desired functions to generate a desired combination of functions.

Similarly, the term “recombinant” polypeptide refers to a polypeptide which is not naturally occurring, e.g., is made by the artificial combination of two otherwise separated segments of amino acid sequence through human intervention. Thus, e.g., a polypeptide that comprises a heterologous amino acid sequence is recombinant.

By “construct” or “vector” is meant a recombinant nucleic acid, generally recombinant DNA, which has been generated for the purpose of the expression and/or propagation of a specific nucleotide sequence(s), or is to be used in the construction of other recombinant nucleotide sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (e.g., DNA exogenous to the cell) into the cell. Genetic change (“modification”) can be accomplished either by incorporation of the new nucleic acid into the genome of the host cell, or by transient or stable maintenance of the new nucleic acid as an episomal element. Where the cell is a eukaryotic cell, a permanent genetic change can be achieved by introduction of new DNA into the genome of the cell. In prokaryotic cells, permanent changes can be introduced into the chromosome or via extrachromosomal elements such as plasmids and expression vectors, which may contain one or more selectable markers to aid in their maintenance in the recombinant host cell. Suitable methods of genetic modification include viral infection, transfection, conjugation, protoplast fusion, electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, evibodies, minobodies, diabodies, and fusion proteins comprising an antigen-binding (also referred to herein as antigen binding) portion of an antibody and a non-antibody protein.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids. In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

As used herein, the term “antibody mimetic” refers to compounds which, like antibodies, can specifically and/or selectively bind antigens or other targets, but which are not structurally related to antibodies. Antibody mimetics are usually artificial peptides or proteins, but they are not limited to such embodiments. Typically, antibody mimetics are smaller than antibodies, with a molar mass of about 3-20 kDa (whereas antibodies are generally about 150 kDa). Non-limiting examples of antibody mimetics include peptide aptamers, affimers, affilins, affibodies, affitins, alphabodies, anticalins, avimers, DARPins, fynomers, Kunitz domain peptides, nanoCLAMPs, affinity reagents and scaffold proteins.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to an individual organism, e.g., a mammal, including, but not limited to, murines, simians, non-human primates, humans, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a guide RNA” includes a plurality of such guide RNAs and reference to “the CRISPR-Cas effector polypeptide” includes reference to one or more CRISPR-Cas effector polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides virus-like particles (VLPs) comprising: i) a CRISPR-Cas effector polypeptide; ii) a recombinant lentivirus comprising a nucleotide sequence encoding a therapeutic polypeptide having a length of from about 250 amino acids to about 3,000 amino acids, where the VLP comprises a pseudotyping viral glycoprotein and/or a polypeptide that provides for binding to a target cell. The present disclosure provides systems for producing a VLP. The present disclosure provides methods of delivering a therapeutic protein, using a VLP of the present disclosure.

### Virus-Like Particles

The present disclosure provides virus-like particles (VLPs) comprising: i) a CRISPR-Cas effector polypeptide; ii) a recombinant lentivirus comprising a nucleotide sequence encoding a therapeutic polypeptide having a length of from about 250 amino acids to about 3,000 amino acids, where the VLP comprises a pseudotyping viral glycoprotein and/or a polypeptide that provides for binding to a target cell. In some cases, a VLP comprises a CRISPR-Cas effector guide RNA (referred to herein as a “guide RNA”), or a nucleic acid comprising a nucleotide sequence encoding a guide RNA. In some cases, the VLP also includes a donor template nucleic acid.

Where a VLP of the present disclosure comprises a guide RNA, in some instances, the guide RNA provides for knockout of a nucleic acid targeted by the guide RNA. Thus, in some cases, a VLP of the present disclosure provides for: i) delivery of a therapeutic protein; and ii) knockout of a target nucleic acid. As one non-limiting example, a VLP of the present disclosure can both: i) provide for delivery of a therapeutic protein (such as a chimeric antigen receptor (CAR)); and ii) knock out an endogenous nucleic acid encoding a beta-2 microglobulin (β2M) polypeptide, where the guide RNA present in the VLP (or encoded by a nucleic acid present in the VLP) would comprise a nucleotide sequence targeting a β2M-encoding nucleic acid in a target cell. Such a VLP would be useful for generating T cells that express a CAR (“CAR-T cells”) that do not express endogenous major histocompatibility complex (MHC) class I antigens on their cell surface and thus could be useful for delivery of allogeneic CAR-T cells. As another non-limiting example, a VLP of the present disclosure can both: i) provide for delivery of a therapeutic protein (such as an antibody, e.g., a cancer-specific antibody or other therapeutic antibody); and ii) knock out an endogenous nucleic acid encoding an antibody light chain (e.g., a kappa light chain) or an immunoglobulin (Ig) Fc polypeptide (e.g., an Ig Fc polypeptide of a particular isotype such as IgG1). Such a VLP would be useful for generating B cells that produce a therapeutic antibody.

### CRISPR-Cas Effector Polypeptides

As noted above, a VLP of the present disclosure comprises a CRISPR-Cas effector polypeptide. The CRISPR-Cas effector polypeptide can be any of a variety of CRISPR-Cas effector polypeptides. Suitable CRISPR-Cas effector polypeptides are described in detail below. For example, in some cases, the CRISPR-Cas effector polypeptide is a type II CRISPR-Cas effector polypeptide. In some cases, the type II CRISPR-Cas effector polypeptide is a Cas9 polypeptide. In some cases, the CRISPR-Cas effector polypeptide is a type V CRISPR-Cas effector polypeptide, e.g., a Cas12a, a Cas12b, a Cas12c, a Cas12d, or a Cas12e polypeptide. In some cases, the CRISPR-Cas effector polypeptide is a type VI CRISPR-Cas effector polypeptide, e.g., a Cas13a polypeptide, a Cas13b polypeptide, a Cas13c polypeptide, or a Cas13d polypeptide. In some cases, the CRISPR-Cas effector polypeptide is a Cas14 polypeptide. In some cases, the CRISPR-Cas effector polypeptide is a Cas14a polypeptide, a Cas14b polypeptide, or a Cas14c polypeptide. Also suitable for use is a variant CRISPR-Cas effector polypeptide, where the variant CRISPR-Cas effector polypeptide has reduced nucleic acid cleavage activity. Also suitable for use is a CRISPR-Cas effector fusion polypeptide comprising: i) a CRISPR-Cas effector polypeptide is a variant that has reduced nucleic acid cleavage activity; and ii) a heterologous fusion polypeptide. In some cases, the heterologous fusion polypeptide is a protein modifying enzyme. In some cases, the heterologous fusion polypeptide is a nucleic acid modifying enzyme. In some cases, the heterologous fusion polypeptide is a transcription factor. In some cases, the heterologous fusion polypeptide is a transcription activator. In some cases, the heterologous fusion polypeptide is a transcription repressor. Suitable protein-modifying enzymes and nucleic acid modifying enzymes are described in detail below. For example, in some cases, the nucleic acid modifying enzyme is a cytidine deaminase. In some cases, the nucleic acid modifying enzyme is an adenosine deaminase. In some cases, the nucleic acid modifying enzyme is a prime editor. As described in more detail below, in some cases, the CRISPR-Cas effector polypeptide comprises one or more nuclear localization signals.

Examples of CRISPR-Cas effector polypeptides are CRISPR-Cas endonucleases (e.g., class 2 CRISPR-Cas effector polypeptide such as a type II, type V, or type VI CRISPR-Cas effector polypeptide). Where a CRISPR-Cas effector polypeptide has endonuclease activity, the CRISPR-Cas effector polypeptide may also be referred to as a “CRISPR-Cas endonuclease.” A CRISPR-Cas effector polypeptide can also have reduced or undetectable endonuclease activity. A CRISPR-Cas effector polypeptide can also be a fusion CRISPR-Cas effector polypeptide comprising a heterologous fusion partner. In some cases, a suitable CRISPR-Cas effector polypeptide is a class 2 CRISPR-Cas effector polypeptide. In some cases, a suitable CRISPR-Cas effector polypeptide is a class 2 type II CRISPR-Cas effector polypeptide (e.g., a Cas9 protein). In some cases, a suitable CRISPR-Cas effector polypeptide is a class 2 type V CRISPR-Cas endonuclease (e.g., a Cpf1 protein, a C2c1 protein, or a C2c3 protein). In some cases, a suitable CRISPR-Cas effector polypeptide is a class 2 type VI CRISPR-Cas effector polypeptide (e.g., a C2c2 protein; also referred to as a “Cas13a” protein). Also suitable for use is a CasX protein. Also suitable for use is a CasY protein.

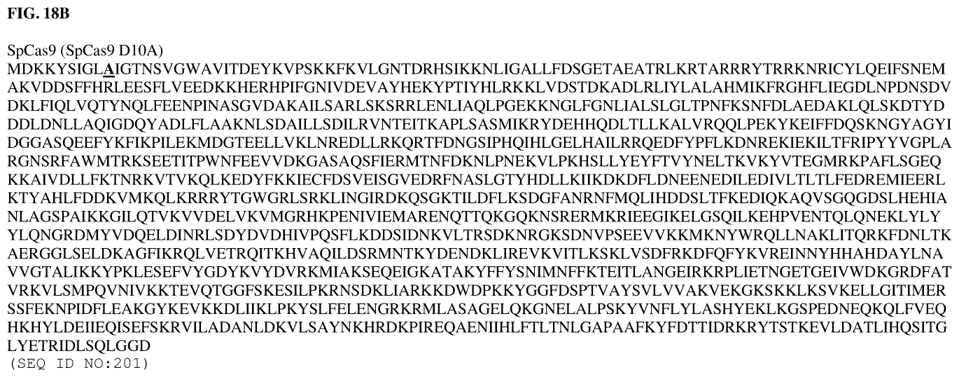

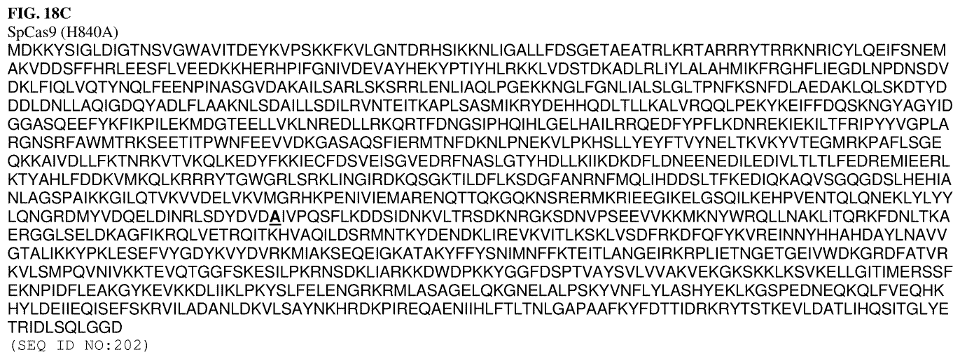

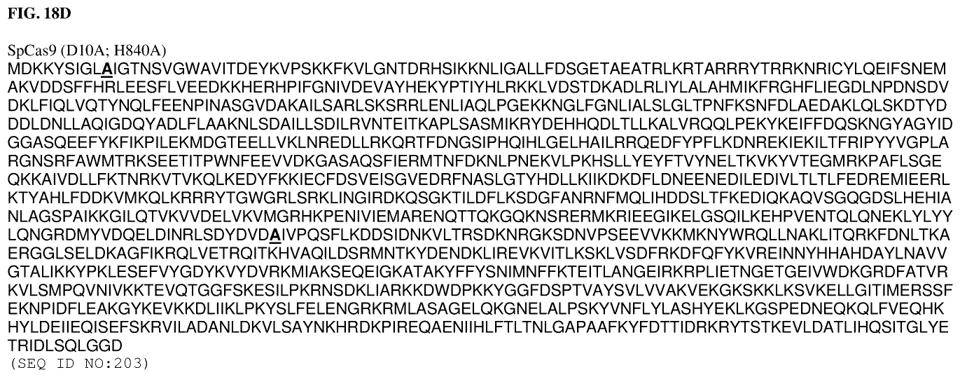

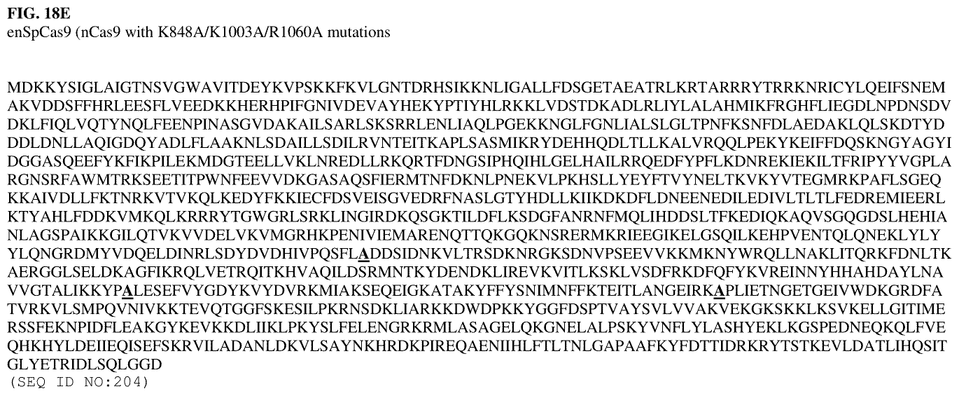

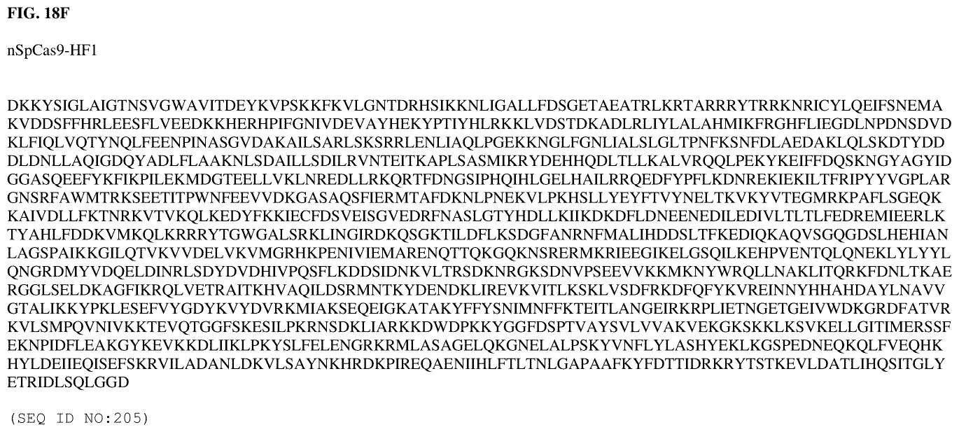

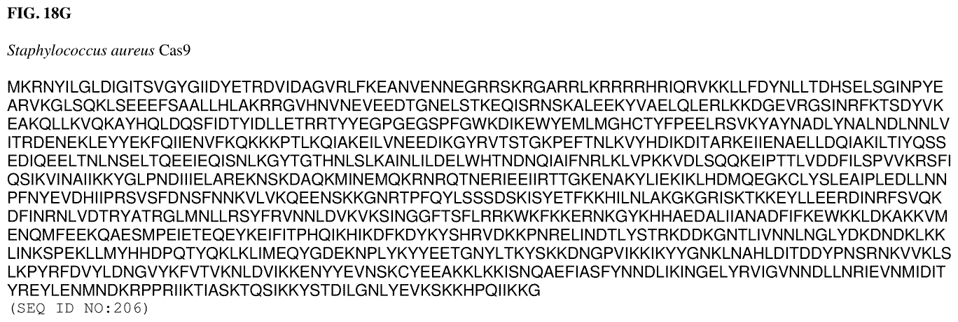

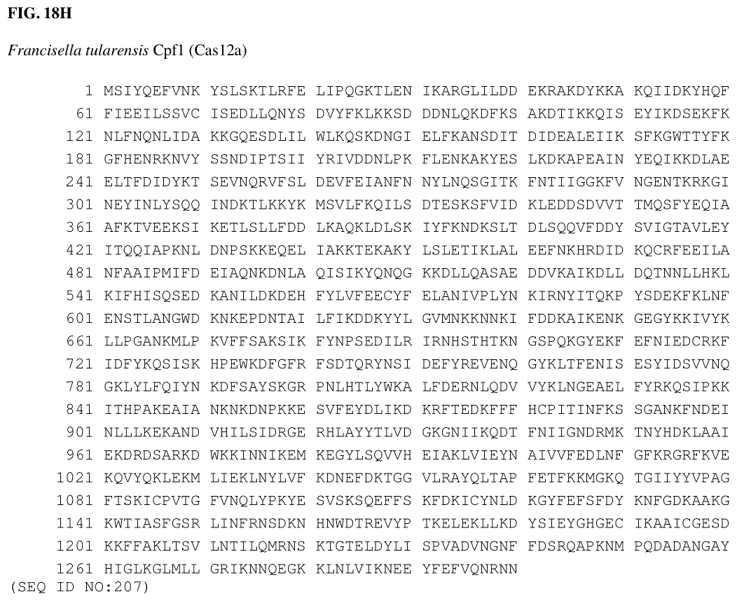

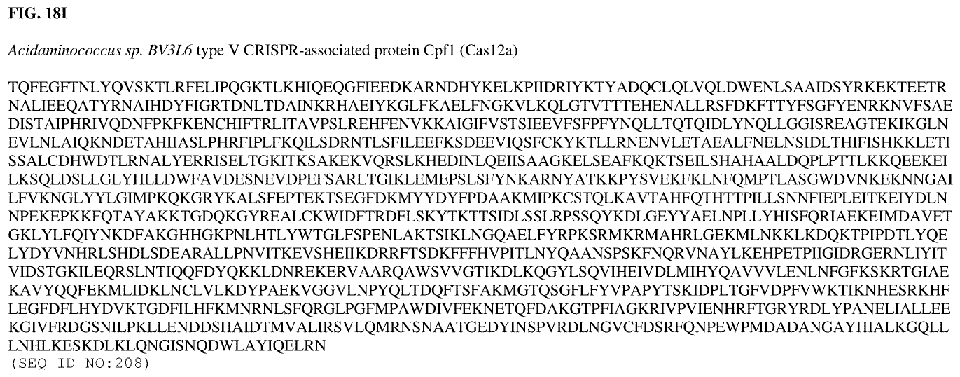

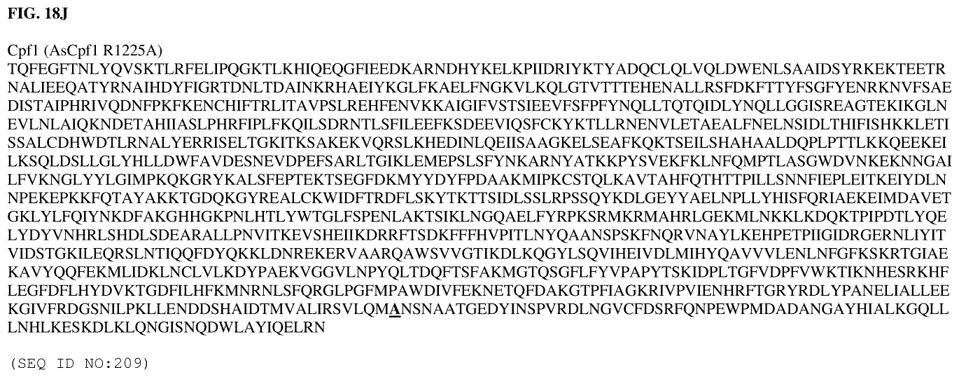

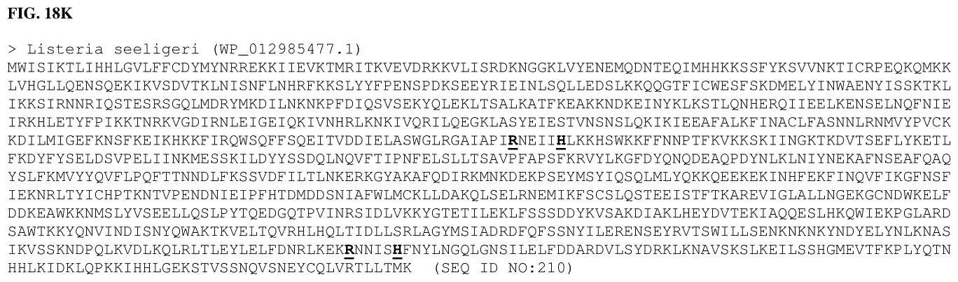

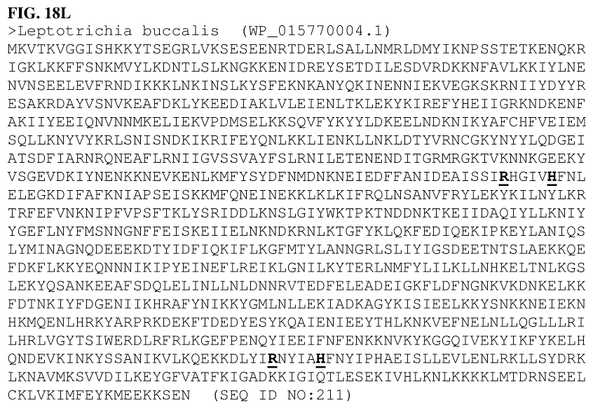

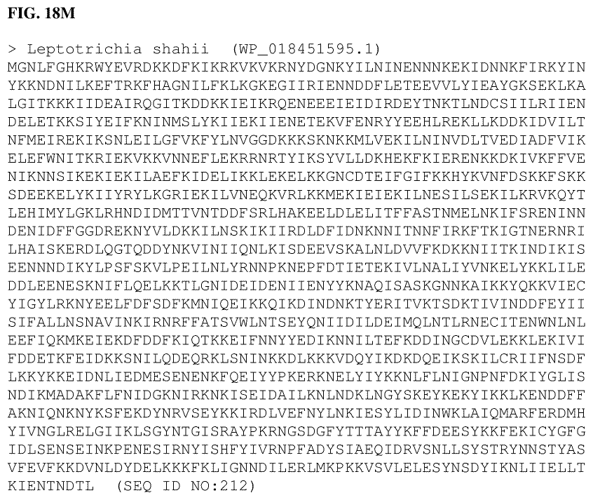

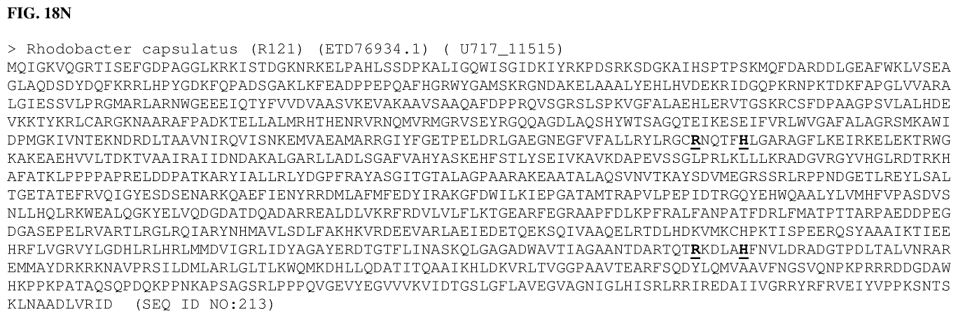

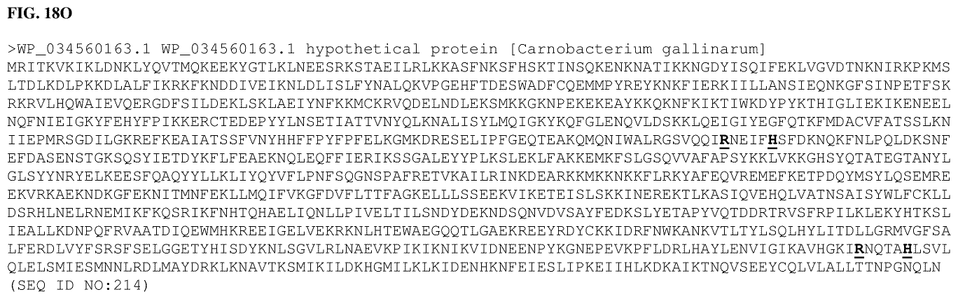

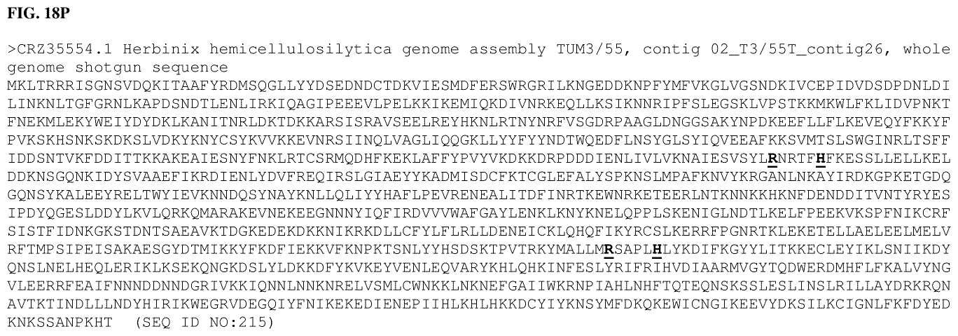

In some cases, a CRISPR/Cas effector polypeptide suitable for inclusion in a fusion polypeptide of the present disclosure comprises an amino acid sequence having at least 85%, at least 90%, at least 95%, at least 98%, at least 99%, or 100%, amino acid sequence identity to the amino acid sequence depicted in any one of FIG. 18A-18P.

In some cases, the CRISPR-Cas effector polypeptide is a Type II CRISPR-Cas effector polypeptide. In some cases, the CRISPR-Cas effector polypeptide is a Cas9 polypeptide. The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, a Cas9 polypeptide comprises an amino acid sequence having at least 50%, at least 60%, at least 70%, at least 80%, at least 90%, at least 95%, at least 98%, at least 99%, or more than 99%, amino acid sequence identity to the Streptococcus pyogenes Cas9 depicted in FIG. 18A.

In some cases, the Cas9 polypeptide is a Staphylococcus aureus Cas9 (saCas9) polypeptide. In some cases, the saCas9 polypeptide comprises an amino acid sequence having at least 85%, at least 90%, at least 95%, at least 98%, at least 99%, or 100%, amino acid sequence identity to the saCas9 amino acid sequence depicted in FIG. 18G.

In some cases, a suitable Cas9 polypeptide is a high-fidelity (HF) Cas9 polypeptide. Kleinstiver et al. (2016) Nature 529:490. For example, amino acids N497, R661, Q695, and Q926 of the amino acid sequence depicted in FIG. 18A are substituted, e.g., with alanine. For example, an HF Cas9 polypeptide can comprise an amino acid sequence having at least 90%, at least 95%, at least 98%, at least 99%, or 100%, amino acid sequence identity to the amino acid sequence depicted in FIG. 18A, where amino acids N497, R661, Q695, and Q926 are substituted, e.g., with alanine. In some cases, a suitable Cas9 polypeptide exhibits altered PAM specificity. See, e.g., Kleinstiver et al. (2015) Nature 523:481.

In some cases, a suitable CRISPR-Cas effector polypeptide is a type V CRISPR-Cas effector polypeptide. In some cases, a type V CRISPR-Cas effector polypeptide is a Cpf1 protein. In some cases, a Cpf1 protein comprises an amino acid sequence having at least 30%, at least 35%, at least 40%, at least 45%, at least 50%, at least 55%, at least 60%, at least 65%, at least 70%, at least 75%, at least 80%, at least 85%, at least 90%, at least 95%, at least 90%, or 100%, amino acid sequence identity to the Cpf1 amino acid sequence depicted in FIG. 18H, FIG. 181, or FIG. 18J.

In some cases, a suitable CRISPR-Cas effector polypeptide is a CasX or a CasY polypeptide. CasX and CasY polypeptides are described in Burstein et al. (2017) Nature 542:237.

In some cases, a suitable CRISPR-Cas effector polypeptide is a fusion protein comprising a CRISPR-Cas effector polypeptide that is fused to a heterologous polypeptide (also referred to as a “fusion partner”). In some cases, a CRISPR-Cas effector polypeptide is fused to an amino acid sequence (a fusion partner) that provides for subcellular localization, i.e., the fusion partner is a subcellular localization sequence (e.g., one or more nuclear localization signals (NLSs) for targeting to the nucleus, two or more NLSs, three or more NLSs, etc.).

A nucleic acid that binds to a class 2 CRISPR-Cas effector polypeptide (e.g., a Cas9 protein; a type V or type VI CRISPR-Cas protein; a Cpf1 protein; etc.) and targets the complex to a specific location within a target nucleic acid is referred to herein as a “guide RNA” or “CRISPR-Cas guide nucleic acid” or “CRISPR-Cas guide RNA.” A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

In some cases, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some cases, the guide RNA is one molecule (e.g., for some class 2 CRISPR-Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

In some cases, a VLP of the present disclosure comprises a CRISPR-Cas effector polypeptide, or both a CRISPR-Cas effector polypeptide and a guide RNA. In some cases, e.g., where a target nucleic acid comprises a deleterious mutation in a defective allele (e.g., a deleterious mutation in a retinal cell target nucleic acid), the CRISPR-Cas effector polypeptide/guide RNA complex, together with a donor nucleic acid comprising a nucleotide sequence that corrects the deleterious mutation (e.g., a donor nucleic acid comprising a nucleotide sequence that encodes a functional copy of the protein encoded by the defective allele), can be used to correct the deleterious mutation, e.g., via homology-directed repair (HDR).

In some cases, a VLP of the present disclosure comprises: i) a CRISPR-Cas effector polypeptide; and ii) one guide RNA. In some cases, the guide RNA is a single-molecule (or “single guide”) guide RNA (an “sgRNA”). In some cases, the guide RNA is a dual-molecule (or “dual-guide”) guide RNA (“dgRNA”).

In some cases, a VLP of the present disclosure comprises: i) a CRISPR-Cas effector polypeptide; and ii) 2 or more gRNAs, where the two or more gRNAs provide for multiplexed gene knockout, e.g., each of the 2 or more guide RNAs is targeted to a different gene. In some cases, the guide RNAs are sgRNAs. In some cases, the guide RNAs are dgRNAs.

In some cases, a VLP of the present disclosure comprises: i) a CRISPR-Cas effector polypeptide; and ii) 2 separate sgRNAs, where the 2 separate sgRNAs provide for deletion of a target nucleic acid via non-homologous end joining (NHEJ). In some cases, the guide RNAs are sgRNAs. In some cases, the guide RNAs are dgRNAs.

### Class 2 CRISPR-Cas Effector Polypeptides

In class 2 CRISPR systems, the functions of the effector complex (e.g., the cleavage of target DNA) are carried out by a single endonuclease (e.g., see Zetsche et al., Cell. 2015 Oct. 22; 163(3):759-71; Makarova et al., Nat Rev Microbiol. 2015 November; 13(11):722-36; Shmakov et al., Mol Cell. 2015 Nov. 5; 60(3):385-97); and Shmakov et al. (2017) Nature Reviews Microbiology 15:169. As such, the term “class 2 CRISPR-Cas protein” is used herein to encompass the CRISPR-Cas effector polypeptide (e.g., the target nucleic acid cleaving protein) from class 2 CRISPR systems. Thus, the term “class 2 CRISPR-Cas effector polypeptide” as used herein encompasses type II CRISPR-Cas effector polypeptides (e.g., Cas9); type V-A CRISPR-Cas effector polypeptides (e.g., Cpf1 (also referred to a “Cas12a”)); type V-B CRISPR-Cas effector polypeptides (e.g., C2c1 (also referred to as “Cas12b”)); type V-C CRISPR-Cas effector polypeptides (e.g., C2c3 (also referred to as “Cas12c”)); type V-U1 CRISPR-Cas effector polypeptides (e.g., C2c4); type V-U2 CRISPR-Cas effector polypeptides (e.g., C2c8); type V-U5 CRISPR-Cas effector polypeptides (e.g., C2c5); type V-U4 CRISPR-Cas proteins (e.g., C2c9); type V-U3 CRISPR-Cas effector polypeptides (e.g., C2c10); type VI-A CRISPR-Cas effector polypeptides (e.g., C2c2 (also known as “Cas13a”)); type VI-B CRISPR-Cas effector polypeptides (e.g., Cas13b (also known as C2c4)); and type VI-C CRISPR-Cas effector polypeptides (e.g., Cas13c (also known as C2c7)). To date, class 2 CRISPR-Cas effector polypeptides encompass type II, type V, and type VI CRISPR-Cas effector polypeptides, but the term is also meant to encompass any class 2 CRISPR-Cas effector polypeptide suitable for binding to a corresponding guide RNA and forming an RNP complex.

In some cases, a CRISPR-Cas effector polypeptide is a fusion polypeptide comprising: i) a CRISPR-Cas effector polypeptide; and ii) one or more heterologous fusion partners (one or more heterologous fusion polypeptides). In some cases, a fusion CRISPR-Cas effector polypeptide comprises one or more localization signal peptides. In some cases, a fusion CRISPR-Cas effector polypeptide comprises one or more localization signal peptides. Suitable localization signals (“subcellular localization signals”) include, e.g., a nuclear localization signal (NLS) for targeting to the nucleus; a sequence to keep the fusion protein out of the nucleus, e.g., a nuclear export sequence (NES); a sequence to keep the fusion protein retained in the cytoplasm; a mitochondrial localization signal for targeting to the mitochondria; a chloroplast localization signal for targeting to a chloroplast; an endoplasmic reticulum (ER) retention signal; and ER export signal; and the like. In some cases, a fusion CRISPR-Cas effector polypeptide does not include a NLS so that the protein is not targeted to the nucleus (which can be advantageous, e.g., when the target nucleic acid is an RNA that is present in the cytosol).

In some cases, a fusion CRISPR-Cas effector polypeptide includes (is fused to) a nuclear localization signal (NLS) (e.g., in some cases 2 or more, 3 or more, 4 or more, or 5 or more NLSs). Thus, in some cases, a fusion polypeptide includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

Non-limiting examples of NLSs include an NLS sequence derived from: the NLS of the SV40 virus large T-antigen, having the amino acid sequence PKKKRKV (SEQ ID NO: 1); the NLS from nucleoplasmin (e.g., the nucleoplasmin bipartite NLS with the sequence KRPAATKKAGQAKKKK (SEQ ID NO:2)); the c-myc NLS having the amino acid sequence PAAKRVKLD (SEQ ID NO:3) or RQRRNELKRSP (SEQ ID NO:4); the hRNPA1 M9 NLS having the sequence NQSSNFGPMKGGNFGGRSSGPYGGGGQYFAKPRNQGGY (SEQ ID NO:5); the sequence RMRIZFKNKGKDTAELRRRRVEVSVELRKAKKDEQILKRRNV (SEQ ID NO:6) of the IBB domain from importin-alpha; the sequences VSRKRPRP (SEQ ID NO:7) and PPKKARED (SEQ ID NO:8) of the myoma T protein; the sequence PQPKKKPL (SEQ ID NO:9) of human p53; the sequence SALIKKKKKMAP (SEQ ID NO: 10) of mouse c-abl IV; the sequences DRLRR (SEQ ID NO: 11) and PKQKKRK (SEQ ID NO: 16) of the influenza virus NS1; the sequence RKLKKKIKKL (SEQ ID NO: 12) of the Hepatitis virus delta antigen; the sequence REKKKFLKRR (SEQ ID NO: 13) of the mouse Mx1 protein; the sequence KRKGDEVDGVDEVAKKKSKK (SEQ ID NO: 14) of the human poly(ADP-ribose) polymerase; and the sequence RKCLQAGMNLEARKTKK (SEQ ID NO: 15) of the steroid hormone receptors (human) glucocorticoid. In some cases, an NLS comprises the amino acid sequence MDSLLMNRRKFLYQFKNVRWAKGRRETYLC (SEQ ID NO: 17). In general, NLS (or multiple NLSs) are of sufficient strength to drive accumulation of the fusion polypeptide in a detectable amount in the nucleus of a eukaryotic cell. Detection of accumulation in the nucleus may be performed by any suitable technique. For example, a detectable marker may be fused to the fusion polypeptide such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

### Guide Nucleic Acid

As noted above, a VLP of the present disclosure comprises a CRISPR-Cas effector polypeptide guide nucleic acid (e.g., RNA) or a nucleic acid comprising a nucleotide sequence encoding a CRISPR-Cas effector polypeptide guide RNA.

A nucleic acid molecule that binds to a CRISPR-Cas effector polypeptide protein and targets the complex to a specific location within a target nucleic acid is referred to herein as a “CRISPR-Cas effector polypeptide guide RNA” or simply a “guide RNA.”

A guide RNA (can be said to include two segments, a first segment (referred to herein as a “targeting segment”); and a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. The “targeting segment” is also referred to herein as a “variable region” of a guide RNA. The “protein-binding segment” is also referred to herein as a “constant region” of a guide RNA. In some cases, the guide RNA is a Cas9 guide RNA.

The first segment (targeting segment) of a guide RNA includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (binds to) a CRISPR-Cas effector polypeptide. The protein-binding segment of a guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the guide RNA (the guide sequence of the guide RNA) and the target nucleic acid.

A guide RNA and a CRISPR-Cas effector polypeptide form a complex (e.g., bind via non-covalent interactions). The guide RNA provides target specificity to the complex by including a targeting segment, which includes a guide sequence (a nucleotide sequence that is complementary to a sequence of a target nucleic acid). The CRISPR-Cas effector polypeptide of the complex provides the site-specific activity (e.g., cleavage activity or an activity provided by the CRISPR-Cas effector polypeptide when the CRISPR-Cas effector polypeptide is a CRISPR-Cas effector polypeptide fusion polypeptide, i.e., has a fusion partner). In other words, the CRISPR-Cas effector polypeptide is guided to a target nucleic acid sequence (e.g. a target sequence in a chromosomal nucleic acid, e.g., a chromosome; a target sequence in an extrachromosomal nucleic acid, e.g. an episomal nucleic acid, a minicircle, an ssRNA, an ssDNA, etc.; a target sequence in a mitochondrial nucleic acid; a target sequence in a chloroplast nucleic acid; a target sequence in a plasmid; a target sequence in a viral nucleic acid; etc.) by virtue of its association with the guide RNA.

The “guide sequence” also referred to as the “targeting sequence” of a guide RNA can be modified so that the guide RNA can target a CRISPR-Cas effector polypeptide to any desired sequence of any desired target nucleic acid, with the exception that the protospacer adjacent motif (PAM) sequence can be taken into account. Thus, for example, a guide RNA can have a targeting segment with a sequence (a guide sequence) that has complementarity with (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

In some embodiments, a guide RNA includes two separate nucleic acid molecules: an “activator” and a “targeter” and is referred to herein as a “dual guide RNA”, a “double-molecule guide RNA”, or a “two-molecule guide RNA” a “dual guide RNA”, or a “dgRNA.” In some embodiments, the activator and targeter are covalently linked to one another (e.g., via intervening nucleotides) and the guide RNA is referred to as a “single guide RNA”, a “Cas9 single guide RNA”, a “single-molecule Cas9 guide RNA,” or a “one-molecule Cas9 guide RNA”, or simply “sgRNA.”

A guide RNA comprises a crRNA-like (“CRISPR RNA”/“targeter”/“crRNA”/“crRNA repeat”) molecule and a corresponding tracrRNA-like (“trans-acting CRISPR RNA”/“activator”/“tracrRNA”) molecule. A crRNA-like molecule (targeter) comprises both the targeting segment (single stranded) of the guide RNA and a stretch (“duplex-forming segment”) of nucleotides that forms one half of the dsRNA duplex of the protein-binding segment of the guide RNA. A corresponding tracrRNA-like molecule (activator/tracrRNA) comprises a stretch of nucleotides (duplex-forming segment) that forms the other half of the dsRNA duplex of the protein-binding segment of the guide nucleic acid. In other words, a stretch of nucleotides of a crRNA-like molecule are complementary to and hybridize with a stretch of nucleotides of a tracrRNA-like molecule to form the dsRNA duplex of the protein-binding domain of the guide RNA. As such, each targeter molecule can be said to have a corresponding activator molecule (which has a region that hybridizes with the targeter). The targeter molecule additionally provides the targeting segment. Thus, a targeter and an activator molecule (as a corresponding pair) hybridize to form a guide RNA. The exact sequence of a given crRNA or tracrRNA molecule is characteristic of the species in which the RNA molecules are found. A dual guide RNA can include any corresponding activator and targeter pair.

The term “activator” or “activator RNA” is used herein to mean a tracrRNA-like molecule (tracrRNA: “trans-acting CRISPR RNA”) of a dual guide RNA (and therefore of a single guide RNA when the “activator” and the “targeter” are linked together by, e.g., intervening nucleotides). Thus, for example, a guide RNA (dgRNA or sgRNA) comprises an activator sequence (e.g., a tracrRNA sequence). A tracr molecule (a tracrRNA) is a naturally existing molecule that hybridizes with a CRISPR RNA molecule (a crRNA) to form a dual guide RNA. The term “activator” is used herein to encompass naturally existing tracrRNAs, but also to encompass tracrRNAs with modifications (e.g., truncations, sequence variations, base modifications, backbone modifications, linkage modifications, etc.) where the activator retains at least one function of a tracrRNA (e.g., contributes to the dsRNA duplex to which Cas9 protein binds). In some cases, the activator provides one or more stem loops that can interact with Cas9 protein. An activator can be referred to as having a tracr sequence (tracrRNA sequence) and in some cases is a tracrRNA, but the term “activator” is not limited to naturally existing tracrRNAs.

The term “targeter” or “targeter RNA” is used herein to refer to a crRNA-like molecule (crRNA: “CRISPR RNA”) of a dual guide RNA (and therefore of a single guide RNA when the “activator” and the “targeter” are linked together, e.g., by intervening nucleotides). Thus, for example, a guide RNA (dgRNA or sgRNA) comprises a targeting segment (which includes nucleotides that hybridize with (are complementary to) a target nucleic acid, and a duplex-forming segment (e.g., a duplex forming segment of a crRNA, which can also be referred to as a crRNA repeat). Because the sequence of a targeting segment (the segment that hybridizes with a target sequence of a target nucleic acid) of a targeter is modified by a user to hybridize with a desired target nucleic acid, the sequence of a targeter will often be a non-naturally occurring sequence. However, the duplex-forming segment of a targeter (described in more detail below), which hybridizes with the duplex-forming segment of an activator, can include a naturally existing sequence (e.g., can include the sequence of a duplex-forming segment of a naturally existing crRNA, which can also be referred to as a crRNA repeat). Thus, the term targeter is used herein to distinguish from naturally occurring crRNAs, despite the fact that part of a targeter (e.g., the duplex-forming segment) often includes a naturally occurring sequence from a crRNA. However, the term “targeter” encompasses naturally occurring crRNAs.

A guide RNA can also be said to include 3 parts: (i) a targeting sequence (a nucleotide sequence that hybridizes with a sequence of the target nucleic acid); (ii) an activator sequence (as described above)(in some cases, referred to as a tracr sequence); and (iii) a sequence that hybridizes to at least a portion of the activator sequence to form a double stranded duplex. A targeter has (i) and (iii); while an activator has (ii).

A guide RNA (e.g. a dual guide RNA or a single guide RNA) can be comprised of any corresponding activator and targeter pair. In some cases, the duplex forming segments can be swapped between the activator and the targeter. In other words, in some cases, the targeter includes a sequence of nucleotides from a duplex forming segment of a tracrRNA (which sequence would normally be part of an activator) while the activator includes a sequence of nucleotides from a duplex forming segment of a crRNA (which sequence would normally be part of a targeter).

As noted above, a targeter comprises both the targeting segment (single stranded) of the guide RNA and a stretch (“duplex-forming segment”) of nucleotides that forms one half of the dsRNA duplex of the protein-binding segment of the guide RNA. A corresponding tracrRNA-like molecule (activator) comprises a stretch of nucleotides (a duplex-forming segment) that forms the other half of the dsRNA duplex of the protein-binding segment of the guide RNA. In other words, a stretch of nucleotides of the targeter is complementary to and hybridizes with a stretch of nucleotides of the activator to form the dsRNA duplex of the protein-binding segment of a guide RNA. As such, each targeter can be said to have a corresponding activator (which has a region that hybridizes with the targeter). The targeter molecule additionally provides the targeting segment. Thus, a targeter and an activator (as a corresponding pair) hybridize to form a guide RNA. The particular sequence of a given naturally existing crRNA or tracrRNA molecule is characteristic of the species in which the RNA molecules are found. Examples of suitable activator and targeter are well known in the art.

### Therapeutic Proteins

As noted above, a VLP of the present disclosure comprises a recombinant lentiviral nucleic acid comprising a nucleotide sequence encoding a therapeutic polypeptide.

A therapeutic polypeptide encoded by a recombinant lentiviral nucleic acid present in a VLP of the present disclosure has a length of from about 250 amino acids to about 3000 amino acids. For example, a therapeutic polypeptide encoded by a recombinant lentiviral nucleic acid present in a VLP of the present disclosure has a length of from about 250 amino acids to about 500 amino acids, from about 500 amino acids to about 1000 amino acids, from about 500 amino acids to about 750 amino acids, from about 750 amino acids to about 1500 amino acids, from about 750 amino acids to about 1000 amino acids, from about 1000 amino acids to about 1250 amino acids, from about 1000 amino acids to about 1500 amino acids, from about 1250 amino acids to about 1500 amino acids, from about 1250 amino acids to about 1750 amino acids, from about 1500 amino acids to about 1750 amino acids, from about 1500 amino acids to about 2000 amino acids, from about 1500 amino acids to about 2500 amino acids, from about 2000 amino acids to about 2500 amino acids, from about 2000 amino acids to about 3000 amino acids, or from about 2500 amino acids to about 3000 amino acids.

Suitable therapeutic proteins include, but are not limited to, a chimeric antigen receptor (CAR), a T cell receptor (TCR), a natural killer cell receptor (NKR), a synNotch polypeptide, an antibody, a Modular Extracellular Sensor Architecture (MESA) receptor, and the like. In some cases, a therapeutic protein is a functional version of a protein, e.g., a cystic fibrosis transmembrane conductance (CFTR) protein, a globin polypeptide (e.g., P-globin), and the like.

### Chimeric Antigen Receptor

In some cases, the therapeutic protein is a chimeric antigen receptor (CAR). A CAR generally comprises: a) an extracellular domain comprising an antigen-binding domain (antigen-binding polypeptide); b) a transmembrane region; and c) a cytoplasmic domain comprising an intracellular signaling domain (intracellular signaling polypeptide). In some cases, a CAR comprises: a) an extracellular domain comprising the antigen-binding domain; b) a transmembrane region; and c) a cytoplasmic domain comprising: i) a co-stimulatory polypeptide; and ii) an intracellular signaling domain. In some cases, a CAR comprises hinge region between the extracellular antigen-binding domain and the transmembrane domain. Thus, in some cases, a CAR comprises: a) an extracellular domain comprising the antigen-binding domain; b) a hinge region; c) a transmembrane region; and d) a cytoplasmic domain comprising an intracellular signaling domain. In some cases, a CAR comprises: a) an extracellular domain comprising the antigen-binding domain; b) a hinge region; c) a transmembrane region; and d) a cytoplasmic domain comprising: i) a co-stimulatory polypeptide; and ii) an intracellular signaling domain.

Exemplary CAR structures are known in the art (See e.g., WO 2009/091826; US 20130287748; WO 2015/142675; WO 2014/055657; WO 2015/090229; and U.S. Pat. No. 9,587,020.

In some cases, a CAR is a single polypeptide chain. In some cases, a CAR comprises two polypeptide chains.

CARs specific for a variety of tumor antigens are known in the art; for example CD171-specific CARs (Park et al., Mol Ther (2007) 15(4):825-833), EGFRvIII-specific CARs (Morgan et al., Hum Gene Ther (2012) 23(10):1043-1053), EGF-R-specific CARs (Kobold et al., J. Natl Cancer Inst (2014) 107(1):364), carbonic anhydrase IX-specific CARs (Lamers et al., Biochem Soc Trans (2016) 44(3):951-959), folate receptor-α (FR-α)-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21(14):3149-3159), IL-13Rα2-specific CARs (Brown et al., Clin Cancer Res (2015) 21(18):4062-4072), ganglioside GD2-specific CARs (Louis et al., Blood (2011) 118(23):6050-6056; Caruana et al., Nat Med (2015) 21(5):524-529; Yu et al. (2018) J. Hematol. Oncol. 11:1), ErbB2-specific CARS (Wilkie et al., J Clin Immunol (2012) 32(5):1059-1070), VEGF-R-specific CARs (Chinnasamy et al., Cancer Res (2016) 22(2):436-447), FAP-specific CARs (Wang et al., Cancer Immunol Res (2014) 2(2): 154-166), mesothelin (MSLN)-specific CARs (Moon et al, Clin Cancer Res (2011) 17(14):4719-30), NKG2D-specific CARs (VanSeggelen et al., Mol Ther (2015) 23(10):1600-1610), CD19-specific CARs (Axicabtagene ciloleucel (Yescarta™) and Tisagenlecleucel (Kymriah™). See also, Li et al., J Hematol and Oncol (2018) 11:22, reviewing clinical trials of tumor-specific CARs; Heyman and Yan (2019) Cancers 11:pii:E191; Baybutt et al. (2019) Clin. Pharmacol. Ther. 105:71.

### Antigen-Binding Domain

As noted above, a CAR comprises an extracellular domain comprising an antigen-binding domain. The antigen-binding domain present in a CAR can be any antigen-binding polypeptide, a wide variety of which are known in the art. In some instances, the antigen-binding domain is a single chain Fv (scFv). Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable. In some cases, the antigen-binding domain is a nanobody.

In some cases, the antigen bound by the antigen-binding domain of a CAR is selected from: a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PRI) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFβ) polypeptide, a MAD-CT-2 polypeptide, or a Fos-related antigen-1 (FOSL) polypeptide. In some cases, the antigen is a human papilloma virus (HPV) antigen. In some cases, the antigen is an alpha-feto protein (AFP) antigen. In some cases, the antigen is a Wilms tumor-1 (WT1) antigen.

The antigen-binding polypeptide of a CAR can bind any of a variety of cancer-associated antigens, including, e.g., antigens of the immunoglobulin superfamily (see, e.g., Barclay (2003) Seminars in Immunology 15:215); antigens of the tumor necrosis factor (TNF) superfamily (see, e.g., Aggarwal et al. (2012) Blood 119:651; Locksley et al. (2001) Cell 104:487; and Hehlgan and Pfeffer (2005) Immunol. 115:1); antigens of the TNF receptor (TNFR) superfamily (see, e.g., Locksley et al. (2001) Cell 104:487; and Hehlgan and Pfeffer (2005) Immunol. 115:1); antigens of the B7 superfamily (see, e.g., Greenwald et al. (2005) Ann. Rev. Immunol. 23:515; and Sharpe and Freeman (2002) Nat. Rev. Immunol. 2:116); and antigens of the lectin superfamily (see, e.g., Zelensky and Gready (2005) FEBS J. 272:6179).

The antigen-binding polypeptide of a CAR can bind any of a variety of cancer-associated antigens, including, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), B-cell maturation antigen (BCMA), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, RORI, SCH 900105, SDC1, SLAMF7, TAG-72, tenascin C, TGF beta 2, TGF-β, TRAIL-Ri, TRAIL-R2, tumor antigen CTAA16.88, VEGF-A, VEGFR-1, VEGFR2, and vimentin.

In some cases, the cancer-associated antigen bound by the antigen-binding polypeptide of a CAR is selected from AFP, BCMA, CD10, CD 117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2. In some cases, the cancer-associated antigen is BCMA. In some cases, the cancer-associated antigen is MUC1. In some cases, the cancer-associated antigen is CD19. In some cases, the cancer-associated antigen is AFP. In some cases, the cancer-associated antigen is Her-2. In some cases, the cancer-associated antigen is mesothelin. In some cases, the cancer-associated antigen is WT-1.

VH and VL amino acid sequences of various cancer-associated antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055.

### Hinge Region

As noted above, a CAR can include a hinge region between the extracellular domain and the transmembrane domain. As used herein, the term “hinge region” refers to a flexible polypeptide connector region (also referred to herein as “hinge” or “spacer”) providing structural flexibility and spacing to flanking polypeptide regions and can consist of natural or synthetic polypeptides. The hinge region can include complete hinge region derived from an antibody of a different class or subclass from that of the CH1 domain. The term “hinge region” can also include regions derived from CD8 and other receptors that provide a similar function in providing flexibility and spacing to flanking regions.

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

As non-limiting examples, an immunoglobulin hinge region can include one of the following amino acid sequences: DKTHT (SEQ ID NO: 18); CPPC (SEQ ID NO: 19); CPEPKSCDTPPPCPR (SEQ ID NO:20); ELKTPLGDTTHT (SEQ ID NO:21); KSCDKTHTCP (SEQ ID NO:22); KCCVDCP (SEQ ID NO:23); KYGPPCP (SEQ ID NO:24); EPKSCDKTHTCPPCP (SEQ ID NO:25) (human IgG1 hinge); ERKCCVECPPCP (SEQ ID NO:26) (human IgG2 hinge); ELKTPLGDTTHTCPRCP (SEQ ID NO:27) (human IgG3 hinge); SPNMVPHAHHAQ (SEQ ID NO:28) (human IgG4 hinge); and the like. The hinge region can comprise an amino acid sequence derived from human CD8; e.g., the hinge region can comprise the amino acid sequence: TTTPAPRPPTPAPTIASQPLSLRPEACRPAAGGAVHTRGLDFACD (SEQ ID NO:29), or a variant thereof.

### Transmembrane Domain

Any transmembrane (TM) domain that provides for insertion of a polypeptide into the cell membrane of a eukaryotic (e.g., mammalian) cell is suitable for use. The transmembrane region of a CAR can be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2Rbeta, IL2Rgamma, IL7R.alpha., ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. The transmembrane domain can be synthetic, in which case it can comprise predominantly hydrophobic residues such as leucine and valine. In some cases, a triplet of phenylalanine, tryptophan and valine will be found at each end of a synthetic transmembrane domain.

As one non-limiting example, the TM sequence IYIWAPLAGTCGVLLLSLVITLYC (SEQ ID NO:30) can be used. Additional non-limiting examples of suitable TM sequences include: a) CD8 beta derived TM: LGLLVAGVLVLLVSLGVAIHLCC (SEQ ID NO:31); b) CD4 derived TM: ALIVLGGVAGLLLFIGLGIFFCVRC (SEQ ID NO:32); c) CD3 zeta derived TM: LCYLLDGILFIYGVILTALFLRV (SEQ ID NO:33); d) CD28 derived TM: WVLVVVGGVLACYSLLVTVAFIIFWV (SEQ ID NO: 34); e) CD134 (OX40) derived TM: VAAILGLGLVLGLLGPLAILLALYLL (SEQ ID NO:35); and f) CD7 derived TM: ALPAALAVISFLLGLGLGVACVLA (SEQ ID NO:36).

### Intracellular Domain—Co-Stimulatory Polypeptide

The intracellular portion (cytoplasmic domain) of a CAR can comprise one or more co-stimulatory polypeptides. Non-limiting examples of suitable co-stimulatory polypeptides include, but are not limited to, 4-1BB (CD137), CD28, ICOS, OX-40, BTLA, CD27, CD30, GITR, and HVEM. Suitable co-stimulatory polypeptides include, e.g.: 1) a 4-1BB polypeptide having at least 90%, at least 95%, at least 98%, or 100%, amino acid sequence identity to the following amino acid sequence: KRGRKKLLYIFKQPFMRPVQTTQEEDGCSCRFPEEEEGGCEL (SEQ ID NO:37); 2) a CD28 polypeptide having at least 90%, at least 95%, at least 98%, or 100%, amino acid sequence identity to the following amino acid sequence: FWVRSKRSRLLHSDYMNMTPRRPGPTRKHYQPYAPPRDFAAYRS (SEQ ID NO:38); 3) an ICOS polypeptide having at least 90%, at least 95%, at least 98%, or 100%, amino acid sequence identity to the following amino acid sequence: TKKKYSSSVHDPNGEYMFMRAVNTAKKSRLTDVTL (SEQ ID NO:39); 4) an OX40 polypeptide having at least 90%, at least 95%, at least 98%, or 100%, amino acid sequence identity to the following amino acid sequence: RRDQRLPPDAHKPPGGGSFRTPIQEEQADAHSTLAKI (SEQ ID NO:40); 5) a BTLA polypeptide having at least 90%, at least 95%, at least 98%, or 100%, amino acid sequence identity to the following amino acid sequence: CCLRRHQGKQNELSDTAGREINLVDAHLKSEQTEASTRQNSQVLLSETGIYDNDPDLCFRMQEG SEVYSNPCLEENKPGIVYASLNHSVIGPNSRLARNVKEAPTEYASICVRS (SEQ ID NO:41); 6) a CD27 polypeptide having at least 90%, at least 95%, at least 98%, or 100%, amino acid sequence identity to the following amino acid sequence: HQRRKYRSNKGESPVEPAEPCRYSCPREEEGSTIPIQEDYRKPEPACSP (SEQ ID NO:42); 7) a CD30 polypeptide having at least 90%, at least 95%, at least 98%, or 100%, amino acid sequence identity to the following amino acid sequence: RRACRKRIRQKLHLCYPVQTSQPKLELVDSRPRRSSTQLRSGASVTEPVAEERGLMSQPLMETC HSVGAAYLESLPLQDASPAGGPSSPRDLPEPRVSTEHTNNKIEKIYIMKADTVIVGTVKAELPEG RGLAGPAEPELEEELEADHTPHYPEQETEPPLGSCSDVMLSVEEEGKEDPLPTAASGK (SEQ ID NO:43); 8) a GITR polypeptide having at least 90%, at least 95%, at least 98%, or 100%, amino acid sequence identity to the following amino acid sequence: HIWQLRSQCMWPRETQLLLEVPPSTEDARSCQFPEEERGERSAEEKGRLGDLWV (SEQ ID NO:44); and 9) an HVEM polypeptide having at least 90%, at least 95%, at least 98%, or 100%, amino acid sequence identity to the following amino acid sequence: CVKRRKPRGDVVKVIVSVQRKRQEAEGEATVIEALQAPPDVTTVAVEETIPSFTGRSPNH (SEQ ID NO:45). The co-stimulatory polypeptide can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

### Intracellular Domain—Signaling Polypeptide

The intracellular portion of a CAR can comprise a signaling polypeptide. Suitable signaling polypeptides include, e.g., an immunoreceptor tyrosine-based activation motif (ITAM)-containing intracellular signaling polypeptide. An ITAM motif is YX₁X₂L/I (SEQ ID NO:46), where X₁ and X₂ are independently any amino acid. In some cases, the intracellular signaling domain of a subject CAR comprises 1, 2, 3, 4, or 5 ITAM motifs. In some cases, an ITAM motif is repeated twice in an intracellular signaling domain, where the first and second instances of the ITAM motif are separated from one another by 6 to 8 amino acids, e.g., (YX₁X₂L/I)(X₃)ₙ(YX₁X₂L/I) (SEQ ID NO:47), where n is an integer from 6 to 8, and each of the 6-8 X₃ can be any amino acid. In some cases, the intracellular signaling domain of a CAR comprises 3 ITAM motifs.

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12; FCER1G (Fc epsilon receptor I gamma chain); CD3D (CD3 delta); CD3E (CD3 epsilon); CD3G (CD3 gamma); CD3Z (CD3 zeta); and CD79A (antigen receptor complex-associated protein alpha chain).

### SynNotch Receptor

In some cases, the therapeutic protein is a SynNotch receptor. A synNotch receptor can specifically bind and/or otherwise interact with a SynNotch receptor ligand. SynNotch receptor systems are programmable orthogonal receptor system that can couple binding, detection, or some other interaction with a specific ligand (e.g. a synNotch polypeptide) to an endogenous or engineered function in a cell. Each synNotch receptor can be composed of a core Notch domain flanked by modular extracellular and intracellular domains. Binding of a ligand to the synNotch receptor can trigger protease mediated cleavage of a transcription factor, which can be exogenous. In some embodiments, the SynNotch Receptor can be any SynNotch receptor or variant thereof as described in e.g. Roybal et al. 2016. Cell. Vol. 164(4):599-600; Morsut et al., 2016. Cell. 164:780-791; Roybal et al. Cell (2016) 167(2):419-432, Roybal et al. Cell (2016) 164(4):770-779; U.S. Pat. App. Pub: 2019/0134093, 2018/0355011; 2018/0079812; 2016/0264665; 2018/0208636; 2017/0233474; 2019/0202918; US2019/0010245; US20190270991; US2018/0346589; US20190269728; U.S. Pat. Nos. 9,670,281; 9,834,608; International Pat. Pub.: WO/2017/1993059; WO/2016/138034; WO2019/175428; WO2019/141270; WO2019/178259; WO 2018/039247; WO/2019/10901; WO/2019/195586; WO/2018/222880; WO/2019/016526; WO/2019/19557; WO/2019/166877.

SynNotch receptors are synthetic receptors that can specifically bind or otherwise interact with a SynNotch ligands. In some cases, the synNotch ligand is not a soluble ligand. In some cases, the synNotch ligand is insoluble and bound to a cell membrane or membrane of a vesicle.

In some embodiments, a synNotch receptor is composed of at least in part an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a Notch receptor. In some instances, the Notch regulatory region of a Notch receptor polypeptide is a mammalian Notch regulatory region, including but not limited to e.g., a mouse Notch (e.g., mouse Notch1, mouse Notch2, mouse Notch3 or mouse Notch4) regulatory region, a rat Notch regulatory region (e.g., rat Notch1, rat Notch2 or rat Notch3), a human Notch regulatory region (e.g., human Notch1, human Notch2, human Notch3 or human Notch4), and the like or a Notch regulatory region derived from a mammalian Notch regulatory region and having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a mammalian Notch regulatory region of a mammalian Notch receptor amino acid sequence.

Such Notch regulatory regions can include or exclude various components (e.g., domains, cleavage sites, etc.) thereof. Examples of such components of Notch regulatory regions that may be present or absent in whole or in part, as appropriate, include e.g., one or more EGF-like repeat domains, one or more Linl2/Notch repeat domains, one or more heterodimerization domains (e.g., HD-N or HD-C), a transmembrane domain, one or more proteolytic cleavage sites (e.g., a furin-like protease site (e.g., an S1 site), an ADAM-family protease site (e.g., an S2 site) and/or a gamma-secretase protease site (e.g., an S3 site)), and the like. Notch receptor polypeptides may, in some instances, exclude all or a portion of one or more Notch extracellular domains, including e.g., Notch-ligand binding domains such as Delta binding domains. Notch receptor polypeptides may, in some instances, include one or more non-functional versions of one or more Notch extracellular domains, including e.g., Notch-ligand binding domains such as Delta-binding domains. Notch receptor polypeptides may, in some instances, exclude all or a portion of one or more Notch intracellular domains, including e.g., Notch Rbp-associated molecule domains (i.e., RAM domains), Notch Ankyrin repeat domains, Notch transactivation domains, Notch PEST domains, and the like. Notch receptor polypeptides may, in some instances, include one or more non-functional versions of one or more Notch intracellular domains, including e.g., non-functional Notch Rbp-associated molecule domains (i.e., RAM domains), non-functional Notch Ankyrin repeat domains, non-functional Notch transactivation domains, non-functional Notch PEST domains, and the like.

In some cases, a synNotch polypeptide comprises, from N-terminus to C-terminus: a) a scFv or a nanobody that specifically binds an antigen; b) a Notch regulatory region comprising a Lin 12-Notch repeat, a heterodimerization domain comprising an S2 proteolytic cleavage site and a transmembrane domain comprising an S3 proteolytic cleavage site; c) an intracellular domain, heterologous to the Notch regulatory region, comprising a transcriptional activator comprising a DNA binding domain, where the transcriptional activator replaces a naturally-occurring intracellular notch domain, and where binding of the scFv or the nanobody to the antigen in trans induces cleavage at the S2 and S3 proteolytic cleavage sites, thereby releasing the intracellular domain and wherein the chimeric Notch polypeptide does not bind its naturally-occurring ligand Delta.

In some cases, the synNotch ligand is expressed on the surface of a target T-cell. In some cases, the synNotch ligand is expressed on the surface of a cell or vesicle that is not a target T-cell.

### Antibodies

In some cases, the therapeutic protein is an antibody. Suitable antibodies include, e.g., therapeutic antibodies. In some cases, the antibody is a single-chain Fv (scFv). In some cases, the antibody is a nanobody.

Suitable antibodies include, e.g., Natalizumab (Tysabri; Biogen Idec/Elan) targeting α4 subunit of α4β1 and α4β7 integrins (as used in the treatment of MS and Crohn's disease); Vedolizumab (MLN2; Millennium Pharmaceuticals/Takeda) targeting α4β7 integrin (as used in the treatment of UC and Crohn's disease); Belimumab (Benlysta; Human Genome Sciences/GlaxoSmithKline) targeting BAFF (as used in the treatment of SLE); Atacicept (TACI-Ig; Merck/Serono) targeting BAFF and APRIL (as used in the treatment of SLE); Alefacept (Amevive; Astellas) targeting CD2 (as used in the treatment of Plaque psoriasis, GVHD); Otelixizumab (TRX4; Tolerx/GlaxoSmithKline) targeting CD3 (as used in the treatment of T1D); Teplizumab (MGA031; MacroGenics/Eli Lilly) targeting CD3 (as used in the treatment of T1D); Rituximab (Rituxan/Mabthera; Genentech/Roche/Biogen Idec) targeting CD20 (as used in the treatment of Non-Hodgkin's lymphoma, RA (in patients with inadequate responses to TNF blockade) and CLL); Ofatumumab (Arzerra; Genmab/GlaxoSmithKline) targeting CD20 (as used in the treatment of CLL, RA); Ocrelizumab (2H7; Genentech/Roche/Biogen Idec) targeting CD20 (as used in the treatment of RA and SLE); Epratuzumab (hLL2; Immunomedics/UCB) targeting CD22 (as used in the treatment of SLE and non-Hodgkin's lymphoma); Alemtuzumab (Campath/MabCampath; Genzyme/Bayer) targeting CD52 (as used in the treatment of CLL, MS); Abatacept (Orencia; Bristol-Myers Squibb) targeting CD80 and CD86 (as used in the treatment of RA and JIA, UC and Crohn's disease, SLE); Eculizumab (Soliris; Alexion pharmaceuticals) targeting C5 complement protein (as used in the treatment of Paroxysmal nocturnal haemoglobinuria); Omalizumab (Xolair; Genentech/Roche/Novartis) targeting IgE (as used in the treatment of Moderate to severe persistent allergic asthma); Canakinumab (Ilaris; Novartis) targeting IL-10 (as used in the treatment of Cryopyrin-associated periodic syndromes, Systemic JIA, neonatal-onset multisystem inflammatory disease and acute gout); Mepolizumab (Bosatria; GlaxoSmithKline) targeting IL-5 (as used in the treatment of Hyper-eosinophilic syndrome); Reslizumab (SCH55700; Ception Therapeutics) targeting IL-5 (as used in the treatment of Eosinophilic oesophagitis); Tocilizumab (Actemra/RoActemra; Chugai/Roche) targeting IL-6R (as used in the treatment of RA, JIA); Ustekinumab (Stelara; Centocor) targeting IL-12 and IL-23 (as used in the treatment of Plaque psoriasis, Psoriatic arthritis, Crohn's disease); Briakinumab (ABT-874; Abbott) targeting IL-12 and IL-23 (as used in the treatment of Psoriasis and plaque psoriasis); Etanercept (Enbrel; Amgen/Pfizer) targeting TNF (as used in the treatment of RA, JIA, psoriatic arthritis, AS and plaque psoriasis); Infliximab (Remicade; Centocor/Merck) targeting TNF (as used in the treatment of Crohn's disease, RA, psoriatic arthritis, UC, AS and plaque psoriasis); Adalimumab (Humira/Trudexa; Abbott) targeting TNF (as used in the treatment of RA, JIA, psoriatic arthritis, Crohn's disease, AS and plaque psoriasis); Certolizumab pegol (Cimzia; UCB) targeting TNF (as used in the treatment of Crohn's disease and RA); Golimumab (Simponi; Centocor) targeting TNF (as used in the treatment of RA, psoriatic arthritis and AS); and the like. In some cases, the antibody whose production is induced by the intracellular domain of a synNotch polypeptide of the present disclosure is a therapeutic antibody for the treatment of cancer. Such antibodies include, e.g., Ipilimumab targeting CTLA-4 (as used in the treatment of Melanoma, Prostate Cancer, RCC); Tremelimumab targeting CTLA-4 (as used in the treatment of CRC, Gastric, Melanoma, NSCLC); Nivolumab targeting PD-1 (as used in the treatment of Melanoma, NSCLC, RCC); MK-3475 targeting PD-1 (as used in the treatment of Melanoma); Pidilizumab targeting PD-1 (as used in the treatment of Hematologic Malignancies); BMS-936559 targeting PD-LI (as used in the treatment of Melanoma, NSCLC, Ovarian, RCC); MEDI4736 targeting PD-Li; MPDL33280A targeting PD-L1 (as used in the treatment of Melanoma); Rituximab targeting CD20 (as used in the treatment of Non-Hodgkin's lymphoma); Ibritumomab tiuxetan and tositumomab (as used in the treatment of Lymphoma); Brentuximab vedotin targeting CD30 (as used in the treatment of Hodgkin's lymphoma); Gemtuzumab ozogamicin targeting CD33 (as used in the treatment of Acute myelogenous leukaemia); Alemtuzumab targeting CD52 (as used in the treatment of Chronic lymphocytic leukaemia); IGN101 and adecatumumab targeting EpCAM (as used in the treatment of Epithelial tumors (breast, colon and lung)); Labetuzumab targeting CEA (as used in the treatment of Breast, colon and lung tumors); huA33 targeting gpA33 (as used in the treatment of Colorectal carcinoma); Pemtumomab and oregovomab targeting Mucins (as used in the treatment of Breast, colon, lung and ovarian tumors); CC49 (minretumomab) targeting TAG-72 (as used in the treatment of Breast, colon and lung tumors); cG250 targeting CAIX (as used in the treatment of Renal cell carcinoma); J591 targeting PSMA (as used in the treatment of Prostate carcinoma); MOv18 and MORAb-003 (farletuzumab) targeting Folate-binding protein (as used in the treatment of Ovarian tumors); 3F8, ch14.18 and KW-2871 targeting Gangliosides (such as GD2, GD3 and GM2) (as used in the treatment of Neuroectodermal tumors and some epithelial tumors); hu3S193 and IgN3II targeting Le y (as used in the treatment of Breast, colon, lung and prostate tumors); Bevacizumab targeting VEGF (as used in the treatment of Tumor vasculature); IM-2C6 and CDP791 targeting VEGFR (as used in the treatment of Epithelium-derived solid tumors); Etaracizumab targeting Integrin_V_3 (as used in the treatment of Tumor vasculature); Volociximab targeting Integrin 51 (as used in the treatment of Tumor vasculature); Cetuximab, panitumumab, nimotuzumab and 806 targeting EGFR (as used in the treatment of Glioma, lung, breast, colon, and head and neck tumors); Trastuzumab and pertuzumab targeting ERBB2 (as used in the treatment of Breast, colon, lung, ovarian and prostate tumors); MM-121 targeting ERBB3 (as used in the treatment of Breast, colon, lung, ovarian and prostate, tumors); AMG 102, METMAB and SCH 900105 targeting MET (as used in the treatment of Breast, ovary and lung tumors); AVE1642, IMC-A12, MK-0646, R1507 and CP 751871 targeting IGF1R (as used in the treatment of Glioma, lung, breast, head and neck, prostate and thyroid cancer); KB004 and IIIA4 targeting EPHA3 (as used in the treatment of Lung, kidney and colon tumors, melanoma, glioma and haematological malignancies); Mapatumumab (HGS-ETR1) targeting TRAILR1 (as used in the treatment of Colon, lung and pancreas tumors and haematological malignancies); HGS-ETR2 and CS-1008 targeting TRAILR2; Denosumab targeting RANKL (as used in the treatment of Prostate cancer and bone metastases); Sibrotuzumab and F19 targeting FAP (as used in the treatment of Colon, breast, lung, pancreas, and head and neck tumors); 81C6 targeting Tenascin (as used in the treatment of Glioma, breast and prostate tumors); Blinatumomab (Blincyto; Amgen) targeting CD3 (as used in the treatment of ALL); pembrolizumab targeting PD-1 as used in cancer immunotherapy; 9E10 antibody targeting c-Myc; and the like.

Suitable antibodies include, e.g., Abagovomab, Abciximab, Abituzumab, Abrilumab, Actoxumab, Aducanumab, Afelimomab, Afutuzumab, Alacizumab pegol, ALD518, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab/tocilizumab, Atorolimumab, Bapineuzumab, Basiliximab, Bavituximab, Bectumomab, Begelomab, Benralizumab, Bertilimumab, Besilesomab, Bevacizumab/Ranibizumab, Bezlotoxumab, Biciromab, Bimagrumab, Bimekizumab, Bivatuzumab mertansine, Blosozumab, Bococizumab, Brentuximabvedotin, Brodalumab, Brolucizumab, Brontictuzumab, Cantuzumab mertansine, Cantuzumab ravtansine, Caplacizumab, Capromab pendetide, Carlumab, Catumaxomab, cBR96-doxorubicin immunoconjugate, Cedelizumab, Ch. 14.18, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Dacetuzumab, Daclizumab, Dalotuzumab, Dapirolizumab pegol, Daratumumab, Dectrekumab, Demcizumab, Denintuzumab mafodotin, Derlotuximab biotin, Detumomab, Dinutuximab, Diridavumab, Dorlimomab aritox, Drozitumab, Duligotumab, Dupilumab, Durvalumab, Dusigitumab, Ecromeximab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab vedotin, Enlimomab pegol, Enoblituzumab, Enokizumab, Enoticumab, Ensituximab, Epitumomab cituxetan, Erlizumab, Ertumaxomab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gevokizumab, Girentuximab, Glembatumumab vedotin, Gomiliximab, Guselkumab, Ibalizumab, Ibalizumab, Icrucumab, Idarucizumab, Igovomab, IMAB362, Imalumab, Imciromab, Imgatuzumab, Inclacumab, Indatuximab ravtansine, Indusatumab vedotin, Inolimomab, Inotuzumab ozogamicin, Intetumumab, Iratumumab, Isatuximab, Itolizumab, Ixekizumab, Keliximab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Margetuximab, Maslimomab, Matuzumab, Mavrilimumab, Metelimumab, Milatuzumab, Minretumomab, Mirvetuximab soravtansine, Mitumomab, Mogamulizumab, Morolimumab, Morolimumab immune, Motavizumab, Moxetumomab pasudotox, Muromonab-CD3, Nacolomab tafenatox, Namilumab, Naptumomab estafenatox, Narnatumab, Nebacumab, Necitumumab, Nemolizumab, Nerelimomab, Nesvacumab, Nofetumomab merpentan, Obiltoxaximab, Obinutuzumab, Ocaratuzumab, Odulimomab, Olaratumab, Olokizumab, Onartuzumab, Ontuxizumab, Opicinumab, Oportuzumab monatox, Orticumab, Otlertuzumab, Oxelumab, Ozanezumab, Ozoralizumab, Pagibaximab, Palivizumab, Pankomab, Panobacumab, Parsatuzumab, Pascolizumab, Pasotuxizumab, Pateclizumab, Patritumab, Perakizumab, Pexelizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Rilotumumab, Rinucumab, Robatumumab, Roledumab, Romosozumab, Rontalizumab, Rovelizumab, Ruplizumab, Sacituzumab govitecan, Samalizumab, Sarilumab, Satumomab pendetide, Secukinumab, Seribantumab, Setoxaximab, Sevirumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teprotumumab, Tesidolumab, Tetulomab, TGN1412, Ticilimumab/tremelimumab, Tigatuzumab, Tildrakizumab, TNX-650, Toralizumab, Tosatoxumab, Tovetumab, Tralokinumab, TRBS07, Tregalizumab, Trevogrumab, Tucotuzumab celmoleukin, Tuvirumab, Ublituximab, Ulocuplumab, Urelumab, Urtoxazumab, Vandortuzumab vedotin, Vantictumab, Vanucizumab, Vapaliximab, Varlilumab, Vatelizumab, Veltuzumab, Vepalimomab, Vesencumab, Visilizumab, Vorsetuzumab mafodotin, Votumumab, Zalutumumab, Zanolimumab, Zatuximab, Ziralimumab, Zolimomab aritox, and the like.

### Pseudotyping Envelope Proteins

As noted above, a VLP of the present disclosure comprises a pseudotyping envelope protein (e.g., a glycoprotein) and/or a polypeptide that provides for binding to a target cell.

Pseudotyped VLPs include heterologous glycoproteins derived from an enveloped virus other than the virus from which the MA, CA, and NC polypeptides are derived. Such a pseudotyped VLP can be targeted to a cell, tissue, or organ that is targeted by the virus from which the heterologous glycoproteins are derived. A pseudotyped VLP can include, e.g., as the heterologous virus protein used for the pseudotyping, a viral envelope protein selected from a vesicular stomatitis virus (VSV) glycoprotein (VSV-G protein), a Measles virus hemagglutinin (HA) protein and/or a measles virus fusion glycoprotein, Influenza virus neuraminidase (NA) protein, a Measles virus F protein, an Influenza virus HA protein, Moloney virus MLV-A protein, a Moloney virus MLV-E protein, a Baboon Endogenous retrovirus (BAEV) envelope protein, an Ebola virus glycoprotein, a foamy virus envelope protein, or a combination or two or more of the foregoing viral envelope proteins.

In some cases, a VSV-G protein is specifically excluded. In some cases, a measles virus hemagglutinin protein is specifically excluded. In some cases, a measles virus F protein is specifically excluded. In some cases, an influenza virus hemagglutinin protein is specifically excluded. In some cases, a Moloney virus MLV-A protein is specifically excluded. In some cases, a Moloney virus MLV-E protein is specifically excluded. In some cases, a baboon endogenous retrovirus envelope protein is specifically excluded. In some cases, an Ebola virus glycoprotein is specifically excluded. In some cases, a foamy virus envelop protein is specifically excluded.

In some cases, the heterologous glycoprotein used for pseudotyping is a VSV-G protein. A suitable VSV-G protein comprises an amino acid sequence having at least 80%, at least 85%, at least 90%, at least 95%, at least 98%, at least 99%, or 100%, amino acid sequence identity to the following amino acid sequence:

(SEQ ID NO: 48)
IMKCLLYLAFLFIGVNCKFTIVFPHNQKGNWKNVPSNYHYCPSSSDLNWH
NDLIGTALQVKMPKSHKAIQADGWMCHASKWVTTCDFRWYGPKYITHSIR
SFTPSVEQCKESIEQTKQGTWLNPGFPPQSCGYATVTDAEAVIVQVTPHH
VLVDEYTGEWVDSQFINGKCSNYICPTVHNSTTWHSDYKVKGLCDSNLIS
MDITFFSEDGELSSLGKEGTGFRSNYFAYETGGKACKMQYCKHWGVRLPS
GVWFEMADKDLFAAARFPECPEGSSISAPSQTSVDVSLIQDVERILDYSL
CQETWSKIRAGLPISPVDLSYLAPKNPGTGPAFTIINGTLKYFETRYIRV
DIAAPILSRMVGMISGTTTERELWDDWAPYEDVEIGPNGVLRTSSGYKFP
LYMIGHGMLDSDLHLSSKAQVFEHPHIQDAASQLPDDESLFFGDTGLSKN
PIELVEGWFSSWKSSIASFFFIIGLIIGLFLVLRVGIHLCIKLKHTKKRQ
IYTDIEMNRLGK.

In some cases, the heterologous glycoprotein used for pseudotyping is a BAEV-G protein. A suitable BAEV-G protein comprises an amino acid sequence having at least 80%, at least 85%, at least 90%, at least 95%, at least 98%, at least 99%, or 100%, amino acid sequence identity to the following amino acid sequence:

(SEQ ID NO: 49)
MGFTTKIIFLYNLVLVYAGFDDPRKAIELVQKRYGRPCDCSGGQVSEPPS
DRVSQVTCSGKTAYLMPDQRWKCKSIPKDTSPSGPLQECPCNSYQSSVHS
SCYTSYQQCRSGNKTYYTATLLKTQTGGTSDVQVLGSTNKLIQSPCNGIK
GQSICWSTTAPIHVSDGGGPLDTTRIKSVQRKLEEIHKALYPELQYHPLA
IPKVRDNLMVDAQTLNILNATYNLLLMSNTSLVDDCWLCLKLGPPTPLAI
PNFLLSYVTRSSDNISCLIIPPLLVQPMQFSNSSCLFSPSYNSTEEIDLG
HVAFSNCTSITNVTGPICAVNGSVFLCGNNMAYTYLPTNWTGLCVLATLL
PDIDIIPGDEPVPIPAIDHFIYRPKRAIQFIPLLAGLGITAAFTTGATGL
GVSVTQYTKLSNQLISDVQILSSTIQDLQDQVDSLAEVVLQNRRGLDLLT
AEQGGICLALQEKCCFYVNKSGIVRDKIKTLQEELERRRKDLASNPLWTG
LQGLLPYLLPFLGPLLTLLLLLTIGPCIFNRLTAFINDKLNIIHAMVLTQ
QYQVLRTDEEAQD.

In some cases, the heterologous glycoprotein used for pseudotyping is an influenza virus HIN1 hemagglutinin glycoprotein. A suitable influenza hemagglutinin protein comprises an amino acid sequence having at least 80%, at least 85%, at least 90%, at least 95%, at least 98%, at least 99%, or 100%, amino acid sequence identity to the following amino acid sequence: MKAILVVLLY TFATANADTL CIGYHANNST DTVDTVLEKN VTVTHSVNLL EDKHNGKLCK LRGVAPLHLG KCNIAGWILG NPECESLSTA SSWSYIVETP SSDNGTCYPG DFIDYEELRE QLSSVSSFER FEIFPKTSSW PNHDSNKGVT AACPHAGAKS FYKNLIWLVK KGNSYPKLSK SYINDKGKEV LVLWGIHHPS TSADQQSLYQ NADAYVFVGS SRYSKKFKPE IAIRPKVRXX EGRMNYYWTL VEPGDKITFE ATGNLVVPRY AFAMERNAGS GIIISDTPVH DCNTTCQTPK GAINTSLPFQ NIHPITIGKC PKYVKSTKLR LATGLRNIPS IQSRGLFGAI AGFIEGGWTG MVDGWYGYHH QNEQGSGYAA DLKSTQNAID EITNKVNSVI EKMNTQFTAV GKEFNHLEKR IENLNKKVDD GFLDIWTYNA ELLVLLENER TLDYHDSNVK NLYEKVRSQL KNNAKEIGNG CFEFYHKCDN TCMESVKNGT YDYPKYSEEA KLNREEIDGV KLESTRIYQI LAIYSTVASS LVLVVSLGAI SFWMCSNGSL QCRICI (SEQ ID NO:50; GenBank Accession No: ACP44189). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and natural killer (NK) cells.

In some cases, the heterologous glycoprotein used for pseudotyping is an influenza virus H3N2 hemagglutinin glycoprotein. A suitable influenza hemagglutinin protein comprises an amino acid sequence having at least 80%, at least 85%, at least 90%, at least 95%, at least 98%, at least 99%, or 100%, amino acid sequence identity to the following amino acid sequence: MKTIIALSYI LCLVFAQKLP GNDNSTATLC LGHHAVPNGT IVKTITNDQI EVTNATELVQ SSSTGGICDS PHQILDGENC TLIDALLGDP QCDGFQNKKW DLFVERSKAY SNCYPYDVPD YASLRSLVAS SGTLEFNNES FNWTGVTQNG TSSACKRRSN NSFFSRLNWL THLKFKYPAL NVTMPNNEKF DKLYIWGVHH PGTDNDQISL YAQASGRITV STKRSQQTVI PSIGSRPRIR DVPSRISIYW TIVKPGDILL INSTGNLIAP RGYFKIRSGK SSIMRSDAPI GKCNSECITP NGSIPNDKPF QNVNRITYGA CPRYVKQNTL KLATGMRNVP EKQTRGIFGA IAGFIENGWE GMVDGWYGFR HQNSEGTGQA ADLKSTQAAI NQINGKLNRL IGKTNEKFHQ IEKEFSEVEG RIQDLEKYVE DTKIDLWSYN AELLVALENQ HTIDLTDSEM NKLFERTKKQ LRENAEDMGN GCFKIYHKCD NACIGSIRNG TYDHDVYRDE ALNNRFQIKG VELKSGYKDW ILWISFAISC FLLCVALLGF IMWACQKGNI RCNICI (SEQ ID NO:51; GenBank Accession No: YP_308839). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and natural killer (NK) cells.

In some cases, the heterologous glycoprotein used for pseudotyping is an influenza virus A H5N1 hemagglutinin glycoprotein. A suitable influenza hemagglutinin protein comprises an amino acid sequence having at least 80%, at least 85%, at least 90%, at least 95%, at least 98%, at least 99%, or 100%, amino acid sequence identity to the following amino acid sequence: MEKIVLLLAI VSLVKSDQIC IGYHANNSTE QVDTIMEKNV TVTHAQDILE KTHNGKLCDL NGVKPLILRD CSVAGWLLGN PMCDEFINVP EWSYIVEKAS PANDLCYPGD FNDYEELKHL LSRTNHFEKI QIIPKSSWSN HDASSGVSSA CPYHGRSSFF RNVVWLIKKN SAYPTIKRSY NNTNQEDLLV LWGIHHPNDA AEQTKLYQNP TTYISVGTST LNQRLVPEIA TRPKVNGQSG RMEFFWTILK PNDAINFESN GNFIAPEYAY KIVKKGDSAI MKSELEYGNC NTKCQTPMGA INSSMPFHNI HPLTIGECPK YVKSNRLVLA TGLRNTPQRE RRRKKRGLFG AIAGFIEGGW QGMVDGWYGY HHSNEQGSGY AADKESTQKA IDGVTNKVNS IIDKMNTQFE AVGREFNNLE RRIENLNKQM EDGFLDVWTY NAELLVLMEN ERTLDFHDSN VKNLYDKVRL QLRDNAKELG NGCFEFYHKC DNECMESVKN GTYDYPQYSE EARLNREEIS GVKLESMGTY QILSIYSTVA SSLALAIMVA GLSLWMCSNG SLQCRICI (SEQ ID NO:52; GenBank Accession No: YP_308669). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is an influenza virus H7N9 hemagglutinin glycoprotein. A suitable influenza hemagglutinin protein comprises an amino acid sequence having at least 80%, at least 85%, at least 90%, at least 95%, at least 98%, at least 99%, or 100%, amino acid sequence identity to the following amino acid sequence: MNTQILVFAL IAIIPTNADK ICLGHHAVSN GTKVNTLTER GVEVVNATET VERTNIPRIC SKGKRTVDLG QCGLLGTITG PPQCDQFLEF SADLIIERRE GSDVCYPGKF VNEEALRQIL RESGGIDKEA MGFTYSGIRT NGATSACRRS GSSFYAEMKW LLSNTDNAAF PQMTKSYKNT RKSPALIVWG IHHSVSTAEQ TKLYGSGNKL VTVGSSNYQQ SFVPSPGARP QVNGLSGRID FHWLMLNPND TVTFSFNGAF IAPDRASFLR GKSMGIQSGV QVDANCEGDC YHSGGTIISN LPFQNIDSRA VGKCPRYVKQ RSLLLATGMK NVPEIPKGRG LFGAIAGFIE NGWEGLIDGW YGFRHQNAQG EGTAADYKST QSAIDQITGK LNRLIEKTNQ QFELIDNEFN EVEKQIGNVI NWTRDSITEV WSYNAELLVA MENQHTIDLA DSEMDKLYER VKRQLRENAE EDGTGCFEIF HKCDDDCMAS IRNNTYDHSK YREEAMQNRI QIDPVKLSSG YKDVILWFSF GASCFILLAI VMGLVFICVK NGNMRCTICI (SEQ ID NO:53; GenBank Accession No: YP_009118475). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is a Hepatitis B Virus (HBV) S glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MENTTSGFLG PLLVLQAGFF LLTRNLTIPQ SLDSWWTSLN FLGGAPTCPG QNSQSPTSNH SPTSCPPICP GYRWMCLRRF IIFLFILLLC LIFLLVLLDY QGMLPVCPLL PGTSTTSTGP CKTCTIPAQG TSMFPSCCCT KPSDGNCTCI PIPSSWAFAR FLWEWASVRF SWLSLLVPFV QWFVGLSPTV WLSVIWMMWY WGPSLYNILS PFLPLLPIFF CLWVYI (SEQ ID NO:54; GenBank Accession No: ABV02793). Such a heterologous glycoprotein may be useful in directing a VLP of the present disclosure to a liver cell.

In some cases, the heterologous glycoprotein used for pseudotyping is a Hepatitis B Virus (HBV) middle S glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MQWNSTAFHQ ALQDPKVRGL YFPAGGSSSG TVNPAPNIAS HISSISARTG DPVTNMENIT SGFLGPLLVL QAGFFLLTRI LTIPQSLDSW WTSLNFLGGS PVCLGQNSQS PTSNHSPTSC PPICPGYRWM CLRRFIIFLF ILLLCLIFLL VLLDYQGMLP VCPLIPGSTT TSTGPCKTCT TPAQGNSMFP SCCCTKPTDG NCTCIPIPSS WAFAKYLWEW ASVRFSWLSL LVPFVQWFVG LSPTVWLSAI WMMWYWGPSL YSIVSPFIPL LPIFFCLWVY I (SEQ ID NO:55; GenBank Accession No: ACJ66136). Such a heterologous glycoprotein may be useful in directing a VLP of the present disclosure to a liver cell.

In some cases, the heterologous glycoprotein used for pseudotyping is a Hepatitis B Virus (HBV) large S glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MGLSWTVPLE WGKNHSTTNP LGFFPDHQLD PAFRANTRNP DWDHNPNKDH WTEANKVGVG AFGPGFTPPH GGLLGWSPQA QGMLKTLPAD PPPASTNRQS GRQPTPITPP LRDTHPQAMQ WNSTTFHQAL QDPKVSALYL PAGGSSSGTV NPVPTTASLI SSIFSRIGDP APNMESITSG FLGPLLVLQA GFFLLTKILT IPQSLDSWWT SLNFLGGAPV CLGQNSQSPT SSHSPTSCPP ICPGYRWMCL RRFIIFLFIL LLCLIFLLVL LDYQGMLPVC PLIPGSSTTS TGPCRTCTTL AQGTSMFPSC CCSKPSDGNC TCIPIPSSWA FGKFLWEWAS ARFSWLSLLV PFVQWFAGLS PTVWLSVIWM MWYWGPSLYN ILSPFIPLLP IFFCLWVYI (SEQ ID NO:56; GenBank Accession No: AGR65633). Such a heterologous glycoprotein may be useful in directing a VLP of the present disclosure to a liver cell.

In some cases, the heterologous glycoprotein used for pseudotyping is a Hepatitis B Virus (HBV) small S glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MENITSGFLG PLLVLQAGFF LLTRILTIPQ SLDSWWTSLN FLGGTTVCLG QNSQSPTSNH SPTSCPPTCP GYRWMCLRRF IIFLFILLLC LIFLLVLLDY QGMLPVCPLI PGSSTTSTGP CRTCTTPAQG TSMYPSCCCT KPSDGNCTCI PIPSSWAFGK FLWEWASARF SWLSLLVPFV QWFVGLSPTV WLSVIWMMWY WAPNLHNILS PFLPLLPIFL CLWVYI (SEQ ID NO:57; GenBank Accession No: AHC69850. Such a heterologous glycoprotein may be useful in directing a VLP of the present disclosure to a liver cell.

In some cases, the heterologous glycoprotein used for pseudotyping is a Hepatitis B Virus (HBV) pre S glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MGGWSSKPRK GMGTNLAVPN PLGFFPDHQL DPAFKANSDN PDWDLNTHKD YWPDAWKVGV GAFGPGFTPP HGGLLGWSPQ AQGLLTTVPA APPPASTNRQ SGRQPTPLSP PLRDTHPQAM KWNSTTFHQT LQDPRVRALY LPAGGSSSGT VSPAQNTVSA ISSILSKTGD PVPNMESIAS GLLGPLLVLQ AGFFLLTKIL TIPQSLDSWW TSLNFLGGTP VCLGQNSQSQ ISSHSPTCCP PTCPGYRWMC LRRFIIFLCI LLLCLIFLLV LLDYQGMLPV CPLIPGSSTT STGPCKTCTA PAQGTSMFPS CCCTKPTDGN CTCIPIPSSW AFAKYLWEWA SVRFSWLSLL VPFVQWFVGL SPTVWLSVIW MMWFWGPSLY NILSPFIPLL PIFFCLWVYI (SEQ ID NO:58; GenBank Accession No: CAA66700). Such a heterologous glycoprotein may be useful in directing a VLP of the present disclosure to a liver cell.

In some cases, the heterologous glycoprotein used for pseudotyping is a Hepatitis B Virus (HBV) preS2 glycoprotein. A suitable HBV protein comprises an amino acid sequence having at least 80%, at least 85%, at least 90%, at least 95%, at least 98%, at least 99%, or 100%, amino acid sequence identity to the following amino acid sequence: MQWNSTTFHQ TLQDPRVRGL YFPAGGSSSG TVNPVPTTVS HISSIFSRIG DPALNMENIT SGFLGPLLVL QAGFFLLTRI LTIPQSLDSW WTSLNFLGGT TVCLGQNSQS PTSNHSPTSC PPTCPGYRWM CLRRFIIFLF ILLLCLIFLL VLLDYQGMLS VCPLIPGSTT TSTGPCKTCTTPAQGTSIHP SCCCTKPSDG NCTWIPIPSS WAFGKFLWEW ASARFSWLSL LVPFVQWFVG LSPTVWLSVI WIMWYWGPSL YSILSPFLPL LPIFFCLWVY I (SEQ ID NO:59; GenBank Accession No: AAO12662). Such a heterologous glycoprotein may be useful in directing a VLP of the present disclosure to a liver cell.

In some cases, the heterologous glycoprotein used for pseudotyping is a Rabies virus. A suitable Rabies virus protein comprises an amino acid sequence having at least 80%, at least 85%, at least 90%, at least 95%, at least 98%, at least 99%, or 100%, amino acid sequence identity to the following amino acid sequence: MVPQALLFVP LLVFPLCFGK FPIYTIPDKL GPWSPIDIHH LSCPNNLVVE DEGCTNLSGF SYMELKVGYI SAIKVNGFTC TGVVTEAETY TNFVGYVTTT FKRKHFRPTP DACRSAYNWK MAGDPRYEES LHNPYPDYHW LRTVKTTKES LVIISPSVAD LDPYDKSLHS RVFPSGKCSG ITVSSTYCST NHDYTIWMPE NLRLGTSCDI FINSRGKRAS KGSQTCGFID ERGLYKSLKG ACKLKLCGVL GLRLMDGTWV AMQTSDETKW CPPDQLVNLH DFRSDEIEHL VVEELVKKRE ECLDALESIM TTKSVSFRRL SHLRKLVPGF GKAYTIFNKT LMEADAHYKS VRTWNEIIPS KGCLRVGGRC HPHVNGVFFN GIILGPEGHV LIPEMQSSLL QQHMELLESS VIPLMHPLAD PSTVFKEGDE AEDFVEVHLP DVHKQVSGVN LGLPNWGKYV LLSAGALIAL MLIIFLLTCC RRVNRPESTQ HSLGGKRRKV SITSQSGKII SSWESYKSGG ETRL (SEQ ID NO:60; GenBank Accession No: AWR88358). Such a glycoprotein may be useful for targeting a VLP of the present disclosure to neurons, astrocytes, oligodendrocyctes, glia, and other cells of the of the central nervous system.

In some cases, the heterologous glycoprotein used for pseudotyping is a Mokola virus glycoprotein. A suitable Mokola virus protein comprises an amino acid sequence having at least 80%, at least 85%, at least 90%, at least 95%, at least 98%, at least 99%, or 100%, amino acid sequence identity to the following amino acid sequence: MNIPCFVVIL SLATTHSLGE FPLYTIPEKI EKWTPIDMIH LSCPNNLLSE EEGCNAESSF TYFELKSGYL AHQKVPGFTC TGVVNEAETY TNFVGYVTTT FKRKHFRPTV AACRDAYNWK VSGDPRYEES LHTPYPDSSW LRTVTTTKES LLIISPSIVE MDIYGRTLHS PMFPSGVCSN VYPSVPSCET NHDYTLWLPE DPSLSLVCDI FTSSNGKKAM NGSRICGFKD ERGFYRSLKG ACKLTLCGRP GIRLFDGTWV SFTKPDVHVW CTPNQLINIH NDRLDEIEHL IVEDIIKKRE ECLDTLETIL MSQSVSFRRL SHFRKLVPGY GKAYTILNGS LMETNVYYKR VDKWADILPS KGCLKVGQQC MEPVKGVLFN GIIKGPDGQI LIPEMQSEQL KQHMDLLKAA VFPLRHPLIS REAVFKKDGD ADDFVDLHMP DVHKSVSDVD LGLPHWGFWM LIGATIVAFV VLVCLLRVCC KRVRRRRSGR ATQEIPLSFP SAPVPRAKVV SSWESYKGLP GT (SEQ ID NO:61; GenBank Accession No: AAB26292). Such a glycoprotein may be useful for targeting a VLP of the present disclosure to neurons, astrocytes, oligodendrocyctes, glia, and other cells of the of the central nervous system.

In some cases, the heterologous glycoprotein used for pseudotyping is a lymphocytic choriomeningitis virus (LCMV) glycoprotein. A suitable LCMV protein comprises an amino acid sequence having at least 80%, at least 85%, at least 90%, at least 95%, at least 98%, at least 99%, or 100%, amino acid sequence identity to the following amino acid sequence: MGQIVTMFEA LPHIIDEVIN IVIIVLIIIT SIKAVYNFAT CGILALISFL LLAGRSCGLY GLDGPDIYKG IYQFKSVEFD MSHLNLTMPN ACSANNSHHY ISMGNSGLEL TFTNDSIISH NFCNLTSAFN KKTFDHTLMS IVSSLHLSIR GNSNYKAVSC DFNSGITIQY NLSFSDAQSA LSQCKTFRGR VLDMFRTAFG GKYMRSGWGW TGSDGKTTWC SQTSYQYLII QNRTWENHCR YAGPFGMARI LFAQEKTKFL TRRLAGTFTW TLSDSSGVDN PGGYCLTRWM ILAADLKCFG NTAVAKCNMN HDEEFCDMLR LIDYNKAALS KFKEDVESAL HLFKVTVNSL VSDQLLMRNH LRDLMGVPYC NYSRFWYLEH TKTGETSVPK CWLVTNGSYL NETHFSDQIE QEADNMITDM LRKDYIKRQG STPLALMDLL MFSTSAYLVS VFLHLVKIPT HRHIKGGSCP KPHRLTNKGI CSCGAFKVPG VKTVWKRR (SEQ ID NO:62; GenBank Accession No: AIW66623).

In some cases, the heterologous glycoprotein used for pseudotyping is a lymphocytic choriomeningitis virus (LCMV) glycoprotein C. A suitable LCMV protein comprises an amino acid sequence having at least 80%, at least 85%, at least 90%, at least 95%, at least 98%, at least 99%, or 100%, amino acid sequence identity to the following amino acid sequence: MGQIVTMFEA LPHIIDEVIN IVIIVLIIIT SIKAVYNFAT CGILALVSFL FLAGRSCGMY GLNGPDIYKG VYQFKSVEFD MSHLNLTMPN ACSANNSHHY ISMGSSGLEL TFTNDSILNH NFCNLTSAFN KKTFDHTLMS IVSSLHLSIR GNSNHKAVSC DFNNGITIQY NLSFSDPQSA ISQCRTFRGR VLDMFRTAFG GKYMRSGWGW AGSDGKTTWC SQTSYQYLII QNRTWENHCR YAGPFGMSRI LFAQEKTKFL TRRLAGTFTW TLSDSSGVEN PGGYCLTKWM ILAAELKCFG NTAVAKCNVN HDEEFCDMLR LIDYNKAALS KFKQDVESAL HVFKTTVNSL ISDQLLMRNH LRDLMGVPYC NYSKFWYLEH AKTGETSVPK CWLVTNGSYL NETHFSDQIE QEADNMITEM LRKDYIKRQG STPLALMDLL MFSTSAYLIS IFLHLVKIPT HRHIKGGSCP KPHRLTNKGI CSCGAFKVPG VKTIWKRR (SEQ ID NO:63; GenBank Accession No: CAC01231).

In some cases, the heterologous glycoprotein used for pseudotyping is a lymphocytic choriomeningitis virus (LCMV) glycoprotein. A suitable LCMV protein comprises an amino acid sequence having at least 80%, at least 85%, at least 90%, at least 95%, at least 98%, at least 99%, or 100%, amino acid sequence identity to the following amino acid sequence: MGQIVTMFEA LPHIIDEVIN IVIIVLIVIT GIKAVYNFAT CGIFALISFL LLAGRSCGMY GLKGPDIYKG VYQFKSVEFD MSHLNLTMPN ACSANNSHHY ISMGTSGLEL TFTNDSIISH NFCNLTSAFN KKTFDHTLMS IVSSLHLSIR GNSNYKAVSC DFNNGITIQY NLTFSDAQSA QSQCRTFRGR VLDMFRTAFG GKYMRSGWGW TGSDGKTTWC SQTSYQYLII QNRTWENHCT YAGPFGMSRI LLSQEKTKFF TRRLAGTFTW TLSDSSGVEN PGGYCLTKWM ILAAELKCFG NTAVAKCNVN HDAEFCDMLR LIDYNKAALS KFKEDVESAL HLFKTTVNSL ISDQLLMRNH LRDLMGVPYC NYSKFWYLEH AKTGETSVPK CWLVTNGSYL NETHFSDQIE QEADNMITEM LRKDYIKRQG STPLALMDLL MFSTSAYLVS IFLHLVKIPT HRHIKGGSCP KPHRLTNKGI CSCGAFKVPG VKTVWKRR (SEQ ID NO:64; GenBank Accession No: P09991).

In some cases, the heterologous glycoprotein used for pseudotyping is a lymphocytic choriomeningitis virus (LCMV) G1 glycoprotein. A suitable LCMV protein comprises an amino acid sequence having at least 80%, at least 85%, at least 90%, at least 95%, at least 98%, at least 99%, or 100%, amino acid sequence identity to the following amino acid sequence: MYGLKGPDIYKG VYQFKSVEFD MSHLNLTMPN ACSANNSHHY ISMGTSGLEL TFTNDSIISH NFCNLTSAFN KKTFDHTLMS IVSSLHLSIR GNSNYKAVSC DFNNGITIQY NLTFSDAQSA QSQCRTFRGR VLDMFRTAFG GKYMRSGWGW TGSDGKTTWC SQTSYQYLII QNRTWENHCT YAGPFGMSRI LLSQEKTKFF TRRLA (SEQ ID NO:65; GenBank Accession No: P09991).

In some cases, the heterologous glycoprotein used for pseudotyping is a lymphocytic choriomeningitis virus (LCMV) G2 glycoprotein. A suitable LCMV protein comprises an amino acid sequence having at least 80%, at least 85%, at least 90%, at least 95%, at least 98%, at least 99%, or 100%, amino acid sequence identity to the following amino acid sequence: GTFTW TLSDSSGVEN PGGYCLTKWM ILAAELKCFG NTAVAKCNVN HDAEFCDMLR LIDYNKAALS KFKEDVESAL HLFKTTVNSL ISDQLLMRNH LRDLMGVPYC NYSKFWYLEH AKTGETSVPK CWLVTNGSYL NETHFSDQIE QEADNMITEM LRKDYIKRQG STPLALMDLL MFSTSAYLVS IFLHLVKIPT HRHIKGGSCP KPHRLTNKGI CSCGAFKVPG VKTVWKRR (SEQ ID NO:66; GenBank Accession No: P09991).

In some cases, the heterologous glycoprotein used for pseudotyping is a Ross River virus E1 glycoprotein. A suitable Ross River virus protein comprises an amino acid sequence having at least 80%, at least 85%, at least 90%, at least 95%, at least 98%, at least 99%, or 100%, amino acid sequence identity to the following amino acid sequence: YEHTATIPNV VGFPYKAHIE RNXFSPMTLQ LEVVXXSLEP TLNLEYITCE YKTVVPSPFI KCCGTSECSS KEQPDYQCKV YTGVYPFMWG GAYCFCDSEN TQLSEAYVDR SDVCKHDHAL AYKAHTASLK ATIRISYGTI NQTTEAFVNG EHAVNVGGSK FIFGPISTAW SPFDNKIVVY KDDVYNQDFP PYGSGQPGRF GDIQSRTVES KDLYANTALK LSRPSPGVVH VPYTQTPSGF KYWLKEKGSS LNTKAPFGCK IKTNPVRAMD CAVGSIPVSM DIPDSAFTRV VDAPAVTDLS CQVAVCTHSS DFGXVATLSY KTDKPGKCAV HSHSNVATLQ EATVDVKEDG KVTVHFSXXS ASPAFKVSVC DAKTTCTAAC EPPKDHIVPY GASHNNQVFP DMSGTAMTWV QRMASGLGGL ALIAVVVLVL VTCITMRR (SEQ ID NO:67; GenBank Accession No: NP_740686). Such a glycoprotein may be useful for targeting a VLP of the present disclosure to skeletal muscle, and cells that make up the joints, joint-associated connective tissue, bone, neurons, and lymphatic cells.

In some cases, the heterologous glycoprotein used for pseudotyping is a Ross River virus E2 glycoprotein. A suitable Ross River virus protein comprises an amino acid sequence having at least 80%, at least 85%, at least 90%, at least 95%, at least 98%, at least 99%, or 100%, amino acid sequence identity to the following amino acid sequence: SVIEHFNVYK ATRPYLAXCA DCGDGYFCYS PVAIEKIRDE ASDGMLKIQV SAQIGLDKAG THAHTKMRYM AGHDVQESKR DSLRVYTSAA CSIHGTMGHF IVAHCPPGDY LKXSFEDANS HVKACKVQYK HDPLPVGREK FVVRPHFGVE LPCTSYQLTT APTDEEIDMH TPPDIPDRTL LSQTAGNVKI TAGGRTIRYN CTCGRDNVGT TSTDKTINTC KIDQCHAAVT SHDKWXFTSP FVPRADQTAR KGKVHVPFPL TNVTCRVPLA RAPDVTYGKK EVTLRLHPDH PTXFSYRSLG AVPHPYEEWV DKFSERIIPV TEEGIEYQWG NNPPVRLWAQ LTTEGKPHGW PHEIIQYYYG LYPAATIAAV SGASLMALLT LAATCCMLAT ARRKCLTPYA LTPGAVVPLT LGLLXCAPRA NA (SEQ ID NO:68; GenBank Accession No: NP_740684). Such a glycoprotein may be useful for targeting a VLP of the present disclosure to skeletal muscle, and cells that make up the joints, joint-associated connective tissue, bone, neurons, and lymphatic cells.

In some cases, the heterologous glycoprotein used for pseudotyping is a Semliki Forest virus E1 glycoprotein. A suitable Semliki Forest virus protein comprises an amino acid sequence having at least 80%, at least 85%, at least 90%, at least 95%, at least 98%, at least 99%, or 100%, amino acid sequence identity to the following amino acid sequence: YEHSTVMPNV VGFPYKAHIE RPGYSPLTLQ MQVVETSLEP TLNLEYITCE YKTVVPSPYV KCCGASECST KEKPDYQCKV YTGVYPFMWG GAYCFCDSEN TQLSEAYVDR SDVCRHDHAS AYKAHTASLK AKVRVMYGNV NQTVDVYVNG DHAVTIGGTQ FIFGPLSSAW TPFDNKIVVY KDEVFNQDFP PYGSGQPGRF GDIQSRTVES NDLYANTALK LARPSPGMVH VPYTQTPSGF KYWLKEKGTA LNTKAPFGCQ IKTNPVRAMN CAVGNIPVSM NLPDSAFTRI VEAPTIIDLT CTVATCTHSS DFGGVLTLTY KTNKNGDCSV HSHSNVATLQ EATAKVKTAG KVTLHFSTAS ASPSFVVSLC SARATCSASC EPPKDHIVPY AASHSNVVFP DMSGTALSWV QKISGGLGAF AIGAILVLVV VTCIGLRR (SEQ ID NO:69; GenBank Accession No: NP_819008). Such a glycoprotein may be useful for targeting a VLP of the present disclosure to muscle, pancreas, neurons, astrocytes, oligodendrocyctes, glia, and other cells of the of the central nervous system.

In some cases, the heterologous glycoprotein used for pseudotyping is a Semliki Forest virus E2 glycoprotein. A suitable Semliki Forest virus protein comprises an amino acid sequence having at least 80%, at least 85%, at least 90%, at least 95%, at least 98%, at least 99%, or 100%, amino acid sequence identity to the following amino acid sequence: SVSQHFNVYK ATRPYIAYCA DCGAGHSCHS PVAIEAVRSE ATDGMLKIQF SAQIGIDKSD NHDYTKIRYA DGHAIENAVR SSLKVATSGD CFVHGTMGHF ILAKCPPGEF LQVSIQDTRN AVRACRIQYH HDPQPVGREK FTIRPHYGKE IPCTTYQQTT AETVEEIDMH MPPDTPDRTL LSQQSGNVKI TVGGKKVKYN CTCGTGNVGT TNSDMTINTC LIEQCHVSVT DHKKWQFNSP FVPRADEPAR KGKVHIPFPL DNITCRVPMA REPTVIHGKR EVTLHLHPDH PTLFSYRTLG EDPQYHEEWV TAAVERTIPV PVDGMEYHWG NNDPVRLWSQ LTTEGKPHGW PHQIVQYYYG LYPAATVSAV VGMSLLALIS IFASCYMLVA ARSKCLTPYA LTPGAAVPWT LGILCCAPRA HA (SEQ ID NO:216; GenBank Accession No: NP_819006). Such a glycoprotein may be useful for targeting a VLP of the present disclosure to muscle, pancreas, neurons, astrocytes, oligodendrocyctes, glia, and other cells of the of the central nervous system.

In some cases, the heterologous glycoprotein used for pseudotyping is a Sindbis virus E1 glycoprotein. A suitable Sindbis virus protein comprises an amino acid sequence having at least 80%, at least 85%, at least 90%, at least 95%, at least 98%, at least 99%, or 100%, amino acid sequence identity to the following amino acid sequence: YEHATTVPNV PQIPYKALVE RAGYAPLNLE ITVMSSEVLP STNQEYITCK FTTVVPSPKI KCCGSLECQP AAHADYTCKV FGGVYPFMWG GAQCFCDSEN SQMSEAYVEL SADCASDHAQ AIKVHTAAMK VGLRIVYGNT TSFLDVYVNG VTPGTSKDLK VIAGPISASF TPFDHKVVIH RGLVYNYDFP EYGAMKPGAF GDIQATSLTS KDLIASTDIR LLKPSAKNVH VPYTQASSGF EMWKNNSGRP LQETAPFGCK IAVNPLRAVD CSYGNIPISI DIPNAAFIRT SDAPLVSTVK CEVSECTYSA DFGGMATLQY VSDREGQCPV HSHSSTATLQ ESTVHVLEKG AVTVHFSTAS PQANFIVSLC GKKTTCNAEC KPPADHIVST PHKNDQEFQA AISKTSWSWL FALFGGASSL LIIGLMIFAC SMMLTSTRR (SEQ ID NO:70; GenBank Accession No: NP_740677). Such a glycoprotein may be useful for targeting a VLP of the present disclosure to muscle, pancreas, neurons, astrocytes, oligodendrocyctes, glia, and other cells of the of the central nervous system.

In some cases, the heterologous glycoprotein used for pseudotyping is a Sindbis virus E2 glycoprotein. A suitable Sindbis virus protein comprises an amino acid sequence having at least 80%, at least 85%, at least 90%, at least 95%, at least 98%, at least 99%, or 100%, amino acid sequence identity to the following amino acid sequence: SVIDDFTLTS PYLGTCSYCH HTVPCFSPVK IEQVWDEADD NTIRIQTSAQ FGYDQSGAAS ANKYRYMSLK QDHTVKEGTM DDIKISTSGP CRRLSYKGYF LLAKCPPGDS VTVSIVSSNS ATSCTLARKI KPKFVGREKY DLPPVHGKKI PCTVYDRLKE TTAGYITMHR PRPHAYTSYL EESSGKVYAK PPSGKNITYE CKCGDYKTGT VSTRTEITGC TAIKQCVAYK SDQTKWVFNS PDLIRHDDHT AQGKLHLPFK LIPSTCMVPV AHAPNVIHGF KHISLQLDTD HLTLLTTRRL GANPEPTTEW IVGKTVRNFT VDRDGLEYIW GNHEPVRVYA QESAPGDPHG WPHEIVQHYY HRHPVYTILA VASATVAMMI GVTVAVLCAC KARRECLTPY ALAPNAVIPT SLALLCCVRS ANA (SEQ ID NO:71; GenBank Accession No: NP_740675). Such a glycoprotein may be useful for targeting a VLP of the present disclosure to skeletal muscle, and cells that make up the joints, joint-associated connective tissue, bone, neurons, and lymphatic cells.

In some cases, the heterologous glycoprotein used for pseudotyping is an Ebola Zaire virus glycoprotein. A suitable Ebola Zaire virus protein comprises an amino acid sequence having at least 80%, at least 85%, at least 90%, at least 95%, at least 98%, at least 99%, or 100%, amino acid sequence identity to the following amino acid sequence: MGVTGILQLP RDRFKRTSFF LWVIILFQRT FSIPLGVIHN STLQVSDVDK LVCRDKLSST NQLRSVGLNL EGNGVATDVP SATKRWGFRS GVPPKVVNYE AGEWAENCYN LEIKKPDGSE CLPAAPDGIR GFPRCRYVHK VSGTGPCAGD FAFHKEGAFF LYDRLASTVI YRGTTFAEGV VAFLILPQAK KDFFSSHPLR EPVNATEDPS SGYYSTTIRY QATGFGTNET EYLFEVDNLT YVQLESRFTP QFLLQLNETI YTSGKRSNTT GKLIWKVNPE IDTTIGEWAF WETKKNLTRK IRSEELSFTV VSNGAKNISG QSPARTSSDP GTNTTTEDHK IMASENSSAM VQVHSQGREA AVSHLTTLAT ISTSPQSLTT KPGPDNSTHN TPVYKLDISE ATQVEQHHRR TDNDSTASDT PSATTAAGPP KAENTNTSKS TDFLDPATTT SPQNHSETAG NNNTHHQDTG EESASSGKLG LITNTIAGVA GLITGGRRTR REAIVNAQPK CNPNLHYWTT QDEGAAIGLA WIPYFGPAAE GIYIEGLMHN QDGLICGLRQ LANETTQALQ LFLRATTELR TFSILNRKAI DFLLQRWGGT CHILGPDCCI EPHDWTKNIT DKIDQIIHDF VDKTLPDQGD NDNWWTGWRQ WIPAGIGVTG VIIAVIALFC ICKFVF (SEQ ID NO:72; GenBank Accession No: AAB81004). Such a glycoprotein may be useful for targeting a VLP of the present disclosure to hepatocytes, endothelial cells, dendritic cells, macrophages, and monocytes.

In some cases, the heterologous glycoprotein used for pseudotyping is an Ebola Zaire virus glycoprotein. A suitable Ebola Zaire virus protein comprises an amino acid sequence having at least 80%, at least 85%, at least 90%, at least 95%, at least 98%, at least 99%, or 100%, amino acid sequence identity to the following amino acid sequence: IPLGVIHN STLQVSDVDK LVCRDKLSST NQLRSVGLNL EGNGVATDVP SATKRWGFRS GVPPKVVNYE AGEWAENCYN LEIKKPDGSECLPAAPDGIR GFPRCRYVHK VSGTGPCAGD FAFHKEGAFF LYDRLASTVI YRGTTFAEGV VAFLILPQAK KDFFSSHPLR EPVNATEDPS SGYYSTTIRY QATGFGTNET EYLFEVDNLT YVQLESRFTP QFLLQLNETI YTSGKRSNTT GKLIWKVNPE IDTTIGEWAF WETKKNLTRK IRSEELSFTV VSNGAKNISG QSPARTSSDP GTNTTTEDHK IMASENSSAM VQVHSQGREA AVSHLTTLAT ISTSPQSLTT KPGPDNSTHN TPVYKLDISE ATQVEQHHRR TDNDSTASDT PSATTAAGPP KAENTNTSKS TDFLDPATTT SPQNHSETAG NNNTHHQDTG EESASSGKLG LITNTIAGVA GLITGGRRTR REAIVNAQPK CNPNLHYWTT QDEGAAIGLA WIPYFGPAAE GIYIEGLMHN QDGLICGLRQ LANETTQALQ LFLRATTELR TFSILNRKAI DFLLQRWGGT CHILGPDCCI EPHDWTKNIT DKIDQIIHDF VDKTLPDQGD NDNWWTGWRQ WIPAGIGVTG VIIAVIALFC ICKFVF (SEQ ID NO:73). Such a glycoprotein may be useful for targeting a VLP of the present disclosure to hepatocytes, endothelial cells, dendritic cells, macrophages, and monocytes.

In some cases, the heterologous glycoprotein used for pseudotyping is an Ebola Reston virus glycoprotein. A suitable Ebola Reston virus protein comprises an amino acid sequence having at least 80%, at least 85%, at least 90%, at least 95%, at least 98%, at least 99%, or 100%, amino acid sequence identity to the following amino acid sequence: MGSGYQLLQL PRERFRKTSF LVWVIILFQR AISMPLGIVT NSTLKATEID QLVCRDKLSS TSQLKSVGLN LEGNGIATDV PSATKRWGFR SGVPPKVVSY EAGEWAENCY NLEIKKSDGS ECLPLPPDGV RGFPRCRYVH KVQGTGPCPG DLAFHKNGAF FLYDRLASTV IYRGTTFAEG VVAFLILSEP KKHFWKATPA HEPVNTTDDS TSYYMTLTLS YEMSNFGGNE SNTLFKVDNH TYVQLDRPHT PQFLVQLNET LRRNNRLSNS TGRLTWTLDP KIEPDVGEWA FWETKKNFSQ QLHGENLHFQ IPSTHTNNSS DQSPAGTVQG KISYHPPANN SELVPTDSPP VVSVLTAGRT EEMSTQGLTN GETITGFTAN PMTTTIAPSP TMTSEVDNNV PSEQPNNTAS IEDSPPSASN ETIYHSEMDP IQGSNNSAQS PQTKTTPAPT TSPMTQDPQE TANSSKPGTS PGSAAGPSQP GLTINTVSKV ADSLSPTRKQ KRSVRQNTAN KCNPDLYYWT AVDEGAAVGL AWIPYFGPAA EGIYIEGVMH NQNGLICGLR QLANETTQAL QLFLRATTEL RTYSLLNRKA IDFLLQRWGG TCRILGPSCC IEPHDWTKNI TDEINQIKHD FIDNPLPDHG DDLNLWTGWR QWIPAGIGII GVIIAIIALL CICKILC (SEQ ID NO:74; GenBank Accession No: NP_690583). Such a glycoprotein may be useful for targeting a VLP of the present disclosure to hepatocytes, endothelial cells, dendritic cells, macrophages, and monocytes.

In some cases, the heterologous glycoprotein used for pseudotyping is a Marburg virus glycoprotein. A suitable Marburg virus protein comprises an amino acid sequence having at least 80%, at least 85%, at least 90%, at least 95%, at least 98%, at least 99%, or 100%, amino acid sequence identity to the following amino acid sequence: MKTTCFLISL ILIQGTKNLP ILEIASNNQP QNVDSVCSGT LQKTEDVHLM GFTLSGQKVA DSPLEASKRW AFRTGVPPKN VEYTEGEEAK TCYNISVTDP SGKSLLLDPP TNIRDYPKCK TIHHIQGQNP HAQGIALHLW GAFFLYDRIA STTMYRGKVF TEGNIAAMIV NKTVHKMIFS RQGQGYRHMN LTSTNKYWTS SNGTQTNDTG CFGALQEYNS TKNQTCAPSK IPPPLPTARP EIKLTSTPTD ATKLNTTDPS SDDEDLATSG SGSGEREPHT TSDAVTKQGL SSTMPPTPSP QPSTPQQGGN NTNHSQDAVT ELDKNNTTAQ PSMPPHNTTT ISTNNTSKHN FSTLSAPLQN TTNDNTQSTI TENEQTSAPS ITTLPPTGNP TTAKSTSSKK GPATTAPNTT NEHFTSPPPT PSSTAQHLVY FRRKRSILWR EGDMFPFLDG LINAPIDFDP VPNTKTIFDE SSSSGASAEE DQHASPNISL TLSYFPNINE NTAYSGENEN DCDAELRIWS VQEDDLAAGL SWIPFFGPGI EGLYTAVLIK NQNNLVCRLR RLANQTAKSL ELLLRVTTEE RTFSLINRHA IDFLLTRWGG TCKVLGPDCC IGIEDLSKNI SEQIDQIKKD EQKEGTGWGL GGKWWTSDWG VLTNLGILLL LSIAVLIALS CICRIFTKYI G (SEQ ID NO:75); GenBank Accession No: CAA78117). Such a glycoprotein may be useful for targeting a VLP of the present disclosure to hepatocytes, endothelial cells, dendritic cells, macrophages, and monocytes.

In some cases, the heterologous glycoprotein used for pseudotyping is a murine leukemia virus (MLV) glycoprotein. A suitable MLV protein comprises an amino acid sequence having at least 80%, at least 85%, at least 90%, at least 95%, at least 98%, at least 99%, or 100%, amino acid sequence identity to the following amino acid sequence: MESTTLSKPF KNQVNPWGPL IVLLILGGVN PVALGNSPHQ VFNLTWEVTN GDRETVWAIA GNHPLWTWWP DLTPDLCMLA LHGPSYWGLE YRAPFSPPPG PPCCSGSSDS TPGCSRDCEE PLTSYTPRCN TAWNRLKLSK VTHAHNEGFY VCPGPHRPRW ARSCGGPESF YCASWGCETT GRASWKPSSS WDYITVSNNL TSDQATPVCK GNEWCNSLTI RFTSFGKQAT SWVTGHWWGL RLYVSGHDPG LIFGIRLKIT DSGPRVPIGP NPVLSDRRPP SRPRPTRSPP PSNSTPTETP LTLPEPPPAG VENRLLNLVK GAYQALNLTS PDKTQECWLC LVSGPPYYEG VAVLGTYSNH TSAPANCSVA SQHKLTLSEV TGQGLCIGAV PKTHQVLCNT TQKTSDGSYY LAAPTGTTWA CSTGLTPCIS TTILDLTTDY CVLVELWPRV TYHSPSYVYH QFEGRAKYKR EPVSLTLALL LGGLTMGGIA AGVGTGTTAL VATQQFQQLQ AAMHDDLKEV EKSITNLEKS LTSLSEVVLQ NRRGLDLLFL KEGGLCAALK EECCFYADHT GLVRDSMAKL RERLSQRQKL FESQQGWFEG LFNKSPWFTT LISTIMGPLI ILLLILLFGP CILNRLVQFI KDRISVVQAL VLTQQYHQLK TIRDCKSRE (SEQ ID NO:76; GenBank Accession No: AAA51037).

In some cases, the heterologous glycoprotein used for pseudotyping is an MLV glycoprotein. A suitable MLV protein comprises an amino acid sequence having at least 80%, at least 85%, at least 90%, at least 95%, at least 98%, at least 99%, or 100%, amino acid sequence identity to the following amino acid sequence: MESTTLSKPF KNQVNPWGPL IVLLILRGVN PVTLGNSPHQ VFNLTWEVTN GDRETVWAIT GNHPLWTWWP DLTPDLCMLA LHGPSYWGLE YRAPFSPPPG PPCCSGSSDS TPGCSRDCEE PLTSYTPRCN TAWNRLKLSK VTHAHNGGFY VCPGPHRPRW ARSCGGPESF YCASWGCETT GRASWKPSSS WDYITVSNNL TSDQATPVCK GNKWCNSLTI RFTSFGKQAT SWVTGHWWGL RLYVSGHDPG LIFGIRLKIT DSGPRVPIGP NPVLSDRRPP SRPRPTRSPP PSNSTPTETP LTLPEPPPAG VENRLLNLVK GAYQALNLTS PDKTQECWLC LVSGPPYYEG VAVLGTYSNH TSAPANCSVA SQHKLTLSEV TGQGLCIGAV PKTHQVLCNT TQKTSDGSYY LAAPTGTTWA CSTGLTPCIS TTILDLTTDY CVLVELWPRV TYHSPSYVYH QFERRAKYKR EPVSLTLALL LGGLTMGGIA AGVGTGTTAL VATQQFQQLQ AAMHDDLKEV EKSITNLEKS LTSLSEVVLQ NRRGLDLLFL KEGGLCAALK EECCFYADHT GLVRDSMAKL RERLSQRQKL FESQQGWFEG LFNKSPWFTT LISTIMGPLI ILLLILLFGP CILNRLVQFI KDRISVVQAL VLTQQYHQLK IIEDCKSRE (SEQ ID NO:77; GenBank Accession No: AID54959).

In some cases, the heterologous glycoprotein used for pseudotyping is an MLV glycoprotein. A suitable MLV protein comprises an amino acid sequence having at least 80%, at least 85%, at least 90%, at least 95%, at least 98%, at least 99%, or 100%, amino acid sequence identity to the following amino acid sequence: MARSTLSKPP QDKINPWKPL IVMGVLLGVG MAESPHQVFN VTWRVTNLMT GRTANATSLL GTVQDAFPKL YFDLCDLVGE EWDPSDQEPY VGYGCKYPAG RQRTRTFDFY VCPGHTVKSG CGGPGEGYCG KWGCETTGQA YWKPTSSWDL ISLKRGNTPW DTGCSKVACG PCYDLSKVSN SFQGATRGGR CNPLVLEFTD AGKKANWDGP KSWGLRLYRT GTDPITMFSL TRQVLNVGPR VPIGPNPVLP DQRLPSSPIE IVPAPQPPSP LNTSYPPSTT STPSTSPTSP SVPQPPPGTG DRLLALVKGA YQALNLTNPD KTQECWLCLV SGPPYYEGVA VVGTYTNHST APANCTATSQ HKLTLSEVTG QGLCMGAVPK THQALCNTTQ SAGSGSYYLA APAGTMWACS TGLTPCLSTT VLNLTTDYCV LVELWPRVIY HSPDYMYGQL EQRTKYKREP VSLTLALLLG GLTMGGIAAG IGTGTTALIK TQQFEQLHAA IQTDLNEVEK SITNLEKSLT SLSEVVLQNR RGLDLLFLKE GGLCAALKEE CCFYADHTGL VRDSMAKLRE RLNQRQKLFE TGQGWFEGLF NRSPWFTTLI STIMGPLIVL LLILLFGPCI LNRLVQFVKD RISVVQALVL TQQYHQLKPI EYEP (SEQ ID NO:78; GenBank Accession No: AAA46515).

In some cases, the heterologous glycoprotein used for pseudotyping is an MLV glycoprotein. A suitable MLV protein comprises an amino acid sequence having at least 80%, at least 85%, at least 90%, at least 95%, at least 98%, at least 99%, or 100%, amino acid sequence identity to the following amino acid sequence: MEGPAFSKPL KDKINPWKSL MVMGVYLRVG MAESPHQVFN VTWRVTNLMT GRTANATSLL GTVQDAFPRL YFDLCDLVGE EWDPSDQEPY VGYGCKYPGG RKRTRTFDFY VCPGHTVKSG CGGPREGYCG EWGCETTGQA YWKPTSSWDL ISLKRGNTPW DTGCSKMACG PCYDLSKVSN SFQGATRGGR CNPLVLEFTD AGKKANWDGP KSWGLRLYRT GTDPITMFSL TRQVLNIGPR IPIGPNPVIT GQLPPSRPVQ IRLPRPPQPP PTGAASIVPE TAPPSQQPGT GDRLLNLVEG AYQALNLTNP DKTQECWLCL VSGPPYYEGV AVVGTYTNHS TAPASCTATS QHKLTLSEVT GQGLCMGALP KTHQALCNTT QSAGSGSYYL AAPAGTMWAC STGLTPCLST TMLNLTTDYC VLVELWPRII YHSPDYMYGQ LEQRTKYKRE PVSLTLALLL GGLTMGGIAA GIGTGTTALI KTQQFEQLHA AIQTDLNEVE KSITNLEKSL TSLSEVVLQN RRGLDLLFLK EGGLCAALKE ECCFYADHTG LVRDSMAKLR ERLNQRQKLF ESGQGWFEGQ FNRSPWFTTL ISTIMGPLIV LLLILLFGPC ILNRLVQFVK DRISVVQALV LTQQYHQLKP IEYEP (SEQ ID NO:79; GenBank Accession No: AAA46514).

In some cases, the heterologous glycoprotein used for pseudotyping is an MLV glycoprotein. A suitable MLV protein comprises an amino acid sequence having at least 80%, at least 85%, at least 90%, at least 95%, at least 98%, at least 99%, or 100%, amino acid sequence identity to the following amino acid sequence: MEGSAFSKPL KDKINPWGPL IVMGILVRAG ASVQRDSPHQ IFNVTWRVTN LMTGQTANAT SLLGTMTDTF PKLYFDLCDL VGDYWDDPEP DIGDGCRTPG GRRRTRLYDF YVCPGHTVPI GCGGPGEGYC GKWGCETTGQ AYWKPSSSWD LISLKRGNTP KDQGPCYDSS VSSGVQGATP GGRCNPLVLE FTDAGRKASW DAPKVWGLRL YRSTGADPVT RFSLTRQVLN VGPRVPIGPN PVITDQLPPS QPVQIMLPRP PHPPPSGTVS MVPGAPPPSQ QPGTGDRLLN LVEGAYQALN LTSPDKTQEC WLCLVSGPPY YEGVAVLGTY SNHTSAPANC SVASQHKLTL SEVTGQGLCV GAVPKTHQAL CNTTQKTSDG SYYLAAPAGT IWACNTGLTP CLSTTVLNLT TDYCVLVELW PKVTYHSPDY VYGQFEKKTK YKREPVSLTL ALLLGGLTMG GIAAGVGTGT TALVATKQFE QLQAAIHTDL GALEKSVSAL EKSLTSLSEV VLQNRRGLDL LFLKEGGLCA ALKEECCFYA DHTGVVRDSM AKLRERLNQR QKLFESGQGW FEGLFNRSPW FTTLISTIMG PLIVLLLILL LGPCILNRLV QFVKDRISVV QALILTQQYH QLKSIEPEEV ESRE (SEQ ID NO:80; GenBank Accession No: AAA46531).

In some cases, the heterologous glycoprotein used for pseudotyping is a polytropic mink cell focus-forming virus glycoprotein. A suitable polytropic mink cell focus-forming virus protein comprises an amino acid sequence having at least 80%, at least 85%, at least 90%, at least 95%, at least 98%, at least 99%, or 100%, amino acid sequence identity to the following amino acid sequence:

(SEQ ID NO: 81; GenBank Accession No: 2016415A)
VQHDSPHQVF NVTWRVTNLM TGQTANATSL LGTMTDAFPK
LYFDLCDLIG DDWDETGLGC RTPGGRKRAR TFDFYVCPGH
TVPTGCGGPR EGYCGKWGCE TTGQAYWKPS SLWDLISLKR
GNTPQNQGPC YDSSAVSSDI KGATPGGRCN PLVLEFTDAG
KKASWDGPKV WGLRLYRSTG TDPVTRFSLT RRVLNIGPRV
PIGPNPVIID QLPPSRPVQI MLPRPPQPPP PGAASIVPET
APPSNQPGTG DRLLNLVDGA YQALNLTSPD KTQECWLCLV
AEPPYYEGVA VLGTYSNHTS APANCSVASQ HKLTLSEVTG
RGLCIGTVPK THQALCNTTL KTNKGSYYLV APAGTTWACN
TGLTPCLSAT VLNRTTDYCV LVELWPRVTY HPPSYVYSQF
EKSYRHKR.

In some cases, the heterologous glycoprotein used for pseudotyping is a gibbon ape leukemia virus (GALV) glycoprotein. A suitable GALV protein comprises an amino acid sequence having at least 80%, at least 85%, at least 90%, at least 95%, at least 98%, at least 99%, or 100%, amino acid sequence identity to the following amino acid sequence: MVLLPGSMLL TSNLHHLRHQ MSPGSWKRLI ILLSCVFGGG GTSLQNKNPH QPMTLTWQVL SQTGDVVWDT KAVQPPWTWW PTLKPDVCAL AASLESWDIP GTDVSSSKRV RPPDSDYTAA YKQITWGAIG CSYPRARTRM ASSTFYVCPR DGRTLSEARR CGGLESLYCK EWDCETTGTG YWLSKSSKDL ITVKWDQNSE WTQKFQQCHQ TGWCNPLKID FTDKGKLSKD WITGKTWGLR FYVSGHPGVQ FTIRLKITNM PAVAVGPDLV LVEQGPPRTS LALPPPLPPR EAPPPSLPDS NSTALATSAQ TPTVRKTIVT LNTPPPTTGD RLFDLVQGAF LTLNATNPGA TESCWLCLAM GPPYYEAIAS SGEVAYSTDL DRCRWGTQGK LTLTEVSGHG LCIGKVPFTH QHLCNQTLSI NSSGDHQYLL PSNHSWWACS TGLTPCLSTS VFNQTRDFCI QVQLIPRIYY YPEEVLLQAY DNSHPRTKRE AVSLTLAVLL GLGITAGIGT GSTALIKGPI DLQQGLTSLQ IAIDADLRAL QDSVSKLEDS LTSLSEVVLQ NRRGLDLLFL KEGGLCAALK EECCFYIDHS GAVRDSMKKL KEKLDKRQLE RQKSQNWYEG WFNNSPWFTT LLSTIAGPLL LLLLLLILGP CIINKLVQFI NDRISAVKIL VLRQKYQALE NEGNL (SEQ ID NO:82; GenBank Accession No: P21415).

In some cases, the heterologous glycoprotein used for pseudotyping is a GALV glycoprotein. A suitable GALV protein comprises an amino acid sequence having at least 80%, at least 85%, at least 90%, at least 95%, at least 98%, at least 99%, or 100%, amino acid sequence identity to the following amino acid sequence: TSLQNKNPH QPMTLTWQVL SQTGDVVWDT KAVQPPWTWW PTLKPDVCAL AASLESWDIP GTDVSSSKRV RPPDSDYTAA YKQITWGAIG CSYPRARTRM ASSTFYVCPR DGRTLSEARR CGGLESLYCK EWDCETTGTG YWLSKSSKDL ITVKWDQNSE WTQKFQQCHQ TGWCNPLKID FTDKGKLSKD WITGKTWGLR FYVSGHPGVQ FTIRLKITNM PAVAVGPDLV LVEQGPPRTS LALPPPLPPR EAPPPSLPDS NSTALATSAQ TPTVRKTIVT LNTPPPTTGD RLFDLVQGAF LTLNATNPGA TESCWLCLAM GPPYYEAIAS SGEVAYSTDL DRCRWGTQGK LTLTEVSGHG LCIGKVPFTH QHLCNQTLSI NSSGDHQYLL PSNHSWWACS TGLTPCLSTS VFNQTRDFCI QVQLIPRIYY YPEEVLLQAY DNSHPRTKRE AVSLTLAVLL GLGITAGIGT GSTALIKGPI DLQQGLTSLQ IAIDADLRAL QDSVSKLEDS LTSLSEVVLQ NRRGLDLLFL KEGGLCAALK EECCFYIDHS GAVRDSMKKL KEKLDKRQLE RQKSQNWYEG WFNNSPWFTT LLSTIAGPLL LLLLLLILGP CIINKLVQFI NDRISAVKIL VLRQKYQALE NEGNL (SEQ ID NO:83).

In some cases, the heterologous glycoprotein used for pseudotyping is a GALV glycoprotein. A suitable GALV protein comprises an amino acid sequence having at least 80%, at least 85%, at least 90%, at least 95%, at least 98%, at least 99%, or 100%, amino acid sequence identity to the following amino acid sequence: TSLQNKNPH QPMTLTWQVL SQTGDVVWDT KAVQPPWTWW PTLKPDVCAL AASLESWDIP GTDVSSSKRV RPPDSDYTAA YKQITWGAIG CSYPRARTRM ASSTFYVCPR DGRTLSEARR CGGLESLYCK EWDCETTGTG YWLSKSSKDL ITVKWDQNSE WTQKFQQCHQ TGWCNPLKID FTDKGKLSKD WITGKTWGLR FYVSGHPGVQ FTIRLKITNM PAVAVGPDLV LVEQGPPRTS LALPPPLPPR EAPPPSLPDS NSTALATSAQ TPTVRKTIVT LNTPPPTTGD RLFDLVQGAF LTLNATNPGA TESCWLCLAM GPPYYEAIAS SGEVAYSTDL DRCRWGTQGK LTLTEVSGHG LCIGKVPFTH QHLCNQTLSI NSSGDHQYLL PSNHSWWACS TGLTPCLSTS VFNQTRDFCI QVQLIPRIYY YPEEVLLQAY DNSHPRTKRE AVSLTLAVLL GLGITAGIGT GSTALIKGPI DLQQGLTSLQ IAIDADLRAL QDSVSKLEDS LTSLSEVVLQ NRRGLDLLFL KEGGLCAALK EECCFYIDHS GAVRDSMKKL KEKLDKRQLE RQKSQNWYEG WFNNSPWFTT LLSTIAGPLL LLLLLLILGP CIINKLVQFI NDRISAVKIL VLRQKYQALE NEGNL (SEQ ID NO:83).

In some cases, the heterologous glycoprotein used for pseudotyping is a GALV glycoprotein. A suitable GALV protein comprises an amino acid sequence having at least 80%, at least 85%, at least 90%, at least 95%, at least 98%, at least 99%, or 100%, amino acid sequence identity to the following amino acid sequence: TSLQNKNPH QPMTLTWQVL SQTGDVVWDT KAVQPPWTWW PTLKPDVCAL AASLESWDIP GTDVSSSKRV RPPDSDYTAA YKQITWGAIG CSYPRARTRM ASSTFYVCPR DGRTLSEARR CGGLESLYCK EWDCETTGTG YWLSKSSKDL ITVKWDQNSE WTQKFQQCHQ TGWCNPLKID FTDKGKLSKD WITGKTWGLR FYVSGHPGVQ FTIRLKITNM PAVAVGPDLV LVEQGPPRTS LALPPPLPPR EAPPPSLPDS NSTALATSAQ TPTVRKTIVT LNTPPPTTGD RLFDLVQGAF LTLNATNPGA TESCWLCLAM GPPYYEAIAS SGEVAYSTDL DRCRWGTQGK LTLTEVSGHG LCIGKVPFTH QHLCNQTLSI NSSGDHQYLL PSNHSWWACS TGLTPCLSTS VFNQTRDFCI QVQLIPRIYY YPEEVLLQAY DNSHPRTKR (SEQ ID NO:84).

In some cases, the heterologous glycoprotein used for pseudotyping is a GALV glycoprotein. A suitable GALV protein comprises an amino acid sequence having at least 80%, at least 85%, at least 90%, at least 95%, at least 98%, at least 99%, or 100%, amino acid sequence identity to the following amino acid sequence: E AVSLTLAVLL GLGITAGIGT GSTALIKGPI DLQQGLTSLQ IAIDADLRAL QDSVSKLEDS LTSLSEVVLQ NRRGLDLLFL KEGGLCAALK EECCFYIDHS GAVRDSMKKL KEKLDKRQLE RQKSQNWYEG WFNNSPWFTT LLSTIAGPLL LLLLLLILGP CIINKLVQFI NDRISAVKIL (SEQ ID NO:85).

In some cases, the heterologous glycoprotein used for pseudotyping is a RD114 retrovirus glycoprotein. A suitable RD114 retrovirus protein comprises an amino acid sequence having at least 80%, at least 85%, at least 90%, at least 95%, at least 98%, at least 99%, or 100%, amino acid sequence identity to the following amino acid sequence: MKLPTGMVIL CSLIIVRAGF DDPRKAIALV QKQHGKPCEC SGGQVSEAPP NSIQQVTCPG KTAYLMTNQK WKCRVTPKNL TPSGGELQNC PCNTFQDSMH SSCYTEYRQC RANNKTYYTA TLLKIRSGSL NEVQILQNPN QLLQSPCRGS INQPVCWSAT APIHISDGGG PLDTKRVWTV QKRLEQIHKA MHPELQYHPL ALPKVRDDLS LDARTFDILN TTFRLLQMSN FSLAQDCWLC LKLGTPTPLA IPTPSLTYSL ADSLANASCQ IIPPLLVQPM QFSNSSCLSS PFINDTEQID LGAVTFTNCT SVANVSSPLC ALNGSVFLCG NNMAYTYLPQ NWTGLCVQAS LLPDIDIIPG DEPVPIPAID HYIHRPKRAV QFIPLLAGLG ITAAFTTGAT GLGVSVTQYT KLSHQLISDV QVLSGTIQDL QDQVDSLAEV VLQNRRGLDL LTAEQGGICL ALQEKCCFYA NKSGIVRNKI RTLQEELQKR RESLASNPLW TGLQGFLPYL LPLLGPLLTL LLILTIGPCV FSRLMAFIND RLNVVHAMVL AQQYQALKAE EEAQD (SEQ ID NO:86; GenBank Accession No: YP_001497149).

In some cases, the heterologous glycoprotein used for pseudotyping is a Sendai virus (SeV) glycoprotein. A suitable SeV protein comprises an amino acid sequence having at least 80%, at least 85%, at least 90%, at least 95%, at least 98%, at least 99%, or 100%, amino acid sequence identity to the following amino acid sequence: MTAYIQRSQC ISTSLLVVLT TLVSCQIPRD RLSNIGVIVD EGKSLKIAGS HESRYIVLSL VPGVDFENGC GTAQVIQYKS LLNRLLIPLR DALDLQEALI TVTNDTTQNA GAPQSRFFGA VIGTIALGVA TSAQITAGIA LAEAREAKRD IALIKESMTK THKSIELLQN AVGEQILALK TLQDFVNDEI KPAISELGCE TAALRLGIKL TQHYSELLTA FGSNFGTIGE KSLTLQALSS LYSANITEIM TTIKTGQSNI YDVIYTEQIK GTVIDVDLER YMVTLSVKIP ILSEVPGVLI HKASSISYNI DGEEWYVTVP SHILSRASFL GGADITDCVE SRLTYICPRD PAQLIPDSQQ KCILGDTTRC PVTKVVDSLI PKFAFVNGGV VANCIASTCT CGTGRRPISQ DRSKGVVFLT HDNCGLIGVN GVELYANRRG HDATWGVQNL TVGPAIAIRP IDISLNLADA TNFLQDSKAE LEKARKILSE VGRWYNSRET VITIIVVMVV ILVVIIVIII VLYRLRRSML MGNPDDRIPR DTYTLEPKIR HMYTNGGFDA MAKER (SEQ ID NO:87; GenBank Accession No: P04855).

In some cases, the heterologous glycoprotein used for pseudotyping is an SeV FO glycoprotein. A suitable SeV protein comprises an amino acid sequence having at least 80%, at least 85%, at least 90%, at least 95%, at least 98%, at least 99%, or 100%, amino acid sequence identity to the following amino acid sequence: QIPRD RLSNIGVIVD EGKSLKIAGS HESRYIVLSL VPGVDFENGC GTAQVIQYKS LLNRLLIPLR DALDLQEALI TVTNDTTQNA GAPQSRFFGA VIGTIALGVA TSAQITAGIA LAEAREAKRD IALIKESMTK THKSIELLQN AVGEQILALK TLQDFVNDEI KPAISELGCE TAALRLGIKL TQHYSELLTA FGSNFGTIGE KSLTLQALSS LYSANITEIM TTIKTGQSNI YDVIYTEQIK GTVIDVDLER YMVTLSVKIP ILSEVPGVLI HKASSISYNI DGEEWYVTVP SHILSRASFL GGADITDCVE SRLTYICPRD PAQLIPDSQQ KCILGDTTRC PVTKVVDSLI PKFAFVNGGV VANCIASTCT CGTGRRPISQ DRSKGVVFLT HDNCGLIGVN GVELYANRRG HDATWGVQNL TVGPAIAIRP IDISLNLADA TNFLQDSKAE LEKARKILSE VGRWYNSRET VITIIVVMVV ILVVIIVIII VLYRLRRSML MGNPDDRIPR DTYTLEPKIR HMYTNGGFDA MAEKR (SEQ ID NO:88; GenBank Accession No: P04855).

In some cases, the heterologous glycoprotein used for pseudotyping is an SeV F2 glycoprotein. A suitable SeV protein comprises an amino acid sequence having at least 80%, at least 85%, at least 90%, at least 95%, at least 98%, at least 99%, or 100%, amino acid sequence identity to the following amino acid sequence: QIPRD RLSNIGVIVD EGKSLKIAGS HESRYIVLSL VPGVDFENGC GTAQVIQYKS LLNRLLIPLR DALDLQEALI TVTNDTTQNA GAPQSR (SEQ ID NO:89; GenBank Accession No: P04855).

In some cases, the heterologous glycoprotein used for pseudotyping is an SeV F1 glycoprotein. A suitable SeV protein comprises an amino acid sequence having at least 80%, at least 85%, at least 90%, at least 95%, at least 98%, at least 99%, or 100%, amino acid sequence identity to the following amino acid sequence: FFGA VIGTIALGVA TSAQITAGIA LAEAREAKRD IALIKESMTK THKSIELLQN AVGEQILALK TLQDFVNDEI KPAISELGCE TAALRLGIKL TQHYSELLTA FGSNFGTIGE KSLTLQALSS LYSANITEIM TTIKTGQSNI YDVIYTEQIK GTVIDVDLER YMVTLSVKIP ILSEVPGVLI HKASSISYNI DGEEWYVTVP SHILSRASFL GGADITDCVE SRLTYICPRD PAQLIPDSQQ KCILGDTTRC PVTKVVDSLI PKFAFVNGGV VANCIASTCT CGTGRRPISQ DRSKGVVFLT HDNCGLIGVN GVELYANRRG HDATWGVQNL TVGPAIAIRP IDISLNLADA TNFLQDSKAE LEKARKILSE VGRWYNSRET VITIIVVMVV ILVVIIVIII VLYRLRRSML MGNPDDRIPR DTYTLEPKIR HMYTNGGFDA MAKER (SEQ ID NO:90; GenBank Accession No: P04855).

In some cases, the heterologous glycoprotein used for pseudotyping is an SeV hemagglutinin-neuraminidase glycoprotein. A suitable SeV protein comprises an amino acid sequence having at least 80%, at least 85%, at least 90%, at least 95%, at least 98%, at least 99%, or 100%, amino acid sequence identity to the following amino acid sequence: MDGDRSKRDS YWSTSPGGST TKLVSDSERS GKVDTWLLIL AFTQWALSIA TVIICIVIAA RQGYSMERYS MTVEALNTSN KEVKESLTSL IRQEVITRAA NIQSSVQTGI PVLLNKNSRD VIRLIEKSCN RQELTQLCDS TIAVHHAEGI APLEPHSFWR CPAGEPYLSS DPEVSLLPGP SLLSGSTTIS GCVRLPSLSI GEAIYAYSSN LITQGCADIG KSYQVLQLGY ISLNSDMFPD LNPVVSHTYD INDNRKSCSV VATGTRGYQL CSMPIVDERT DYSSDGIEDL VLDILDLKGR TKSHRYSNSE IDLDHPFSAL YPSVGSGIAT EGSLIFLGYG GLTTPLQGDT KCRIQGCQQV SQDTCNEALK ITWLGGKQVV SVLIQVNDYL SERPRIRVTT IPITQNYLGA EGRLLKLGDQ VYIYTRSSGW HSQLQIGVLD VSHPLTISWT PHEALSRPGN EDCNWYNTCP KECISGVYTD AYPLSPDAAN VATVTLYANT SRVNPTIMYS NTTNIINMLR IKDVQLEAAY TTTSCITHFG KGYCFHIIEI NQKSLNTLQP MLFKTSIPKL CKAES (SEQ ID NO:91; GenBank Accession No: BAA24391).

In some cases, the heterologous glycoprotein used for pseudotyping is a Jaagsiekte sheep retrovirus (JSRV) glycoprotein. A suitable JSRV protein comprises an amino acid sequence having at least 80%, at least 85%, at least 90%, at least 95%, at least 98%, at least 99%, or 100%, amino acid sequence identity to the following amino acid sequence: MPKRRAGFRK GWYARQRNSL THQMQRMTLS EPTSELPTQR QIEALMRYAW NEAHVQPPVT PTNILIMLLL LLQRIQNGAA ATFWAYIPDP PMLQSLGWDK ETVPVYVNDT SLLGGKSDIH ISPQQANISF YGLTTQYPMC FSYQSQHPHC IQVSADISYP RVTISGIDEK TGMRSYRDGT GPLDIPFCDK HLSIGIGIDT PWTLCRARIA SVYNINNANT TLLWDWAPGG TPDFPEYRGQ HPPISSVNTA PIYQTELWKL LAAFGHGNSL YLQPNISGSK YGDVGVTGFL YPRACVPYPF MVIQGHMEIT PSLNIYYLNC SNCILTNCIR GVAKGEQVII VKQPAFVMLP VEITEEWYDE TALELLQRIN TALSRPKRGL SLIILGIVSL ITLIATAVTA SVSLAQSIQV AHTVDSLSSN VTKVMGTQEN IDKKIEDRLP ALYDVVRVLG EQVQSINFRM KIQCHANYKW ICVTKKPYNT SDFPWDKVKK HLQGIWFNTT VSLDLLQLHN EILDIENSPK ATLNIADTVD NFLQNLFSNF PSLHSLWRSI IAMGAVLTFV LIIICLAPCL IRSIVKEFLH MRVLIHKNML QHQHLMELLN NKERGAAGDD P (SEQ ID NO:92; GenBank Accession No: AB1I50237).

In some cases, the heterologous glycoprotein used for pseudotyping is a baculovirus gp64 glycoprotein. A suitable baculovirus protein comprises an amino acid sequence having at least 80%, at least 85%, at least 90%, at least 95%, at least 98%, at least 99%, or 100%, amino acid sequence identity to the following amino acid sequence: MFHLLTLLLL LFINMNLYLA GEHCNVQMKN GPYRIKNLAI TPPRETLKKD VTVTIVETDY EENVLIGYKG YYQAYGYNGG SLDANTRLEE TMESLPLTKE DLLTWTYRQE CEVGEELIDR WGSDSDDCYR NKDGRGVWVK TKELVKRQNN NHFAHHTCNR SWRCGFSTAK MYSKLVCDDE TNDCKVFILD NTGKPINITT NEVLYRDGVN MMLKSKPTFT RREEKVACLL VKDELNPDKT REHCLIDSDI YDLSNNNWFC MFNKCIKRNV DSVVKKRPNK WMHNLAPKYS EGATATKGDM MHIQEELMYE NDLLKMNIEL VHAHMNKLNN IIHDLIVSIA KVDERLIGNL MNISVSSVFL SDDTFLLMPC TNPPQHTSNC YNNSIYREGR WVFNEDTSEC IDFNNYRELS IDDDIEFWIP TIGNTTYHDS WKDASGWSFV AQQKSNLIMT MENTKFGGVG TSLSDITSMS EGELTAKLTT FVFSHIVTFI LIIILIILCI CLLKK (SEQ ID NO:93; GenBank Accession No: YP_009182316).

In some cases, the heterologous glycoprotein used for pseudotyping is a baculovirus gp64 glycoprotein. A suitable baculovirus protein comprises an amino acid sequence having at least 80%, at least 85%, at least 90%, at least 95%, at least 98%, at least 99%, or 100%, amino acid sequence identity to the following amino acid sequence: MLRITLLILF LVRFVSGAEH CNAQMKSGPW RIKNLPIAPP KETLQKDVDV EIVETDLDEN VIIGYKGYYQ AYAYNGGSLD PNTSVDETTQ TLNIDKDDLI TWGDRRKCEV GEELIDQWGS DSDSCFKDKL GRGVWVAGKE LVKRKNNNHF AHHTCNRSWR CGVSTAKMYT RLECDNETDD CKVTILDING TVINVTENEV LHRDGVSMIL KQKSTFTRRT EKVACLLIKD DKSDPYSITR EHCLIDNDIF DLSKNTWNCK FNRCIKRRSE NVVKKRPPTW RHNEPPKHSE GTTATKGDLM HIQEELMYEN DLLRMNLELL HAHINKLNNM MHDLIVSVAK VDERLIGNLM NNSVSSTFLS DDTFLLMPCT NPPPHTSNCY NNSIYKEGRW VANTDSSQCI DFRNYKELAI DDDIEFWIPT IGNTSYHESW KDASGWSFIA QQKSNLISTM ENTKFGGHTT SLSDIGDMAK GELNATLYSF MLGHGFSFFL IIGVIVFLIC MVRSRVRAF (SEQ ID NO:94; GenBank Accession No: YP_473216).

In some cases, the heterologous glycoprotein used for pseudotyping is a Chandipura virus glycoprotein. A suitable Chandipura virus protein comprises an amino acid sequence having at least 80%, at least 85%, at least 90%, at least 95%, at least 98%, at least 99%, or 100%, amino acid sequence identity to the following amino acid sequence: MTSSVTISVI LLISFIAPSY SSLSIAFPEN TKLDWKPVTK NTRYCPMGGE WFLEPGLQEE SFLSSTPIGA TPSKSDGFLC HAAKWVTTCD FRWYGPKYIT HSIHNIKPTR SDCDTALASY KSGTLVSPGF PPESCGYASV TDSEFLVIMI TPHHVGVDDY RGHWVDPLFV GGECDQSYCD TIHNSSVWIP ADQTKKNICG QSFTPLTVTV AYDKTKEIAA GAIVFKSKYH SHMEGARTCR LSYCGRNGIK FPNGEWVSLD VKTKIQEKPL LPLFKECPAG TEVRSTLQSD GAQVLTSEIQ RILDYSLCQN TWDKVERKEP LSPLDLSYLA SKSPGKGLAY TVINGTLSFA HTRYVRMWID GPVLKEMKGK RESPSGISSD IWTQWFKYGD MEIGPNGLLK TAGGYKFPWH LIGMGIVDNE LHELSEANPL DHPQLPHAQS IADDSEEIFF GDTGVSKNPV ELVTGWFTSW KESLAAGVVL ILVVVLIYGV LRCFPVLCTT CRKPKWKKGV ERSDSFEMRI FKPNNMRARV (SEQ ID NO:95; GenBank Accession No: YP_007641380).

In some cases, the heterologous glycoprotein used for pseudotyping is a Venezuelan equine encephalitis virus glycoprotein. A suitable Venezuelan equine encephalitis virus protein comprises an amino acid sequence having at least 80%, at least 85%, at least 90%, at least 95%, at least 98%, at least 99%, or 100%, amino acid sequence identity to the following amino acid sequence: MFPFQPMYPM QPMPYRNPFA APRRPWFPRT DPFLAMQVQE LTRSMANLTF KQRRDAPPEG PSAKKPKKEA SQKQKGGGQG KKKKNQGKKK AKTGPPNPKA QNGNKKKTNK KPGKRQRMVM KLESDKTFPI MLEGKINGYA CVVGGKLFRP MHVEGKIDND VLAALKTKKA SKYDLEYADV PQNMRADTFK YTHEKPQGYY SWHHGAVQYE NGRFTVPKGV GAKGDSGRPI LDNQGRVVAI VLGGVNEGSR TALSVVMWNE KGVTVKYTPE NCEQWSLVTT MCLLANVTFP CAQPPICYDR KPAETLAMLS VNVDNPGYDE LLEAAVKCPG STEELFKEYK LTRPYMARCI RCAVGSCHSP IAIEAVKSDG HDGYVRLQTS SQYGLDSSGN LKGRTMRYDM HGTIKEIPLH QVSLHTSRPC HIVDGHGYFL LARCPAGDSI TMEFKKDSVT HSCSVPYEVK FNPVGRELYT HPPEHGVEQA CQVYAHDAQN RGAYVEMHLP GSEVDSSLVS LSGSSVTVTP PVGTSALVEC ECGGTKISET INKTKQFSQC TKKEQCRAYR LQNDKWVYIS DKLPKAAGAT LKGKLHVPFL LADGKCTVPL APEPMITFGF RSVSLKLHPK NPTYLTTRQL ADEPHYTHEL ISEPAVRNFT VTGKGWEFVW GNHPPKRFWA QETAPGNPHG LPHEVITHYY HRYPMSTILG LSICAAIATV SVAASTWLFC RSRVACLTPY RLTPNARIPF CLAVLCCART ARAETTWESL DHLWNNNQQM FWIQLLIPLA ALIVVTRLLR CVCCVVPFLV MAGAAGAGAY EHATTMPSQA GISYNTIVNR AGYAPLPISI TPTKIKLIPT VNLEYVTCHY KTGMDSPAIK CCGSQECTPT YRPDEQCKVF TGVYPFMWGG AYCFCDTENT QVSKAYVMKS DDCLADHAEA YKAHTASVQA FLNITVGEHS IVTTVYVNGE TPVNFNGVKL TAGPLSTAWT PFDRKIVQYA GEIYNYDFPE YGAGQPGAFG DIQSRTVSSS DLYANTNLVL QRPKAGAIHV PYTQAPSGFE QWKKDKAPSL KSTAPFGCEI YTNPIRAENC AVGSIPLAFD IPDALFTRVS ETPTLSAAEC TLNECVYSSD FGGIATVKYS ASKSGKCAVH VPSGTATLKE AAVELTEQGS ATIHFSTANI HPEFRLQICT SYVTCKGDCH PPKDHIVTHP QYHAQTFTAA VSKTAWTWLT SLLGGSAVII IIGLVLATIV AMYVLTNQKH N (SEQ ID NO:96; GenBank Accession No: AAU89534). Such a glycoprotein may be useful for targeting a VLP of the present disclosure to dendritic cells, macrophages, and cells of the spleen, lymph node, thymus, pancreas, skeletal muscle, and central nervous system.

In some cases, the heterologous glycoprotein used for pseudotyping is a Venezuelan equine encephalitis virus E2 glycoprotein. A suitable Venezuelan equine encephalitis virus protein comprises an amino acid sequence having at least 80%, at least 85%, at least 90%, at least 95%, at least 98%, at least 99%, or 100%, amino acid sequence identity to the following amino acid sequence: STEELFKEYK LTRPYMARCI RCAVGSCHSP IAIEAVKSDG HDGYVRLQTS SQYGLDSSGN LKGRTMRYDM HGTIKEIPLH QVSLHTSRPC HIVDGHGYFL LARCPAGDSI TMEFKKDSVT HSCSVPYEVK FNPVGRELYT HPPEHGVEQA CQVYAHDAQN RGAYVEMHLP GSEVDSSLVS LSGSSVTVTP PVGTSALVEC ECGGTKISET INKTKQFSQC TKKEQCRAYR LQNDKWVYIS DKLPKAAGAT LKGKLHVPFL LADGKCTVPL APEPMITFGF RSVSLKLHPK NPTYLTTRQL ADEPHYTHEL ISEPAVRNFT VTGKGWEFVW GNHPPKRFWA QETAPGNPHG LPHEVITHYY HRYPMSTILG LSICAAIATV SVAASTWLFC RSRVACLTPY RLTPNARIPF CLAVLCCART ARA (SEQ ID NO:97; GenBank Accession No: AAU89534). Such a glycoprotein may be useful for targeting a VLP of the present disclosure to dendritic cells, macrophages, and cells of the spleen, lymph node, thymus, pancreas, skeletal muscle, and central nervous system.

In some cases, the heterologous glycoprotein used for pseudotyping is a Venezuelan equine encephalitis virus E1 glycoprotein. A suitable Venezuelan equine encephalitis virus protein comprises an amino acid sequence having at least 80%, at least 85%, at least 90%, at least 95%, at least 98%, at least 99%, or 100%, amino acid sequence identity to the following amino acid sequence: Y EHATTMPSQA GISYNTIVNR AGYAPLPISI TPTKIKLIPT VNLEYVTCHY KTGMDSPAIK CCGSQECTPT YRPDEQCKVF TGVYPFMWGG AYCFCDTENT QVSKAYVMKS DDCLADHAEA YKAHTASVQA FLNITVGEHS IVTTVYVNGE TPVNFNGVKL TAGPLSTAWT PFDRKIVQYA GEIYNYDFPE YGAGQPGAFG DIQSRTVSSS DLYANTNLVL QRPKAGAIHV PYTQAPSGFE QWKKDKAPSL KSTAPFGCEI YTNPIRAENC AVGSIPLAFD IPDALFTRVS ETPTLSAAEC TLNECVYSSD FGGIATVKYS ASKSGKCAVH VPSGTATLKE AAVELTEQGS ATIHFSTANI HPEFRLQICT SYVTCKGDCH PPKDHIVTHP QYHAQTFTAA VSKTAWTWLT SLLGGSAVII IIGLVLATIV AMYVLTNQKH N (SEQ ID NO:98; GenBank Accession No: AAU89534). Such a glycoprotein may be useful for targeting a VLP of the present disclosure to dendritic cells, macrophages, and cells of the spleen, lymph node, thymus, pancreas, skeletal muscle, and central nervous system.

In some cases, the heterologous glycoprotein used for pseudotyping is a Lassa virus glycoprotein. A suitable Lassa virus protein comprises an amino acid sequence having at least 80%, at least 85%, at least 90%, at least 95%, at least 98%, at least 99%, or 100%, amino acid sequence identity to the following amino acid sequence: MGQIVTFFQE VPHVIEEVMN IVLIALSVLA VLKGLYNFAT CGLVGLVTFL LLCGRSCTTS LYKGVYELQT LELNMETLNM TMPLSCTKNN SHHYIMVGNE TGLELTLTNT SIINHKFCNL SDAHKKNLYD HALMSIISTF HLSIPNFNQY EAMSCDFNGG KISVQYNLSH SYAGDAANHC GTVANGVLQT FMRMAWGGSY IALDSGRGNW DCIMTSYQYL IIQNTTWEDH CQFSRPSPIG YLGLLSQRTR DIYISRRLLG TFTWTLSDSE GKDTPGGYCL TRWMLIEAEL KCFGNTAVAK CNEKHDEEFC DMLRLFDFNK QAIQRLKAEA QMSIQLINKA VNALINDQLI MKNHLRDIMG IPYCNYSKYW YLNHTTTGRT SLPKCWLVSN GSYLNETHFS DDIEQQADNM ITEMLQKEYM ERQGKTPLGL VDLFVFSTSF YLISIFLHLV KIPTHRHIVG KSCPKPHRLN HMGICSCGLY KQPGVPVKWK R (SEQ ID NO:99; GenBank Accession No: ADY11070).

In some cases, the heterologous glycoprotein used for pseudotyping is an avian leukosis virus glycoprotein. A suitable avian leukosis virus protein comprises an amino acid sequence having at least 80%, at least 85%, at least 90%, at least 95%, at least 98%, at least 99%, or 100%, amino acid sequence identity to the following amino acid sequence: MEAVIKMRRA LFLQAFLTGR PGKASKKDPK KNPLATSKKD PEKTPLLPTR VNYILIIGVL VLCEVTGVRA DVHLLEQPGN LWITWANRTG QTDFCLSTQS ATSPFQTCLI GIPSPISEGD FKGYVSDNCT TLGTDRLVSS ASITGGPDNS TTLTYRKVSC LLLKLNVSMW NEPPELQLLG SQSLPNITDI TQISGVAGGC VGFRPKGVPW YLGWSQGEAT RFLLRHPSFS NLTGPFTVVT ADRHNLFMGS EYCGAYGYRF WEIYNCSQEG QQYRCGKARR PRPQSPETQC TRQGGIWVNR SKEINETEPF SFTVNCTASN LGNASGCCGK AGTILPGIWV DSTQGNFTKP KALPPAIFLI CGDRAWQGIP SRPVGGPCYL GKLTMLAPNH TDILKILANS SRTGIRRRRS VSHLDDTCSD EVQLWGPTAR IFASILAPGV AAAQALREIE RLACWSVKQA NLTTSLLGDL LDDVTSIRHA VLQNRAAIDF LLLAHGHGCE DIAGMCCFNL SDHSESIQKK FQLMKEHVNK IGVDSDPIGS WLRGLFGGIG GWAVHLLKGL LLGLVVILLL VVCLPCFLQF VSSSIRKMIN NSVSYHTEYR KMQGGAV (SEQ ID NO: 100; GenBank Accession No: AD034853).

In some cases, the heterologous glycoprotein used for pseudotyping is an avian leukosis virus glycoprotein. A suitable avian leukosis virus protein comprises an amino acid sequence having at least 80%, at least 85%, at least 90%, at least 95%, at least 98%, at least 99%, or 100%, amino acid sequence identity to the following amino acid sequence: MEAVIKMRRA LFLQAFLTGH PGKVSKKDSK KKPPATGKRD PEKTPLLPTR VNYILIIGVL VLCEVTGVRA DVHLLEQPGN LWITWANRTG QTDFCLSTQS ATSPFQTCLI GIPSPISEGD FKGYVSGNCT ALGTHRLVSS GIHGGPDNST TLTYRKVSCL LLKLNVSLLD EPSELQLLGS QSLPNITNIT QIPSVAGGCI GFTPYGSPAG VYGWDRRQVT HILLTDPGSN PFFNKASNSS KPFTVVTADR HNLFMGSEYC GAYGYRFWEM YNCSQMRQNW SICMDVWGRG LPESWCTSTG GIWVNQSKEI NETEPFSFTA NCTGSNLGNV SGCCGESITI LPPGAWVDST QGSFTKPKAL PPGIFLICGD RAWQGIPSRP VGGPCYLGKL TMLAPNHTDI LKILANSSQT GVRHKRSVTH LDDTCSDEVQ LWGPTARIFA SILAPGVAAA QALREIERLA CWSVKQANLT TSLLGDLLDD VTSIRHAVLQ NRAAIDFLLL AHGHGCEDIA GMCCFNLSDH SESIQKKFQL MKEHVNKIGV DSDPIGSWLR GLFGGIGEWA VHLLKGLLLG LVVILLLVVC LPCFLQFVSS SIRKMINNSI SYHTEYRKMQ GGAV (SEQ ID NO:101; GenBank Accession No: AEF97639).

In some cases, the heterologous glycoprotein used for pseudotyping is an avian leukosis virus glycoprotein. A suitable avian leukosis virus protein comprises an amino acid sequence having at least 80%, at least 85%, at least 90%, at least 95%, at least 98%, at least 99%, or 100%, amino acid sequence identity to the following amino acid sequence: MEAVIKAFLT GHPGKVSKKD SKKKPPATSK KDPEKTPLLP SRGYFFFPTI LVCVVIISVV PGVGGVHLLR QPGNVWVTWA NKTGRTDFCL SLQSATSPFR TCLIGIPQYP LNTFKGYVTN VTACDNDADL ASQTACLIKA LNTTLPWDPQ ELDILGSQMI KNGTTRTCVT FGSVCYKENN RSRVCHNFDG NFNGTGGAEA ELRDFIAKWK SDDLLIRPYV NQSWTMVSPI NVESFSISRR YCGFTSNETR YYRGDLSNWC GSKRGKWSAG YSNRTKCSSN TTGCGGNCTT EWNYYAYGFT FGKQPEVLWN NGTAKALPPG IFLICGDRAW QGIPRNALGG PCYLGQLTML SPNFTTWITY GPNITGHRRS RRAIRGLSPD CSDEVQLWSA TARIFASFFA PGVAAAQALK EIERLACWSV KQANLTSLIL NAMLEDMNSI RHAVLQNRAA IDFLLLAQGH GCQDVEGMCC FNLSDHSESI HKALQAMKEH TEKIQVEDDP IGDWFTRTFG DLGRWLAKGV KTLLFALLVI VCLLAIIPCI IKCFQDCLSR TMNQFMDERI RYHRIREQL (SEQ ID NO: 102; GenBank Accession No: AWM62167).

In some cases, the heterologous glycoprotein used for pseudotyping is a human T-lymphotropic virus 1 (HTLV-1) glycoprotein. A suitable HTLV-1 protein comprises an amino acid sequence having at least 80%, at least 85%, at least 90%, at least 95%, at least 98%, at least 99%, or 100%, amino acid sequence identity to the following amino acid sequence: MGKFLATLIL FFQFCPLILG DYSPSCCTLT VGVSSYHSKP CNPAQPVCSW TLDLLALSAD QALQPPCPNL VSYSSYHATY SLYLFPHWIK KPNRNGGGYY SASYSDPCSL KCPYLGCQSW TCPYTGAVSS PYWKFQQDVN FTQEVSHLNI NLHFSKCGFP FSLLVDAPGY DPIWFLNTEP SQLPPTAPPL LSHSNLDHIL EPSIPWKSKL LTLVQLTLQS TNYTCIVCID RASLSTWHVL YSPNVSVPSL SSTPLLYPSL ALPAPHLTLP FNWTHCFDPQ IQAIVSSPCH NSLILPPFSL SPVPTLGSRS RRAVPVAVWL VSALAMGAGV AGGITGSMSL ASGKSLLHEV DKDISQLTQA IVKNHKNLLK IAQYAAQNRR GLDLLFWEQG GLCKALQEQC CFLNITNSHV SILQERPPLE NRVLTGWGLN WDLGLSQWAR EALQTGITLV ALLLLVILAG PCILRQLRHL PSRVRYPHYS LINPESSL (SEQ ID NO: 103; GenBank Accession No: AAU04884). Such a glycoprotein may be useful for targeting a VLP of the present disclosure to CD4+ and CD8+ T cells.

In some cases, the heterologous glycoprotein used for pseudotyping is a human foamy virus gp130 glycoprotein. A suitable human foamy virus protein comprises an amino acid sequence having at least 80%, at least 85%, at least 90%, at least 95%, at least 98%, at least 99%, or 100%, amino acid sequence identity to the following amino acid sequence: MAPPMTLQQW IIWKKMNKAH EALQNTTTVT EQQKEQIILD IQNEEVQPTR RDKFRYLLYT CCATSSRVLA WMFLVCILLI IVLVSCFVTI SRIQWNKDIQ VLGPVIDWNV TQRAVYQPLQ TRRIARSLRM QHPVPKYVEV NMTSIPQGVY YEPHPEPIVV KERVLGLSQI LMINSENIAN NANLTQEVKK LLTEMVNEEM QSLSDVMIDF EIPLGDPRDQ EQYIHRKCYQ EFANCYLVKY KEPKPWPKEG LIADQCPLPG YHAGLTYNRQ SIWDYYIKVE SIRPANWTTK SKYGQARLGS FYIPSSLRQI NVSHVLFCSD QLYSKWYNIE NTIEQNERFL LNKLNNLTSG TSVLKKRALP KDWSSQGKNA LFREINVLDI CSKPESVILL NTSYYSFSLW EGDCNFTKDM ISQLVPECDG FYNNSKWMHM HPYACRFWRS KKNEKEETKC RDGETKRCLY YPLWDSPEST YDFGYLAYQK NFPSPICIEQ QKIRDQDYEV YSLYQERKIA SKAYGIDTVL FSLKNFLNYT GTPVNEMPNA RAFVGLIDPK FPPSYPNVTR EHYTSCNNRK RRSVDNNYAK LRSMGYALTG AVQTLSQISD INDENLQQGI YLLRDHVITL MEATLHDISV MEGMFAVQHL HTHLNHLKTM LLERRIDWTY MSSTWLQQQL QKSDDEMKVI KRIARSLVYY VKQTHSSPTA TAWEIGLYYE LVIPKHIYLN NWNVVNIGHL VKSAGQLTHV TIAHPYEIIN KECVETIYLH LEDCTRQDYV ICDVVKIVQP CGNSSDTSDC PVWAEAVKEP FVQVNPLKNG SYLVLASSTD CQIPPYVPSI VTVNETTSCF GLDFKRPLVA EERLSFEPRL PNLQLRLPHL VGIIAKIKGI KIEVTSSGES IKEQIERAKA ELLRLDIHEG DTPAWIQQLA AATKDVWPAA ASALQGIGNF LSGTAQGIFG TAFSLLGYLK PILIGVGVIL LVILIFKIVS WIPTKKKNQ (SEQ ID NO: 104; GenBank Accession No: P14351).

In some cases, the heterologous glycoprotein used for pseudotyping is a human foamy virus glycoprotein. A suitable human foamy virus protein comprises an amino acid sequence having at least 80%, at least 85%, at least 90%, at least 95%, at least 98%, at least 99%, or 100%, amino acid sequence identity to the following amino acid sequence: SLRM QHPVPKYVEV NMTSIPQGVY YEPHPEPIVV KERVLGLSQI LMINSENIAN NANLTQEVKK LLTEMVNEEM QSLSDVMIDF EIPLGDPRDQ EQYIHRKCYQ EFANCYLVKY KEPKPWPKEG LIADQCPLPG YHAGLTYNRQ SIWDYYIKVE SIRPANWTTK SKYGQARLGS FYIPSSLRQI NVSHVLFCSD QLYSKWYNIE NTIEQNERFL LNKLNNLTSG TSVLKKRALP KDWSSQGKNA LFREINVLDI CSKPESVILL NTSYYSFSLW EGDCNFTKDM ISQLVPECDG FYNNSKWMHM HPYACRFWRS KKNEKEETKC RDGETKRCLY YPLWDSPEST YDFGYLAYQK NFPSPICIEQ QKIRDQDYEV YSLYQERKIA SKAYGIDTVL FSLKNFLNYT GTPVNEMPNA RAFVGLIDPK FPPSYPNVTR EHYTSCNNRK RR (SEQ ID NO: 105).

In some cases, the heterologous glycoprotein used for pseudotyping is a human foamy virus glycoprotein. A suitable human foamy virus protein comprises an amino acid sequence having at least 80%, at least 85%, at least 90%, at least 95%, at least 98%, at least 99%, or 100%, amino acid sequence identity to the following amino acid sequence: SVDNNYAK LRSMGYALTG AVQTLSQISD INDENLQQGI YLLRDHVITL MEATLHDISV MEGMFAVQHL HTHLNHLKTM LLERRIDWTY MSSTWLQQQL QKSDDEMKVI KRIARSLVYY VKQTHSSPTA TAWEIGLYYE LVIPKHIYLN NWNVVNIGHL VKSAGQLTHV TIAHPYEIIN KECVETIYLH LEDCTRQDYV ICDVVKIVQP CGNSSDTSDC PVWAEAVKEP FVQVNPLKNG SYLVLASSTD CQIPPYVPSI VTVNETTSCF GLDFKRPLVA EERLSFEPRL PNLQLRLPHL VGIIAKIKGI KIEVTSSGES IKEQIERAKA ELLRLDIHEG DTPAWIQQLA AATKDVWPAA ASALQGIGNF LSGTAQGIFG TAFSLLGYLK PILIGVGVIL LVILIFKIVS WIPTKKKNQ (SEQ ID NO: 106).

In some cases, the heterologous glycoprotein used for pseudotyping is a visna-maedi virus gp160 glycoprotein. A suitable visna-maedi virus protein comprises an amino acid sequence having at least 80%, at least 85%, at least 90%, at least 95%, at least 98%, at least 99%, or 100%, amino acid sequence identity to the following amino acid sequence: MASKESKPSR TTRRGMEPPL RETWNQVLQE LVKRQQQEEE EQQGLVSGKK KSWVSIDLLG TEGKDIKKVN IWEPCEKWFA QVVWGVLWVL QIVLWGCLMW EVRKGNQCQA EEVIALVSDP GGFQRVQHVE TVPVTCVTKN FTQWGCQPEG AYPDPELEYR NISREILEEV YKQDWPWNTY HWPLWQMENM RQWMKENEKE YKERTNKTKE DIDDLVAGRI RGRFCVPYPY ALLRCEEWCW YPESINQETG HAEKIKINCT KAKAVSCTEK MSLAAVQRVY WEKEDEESMK FLNIKACNIS LRCQDEGKSP GGCVQGYPIP KGAEIIPEAM KYLRGKKSRY GGIKDKNGEL KLPLSVRVWV RMANLSGWVN GTPPYWSARI NGSTGINGTR WYGIGTLHHL GCNISSNPER GICNFTGELW IGGDKFPYYY TPSWNCSQNW TGHPVWHVFR YLDMTEHMTS RCIQRPKRHN ITVGNGTITG NCSVTNWDGC NCTRSGNHLY NSTSGGLLVI ICRQNSTITG IMGTNTNWTT MWNIYQNCSR CNNSSLDRTG SGTLGTVNNL KCSLPHRNES NKWTCKSQRD SYIAGRDFWG KVKAKYSCES NLGGLDSMMH QQMLLQRYQV IRVRAYTYGV VEMPQSYMEA QGENKRSRRN LQRKKRGIGL VIVLAIMAII AAAGAGLGVA NAVQQSYTRT AVQSLANATA AQQEVLEASY AMVQHIAKGI RILEARVARV EALVDRMMVY QELDCWHYQH YCVTSTRSEV ANYVNWTRFK DNCTWQQWEE EIEQHEGNLS LLLREAALQV HIAQRDARRI PDAWKAIQEA FNWSSWFSWL KYIPWIIMGI VGLMCFRILM CVISMCLQAY KQVKQIRYTQ VTVVIEAPVE LEEKQKRNGD GTNGCASLER ERRTSHRSFI QIWRATWWAW KTSPWRHNWR TMPYITLLPI LVIWQWMEEN GWNGENQHKK KKERVDCQDR EQMPTLENDY VEL (SEQ ID NO: 107; GenBank Accession No: P35954).

In some cases, the heterologous glycoprotein used for pseudotyping is a visna-maedi virus glycoprotein. A suitable visna-maedi virus protein comprises an amino acid sequence having at least 80%, at least 85%, at least 90%, at least 95%, at least 98%, at least 99%, or 100%, amino acid sequence identity to the following amino acid sequence: QCQA EEVIALVSDP GGFQRVQHVE TVPVTCVTKN FTQWGCQPEG AYPDPELEYR NISREILEEV YKQDWPWNTY HWPLWQMENM RQWMKENEKE YKERTNKTKE DIDDLVAGRI RGRFCVPYPY ALLRCEEWCW YPESINQETG HAEKIKINCT KAKAVSCTEK MSLAAVQRVY WEKEDEESMK FLNIKACNIS LRCQDEGKSP GGCVQGYPIP KGAEIIPEAM KYLRGKKSRY GGIKDKNGEL KLPLSVRVWV RMANLSGWVN GTPPYWSARI NGSTGINGTR WYGIGTLHHL GCNISSNPER GICNFTGELW IGGDKFPYYY TPSWNCSQNW TGHPVWHVFR YLDMTEHMTS RCIQRPKRHN ITVGNGTITG NCSVTNWDGC NCTRSGNHLY NSTSGGLLVI ICRQNSTITG IMGTNTNWTT MWNIYQNCSR CNNSSLDRTG SGTLGTVNNL KCSLPHRNES NKWTCKSQRD SYIAGRDFWG KVKAKYSCES NLGGLDSMMH QQMLLQRYQV IRVRAYTYGV VEMPQSYMEA QGENKRSRRN LQRKKRGIGL VIVLAIMAII AAAGAGLGVA NAVQQSYTRT AVQSLANATA AQQEVLEASY AMVQHIAKGI RILEARVARV EALVDRMMVY QELDCWHYQH YCVTSTRSEV ANYVNWTRFK DNCTWQQWEE EIEQHEGNLS LLLREAALQV HIAQRDARRI PDAWKAIQEA FNWSSWFSWL KYIPWIIMGI VGLMCFRILM CVISMCLQAY KQVKQIRYTQ VTVVIEAPVE LEEKQKRNGD GTNGCASLER ERRTSHRSFI QIWRATWWAW KTSPWRHNWR TMPYITLLPI LVIWQWMEEN GWNGENQHKK KKERVDCQDR EQMPTLENDY VEL (SEQ ID NO: 108).

In some cases, the heterologous glycoprotein used for pseudotyping is a visna-maedi virus glycoprotein. A suitable visna-maedi virus protein comprises an amino acid sequence having at least 80%, at least 85%, at least 90%, at least 95%, at least 98%, at least 99%, or 100%, amino acid sequence identity to the following amino acid sequence: QCQA EEVIALVSDP GGFQRVQHVE TVPVTCVTKN FTQWGCQPEG AYPDPELEYR NISREILEEV YKQDWPWNTY HWPLWQMENM RQWMKENEKE YKERTNKTKE DIDDLVAGRI RGRFCVPYPY ALLRCEEWCW YPESINQETG HAEKIKINCT KAKAVSCTEK MSLAAVQRVY WEKEDEESMK FLNIKACNIS LRCQDEGKSP GGCVQGYPIP KGAEIIPEAM KYLRGKKSRY GGIKDKNGEL KLPLSVRVWV RMANLSGWVN GTPPYWSARI NGSTGINGTR WYGIGTLHHL GCNISSNPER GICNFTGELW IGGDKFPYYY TPSWNCSQNW TGHPVWHVFR YLDMTEHMTS RCIQRPKRHN ITVGNGTITG NCSVTNWDGC NCTRSGNHLY NSTSGGLLVI ICRQNSTITG IMGTNTNWTT MWNIYQNCSR CNNSSLDRTG SGTLGTVNNL KCSLPHRNES NKWTCKSQRD SYIAGRDFWG KVKAKYSCES NLGGLDSMMH QQMLLQRYQV IRVRAYTYGV VEMPQSYMEA QGENKRSRRN LQRKKRGIGL VIVLAIMAII AAAGAGLGVA NAVQQSYTRT AVQSLANATA AQQEVLEASY AMVQHIAKGI RILEARVARV EALVDRMMVY QELDCWHYQH YCVTSTRSEV ANYVNWTRFK DNCTWQQWEE EIEQHEGNLS LLLREAALQV HIAQRDARRI PDAWKAIQEA FNWSSWFSWL KYIPWIIMGI VGLMCFRILM CVISMCLQAY KQVKQIRYTQ VTVVIEAPVE LEEKQKRNGD GTNGCASLER ERRTSHRSFI QIWRATWWAW KTSPWRHNWR TMPYITLLPI LVIWQWMEEN GWNGENQHKK KKERVDCQDR EQMPTLENDY VEL (SEQ ID NO: 108).

In some cases, the heterologous glycoprotein used for pseudotyping is a visna-maedi virus glycoprotein. A suitable visna-maedi virus protein comprises an amino acid sequence having at least 80%, at least 85%, at least 90%, at least 95%, at least 98%, at least 99%, or 100%, amino acid sequence identity to the following amino acid sequence: QCQA EEVIALVSDP GGFQRVQHVE TVPVTCVTKN FTQWGCQPEG AYPDPELEYR NISREILEEV YKQDWPWNTY HWPLWQMENM RQWMKENEKE YKERTNKTKE DIDDLVAGRI RGRFCVPYPY ALLRCEEWCW YPESINQETG HAEKIKINCT KAKAVSCTEK MSLAAVQRVY WEKEDEESMK FLNIKACNIS LRCQDEGKSP GGCVQGYPIP KGAEIIPEAM KYLRGKKSRY GGIKDKNGEL KLPLSVRVWV RMANLSGWVN GTPPYWSARI NGSTGINGTR WYGIGTLHHL GCNISSNPER GICNFTGELW IGGDKFPYYY TPSWNCSQNW TGHPVWHVFR YLDMTEHMTS RCIQRPKRHN ITVGNGTITG NCSVTNWDGC NCTRSGNHLY NSTSGGLLVI ICRQNSTITG IMGTNTNWTT MWNIYQNCSR CNNSSLDRTG SGTLGTVNNL KCSLPHRNES NKWTCKSQRD SYIAGRDFWG KVKAKYSCES NLGGLDSMMH QQMLLQRYQV IRVRAYTYGV VEMPQSYMEA QGENKRSRRN LQRKKR (SEQ ID NO: 109).

In some cases, the heterologous glycoprotein used for pseudotyping is a visna-maedi virus glycoprotein. A suitable visna-maedi virus protein comprises an amino acid sequence having at least 80%, at least 85%, at least 90%, at least 95%, at least 98%, at least 99%, or 100%, amino acid sequence identity to the following amino acid sequence: GIGL VIVLAIMAII AAAGAGLGVA NAVQQSYTRT AVQSLANATA AQQEVLEASY AMVQHIAKGI RILEARVARV EALVDRMMVY QELDCWHYQH YCVTSTRSEV ANYVNWTRFK DNCTWQQWEE EIEQHEGNLS LLLREAALQV HIAQRDARRI PDAWKAIQEA FNWSSWFSWL KYIPWIIMGI VGLMCFRILM CVISMCLQAY KQVKQIRYTQ VTVVIEAPVE LEEKQKRNGD GTNGCASLER ERRTSHRSFI QIWRATWWAW KTSPWRHNWR TMPYITLLPI LVIWQWMEEN GWNGENQHKK KKERVDCQDR EQMPTLENDY VEL (SEQ ID NO:110).

In some cases, the heterologous glycoprotein used for pseudotyping is a severe acute respiratory syndrome-associated coronavirus (SARS-CoV) spike glycoprotein. A suitable SARS-CoV protein comprises an amino acid sequence having at least 80%, at least 85%, at least 90%, at least 95%, at least 98%, at least 99%, or 100%, amino acid sequence identity to the following amino acid sequence: MFIFLLFLTL TSGSDLDRCT TFDDVQAPNY TQHTSSMRGV YYPDEIFRSD TLYLTQDLFL PFYSNVTGFH TINHTFGNPV IPFKDGIYFA ATEKSNVVRG WVFGSTMNNK SQSVIIINNS TNVVIRACNF ELCDNPFFAV SKPMGTQTHT MIFDNAFNCT FEYISDAFSL DVSEKSGNFK HLREFVFKNK DGFLYVYKGY QPIDVVRDLP SGFNTLKPIF KLPLGINITN FRAILTAFSP AQDIWGTSAA AYFVGYLKPT TFMLKYDENG TITDAVDCSQ NPLAELKCSV KSFEIDKGIY QTSNFRVVPS GDVVRFPNIT NLCPFGEVFN ATKFPSVYAW ERKKISNCVA DYSVLYNSTF FSTFKCYGVS ATKLNDLCFS NVYADSFVVK GDDVRQIAPG QTGVIADYNY KLPDDFMGCV LAWNTRNIDA TSTGNYNYKY RYLRHGKLRP FERDISNVPF SPDGKPCTPP ALNCYWPLND YGFYTTTGIG YQPYRVVVLS FELLNAPATV CGPKLSTDLI KNQCVNFNFN GLTGTGVLTP SSKRFQPFQQ FGRDVSDFTD SVRDPKTSEI LDISPCSFGG VSVITPGTNA SSEVAVLYQD VNCTDVSTAI HADQLTPAWR IYSTGNNVFQ TQAGCLIGAE HVDTSYECDI PIGAGICASY HTVSLLRSTS QKSIVAYTMS LGADSSIAYS NNTIAIPTNF SISITTEVMP VSMAKTSVDC NMYICGDSTE CANLLLQYGS FCTQLNRALS GIAAEQDRNT REVFAQVKQM YKTPTLKYFG GFNFSQILPD PLKPTKRSFI EDLLFNKVTL ADAGFMKQYG ECLGDINARD LICAQKFNGL TVLPPLLTDD MIAAYTAALV SGTATAGWTF GAGAALQIPF AMQMAYRFNG IGVTQNVLYE NQKQIANQFN KAISQIQESL TTTSTALGKL QDVVNQNAQA LNTLVKQLSS NFGAISSVLN DILSRLDKVE AEVQIDRLIT GRLQSLQTYV TQQLIRAAEI RASANLAATK MSECVLGQSK RVDFCGKGYH LMSFPQAAPH GVVFLHVTYV PSQERNFTTA PAICHEGKAY FPREGVFVFN GTSWFITQRN FFSPQIITTD NTFVSGNCDV VIGIINNTVY DPLQPELDSF KEELDKYFKN HTSPDVDLGD ISGINASVVN IQKEIDRLNE VAKNLNESLI DLQELGKYEQ YIKWPWYVWL GFIAGLIAIV MVTILLCCMT SCCSCLKGAC SCGSCCKFDE DDSEPVLKGV KLHYT (SEQ ID NO:111; GenBank Accession No: ABA02260). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is a SARS-CoV S2 glycoprotein. A suitable SARS-CoV protein comprises an amino acid sequence having at least 80%, at least 85%, at least 90%, at least 95%, at least 98%, at least 99%, or 100%, amino acid sequence identity to the following amino acid sequence: CDI PIGAGICASY HTVSLLRSTS QKSIVAYTMS LGADSSIAYS NNTIAIPTNF SISITTEVMP VSMAKTSVDC NMYICGDSTE CANLLLQYGS FCTQLNRALS GIAAEQDRNT REVFAQVKQM YKTPTLKYFG GFNFSQILPD PLKPTKRSFI EDLLFNKVTL ADAGFMKQYG ECLGDINARD LICAQKFNGL TVLPPLLTDD MIAAYTAALV SGTATAGWTF GAGAALQIPF AMQMAYRFNG IGVTQNVLYE NQKQIANQFN KAISQIQESL TTTSTALGKL QDVVNQNAQA LNTLVKQLSS NFGAISSVLN DILSRLDKVE AEVQIDRLIT GRLQSLQTYV TQQLIRAAEI RASANLAATK MSECVLGQSK RVDFCGKGYH LMSFPQAAPH GVVFLHVTYV PSQERNFTTA PAICHEGKAY FPREGVFVFN GTSWFITQRN FFSPQIITTD NTFVSGNCDV VIGIINNTVY DPLQPELDSF KEELDKYFKN HTSPDVDLGD ISGINASVVN IQKEIDRLNE VAKNLNESLI DLQELGKYEQ YIKWPWYVWL GFIAGLIVIV MVTILLCCMT SCCSCLKGAC SCGSCCKFDE DDSEPVLKGV KL (SEQ ID NO: 112; GenBank Accession No: ABD73002). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is a SARS-CoV spike receptor binding domain glycoprotein. A suitable SARS-CoV protein comprises an amino acid sequence having at least 80%, at least 85%, at least 90%, at least 95%, at least 98%, at least 99%, or 100%, amino acid sequence identity to the following amino acid sequence: PNIT NLCPFGEVFN ATKFPSVYAW ERKKISNCVA DYSVLYNSTF FSTFKCYGVS ATKLNDLCFS NVYADSFVVK GDDVRQIAPG QTGVIADYNY KLPDDFMGCV LAWNTRNIDA TSTGNYNYKY RYLRHGKLRP FERDISNVPF SPDGKPCTPP ALNCYWPLND YGFYTTTGIG YQPYRVVVLS FELLNAPATV CGPKLSTDLI KNQCVNFNFN GLTGTGVLTP SSKRFQPFQQ FGRDVSDFTD SVRDPKTSE (SEQ ID NO:113; GenBank Accession No: ABD73002). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is a respiratory syncytial virus (RSV) glycoprotein G. A suitable RSV protein comprises an amino acid sequence having at least 80%, at least 85%, at least 90%, at least 95%, at least 98%, at least 99%, or 100%, amino acid sequence identity to the following amino acid sequence: MSKNKDQRTA KTLERTWDTL NHLLFISSCL YKLNLKSVAQ ITLSILAMII STSLIIAAII FIASANHKVT PTTAIIQDAT SQIKNTTPTY LTQNPQLGIS PSNPSEITSQ ITTILASTTP GVKSTLQSTT VKTKNTTTTQ TQPSKPTTKQRQNKPPSKPN NDFHFEVFNF VPCSICSNNP TCWAICKRIP NKKPGKKTTTKPTKKPTLKT TKKDPKPQTT KSKEVPTTKP TEEPTINTTK TNIITTLLTS NTTGNPELTS QMETFHSTSS EGNPSPSQVS TTSEYPSQPS SPPNTPRQ (SEQ ID NO: 114; UniProtKB: P03423-1). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is an RSV glycoprotein F. A suitable RSV protein comprises an amino acid sequence having at least 80%, at least 85%, at least 90%, at least 95%, at least 98%, at least 99%, or 100%, amino acid sequence identity to the following amino acid sequence: MELLILKANA ITTILTAVTF CFASGQNITE EFYQSTCSAV SKGYLSALRT GWYTSVITIE LSNIKENKCN GTDAKVKLIK QELDKYKNAV TELQLLMQST PPTNNRARRE LPRFMNYTLN NAKKTNVTLS KKRKRRFLGF LLGVGSAIAS GVAVSKVLHL EGEVNKIKSA LLSTNKAVVS LSNGVSVLTS KVLDLKNYID KQLLPIVNKQ SCSISNIETV IEFQQKNNRL LEITREFSVN AGVTTPVSTY MLTNSELLSL INDMPITNDQ KKLMSNNVQI VRQQSYSIMS IIKEEVLAYV VQLPLYGVID TPCWKLHTSP LCTTNTKEGS NICLTRTDRG WYCDNAGSVS FFPQAETCKV QSNRVFCDTM NSLTLPSEIN LCNVDIFNPK YDCKIMTSKT DVSSSVITSL GAIVSCYGKT KCTASNKNRG IIKTFSNGCD YVSNKGMDTV SVGNTLYYVN KQEGKSLYVK GEPIINFYDP LVFPSDEFDA SISQVNEKIN QSLAFIRKSD ELLHNVNAGK STTNIMITTI IIVIIVILLS LIAVGLLLYC KARSTPVTLS KDQLSGINNI AFSN (SEQ ID NO: 115; GenBank Accession No: P03420). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is an RSV glycoprotein. A suitable RSV protein comprises an amino acid sequence having at least 80%, at least 85%, at least 90%, at least 95%, at least 98%, at least 99%, or 100%, amino acid sequence identity to the following amino acid sequence: QNITE EFYQSTCSAV SKGYLSALRT GWYTSVITIE LSNIKENKCN GTDAKVKLIK QELDKYKNAV TELQLLMQST PPTNNRARRE LPRFMNYTLN NAKKTNVTLS KKRKRRFLGF LLGVGSAIAS GVAVSKVLHL EGEVNKIKSA LLSTNKAVVS LSNGVSVLTS KVLDLKNYID KQLLPIVNKQ SCSISNIETV IEFQQKNNRL LEITREFSVN AGVTTPVSTY MLTNSELLSL INDMPITNDQ KKLMSNNVQI VRQQSYSIMS IIKEEVLAYV VQLPLYGVID TPCWKLHTSP LCTTNTKEGS NICLTRTDRG WYCDNAGSVS FFPQAETCKV QSNRVFCDTM NSLTLPSEIN LCNVDIFNPK YDCKIMTSKT DVSSSVITSL GAIVSCYGKT KCTASNKNRG IIKTFSNGCD YVSNKGMDTV SVGNTLYYVN KQEGKSLYVK GEPIINFYDP LVFPSDEFDA SISQVNEKIN QSLAFIRKSD ELLHNVNAGK STTNIMITTI IIVIIVILLS LIAVGLLLYC KARSTPVTLS KDQLSGINNI AFSN (SEQ ID NO: 116). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is an RSV FO glycoprotein. A suitable RSV protein comprises an amino acid sequence having at least 80%, at least 85%, at least 90%, at least 95%, at least 98%, at least 99%, or 100%, amino acid sequence identity to the following amino acid sequence: QNITE EFYQSTCSAV SKGYLSALRT GWYTSVITIE LSNIKENKCN GTDAKVKLIK QELDKYKNAV TELQLLMQST PPTNNRARRE LPRFMNYTLN NAKKTNVTLS KKRKRRFLGF LLGVGSAIAS GVAVSKVLHL EGEVNKIKSA LLSTNKAVVS LSNGVSVLTS KVLDLKNYID KQLLPIVNKQ SCSISNIETV IEFQQKNNRL LEITREFSVN AGVTTPVSTY MLTNSELLSL INDMPITNDQ KKLMSNNVQI VRQQSYSIMS IIKEEVLAYV VQLPLYGVID TPCWKLHTSP LCTTNTKEGS NICLTRTDRG WYCDNAGSVS FFPQAETCKV QSNRVFCDTM NSLTLPSEIN LCNVDIFNPK YDCKIMTSKT DVSSSVITSL GAIVSCYGKT KCTASNKNRG IIKTFSNGCD YVSNKGMDTV SVGNTLYYVN KQEGKSLYVK GEPIINFYDP LVFPSDEFDA SISQVNEKIN QSLAFIRKSD ELLHNVNAGK STTNIMITTI IIVIIVILLS LIAVGLLLYC KARSTPVTLS KDQLSGINNI AFSN (SEQ ID NO: 116; GenBank Accession No: P03420). Such a glycoprotein may be useful for targeting a VLP of the present disclosure to cells of respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is an RSV F2 glycoprotein. A suitable RSV protein comprises an amino acid sequence having at least 80%, at least 85%, at least 90%, at least 95%, at least 98%, at least 99%, or 100%, amino acid sequence identity to the following amino acid sequence: QNITE EFYQSTCSAV SKGYLSALRT GWYTSVITIE LSNIKENKCN GTDAKVKLIK QELDKYKNAV TELQLLMQST PPTNNRARRE LPRFMNYTLN NAKKTNVTLS KKRKRR (SEQ ID NO: 117; GenBank Accession No: P03420). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is an RSV F1 glycoprotein. A suitable RSV protein comprises an amino acid sequence having at least 80%, at least 85%, at least 90%, at least 95%, at least 98%, at least 99%, or 100%, amino acid sequence identity to the following amino acid sequence: FLGF LLGVGSAIAS GVAVSKVLHL EGEVNKIKSA LLSTNKAVVS LSNGVSVLTS KVLDLKNYID KQLLPIVNKQ SCSISNIETV IEFQQKNNRL LEITREFSVN AGVTTPVSTY MLTNSELLSL INDMPITNDQ KKLMSNNVQI VRQQSYSIMS IIKEEVLAYV VQLPLYGVID TPCWKLHTSP LCTTNTKEGS NICLTRTDRG WYCDNAGSVS FFPQAETCKV QSNRVFCDTM NSLTLPSEIN LCNVDIFNPK YDCKIMTSKT DVSSSVITSL GAIVSCYGKT KCTASNKNRG IIKTFSNGCD YVSNKGMDTV SVGNTLYYVN KQEGKSLYVK GEPIINFYDP LVFPSDEFDA SISQVNEKIN QSLAFIRKSD ELLHNVNAGK STTNIMITTI IIVIIVILLS LIAVGLLLYC KARSTPVTLS KDQLSGINNI AFSN (SEQ ID NO: 118; GenBank Accession No: P03420). Such a glycoprotein may be useful for targeting a VLP of the present disclosure to cells of the lung/respiratory tract.

In some cases, the heterologous glycoprotein used for pseudotyping is an RSV glycoprotein. A suitable RSV protein comprises an amino acid sequence having at least 80%, at least 85%, at least 90%, at least 95%, at least 98%, at least 99%, or 100%, amino acid sequence identity to the following amino acid sequence: QNITE EFYQSTCSAV SKGYLSALRT GWYTSVITIE LSNIKENKCN GTDAKVKLIK QELDKYKNAV TELQLLMQST PPTNNRARRE LPRFMNYTLN NAKKTNVTLS KKRKRRFLGF LLGVGSAIAS GVAVSKVLHL EGEVNKIKSA LLSTNKAVVS LSNGVSVLTS KVLDLKNYID KQLLPIVNKQ SCSISNIETV IEFQQKNNRL LEITREFSVN AGVTTPVSTY MLTNSELLSL INDMPITNDQ KKLMSNNVQI VRQQSYSIMS IIKEEVLAYV VQLPLYGVID TPCWKLHTSP LCTTNTKEGS NICLTRTDRG WYCDNAGSVS FFPQAETCKV QSNRVFCDTM NSLTLPSEIN LCNVDIFNPK YDCKIMTSKT DVSSSVITSL GAIVSCYGKT KCTASNKNRG IIKTFSNGCD YVSNKGMDTV SVGNTLYYVN KQEGKSLYVK GEPIINFYDP LVFPSDEFDA SISQVNEKIN QSLAFIRKSD ELLHNVNAGK STTNIMITTI IIVIIVILLS LIAVGLLLYC KARSTPVTLS KDQLSGINNI AFSN (SEQ ID NO: 116). Such a glycoprotein may be useful for targeting a VLP of the present disclosure to cells of the lung/respiratory tract.

In some cases, the heterologous glycoprotein used for pseudotyping is a human parainfluenza virus type 3 hemagglutinin-neuraminidase glycoprotein. A suitable human parainfluenza virus type 3 protein comprises an amino acid sequence having at least 80%, at least 85%, at least 90%, at least 95%, at least 98%, at least 99%, or 100%, amino acid sequence identity to the following amino acid sequence: MEYWKHTNHG KDAGNELETS MATHGNKLTN KITYILWTII LVLLSIVFII VLINSIKSEK AHESLLQNIN NEFMEITEKI QMASDNTNDL IQSGVNTRLL TIQSHVQNYI PISLTQQMSD LRKFISEITI RNDNQEVLPQ RITHDVGIKP LNPDDFWRCT SGLPSLMKTP KIRLMPGPGL LAMPTTVDGC IRTPSLVIND LIYAYTSNLI TRGCQDIGKS YQVLQIGIIT VNSDLVPDLN PRISHTFNIN DNRKSCSLAL LNTDVYQLCS TPKVDERSDY ASPGIEDIVL DIVNYDGSIS TTRFKNNNIS FDQPYAALYP SVGPGIYYKG KIIFLGYGGL EHPINENVIC NTTGCPGKTQ RDCNQASHSP WFSDRRMVNS IIVVDKGLNS IPKLKVWTIS MRQNYWGSEG RLLLLGNKIY IYTRSTSWHS KLQLGIIDIT DYSDIRIKWT WHNVLSRPGN NECPWGHSCP DGCITGVYTD AYPLNPTGSI VSSVILDSQK SRVNPVITYS TATERVNELA ILNRTLSAGY TTTSCITHYN KGYCFHIVEI NHKSLNTLQP MLFKTEIPKS CS (SEQ ID NO: 119; GenBank Accession No: AAP35240). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is a human parainfluenza virus type 3 glycoprotein F0. A suitable human parainfluenza virus type 3 protein comprises an amino acid sequence having at least 80%, at least 85%, at least 90%, at least 95%, at least 98%, at least 99%, or 100%, amino acid sequence identity to the following amino acid sequence: MPISILLIIT TMIMASHCQI DITKLQHVGV LVNSPKGMKI SQNFETRYLI LSLIPKIDDS NSCGDQQIKQ YKRLLDRLII PLYDGLRLQK DVIVANQESN ENTDPRTERF FGGVIGTIAL GVATSAQITA AVALVEAKQA RSDIEKLKEA IRDTNKAVQS VQSSVGNLIV AIKSVQDYVN KEIVPSIARL GCEAAGLQLG IALTQHYSEL TNIFGDNIGS LQEKGIKLQG IASLYRTNIT EIFTTSTVDK YDIYDLLFTE SIKVRVIDVD LNDYSITLQV RLPLLTRLLN TQIYKVDSIS YNIQNREWYI PLPSHIMTKG AFLGGADVKE CIEAFSSYIC PSDPGFVLNH EMESCLSGNI SQCPRTTVTS DIVPRYAFVN GGVVANCITT TCTCNGIGNR INQPPDQGVK IITHKECNTI GINGMLFNTN KEGTLAFYTP ADITLNNSVA LDPIDISIEL NKAKSDLEES KEWIRRSNQK LDSIGSWHQS STTIIVILIM MIILFIINIT IITIAIKYYR IQKRNRVDQN DKPYVLTNK (SEQ ID NO: 120; GenBank Accession No: AXA52708). Such a glycoprotein may be useful for targeting a VLP of the present disclosure to cells of the respiratory tract (e.g., cells of the lung), where such cells include, e.g., epithelial cells, goblet cells, club cells, type I pneumocytes, type II pneumocytes, monocytes, macrophages, dendritic cells, neutrophils, and NK cells.

In some cases, the heterologous glycoprotein used for pseudotyping is a Hepatitis C virus (HCV) E1 glycoprotein. A suitable HCV protein comprises an amino acid sequence having at least 80%, at least 85%, at least 90%, at least 95%, at least 98%, at least 99%, or 100%, amino acid sequence identity to the following amino acid sequence: YQVRNSSGLY HVTNDCPNSS IVYEAADAIL HTPGCVPCVR EGNASRCWVA VTPTVATRDG KLPTTQLRRH IDLLVGSATL CSALYVGDLC GSVFLVGQLF TFSPRRHWTT QDCNCSIYPG HITGHRMAWD MMMNWSPTAA LVVAQLLRIP QAIMDMIAGA HWGVLAGIAY FSMVGNWAKV LVVLLLFAGV DA (SEQ ID NO:121; GenBank Accession No: NP_751920). Such a glycoprotein may be useful for targeting a VLP of the present disclosure to a liver cell.

In some cases, the heterologous glycoprotein used for pseudotyping is an HCV E2 glycoprotein. A suitable HCV protein comprises an amino acid sequence having at least 80%, at least 85%, at least 90%, at least 95%, at least 98%, at least 99%, or 100%, amino acid sequence identity to the following amino acid sequence: ETHVTGGSAG RTTAGLVGLL TPGAKQNIQL INTNGSWHIN STALNCNESL NTGWLAGLFY QHKFNSSGCP ERLASCRRLT DFAQGWGPIS YANGSGLDER PYCWHYPPRP CGIVPAKSVC GPVYCFTPSP VVVGTTDRSG APTYSWGAND TDVFVLNNTR PPLGNWFGCT WMNSTGFTKV CGAPPCVIGG VGNNTLLCPT DCFRKHPEAT YSRCGSGPWI TPRCMVDYPY RLWHYPCTIN YTIFKVRMYV GGVEHRLEAA CNWTRGERCD LEDRDRSELS PLLLSTTQWQ VLPCSFTTLP ALSTGLIHLH QNIVDVQYLY GVGSSIASWA IKWEYVVLLF LLLADARVCS CLWMMLLISQ AEA (SEQ ID NO: 122; GenBank Accession No: NP_751921). Such a glycoprotein may be useful for targeting a VLP of the present disclosure to a liver cell.

In some cases, the heterologous glycoprotein used for pseudotyping is a fowl plague virus glycoprotein. A suitable fowl plague virus protein comprises an amino acid sequence having at least 80%, at least 85%, at least 90%, at least 95%, at least 98%, at least 99%, or 100%, amino acid sequence identity to the following amino acid sequence: MNTQILVFAL VAVIPTNADK ICLGHHAVSN GTKVNTLTER GVEVVNATET VERTNIPKIC SKGKRTTDLG QCGLLGTITG PPQCDQFLEF SADLIIERRE GNDVCYPGKF VNEEALRQIL RGSGGIDKET MGFTYSGIRT NGTTSACRRS GSSFYAEMEW LLSNTDNASF PQMTKSYKNT RRESALIVWG IHHSGSTTEQ TKLYGSGNKL ITVGSSKYHQ SFVPSPGTRP QINGQSGRID FHWLILDPND TVTFSFNGAF IAPNRASFLR GKSMGIQSDV QVDANCEGEC YHSGGTITSR LPFQNINSRA VGKCPRYVKQ ESLLLATGMK NVPEPSKKRE KRGLFGAIAG FIENGWEGLV DGWYGFRHQN AQGEGTAADY KSTQSAIDQI TGKLNRLIEK TNQQFELIDN EFTEVEKQIG NLINWTKDFI TEVWSYNAEL LVAMENQHTI DLADSEMNKL YERVRKQLRE NAEEDGTGCF EIFHKCDDDC MASIRNNTYD HSKYREEAMQ NRIQIDPVKL SSGYKDVILW FSFGASCFLL LAIAVGLVFI CVKNGNMRCT ICI (SEQ ID NO: 123; GenBank Accession No: 0601245A).

In some cases, the heterologous glycoprotein used for pseudotyping is an Autographa californica nuclear polyhedrosis virus (AcMNPV) major envelope glycoprotein gp64. A suitable AcMNPV protein comprises an amino acid sequence having at least 80%, at least 85%, at least 90%, at least 95%, at least 98%, at least 99%, or 100%, amino acid sequence identity to the following amino acid sequence: MVSAIVLYVL LAAAAHSAFA AEHCNAQMKT GPYKIKNLDI TPPKETLQKD VEITIVETDY NENVIIGYKG YYQAYAYNGG SLDPNTRVEE TMKTLNVGKE DLLMWSIRQQ CEVGEELIDR WGSDSDDCFR DNEGRGQWVK GKELVKRQNN NHFAHHTCNK SWRCGISTSK MYSRLECQDD TDECQVYILD AEGNPINVTV DTVLHRDGVS MILKQKSTFT TRQIKAACLL IKDDKNNPES VTREHCLIDN DIYDLSKNTW NCKFNRCIKR KVEHRVKKRP PTWRHNVRAK YTEGDTATKG DLMHIQEELM YENDLLKMNI ELMHAHINKL NNMLHDLIVS VAKVDERLIG NLMNNSVSST FLSDDTFLLM PCTNPPAHTS NCYNNSIYKE GRWVANTDSS QCIDFSNYKE LAIDDDVEFW IPTIGNTTYH DSWKDASGWS FIAQQKSNLI TTMENTKFGG VGTSLSDITS MAEGELAAKL TSFMFGHVVN FVIILIVILF LYCMIRNRNR QY (SEQ ID NO:217; UniProt Accession No: P17501-1).

In some cases, the heterologous glycoprotein used for pseudotyping is an AcMNPV glycoprotein. A suitable AcMNPV protein comprises an amino acid sequence having at least 80%, at least 85%, at least 90%, at least 95%, at least 98%, at least 99%, or 100%, amino acid sequence identity to the following amino acid sequence: AEHCNAQMKT GPYKIKNLDI TPPKETLQKD VEITIVETDY NENVIIGYKG YYQAYAYNGG SLDPNTRVEE TMKTLNVGKE DLLMWSIRQQ CEVGEELIDR WGSDSDDCFR DNEGRGQWVK GKELVKRQNN NHFAHHTCNK SWRCGISTSK MYSRLECQDD TDECQVYILD AEGNPINVTV DTVLHRDGVS MILKQKSTFT TRQIKAACLL IKDDKNNPES VTREHCLIDN DIYDLSKNTW NCKFNRCIKR KVEHRVKKRP PTWRHNVRAK YTEGDTATKG DLMHIQEELM YENDLLKMNI ELMHAHINKL NNMLHDLIVS VAKVDERLIG NLMNNSVSST FLSDDTFLLM PCTNPPAHTS NCYNNSIYKE GRWVANTDSS QCIDFSNYKE LAIDDDVEFW IPTIGNTTYH DSWKDASGWS FIAQQKSNLI TTMENTKFGG VGTSLSDITS MAEGELAAKL TSFMFGHVVN FVIILIVILF LYCMIRNRNR QY (SEQ ID NO: 124).

In some cases, the heterologous glycoprotein used for pseudotyping is a measles virus hemagglutinin (H) polypeptide. See, e.g., Levy et al. (2017) Blood Adv. 1:2088. A suitable measles virus H polypeptide comprises an amino acid sequence having at least 80%, at least 85%, at least 90%, at least 95%, at least 98%, at least 99%, or 100%, amino acid sequence identity to the following amino acid sequence: MSPQRDRINA FYKDNPHPKG SRIVINREHL MIDRPYVLLA VLFVMFLSLI GLLAIAGIRL HRAAIYTAEI HKSLSTNLDV TNSIEHQVKD VLTPLFKIIG DEVGLRTPQR FTDLVKFISD KIKFLNPDRE YDFRDLTWCI NPPERIKLDY DQYCADVAAE ELMNALVNST LLETRTTNQF LAVSKGNCSG PTTIRGQFSN MSLSLLDLYL SRGYNVSSIV TMTSQGMYGG TYLVEKPNLS SKGSELSQLS MYRVFEVGVI RNPGLGAPVF HMTNYFEQPV SNDLSNCMVA LGELKLAALC HGGDSITIPY QGSGKGVSFQ LVKLGVWKSP TDMQSWVPLS TDDPVIDRLY LSSHRGVIAD NQAKWAVPTT RTDDKLRMET CFQQACKGKI QALCENPEWA PLKDNRIPSY GVLSVDLSLT VELKIKIASG FGPLITHGSG MDLYKSNHNN VYWLTIPPMK NLALGVINTL EWIPRFKVSP YLFTVPIKEA GEDCHAPTYL PAEVDGDVKL SSNLVILPGQ DLQYVLATYD TSRVEHAVVY YVYSPSRSFS YFYPFRLPIK GIPIELQVEC FTWDQKLWCR HFCVLADSES GGHITHSGMV GMGVSCTVTR EDGTNSR (SEQ ID NO: 125). Such a glycoprotein may be useful for targeting a VLP of the present disclosure to T cells, B cells, monocytes, macrophages, dendritic cells, and hematopoietic stem cells (e.g., CD34⁺ cells).

In some cases, the heterologous glycoprotein used for pseudotyping is a measles virus fusion (F) polypeptide. A suitable measles virus F polypeptide comprises an amino acid sequence having at least 80%, at least 85%, at least 90%, at least 95%, at least 98%, at least 99%, or 100%, amino acid sequence identity to the following amino acid sequence: MSIMGLKVNV SAIFMAVLLT LQTPTGQIHW GNLSKIGVVG IGSASYKVMT RSSHQSLVIK LMPNITLLNN CTRVEIAEYR RLLRTVLEPI RDALNAMTQN IRPVQSVASS RRHKRFAGVV LAGAALGVAT AAQITAGIAL HQSMLNSQAI DNLRASLETT NQAIETIRQA GQEMILAVQG VQDYINNELI PSMNQLSCDL IGQKLGLKLL RYYTEILSLF GPSLRDPISA EISIQALSYA LGGDINKVLE KLGYSGGDLL GILESGGIKA RITHVDTESY FIVLSIAYPT LSEIKGVIVH RLEGVSYNIG SQEWYTTVPK YVATQGYLIS NFDESSCTFM PEGTVCSQNA LYPMSPLLQE CLRGYTKSCA RTLVSGSFGN RFILSQGNLI ANCASILCKC YTTGTIINQD PDKILTYIAA DHCPVVEVNG VTIQVGSRRY PDAVYLHRID LGPPISLERL DVGTNLGNAI AKLEDAKELL ESSDQILRSM KGLSSTSIVY ILIAVCLGGL IGIPALICCC RGRCNKKGEQ VGMSRPGLKP DLTGTSKSYV RSL (SEQ ID NO: 126). Such a glycoprotein may be useful for targeting a VLP of the present disclosure to T cells, B cells, monocytes, macrophages, dendritic cells, and hematopoietic stem cells (e.g., CD34⁺ cells). In some cases, both measles virus hemagglutinin and measles virus F protein are used to pseudotype a VLP of the present disclosure.

In some cases, both measles virus L and measles virus H polypeptides are used to pseudotype a VLP of the present disclosure.

Polypeptides that Bind to a Target Cell or Target Cell Type

In some cases, a VLP of the present disclosure comprises a polypeptide that provides for binding to a target cell or target cell type. Such polypeptides include antibodies (e.g., scFv; nanobody; and the like) and antibody mimetics (e.g., DARPins).

In some cases, the antibody targets a cancer antigen, thereby targeting the VLP to a cancerous cell that displays the cancer antigen on its cell surface. In some cases, the antibody provides for selective binding to an organ such as kidney, liver, bone, pancreas, brain, lung, heart, and the like. In some cases, the antibody provides for selective binding to a particular cell type. For example, in some cases, the antibody provides for selective binding to a cell such as a skeletal muscle cell, a cardiomyocyte, an adipocyte, an epithelial cell, an endothelial cell, a macrophage, a beta islet cell, or an immune cell (e.g., a T cell, a B cell, a monocyte, a natural killer cell, a dendritic cell, etc.). In some cases, the antibody provides for selective binding to a diseased cell, relative to a non-diseased cell of the same cell type.

Suitable antigens bound by an antibody present in a VLP of the present disclosure include, e.g., CD3, epidermal growth factor receptor (EGFR), CA-125 (highly expressed on epithelial ovarian cancer cells), CD80, CD86, glycoprotein IIb/IIIa receptor, CD51, TNF-α, epithelial adhesion molecule EpcAM (CD326), vascular endothelial growth factor receptor-2 (VEGFR-2), CD52, mesothelin, activin receptor-like kinase 1 (ALK-1), phosphatidyl serine, CD19, vascular endothelial growth factor A (VEGF-A), IL-6 receptor, CD11a, CD25, CD2, CD3 receptor, and the like.

Suitable antigens bound by an antibody present in a VLP of the present disclosure include, e.g., carbonic anhydrase IX, alpha-fetoprotein (AFP), a-actinin-4, A3, ART-4, B7, Ba 733, BAGE, BrE3-antigen, CA125, CAMEL, CAP-1, CASP-8/m, CCL19, CCL21, CD1, CDla, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD80, CD83, CD95, CD126, CD132, CD133, CD138, CD147, CD154, CDC27, CDK-4/m, CDKN2A, CTLA-4, CXCR4, CXCR7, CXCL12, HIF-1α, colon-specific antigen-p (CSAp), CEACAM5, CEACAM6, c-Met, DAM, epidermal growth factor receptor (EGFR), EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-P, HLA-DR, HM1.24, human chorionic gonadotropin (HCG) and its subunits, HER2/neu, histone H2B, histone H3, histone H4, HMGB-1, hypoxia inducible factor (HIF-1), HSP70-2M, HST-2, insulin-like growth factor-1 receptor (IGF-1R), IFN-γ IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-IA, MIP-1B, MIF, MUC1, MUC2, MUC3, MUC4, MUC5ac, MUC13, MUC16, MUM-1/2, MUM-3, NCA66, NCA95, NCA90, PAM4 antigen, PD-1, PD-L1, PD-1 receptor, placental growth factor, p53, PLAGL2, prostatic acid phosphatase, PSA, PRAME, PSMA, P1GF, ILGF, ILGF-1R, IL-6, IL-25, RS5, RANTES, T101, SAGE, 5100, survivin, survivin-2B, TAC, TAG-72, tenascin, TRAIL receptors, TNF-α, Tn antigen, tumor necrosis antigens, VEGFR, ED-B fibronectin, WT-1, 17-1A-antigen, complement factors C3, C3a, C3b, C5a, C5; and the like.

Examples of suitable antibodies include, e.g., abciximab (anti-glycoprotein IIb/IIIa), alemtuzumab (anti-CD52), bevacizumab (anti-VEGF), cetuximab (anti-EGFR), gemtuzumab (anti-CD33), ibritumomab (anti-CD20), panitumumab (anti-EGFR), rituximab (anti-CD20), tositumomab (anti-CD20), trastuzumab (anti-ErbB2), lambrolizumab (anti-PD-1 receptor), nivolumab (anti-PD-1 receptor), ipilimumab (anti-CTLA-4), abagovomab (anti-CA-125), adecatumumab (anti-EpCAM), atlizumab (anti-IL-6 receptor), benralizumab (anti-CD125), obinutuzumab (GA101, anti-CD20), CC49 (anti-TAG-72), tocilizumab (anti-IL-6 receptor), basiliximab (anti-CD25), daclizumab (anti-CD25), efalizumab (anti-CD I1a), GA101 (anti-CD20; Glycart Roche), muromonab-CD3 (anti-CD3 receptor), natalizumab (anti-α-4 integrin), and the like.

### Antibody Mimetics

In some cases, a VLP of the present disclosure comprises an antibody mimetic. Non-limiting examples of antibody mimetics include peptide aptamers, affimers, affilins, affibodies, affitins, alphabodies, anticalins, avimers, DARPins, fynomers, Kunitz domain peptides, nanoCLAMPs, affinity reagents, and scaffold proteins.

### Compositions Comprising a Vlp

The present disclosure provides compositions, including pharmaceutical compositions, comprising a VLP of the present disclosure. The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A composition of the present disclosure can include: a) a VLP of the present disclosure; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc.

In some cases, the composition is sterile. In some cases, the composition is suitable for administration to a human subject, e.g., where the composition is sterile and is free of detectable pyrogens and/or other toxins.

In some cases, a composition of the present disclosure comprises: i) a VLP that does not include a donor template nucleic acid; and ii) a donor template nucleic acid (provided separately from the VLP).

### Systems

The present disclosure provides a system that can be used to generate a VLP of the present disclosure. A system of the present disclosure comprises: a) a first nucleic acid comprising a nucleotide sequence encoding a fusion polypeptide comprising: i) a lentiviral gag polyprotein comprising a matrix (MA) polypeptide, a capsid (CA) polypeptide, and a nucleocapsid (NC) polypeptide; and ii) a CRISPR-Cas effector polypeptide; wherein the fusion polypeptide comprises proteolytically cleavable linker between the gag polyprotein and the CRISPR-Cas effector polypeptide; b) a second nucleic acid comprising a nucleotide sequence encoding a therapeutic polypeptide, wherein the second nucleic acid is a recombinant lentiviral nucleic acid; c) a third nucleic acid comprising a nucleotide sequence encoding a pseudotyping viral envelope protein and/or a polypeptide that provides for binding to a target cell; and d) a fourth nucleic acid comprising a nucleotide sequence encoding a lentiviral pol polyprotein comprising a reverse transcriptase and an integrase. The system also comprises a nucleic acid comprising a nucleotide sequence encoding a CRISPR-Cas guide RNA. The CRISPR-Cas guide RNA-encoding nucleic acid is in some cases a separate (fifth) nucleic acid. In other cases, the CRISPR-Cas guide RNA-encoding nucleic acid part of the second nucleic acid; in other words, in some cases, the second nucleic acid comprises: i) a nucleotide sequence encoding a therapeutic polypeptide; and ii) a nucleotide sequence encoding the CRISPR-Cas guide RNA. In some cases, the fourth nucleic acid comprise a nucleotide sequence encoding lentivirus Gag and Pol.

In some cases, a system of the present disclosure comprises: a) a first nucleic acid comprising a nucleotide sequence encoding a fusion polypeptide comprising: i) a lentiviral gag polyprotein comprising a MA polypeptide, a CA polypeptide, and an NC polypeptide; and ii) a CRISPR-Cas effector polypeptide; wherein the fusion polypeptide comprises proteolytically cleavable linker between the gag polyprotein and the CRISPR-Cas effector polypeptide; b) a second nucleic acid comprising: i) a first nucleotide sequence encoding a therapeutic polypeptide; and ii) a second nucleotide sequence encoding a CRISPR-Cas guide RNA, wherein the second nucleic acid is a recombinant lentiviral nucleic acid; c) a third nucleic acid comprising a nucleotide sequence encoding a pseudotyping viral envelope protein and/or a polypeptide that provides for binding to a target cell; and d) a fourth nucleic acid comprising a nucleotide sequence encoding a lentiviral pol polyprotein comprising a reverse transcriptase and an integrase. In some cases, the fourth nucleic acid also comprises a nucleotide sequence encoding the lentiviral gag polyprotein.

In some cases, a system of the present disclosure comprises: a) a first nucleic acid comprising a nucleotide sequence encoding a fusion polypeptide comprising: i) a lentiviral gag polyprotein comprising a MA polypeptide, a CA polypeptide, and an NC polypeptide; and ii) a CRISPR-Cas effector polypeptide, wherein the fusion polypeptide comprises proteolytically cleavable linker between the gag polyprotein and the CRISPR-Cas effector polypeptide; b) a second nucleic acid comprising a nucleotide sequence encoding a therapeutic polypeptide; c) a third nucleic acid comprising a nucleotide sequence encoding a pseudotyping viral envelope protein and/or a polypeptide that provides for binding to a target cell; d) a fourth nucleic acid comprising a nucleotide sequence encoding a lentiviral pol polyprotein comprising a reverse transcriptase and an integrase; and e) a fifth nucleic acid comprising a nucleotide sequence encoding a CRISPR-Cas guide RNA. In some cases, the fourth nucleic acid also comprises a nucleotide sequence encoding the lentiviral gag polyprotein.

As noted above, in some cases, the fourth nucleic acid also comprises a nucleotide sequence encoding the lentiviral gag polyprotein; in other words, in some cases, the fourth nucleic acid comprises nucleotide sequences encoding lentiviral Gag and Pol polyproteins. Thus, e.g., in some cases, the first nucleic acid comprises a nucleotide sequence encoding a Gag-CRISPR-Cas fusion polypeptide, wherein the fusion polypeptide comprises proteolytically cleavable linker between the gag polyprotein and the CRISPR-Cas effector polypeptide; and the fourth nucleic acid comprises nucleotide sequences encoding lentiviral Gag and Pol polyproteins. In some cases, the fourth nucleic acid and the first nucleic acid are present in the system in a ratio of from 1.5:1 to 3:1. In some cases, the fourth nucleic acid and the first nucleic acid are present in the system in a ratio of about 2:1. In some cases, a system of the present disclosure comprises: i) about 3 μg of the first nucleic acid; and ii) about 6-7 μg of the fourth nucleic acid.

In some cases, a system of the present disclosure comprises: i) about 3 μg of a first nucleic acid comprising a nucleotide sequence encoding a Gag-CRISPR-Cas fusion polypeptide, wherein the fusion polypeptide comprises proteolytically cleavable linker between the gag polyprotein and the CRISPR-Cas effector polypeptide; about 6 or 7 μg of a fourth nucleic acid comprising nucleotide sequences encoding lentiviral Gag and Pol polyproteins; iii) about 2-3 μg of a second nucleic acid comprising a nucleotide sequence encoding a therapeutic protein; and iv) about 7-8 μg of a fifth nucleic acid comprising a nucleotide sequence encoding a CRISPR-Cas guide RNA.

In some cases, retroviral (e.g. lentiviral) Gag polypeptides include CA (p24), MA (p17) and NC (p7) polypeptides. In some cases, retroviral Gag polypeptides include CA, MA, and NC polypeptides, and in addition one or more of p1, p2, and p6 polypeptides. In some cases, retroviral Gag polypeptides include CA, MA, NC, and p6 polypeptides. In some cases, retroviral Gag polypeptides include CA, MA, NC, p1, p2, and p6 polypeptides. See, e.g., Muriaux and Darlix (2010) RNA Biol. 7:744.

As noted above, the first nucleic acid comprises a nucleotide sequence encoding a fusion polypeptide comprising: i) a lentiviral gag polyprotein comprising a MA polypeptide, a CA polypeptide, and an NC polypeptide; and ii) a CRISPR-Cas effector polypeptide, wherein the fusion polypeptide comprises proteolytically cleavable linker between the gag polyprotein and the CRISPR-Cas effector polypeptide. One of the nucleic acids in the system comprises a nucleotide sequence encoding a protease that cleaves the proteolytically cleavable linker. In some cases, the second nucleic acid of the system comprises a nucleotide sequence encoding a protease that cleaves the proteolytically cleavable linker. In some cases, the fourth nucleic acid of the system comprises a nucleotide sequence encoding a protease that cleaves the proteolytically cleavable linker. The proteolytically cleavable linker can be one that is cleaved by a lentiviral protease. The proteolytically cleavable linker can be one that is cleaved by a protease other than a lentiviral protease (i.e., the protease is heterologous to the lentivirus).

A proteolytically cleavable linker comprises a protease cleavage site. A proteolytically cleavable linker can comprise a matrix metalloproteinase cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (wherein, X represents an arbitrary residue; Hy, a hydrophobic residue (SEQ ID NO:218)), e.g., Pro-X-X-Hy-(Ser/Thr) (SEQ ID NO:219), e.g., Pro-Leu/Gln-Gly-Met-Thr-Ser (SEQ ID NO:220) or Pro-Leu/Gln-Gly-Met-Thr (SEQ ID NO:221). Another example of a protease cleavage site is a plasminogen activator cleavage site, e.g., a uPA or a tissue plasminogen activator (tPA) cleavage site. In some cases, the cleavage site is a furin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYTQS (SEQ ID NO: 127), where the protease cleaves between the glutamine and the serine. Another example of a protease cleavage site that can be included in a proteolytically cleavable linker is an enterokinase cleavage site, e.g., DDDDK (SEQ ID NO: 128), where cleavage occurs after the lysine residue. Another example of a protease cleavage site that can be included in a proteolytically cleavable linker is a thrombin cleavage site, e.g., LVPR (SEQ ID NO: 129). Additional suitable linkers comprising protease cleavage sites include linkers comprising one or more of the following amino acid sequences: LEVLFQGP (SEQ ID NO: 130), cleaved by PreScission protease (a fusion protein comprising human rhinovirus 3C protease and glutathione-S-transferase; Walker et al. (1994) Biotechnol. 12:601); a thrombin cleavage site, e.g., CGLVPAGSGP (SEQ ID NO:131); SLLKSRMVPNFN (SEQ ID NO: 132) or SLLIARRMPNFN (SEQ ID NO: 133), cleaved by cathepsin B; SKLVQASASGVN (SEQ ID NO: 134) or SSYLKASDAPDN (SEQ ID NO:135), cleaved by an Epstein-Barr virus protease; RPKPQQFFGLMN (SEQ ID NO: 136) cleaved by MMP-3 (stromelysin); SLRPLALWRSFN (SEQ ID NO: 137) cleaved by MMP-7 (matrilysin); SPQGIAGQRNFN (SEQ ID NO: 138) cleaved by MMP-9; DVDERDVRGFASFL SEQ ID NO:139) cleaved by a thermolysin-like MMP; SLPLGLWAPNFN (SEQ ID NO: 140) cleaved by matrix metalloproteinase 2 (MMP-2); SLLIFRSWANFN (SEQ ID NO:141) cleaved by cathespin L; SGVVIATVIVIT (SEQ ID NO:142) cleaved by cathepsin D; SLGPQGIWGQFN (SEQ ID NO: 143) cleaved by matrix metalloproteinase 1(MMP-1); KKSPGRVVGGSV (SEQ ID NO: 144) cleaved by urokinase-type plasminogen activator; PQGLLGAPGILG (SEQ ID NO: 145) cleaved by membrane type 1 matrix metalloproteinase (MT-MMP); HGPEGLRVGFYESDVMGRGHARLVHVEEPHT (SEQ ID NO: 146) cleaved by stromelysin 3 (or MMP-11), thermolysin, fibroblast collagenase and stromelysin-1; GPQGLAGQRGIV (SEQ ID NO: 147) cleaved by matrix metalloproteinase 13 (collagenase-3); GGSGQRGRKALE (SEQ ID NO: 148) cleaved by tissue-type plasminogen activator(tPA); SLSALLSSDIFN (SEQ ID NO: 149) cleaved by human prostate-specific antigen; SLPRFKIIGGFN (SEQ ID NO: 150) cleaved by kallikrein (hK3); SLLGIAVPGNFN (SEQ ID NO:151) cleaved by neutrophil elastase; and FFKNIVTPRTPP (SEQ ID NO:152) cleaved by calpain (calcium activated neutral protease). In some cases, the protease cleavage site is a TEV protease cleavage site, e.g., ENLYFQS (SEQ ID NO: 153), where cleavage occurs between the Gln and the Ser. In some cases, the protease cleavage site is the TEV protease cleavage site ENLYFQP (SEQ ID NO: 154). ENLYFQS (SEQ ID NO: 153) and ENLYFQP (SEQ ID NO: 154) are wildtype recognition sequences (cleavage substrates) for TEV protease (see e.g. Stols et al. (2002) Prot. Exp. Purif 25: 8-12). In some cases, the proteolytically cleavable linker comprises an HIV-1 protease cleavage site (e.g. SQNYPIVQ (SEQ ID NO: 155)), where cleavage occurs between the tyrosine and the proline. In some cases, an HIV-1 protease cleavage site (e.g. SQNYPIVQ (SEQ ID NO:155)) is specifically excluded.

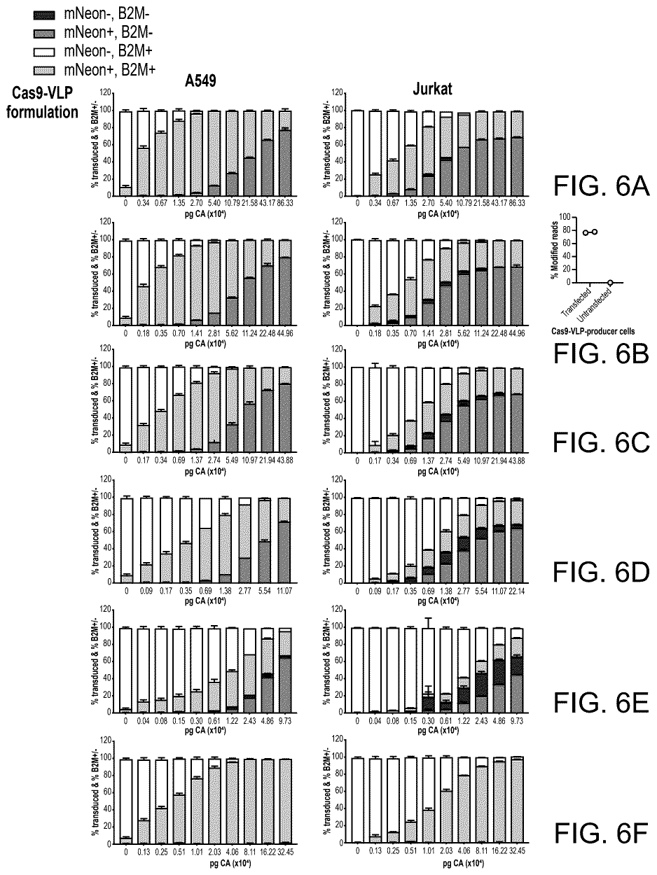

In some cases, the protease cleavage site is a TEV protease cleavage site, e.g., ENLYTQS (SEQ ID NO: 127), where the protease cleaves between the glutamine and the serine. In some cases, the protease cleavage site is a variant TEV-cleavage substrate, where the variant TEV cleavage site is cleaved by a TEV protease (e.g., a TEV protease comprising the TEV protease amino acid sequence provided in FIG. 6B) less efficiently than cleavage of ENLYTQS (SEQ ID NO: 127) by the TEV protease. In some cases, a variant TEV-cleavage site can: (1) mimic the temporal cleavage observed with wild-type gag polyprotein maturation; and/or (2) maximize packaging of a CRISPR/Cas effector polypeptide into a VLP. Suitable variant TEV cleavage sites are described in Tözsér et al. (2005) FEBS J. 272:514. Suitable variant TEV cleavage sites include: ENAYFQS (SEQ ID NO: 156), ENLRFQS (SEQ ID NO: 157), ENLFFQS (SEQ ID NO:158), ETVRFQS (SEQ ID NO: 159), ETLRFQS (SEQ ID NO: 160), ETARFQS (SEQ ID NO:161), ETVYFQS (SEQ ID NO: 162), and ENVYFQS (SEQ ID NO:163).

In some cases, a system of the present disclosure comprises a CRISPR/Cas effector guide RNA. For example, a VLP produced using a system of the present disclosure can comprise, encapsulated within the VLP a guide RNA. In some cases, the guide RNA is a dual guide RNA, e.g., two separate nucleic acids that together comprise a guide RNA. In other instances, the guide RNA is a single-molecule guide RNA (also referred to herein as a “single guide RNA” or “sgRNA”). Suitable guide RNAs are described hereinbelow. In some cases, the guide RNA comprises one or more of: i) a modified base; ii) a modified sugar; and iii) a modified backbone.

A coding sequence (e.g., a nucleotide sequence encoding a Gag-CRISPR-Cas fusion polypeptide; a nucleotide sequence encoding a CRISPR-Cas guide RNA; a nucleotide sequence encoding a therapeutic protein) present in a nucleic acid in a system of the present disclosure can be operably linked to a transcriptional control element (e.g., a promoter). The transcriptional control element can be a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

In some cases, a protein-encoding nucleotide sequence present in a nucleic acid of a system of the present disclosure is operably linked to (under the control of) a promoter operable in a eukaryotic cell (e.g., a U6 promoter, an enhanced U6 promoter, an H1 promoter, and the like). As would be understood by one of ordinary skill in the art, when expressing an RNA (e.g., a guide RNA) from a nucleic acid (e.g., an expression vector) using a U6 promoter (e.g., in a eukaryotic cell), or another PolIII promoter, the RNA may need to be mutated if there are several Ts in a row (coding for Us in the RNA). This is because a string of Ts (e.g., 5 Ts) in DNA can act as a terminator for polymerase III (PolIII). Thus, in order to ensure transcription of a guide RNA in a eukaryotic cell it may sometimes be necessary to modify the sequence encoding the guide RNA to eliminate runs of Ts. In some cases, a nucleotide sequence encoding guide RNA is operably linked to a promoter operable in a eukaryotic cell (e.g., a CMV promoter, an EFIa promoter, an estrogen receptor-regulated promoter, and the like).

Examples of inducible promoters include, but are not limited toT7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; estrogen and/or an estrogen analog; IPTG; etc.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used as long as the promoter is functional in the targeted host cell (e.g., eukaryotic cell; prokaryotic cell).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

### Donor Nucleic Acid

In some cases, a system of the present disclosure comprises a donor nucleic acid. By a “donor nucleic acid” or “donor sequence” or “donor polynucleotide” or “donor template” it is meant a nucleic acid sequence to be inserted at the site cleaved by a CRISPR/Cas effector protein (e.g., after dsDNA cleavage, after nicking a target DNA, after dual nicking a target DNA, and the like). The donor polynucleotide can contain sufficient homology to a genomic sequence at the target site, e.g. 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) can support homology-directed repair. Donor polynucleotides can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair (e.g., for gene correction, e.g., to convert a disease-causing base pair or a non disease-causing base pair). In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

In some cases, the donor sequence is provided to the cell as single-stranded DNA. In some cases, the donor sequence is provided to the cell as double-stranded DNA. It may be introduced into a cell in linear or circular form. If introduced in linear form, the ends of the donor sequence may be protected (e.g., from exonucleolytic degradation) by any convenient method and such methods are known to those of skill in the art. For example, one or more dideoxynucleotide residues can be added to the 3′ terminus of a linear molecule and/or self-complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance.

### Compositions Comprising a System

The present disclosure provides a composition comprising a system of the present disclosure. A composition of the present disclosure can include: a) a system of the present disclosure; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, (such as N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris), etc.). Suitable salts include, e.g., NaCl, MgCl₂, KCl, MgSO₄, etc. In some cases, the composition is sterile. In some cases, the composition is sterile and is free of detectable pyrogens and/or other toxins.

### Methods of Making a Vlp

The present disclosure provides methods of making a VLP of the present disclosure. The methods generally involve introducing into a packaging cell a system of the present disclosure; and harvesting the VLPs produced by the packaging cell. In some cases, the VLPs are harvested from the supernatant (e.g., the cell culture medium) in which the packaging cells are cultures. In some cases, the cell culture medium is filtered (e.g., with a 0.45 μm filter).

Any suitable permissive or packaging cell known in the art may be employed in the production of a VLP of the present disclosure. In some cases, the cell is a mammalian cell. In some cases, the cell is an insect cell. Examples of cells suitable for production of a VLP of the present disclosure include, e.g., human cell lines, such as VERO, W138, MRC5, A549, HEK293, HEK293T, B-50 or any other HeLa cells, HepG2, Saos-2, HuH7, Chinese Hamster Ovary (CHO) cells, and HT1080 cell lines.

Also suitable for use as packaging cells are insect cell lines. Any insect cell that allows for production of a VLP of the present disclosure and which can be maintained in culture can be used. Examples include Spodoptera frugiperda, such as the Sf9 or Sf21 cell lines, Drosophila spp. cell lines, or mosquito cell lines, e.g., Aedes albopictus derived cell lines.

The nucleic acids present in a system of the present disclosure can extra-chromosomal or integrated into the cell's chromosomal DNA. In some cases, the packaging cell is a cell line with one or more packaging functions incorporated extrachromosomally or integrated into the cell's chromosomal DNA, or a cell line with helper functions incorporated extra-chromosomally or integrated into the cell's chromosomal DNA.

### Cells

The present disclosure provides a eukaryotic cell comprising a system of the present disclosure, where the cell is a packaging cell. Any suitable permissive or packaging cell known in the art is suitable. In some cases, the cell is a mammalian cell. In some cases, the cell is an insect cell. Examples of cells suitable for production of a VLP of the present disclosure include, e.g., human cell lines, such as VERO, WI38, MRC5, A549, HEK293, HEK293T, B-50 or any other HeLa cells, HepG2, Saos-2, HuH7, Chinese Hamster Ovary (CHO) cells, and HT1080 cell lines.

Also suitable for use as packaging cells are insect cell lines. Any insect cell that allows for production of a VLP of the present disclosure and which can be maintained in culture can be used. Examples include Spodoptera frugiperda, such as the Sf9 or Sf21 cell lines, Drosophila spp. cell lines, or mosquito cell lines, e.g., Aedes albopictus derived cell lines.

### Methods of Delivering a Therapeutic Protein

The present disclosure provides methods of delivering a therapeutic protein, e.g. to a eukaryotic cell (e.g., a target cell) or to an organism (e.g., an individual). The methods generally involve contacting the cell with a VLP of the present disclosure or administering a VLP to an organism. In some cases, the target cell is in vitro. In some cases, the target cell is in vivo and the method comprises administering the VLP to an individual.

Where a VLP of the present disclosure comprises a guide RNA, in some instances, the guide RNA provides for knockout of a nucleic acid targeted by the guide RNA. Thus, in some cases, a VLP of the present disclosure provides for: i) delivery of a therapeutic protein; and ii) knockout of a target nucleic acid. As one non-limiting example, a VLP of the present disclosure can both: i) provide for delivery of a therapeutic protein (such as a chimeric antigen receptor (CAR)); and ii) knock out an endogenous nucleic acid encoding a beta-2 microglobulin (β2M) polypeptide, where the guide RNA present in the VLP (or encoded by a nucleic acid present in the VLP) would comprise a nucleotide sequence targeting a β2M-encoding nucleic acid in a target cell. Such a VLP would be useful for generating T cells that express a CAR (“CAR-T cells”) that do not express endogenous major histocompatibility complex (MHC) class I antigens on their cell surface and thus could be useful for delivery of allogeneic CAR-T cells. As another non-limiting example, a VLP of the present disclosure can both: i) provide for delivery of a therapeutic protein (such as an antibody, e.g., a cancer-specific antibody or other therapeutic antibody); and ii) knock out an endogenous nucleic acid encoding an antibody light chain (e.g., a kappa light chain) or an immunoglobulin (Ig) Fc polypeptide (e.g., an Ig Fc polypeptide of a particular isotype such as IgG1). Such a VLP would be useful for generating B cells that produce a therapeutic antibody.

In some cases, a VLP of the present disclosure provides for homology directed repair (HDR) of a defective target nucleic acid. In some cases, a VLP of the present disclosure provides for non-homologous end joining (NHEJ) of a target nucleic acid, e.g., to provide for a knockout of a target nucleic acid.

In some cases, a method of the present disclosure comprises: a) electroporating VLPs with a donor DNA template (e.g., a single-stranded donor DNA template); and b) contacting target cells with the electroporated VLP/donor DNA template mixture. Electroporating VLPs with a donor DNA template (e.g., a single-stranded donor DNA template) prior to contacting with target cells can increase HDR, compared to the level of HDR when the VLPs are simply admixed with the donor DNA template (not electroporated).

A cell that serves as a recipient for a VLP of the present disclosure can be any of a variety of eukaryotic cells, including, e.g., in vitro cells; in vivo cells; ex vivo cells; primary cells; cancer cells; animal cells; plant cells; algal cells; fungal cells; etc. A cell that serves as a recipient for a VLP of the present disclosure is referred to as a “host cell” or a “target cell.”

In some cases, the target cell is in vitro. In some cases, cells are removed from an individual, contacted with a VLP of the present disclosure in vitro, such that the cells are modified to produce the therapeutic protein encoded by the recombinant lentiviral nucleic acid present in the VLP; and returning the modified cells to the individual from whom the cells were obtained. In some cases, cells are removed from an individual, contacted with a VLP of the present disclosure in vitro, such that the cells are modified to produce the therapeutic protein encoded by the recombinant lentiviral nucleic acid present in the VLP; and administering the modified cells to an individual other than the individual from whom the cells were obtained.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogeneic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some embodiments, the stem cell is a hematopoietic stem cell (HSC). HSCs are mesoderm-derived cells that can be isolated from bone marrow, blood, cord blood, fetal liver and yolk sac. HSCs are characterized as CD34+ and CD3−. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

In some cases, the pseudotyping viral glycoprotein is selected from an influenza virus hemagglutinin, a SARS-CoV glycoprotein, a respiratory syncytial virus glycoprotein, a human parainfluenza virus glycoprotein, and a VSV-G; and the target cell is a lung cell. In some cases, the VLP comprises a guide RNA, or a nucleic acid comprising a nucleotide sequence encoding a guide RNA, where the guide RNA comprises a targeting sequence that targets a CFTR (cystic fibrosis transmembrane conductance regulator) gene. For example, targeting a CFTR gene can treat cystic fibrosis. Where the target gene comprises a defect that leads to pathology, a donor nucleic acid comprising a nucleotide sequence without the defect can be included in the VLP, such that the defect is corrected.

In some cases, the pseudotyping viral glycoprotein is a measles virus hemagglutinin and/or a measles virus fusion glycoprotein, and the target cell is a CD34⁺ cell. In some cases, the VLP comprises a guide RNA, or a nucleic acid comprising a nucleotide sequence encoding a guide RNA, where the guide RNA comprises a targeting sequence that targets an HbF (fetal hemoglobin) gene. For example, targeting an HbF gene can treat sickle cell disease or beta-thalassemia. Where the target gene comprises a defect that leads to pathology, a donor nucleic acid comprising a nucleotide sequence without the defect can be included in the VLP, such that the defect is corrected.

In some cases, the pseudotyping viral glycoprotein is selected from a measles virus hemagglutinin and/or a measles virus fusion glycoprotein, an HTLV-1 glycoprotein, and a VSV-G glycoprotein; and the target cell is a CD8⁺ T cell. In some cases, the VLP comprises a guide RNA, or a nucleic acid comprising a nucleotide sequence encoding a guide RNA, where the guide RNA comprises a targeting sequence that targets a gene selected from PD1 (programmed cell death 1), CTLA4 (cytotoxic T-lymphocyte-associated protein 4), and TCR (T-cell receptor). For example, targeting a PD-1 gene, a CTLA-4 gene, or a TCR gene, can be used in the generation of chimeric antigen receptor (CAR)-T cells.

In some cases, the pseudotyping viral glycoprotein is selected from a HIV-1 envelope, a HTLV-1 glycoprotein, a measles virus hemagglutinin, and a VSV-G glycoprotein; and the target cell is a CD4+ T cell. In some cases, the VLP comprises a guide RNA, or a nucleic acid comprising a nucleotide sequence encoding a guide RNA, where the guide RNA comprises a targeting sequence that targets a CCR5 gene, or targets an integrated and proviral HIV-1. Targeting a CCR5 gene can be used to enhance resistance to HIV. Targeting an integrated and proviral HIV-1 can be used to reduce the pool of T cells that are reservoirs for latent HIV.

In some cases, the pseudotyping viral glycoprotein is a Ross River virus glycoprotein or a VSV-G; and the target cell is a skeletal muscle cell. In some cases, the VLP comprises a guide RNA, or a nucleic acid comprising a nucleotide sequence encoding a guide RNA, where the guide RNA comprises a targeting sequence that targets a Duchenne muscular dystrophy (DMD) gene. Targeting a DMD gene can be used to treat Duchenne muscular dystrophy. Where the target gene comprises a defect that leads to pathology, a donor nucleic acid comprising a nucleotide sequence without the defect can be included in the VLP, such that the defect is corrected.

In some cases, the pseudotyping viral glycoprotein is selected from an Ebola virus glycoprotein, a Marburg virus glycoprotein, and a VSV-G; and the target cell is an ocular cell (e.g., in a retinal cell, a photoreceptor cell, etc.). In some cases, the VLP comprises a guide RNA, or a nucleic acid comprising a nucleotide sequence encoding a guide RNA, and wherein the guide RNA comprises a targeting sequence that targets a CEP290 (centrosomal protein 290) gene. Targeting a CEP290 gene can be used to treat Leber congenital amaurosis 10 (LCA10). Where the target gene comprises a defect that leads to pathology, a donor nucleic acid comprising a nucleotide sequence without the defect can be included in the VLP, such that the defect is corrected.

In some cases, the pseudotyping viral glycoprotein is selected from an Ebola virus glycoprotein, a Marburg virus glycoprotein, and a VSV-G; and the target cell is an auditory cell (e.g., hair cells, cochlear cells, etc.). In some cases, the VLP comprises a guide RNA, or a nucleic acid comprising a nucleotide sequence encoding a guide RNA, where the guide RNA comprises a targeting sequence that targets a USH2A (Usher syndrome 2A) gene. Targeting a USH2A gene can be used to treat Usher Syndrome type 2A. Where the target gene comprises a defect that leads to pathology, a donor nucleic acid comprising a nucleotide sequence without the defect can be included in the VLP, such that the defect is corrected.

In some cases, the pseudotyping viral glycoprotein is selected from aa rabies glycoprotein, a Mokola virus glycoprotein, a Semliki Forest virus glycoprotein, a Sindbis virus glycoprotein, a Venezuelan equine encephalitis virus glycoprotein, an influenza hemagglutinin glycoprotein, and a VSV-G; and wherein the target cell is a central nervous system cell (e.g., neurons (e.g., excitatory and inhibitory neurons); and glial cells (e.g., oligodendrocytes, astrocytes and microglia)). In some cases, the VLP comprises a guide RNA, or a nucleic acid comprising a nucleotide sequence encoding a guide RNA, and wherein the guide RNA comprises a targeting sequence that targets a gene selected from Tau/MAPT-1, HTT (Huntingtin), SOD1 (superoxide dismutase 1), SOCS3 (suppressor of cytokine signaling 3), USP8 (ubiquitin specific peptidase 8), DOTIL (DOTi-like histone lysine methyltransferase), UFM1 (ufmnylation; ubiquitin fold modifier 1), SOCS2 (suppressor of cytokine signaling 2), SOCS9 (suppressor of cytokine signaling 9), SOCS13 (suppressor of cytokine signaling 13), SOCS11 (suppressor of cytokine signaling 11), and SOCS5 (suppressor of cytokine signaling 5). For example, targeting a Tau gene can treat Alzheimer's disease. As another example, targeting an HTT gene can treat Huntington Disease. As another example, targeting a SOD1 gene can treat amyotrophic lateral sclerosis. As another example, targeting a Ufmylation, USP8, DOT1L, SOCS2, SOCS3, SOCS9, SOCS13, SOCS11, or SOCS5 gene can treat glioblastoma. Where the target gene comprises a defect that leads to pathology, a donor nucleic acid comprising a nucleotide sequence without the defect can be included in the VLP, such that the defect is corrected.

In some cases, a single dose of a composition comprising a VLP of the present disclosure comprises from about 10² VLPs to about 10⁹ VLPs. For example, a single dose of a composition comprising a VLP of the present disclosure comprises from about 10² VLPs to about 10³ VLPs, from about 10³ VLPs to about 10⁴ VLPs, from about 10⁴ VLPs to about 10⁵ VLPs, from about 10⁵ VLPs to about 10⁶ VLPs, from about 10⁶ VLPs to about 10⁷ VLPs, from about 10⁷ VLPs to about 10⁸ VLPs, from about 10⁸ VLPs to about 10⁹ VLPs, or from about 10⁹ VLPs to about 10¹⁰ VLPs.

A composition comprising a VLP of the present disclosure can be administered via any of a variety of parenteral and non-parenteral routes of administration. For example, a composition comprising a VLP of the present disclosure can be administered intravenously, intramuscularly, intratumorally, peritumorally, subcutaneously, intraperitoneally, and the like. A VLP of the present disclosure can be administered via convection enhanced delivery (CED) injection.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A system comprising:
a) a first nucleic acid comprising a nucleotide sequence encoding a fusion polypeptide comprising:
   i) a lentiviral gag polyprotein comprising a matrix (MA) polypeptide, a capsid (CA) polypeptide, and a nucleocapsid (NC) polypeptide; and
   ii) a CRISPR-Cas effector polypeptide;
wherein the fusion polypeptide comprises proteolytically cleavable linker between the gag polyprotein and the CRISPR-Cas effector polypeptide;
b) a second nucleic acid comprising a nucleotide sequence encoding a therapeutic polypeptide, wherein the second nucleic acid comprises a recombinant lentiviral nucleic acid;
c) a third nucleic acid comprising a nucleotide sequence encoding a pseudotyping viral envelope protein and/or a polypeptide that provides for binding to a target cell; and
d) a fourth nucleic acid comprising a nucleotide sequence encoding a lentiviral pol polyprotein comprising a reverse transcriptase and an integrase;
wherein the system comprises a nucleic acid comprising a nucleotide sequence encoding a CRISPR-Cas guide RNA, wherein the CRISPR-Cas guide RNA-encoding nucleic acid is either:
   i) part of the second nucleic acid; or
   ii) a fifth nucleic acid.

Aspect 2. The system of aspect 1, wherein the therapeutic polypeptide has a length of from about 250 amino acids to about 3,000 amino acids.

Aspect 3. The system of aspect 2, wherein the therapeutic polypeptide has a length of from about 500 amino acids to about 2,500 amino acids.

Aspect 4. The system of any one of aspects 1-3, wherein the therapeutic polypeptide is a chimeric antigen receptor (CAR), a T-cell receptor, a synNotch polypeptide, a natural killer cell receptor, or an antibody.

Aspect 5. The system of aspect 4, wherein the CAR comprises an antigen-binding domain specific for a cancer-associated antigen.

Aspect 6. The system of any one of aspects 1-5, wherein the recombinant lentiviral nucleic acid is a recombinant human immunodeficiency virus-1 nucleic acid.

Aspect 7. The system of any one of aspects 1-6, wherein the lentiviral gag polyprotein is a human immunodeficiency virus (HIV) gag polyprotein comprising a MA polypeptide, a CA polypeptide, a p2 polypeptide, an NC polypeptide, a p1 polypeptide, and a p6 polypeptide.

Aspect 8. The system of any one of aspects 1-7, wherein the CRISPR-Cas effector polypeptide is a type II CRISPR-Cas effector polypeptide, a type V CRISPR-Cas effector polypeptide, or a type VI CRISPR-Cas effector polypeptide.

Aspect 9. The system of aspect 8, wherein the type II CRISPR/Cas effector polypeptide is a Cas9 polypeptide.

Aspect 10. The system of any one of aspects 1-9, wherein the CRISPR/Cas effector polypeptide is a fusion polypeptide comprising one or more nuclear localization signals.

Aspect 11. The system of any one of aspects 1-10, further comprising a donor template nucleic acid, or a nucleotide sequence encoding the donor template nucleic acid.

Aspect 12. A eukaryotic cell comprising the system of any one of aspects 1-11.

Aspect 13. The eukaryotic cell of aspect 12, wherein the cell is a packaging cell.

Aspect 14. A virus-like particle (VLP) comprising:
a) a CRISPR-Cas effector polypeptide; and
b) a recombinant lentivirus comprising a nucleotide sequence encoding a therapeutic polypeptide having a length of from about 250 amino acids to about 3,000 amino acids, wherein the VLP comprises a pseudotyping viral glycoprotein and/or a polypeptide that provides for binding to a target cell.

Aspect 15. The VLP of aspect 14, wherein the therapeutic polypeptide has a length of from about 500 amino acids to about 2,500 amino acids.

Aspect 16. The VLP of aspect 14 or 15, wherein the therapeutic polypeptide is a chimeric antigen receptor (CAR), a T-cell receptor, a synNotch polypeptide, a natural killer cell receptor, or an antibody.

Aspect 17. The VLP of aspect 16, wherein the CAR comprises an antigen-binding domain specific for a cancer-associated antigen.

Aspect 18. The VLP of any one of aspects 14-17, wherein the pseudotyping viral glycoprotein is a human immunodeficiency virus-1 envelope protein, a measles virus hemagglutinin, an HTLV-1 glycoprotein, or a VSV-G glycoprotein.

Aspect 19. The VLP of any one of aspects 14-17, wherein the polypeptide that provides for binding to a target cell is an antibody, optionally wherein the antibody is a single-chain Fv or a nanobody.

Aspect 20. The VLP of any one of aspects 14-17, wherein the polypeptide that provides for binding to a target cell is a DARPin.

Aspect 21. The VLP of any one of aspects 14-20, wherein the CRISPR-Cas effector polypeptide is a type II CRISPR-Cas effector polypeptide, a type V CRISPR-Cas effector polypeptide, or a type VI CRISPR-Cas effector polypeptide.

Aspect 22. The VLP of aspect 21, wherein the type II CRISPR/Cas effector polypeptide is a Cas9 polypeptide.

Aspect 23. The VLP of any one of aspects 14-22, wherein the CRISPR/Cas effector polypeptide is a fusion polypeptide comprising one or more nuclear localization signals.

Aspect 24. The VLP of any one of aspects 14-23, comprising a CRISPR/Cas guide RNA.

Aspect 25. The VLP of any one of aspects 14-24, comprising a donor template nucleic acid.

Aspect 26. A composition comprising the VLP of any one of aspects 14-25.

Aspect 27. The composition of aspect 26, further comprising a donor template nucleic acid.

Aspect 28. A method of delivering a therapeutic polypeptide to a eukaryotic cell, the method comprising contacting the cell with the VLP of any one of aspects 14-25, or the composition of aspect 26 or aspect 27.

Aspect 29. The method of aspect 28, wherein the eukaryotic cell is in vivo.

Aspect 30. The method of aspect 24, wherein the eukaryotic cell is in vitro.

Aspect 31. The method of any one of aspects 28-30 wherein the eukaryotic cell is a T cell.

Aspect 32. The method of any one of aspects 28-30 wherein the eukaryotic cell is a B cell.

Aspect 33. The method of any one of aspects 28-30 wherein the eukaryotic cell is a stem cell.

Aspect 34. A method of making a virus-like particle (VLP) comprising a therapeutic polypeptide, the method comprising: a) introducing the system of any one of aspects 1-11 into a packaging cell; and b) harvesting VLPs produced by the packaging cell.

Aspect 35. A method of delivering a therapeutic polypeptide to a eukaryotic cell, and carrying out homology-directed repair (HDR) in the cell, the method comprising:
a) electroporating a solution comprising: i) a VLP of any one of aspects 14-24; and ii) a donor template nucleic acid, forming an electroporated VLP/donor template solution; and
b) contacting the cell with the electroporated VLP/donor template solution, wherein said contacting results in HDR and in synthesis of the therapeutic polypeptide in the cell.

Aspect 36. The method of aspect 35, wherein the eukaryotic cell is a T cell, a B cell, or a stem cell.

Aspect 37. The method of aspect 35, wherein the eukaryotic cell is a T cell, wherein the therapeutic polypeptide is a chimeric antigen receptor, and wherein the guide RNA present in the VLP provides for knockout, via HDR, of endogenous 3-2 microglobulin such that, following HDR, the cell does not substantially display MHC class I polypeptides on its surface.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Generating VLPs

VLPs are an unexplored strategy for linking the delivery of pre-formed Cas9 RNPs with a clinically relevant transgene and leveraging viral glycoprotein pseudotyping to direct genome editing to specific cell types. Here, it is demonstrated that engineered lentiviral particles can deliver Cas9 RNP complexes for genome editing, either tracelessly or while simultaneously integrating a lentiviral-encoded transgene (Cas9-VLPs) in immortalized cell lines and primary human T cells. Treatment of primary human T cells with Cas9-VLPs packaging a lentiviral-encoded CAR resulted in simultaneous knockout of genetic targets relevant to allogeneic T cell production while effectively mediating CAR expression, an approach that was amenable to multiplexing. Additionally, treatment of T cells with broadly-transducing Cas9-VLPs resulted in targeted genetic knockout in CD4+ and CD8+ T cells, while treatment with Cas9-VLPs pseudotyped with the CD4-tropic HIV-1 envelope glycoprotein drove the exclusive transduction and genome editing of CD4+ T cells within a mixed cell population. These data establish Cas9-VLPs as an effective approach for mediating cell-type specific genome editing using Cas9 RNPs. As Cas9-VLPs circumvent the requirement for ex vivo Cas9 RNP delivery via electroporation, this strategy indicates a path forward for leveraging the tropism of viral glycoproteins in targeting specific cell types for genome engineering in vivo.

### Materials and Methods

### Culture of Human Cell Lines

Lenti-X, 293T, A549, CCRF-CEM, HuT 78 and Jurkat cell lines were obtained from the UC Berkeley Cell Culture Facility. All cells were cultured with 10% fetal bovine sera (VWR) and 100 U/mL penicillin-streptomycin (Gibco). LentiX, 293T, and A549 cells were cultured in DMEM (Corning), Jurkat and CCRF-CEM cells were cultured in RPMI 1640 (Thermo Fisher) and 1 mM sodium pyruvate, while HuT 78 cells were cultured in IMEM (Thermo Fisher). Cell lines were routinely checked for mycoplasma using the MycoAlert mycoplasma detection kit (Lonza) according to the manufacturer's instructions.

### Isolation and Culture of Human Primary T Cells

Primary adult blood cells were obtained from anonymous healthy human donors as a leukoreduction pack purchased from Stemcell Technologies or Allcells Inc, or as a Trima residual from Vitalant, under a protocol approved by the University of California San Francisco Institutional Review Board (IRB). If needed, peripheral blood mononuclear cells were isolated by Ficoll-Paque (GE Healthcare) centrifugation. Bulk CD3+ T lymphocytes were then further isolated by magnetic negative selection using an EasySep magnetic Cell Isolation kit (STEMCELL, as per the manufacturer's instructions). 96-well flat bottom plates were primed for stimulation by incubating with anti-human CD3 (10 μg/mL) and anti-human CD28 (5 μg/mL) antibodies in PBS for 1 hour at 37° C. prior to washing. Primary T cells were activated by plating at 250,000 cells/mL and culturing for one day in XVivo15 medium (Lonza) containing fetal bovine serum (5%), 2-mercaptoethanol (50 μM), N-acetyl L-cysteine (10 mM), IL-2 (300 U/mL), IL-7 (5 ng/mL), and IL-15 (5 ng/mL). Cas9-VLPs in RPMI 1640 were added to primary human T cells 24 hours later along with IL-2 (500 U/mL) and protamine sulfate (4 g/mL). Media and growth factors were replaced as needed, approximately every 5-6 days. The number of unique primary human T cell donors used for each experiment is listed in Supplementary Table 4 (FIG. 17).

### Plasmid Construction

The Gag-pol expression plasmid psPax2 was a gift from Didier Trono (Addgene plasmid #12260). pCMV-VSV-G was a gift from Bob Weinberg (Addgene plasmid #8454). Gag-Cas9 was constructed by amplifying Gag from psPax2 and Cas9 from pMJ920 (Addgene plasmid #42234). HIV-1 Env amino acid sequence was obtained from UniProt (P04578), human codon optimized (IDT), and ordered as a gBlock (IDT). In-Fusion (Takara Bio) cloning was used to clone Gag-Cas9 and Env into the pCAGGS expression vector. pCF221 (Addgene plasmid #121669) was modified to express mNeonGreen (Allele Biotechnology) orthe a-CD19-4-1BBζ-P2A-mCherry (CAR-P2A-mCherry) construct (Hill et al., 2018; Muller et al., 2020) in place of mCherry and was used as the sgRNA-expressing lentiviral transfer plasmid. For generation of hybrid Cas9-VLPs, the guide RNA expression cassette was removed from the CAR-P2A-mCherry lentiviral plasmid via digestion with EcoRI and KpnI (NEB). The following primers (IDT) were phosphorylated, annealed, and ligated into the digested vector: 5′-cATCGATCTTAAGTCGCGACTCGAg (SEQ ID NO: 164) and 5′-aattcTCGAGTCGCGACTTAAGATCGATggtac (SEQ ID NO: 165). The U6-sgRNA CAG-mTagBFP2 expression plasmid used for traceless Cas9-VLP and CAR-Cas9 VLP production was a gift from Benjamin Oakes. Oligos encoding guide RNA spacers were ordered from IDT, phosphorylated, annealed and ligated into digested sgRNA expression vectors.

### Cas9-VLP Production

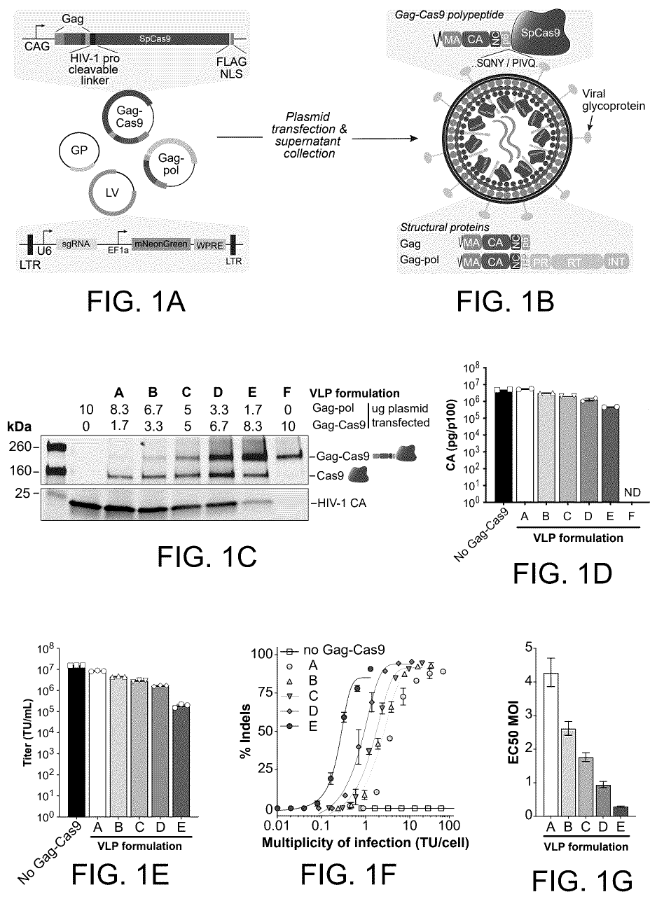

Cas9-VLPs were produced in mammalian cell culture by transient transfection of Lenti-X cells (Takara Bio). 3.5-4 million cells were seeded into 10 cm tissue culture dishes (Corning). The following day, cells were transfected with psPax2, Gag-Cas9, 1 μg pCMV-VSV-G or 0.2 μg HIV Env glycoprotein, and 10 μg of plasmid encoding the sgRNA-expression cassette (either transiently or in the context of a lentiviral transfer plasmid). Plasmids were diluted in Opti-MEM (Gibco) and mixed with polyethylenimine (PEI, Polysciences Inc.) at a 3:1 PEI:plasmid ratio. Quantities of transfected Gag-Cas9 and psPax2 plasmid are listed in FIG. 1C for VLP formulations A-F. A549 and Jurkat experiments used Cas9-VLP formulation D, unless indicated otherwise, and supernatant was harvested at 48 hours post transfection. Cas9-VLP experiments with primary human T cells used Cas9-VLP formulation B, where the Lenti-X media was replaced with Opti-MEM 6-18 hours post transfection. Cas9-VLP-containing Opti-MEM was collected at 48 and 96 hours post media change, with fresh Opti-MEM being added to the cells after 48 hours. Harvested supernatants were centrifuged at 1,500 rpm for 10 minutes and filtered through a 0.45 μM PES membrane bottle top filter (Thermo Fisher) or syringe filter (VWR). Cas9-VLPs were concentrated via ultracentrifugation by floating Cas9-VLP-containing supernatant on top of a cushioning buffer of 30% (w/v) sucrose in 100 mM NaCl, 10 mM Tris-HCl pH 7.5, 1 mM EDTA pH 8.0, at 25,000 rpm with a SW28 or SW41 Ti rotor (Beckman Coulter) for 2 hours at 4° C. in polypropylene tubes (Beckman Coulter). After ultracentrifugation, the Cas9-VLP pellet was resuspended in RPMI 1640 (Gibco) or XVivo15 (Lonza) for treatment of primary T cells or Opti-MEM. Cas9-VLPs were either stored at 4° C. or frozen at −80° C. within a isopropanol-filled freezing container until use.

### Cas9-VLP Quantification

Western blots were performed to assess protein components of Cas9-VLPs. Cas9-VLPs were denatured by mixing with Laemmli buffer with 10% 2-mercaptoethanol and heating at 90° C. for 5 minutes. Samples were run on 4-20% SDS-PAGE gels (Bio-Rad) prior to transfer onto a methanol soaked polyvinylidene difluoride (PVDF, Bio-Rad) membrane. PVDF membranes were blocked with 5% non-fat milk (Apex) in PBS (Gibco) with 0.1% Tween (Sigma) (PBS-T) for one hour at room temperature (˜22-25° C.). The solution was replaced with 1% non-fat milk in PBS-T and a 1:5000 primary antibody dilution containing anti-FLAG (Sigma) or a 1:2000 dilution of anti-p24 (Abcam) antibodies prior to shaking at 4° C. overnight. The following day, the solution was replaced with 1% non-fat milk in PBS-T and a 1:5000 secondary antibody dilution containing IR680 or IR800 conjugated antibodies (LI-COR) and shaken for 1 hour. Western blot membranes were washed with PBS-T three times prior to imaging on a LI-COR OdysseyCLx.

Lenti-X p24 rapid titer kits (Takara Bio) were used to quantify the titer of Cas9-VLPs after concentration. Cas9-VLPs were diluted 1:1,000-100,000 and the ELISA was performed according to the manufacturer's directions. Absorbance was measured at 450 nm on a BioTek plate reader. Cas9-VLP p24 content was calculated by comparison to serial dilution of a p24 standard (Takara Bio). To calculate transducing units per mL (TU/mL), Cas9-VLP preps were serially diluted and used to treat 15 k Jurkats or 25 k primary T cells in 96-well u-bottom plates. The percent of cells transduced (mNeonGreen+) was quantified at 6-7 days post treatment using an Attune NxT flow cytometer with a 96-well autosampler (Thermo Fisher Scientific) and titer was quantified as TU/mL=(number of cells transduced x percent mNeonGreen+)/(virus treatment volume). Wells where Cas9-VLP transduction was <25% were used for titer calculation. MOI was plotted against indels and a sigmoidal four parameter logistic fit was applied to each data set to interpolate the MOI at which 50% indels would be expected, using a 95% confidence interval.

### Cas-VLP Homology-Directed Repair

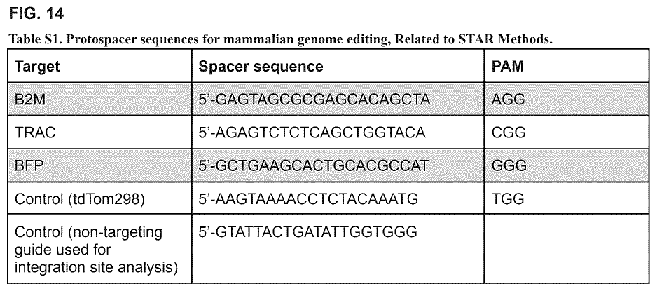

Cas9-VLPs targeting BFP were produced as previously described (see Supplementary Table 1 (FIG. 14) for guide sequence). Cas9-VLPs were mixed with a single-stranded DNA template (IDT) in either DPBS (Thermo Fisher Scientific), Opti-MEM (Thermo Fisher Scientific), or SE/SF/SG buffer (Lonza). Unless otherwise noted, SE buffer (Lonza) with pulse code CM-150 was utilized. The mixture was electroporated using a 4D-nucleofector (Lonza) before immediately adding to 293T cells stably expressing a BFP-to-GFP reporter (Addgene plasmid #71825). A three-nucleotide conversion within the BFP gene results in GFP expression. Cells were analyzed for loss of BFP (non-homologous end joining) and gain of GFP (homology-directed repair) expression after 5-7 days on a Attune NxT flow cytometer with a 96-well autosampler (Thermo Fisher Scientific).

### Targeted Integration Analysis

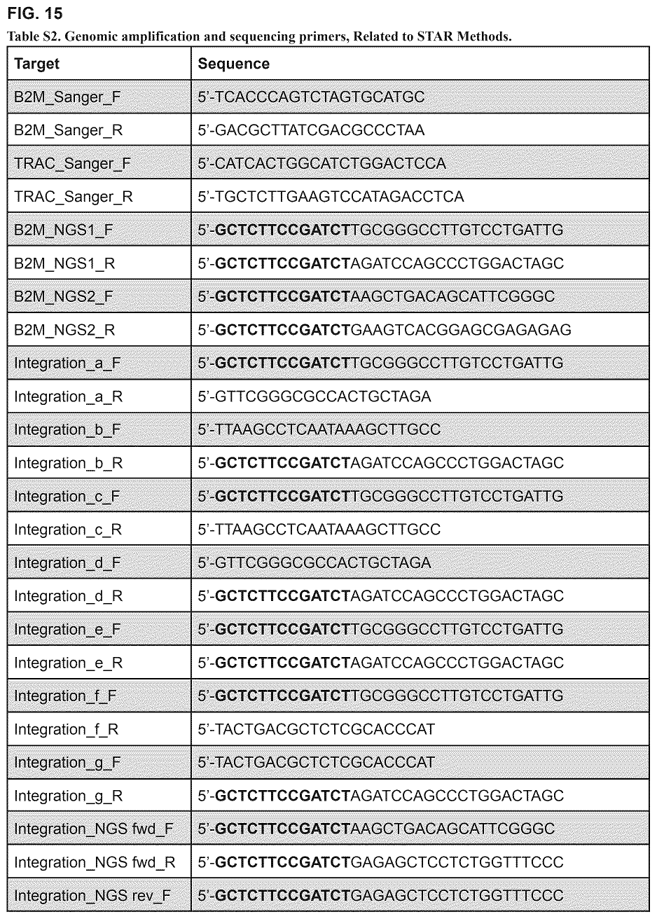

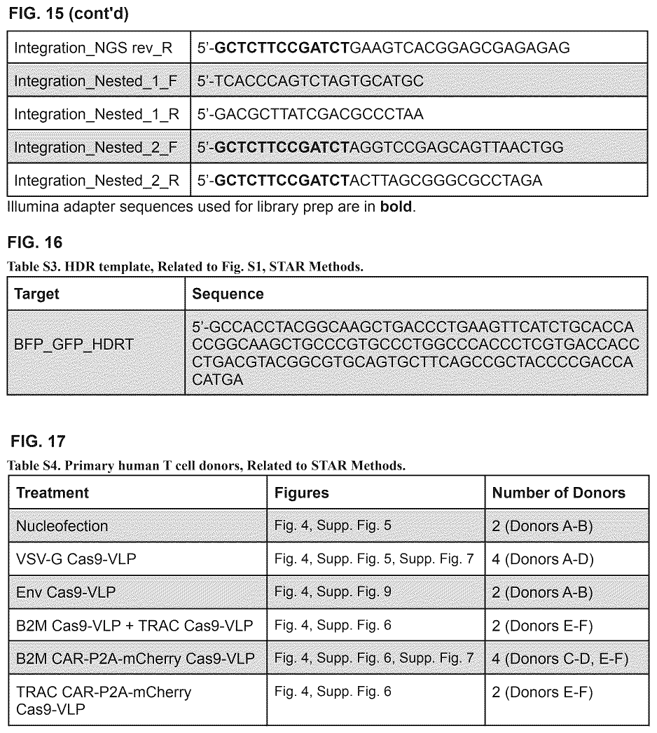

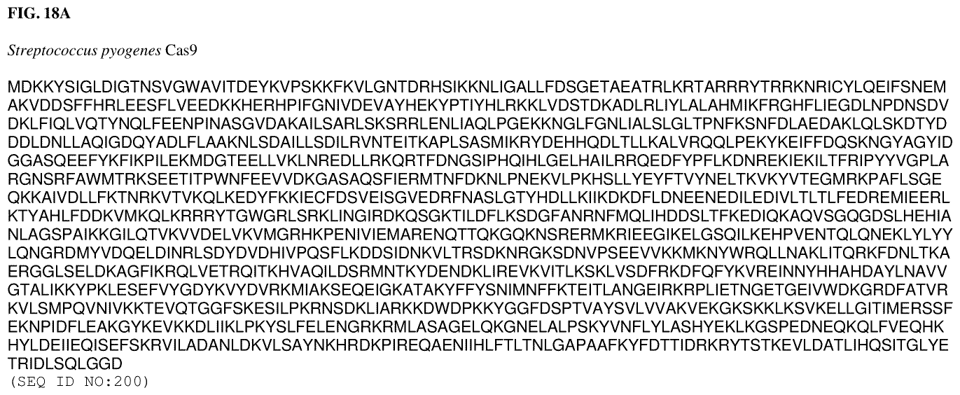

15 k 293T cells treated with B2M-targeting or non-targeting Cas9-VLPs and DNA was isolated 3 days post treatment by resuspending in Quick Extract (Lucigen) and heating at 65° C. for 20 minutes followed by 95° C. for 20 minutes before storing at −20° C. A nested PCR approach using PrimeStar GXL DNA polymerase (Takara Bio) was used to detect integration of the lentiviral genome into the B2M genomic site targeted by Cas9. For PCR analysis of lentiviral integration, the B2M targeted region was first amplified using nested primer set #1 and cleaned up (NucleoSpin Gel and PCR Clean-Up kit, Takara Bio) followed by amplification with primer sets a-g (Supp. Table 2 (FIG. 15)). For MiSeq next generation sequencing analysis of targeted integration, the B2M targeted region was first amplified with nested primer set #2 and cleaned up (SPRI beads, UC Berkeley Sequencing Core) followed by amplification with primer sets to detect both integration orientations (primer pairs NGS Fwd and NGS Rev, Supp. Table 2 (FIG. 15)). Pair-end reads were merged, trimmed, and aligned to the expected sequence of lentiviral insertion into the expected Cas9 target site in the B2M gene (Geneious).

### RNP Nucleofection

Cas9 RNPs were formed as previously described (Nguyen et al., 2020) at a 1:2 molar ratio between Cas9-NLS (UC Berkeley QB3 MacroLab) and annealed crRNA and tracrRNA (Horizon Discovery) in IDT duplex buffer with a polyglutamic acid electroporation enhancer, aliquoted and stored frozen at −80° C. until use. Cas9 RNPs (50 μmol) were electroporated into primary human T cells using a 96-well format 4D-nucleofector (Lonza) with the P3 buffer and the EH-115 pulse code. Immediately after electroporation cells were rescued by adding growth media and incubating for 20 minutes prior to diluting to 0.5 to 1e6 cells/mL for culturing.

### Flow Cytometry

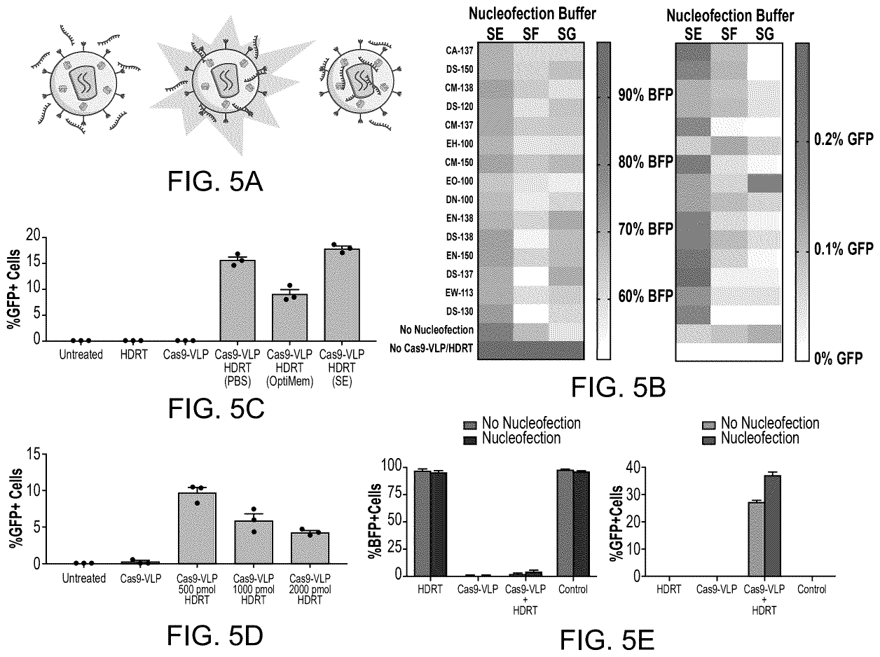

All flow cytometry was performed on an Attune NxT flow cytometer with a 96-well autosampler (Thermo Fisher Scientific). Cells were resuspended in FACS buffer (1-2% BSA in PBS) and stained with the surface marker-targeting antibodies: B2M-FITC (Biolegend), B2M-PE (Biolegend), B2M-APC (Biolegend), CD4-FITC (Biolegend), CD8-PeCy7 (BD Biosciences), and TCRa/b-BV421 (Biolegend) and live/dead stains Ghost Dye red 780 (Tonbo) or Ghost Dye violet 450 (Tonbo), prior to analyzing. All analysis was done using the FlowJo v10 software. The gating strategy for flow cytometry can be seen in Supplementary FIGS. 5, 6, and 9.

### Cytotoxicity Assay

Nalm-6 target cells were labelled using CellTrace Violet Cell Proliferation Kit (Thermo Fisher Scientific) according to the supplier's information. T cells were co-cultured with labelled target cells at various Effector:Target ratios for 16-24 hours. The percent of transduced cells were normalized by adding untransduced T cells. Absolute count of remaining living target cells was analyzed and percent killing was calculated by comparing to control wells (target cells only). Measurement was performed on an Attune NxT Flow Cytometer (Thermo Fisher Scientific).

### Intracellular Cytokine and Activation Assay

Cells were stimulated with Nalm-6 target cells at an E:T ratio of 1:1. Transduction rates were normalized by adding untransduced T cells. 24 hours later, eBioscience Brefeldin A Solution (1000×) was added and incubated for 4 hours at 37° C. Cells were stained with extracellular antibodies eBioscience Fixable Viability Dye eFluor 780 (Thermo Fisher Scientific), CD25 PE-Cy7 (BD), CD69 PerCP (BioLegend), 4-1BB BV711 (BioLegend) and intracellular antibodies TNF-α Pacific Blue (BioLegend), IL-2 APC (BD) and IFN-g FITC (BioLegend) using the FIX & PERM Cell Fixation & Cell Permeabilization Kit (Thermo Fisher Scientific). CAR samples were gated on mCherry+ cells. Measurement was performed on an Attune NxT Flow Cytometer (Thermo Fisher Scientific).

### Amplicon Sequencing

Genome editing was determined either by Sanger sequencing or next-generation sequencing; in both cases, the presence of insertions or deletions around the Cas9-targeted sequence was used to determine genome editing efficiency. Cells were pelleted and resuspended in QuickExtract (Lucigen) and heated at 65° C. for 20 minutes followed by 95° C. for 20 minutes before storing at −80° C. An amplicon containing the target sequence was amplified via PCR with Q5 polymerase (NEB) or PrimeStar GXL DNA polymerase (Takara Bio) and the resulting sample was cleaned with magnetic SPRI beads (UC Berkeley Sequencing Core). PCR amplicons were analyzed via Sanger sequencing (UC Berkeley Sequencing Core) and the resulting traces were deconvolved with Synthego's Inference of CRISPR Edits (ICE) program (https://ice.synthego.com). NGS sequencing was prepared similarly, but with PCR primers containing Illumina adapter sequences. PCR amplicons were analyzed on an Illumina MiSeq by QB3 Genomics at UC Berkeley. Paired-end NGS reads were analyzed for indels with CRISPResso2 (https://crispresso.pinellolab.partners.org).

### Statistical Analysis

Statistical analysis was performed in Prism v7, v8, and v9. Statistical details for all experiments, including value and definition of n, error bars, and significance thresholds can be found in the Figure Legends.

### Results

### Engineering Lentivirus-Based VLPs for the Controlled Delivery of Cas9 RNP Complexes

Lentiviral production involves the co-transfection of producer cells with plasmids encoding the viral structural components, viral glycoprotein, and lentiviral transfer plasmid with a transgenic sequence flanked by long terminal repeat (LTR) sequences. To promote packaging of Cas9 protein in HIV-1 VLPs (Cas9-VLPs), a plasmid was constructed to express S. pyogenes Cas9 fused to the C-terminus of the Gag polypeptide and included this during lentiviral production. A lentiviral transfer plasmid encoding expression cassettes for both an mNeonGreen fluorescent reporter and a single guide RNA (sgRNA) was included (FIG. 1A). To promote the separation of Cas9 from Gag during proteolytic virion maturation, an HIV-1 protease-cleavable linker was inserted between Gag and Cas9 (FIG. 1B). Cas9-VLPs pseudotyped with the broadly-transducing vesicular stomatitis virus glycoprotein (VSV-G) was produced. The ratio of Gag-pol and Gag-Cas9 plasmids was varied to optimize Cas9 incorporation in budded particles. Bands corresponding to the expected size of Cas9 fused to Gag (55 kDa+160 kDa=215 kDa) and proteolytically released Cas9 (160 kDa) were detectable by western blot in all Cas9-VLP formulations tested (FIG. 1C). A component of the Gag polypeptide, capsid (CA), was used for quantifying Cas9-VLP production by ELISA. CA-containing particles were detected for all formulations except for Cas9-VLP formulation F (FIG. 1D). Formulation F is composed entirely of Gag-Cas9 polypeptides which may interfere with the successful budding of Cas9-VLPs from transfected cells.

It was hypothesized that Cas9-VLPs packaging relatively high Gag-Cas9 polypeptide content would require fewer individual Cas9-VLPs to deliver a sufficient quantity of Cas9 RNPs for successful genome editing. To assess genome editing activity, Cas9-VLPs were produced with a lentiviral transfer plasmid expressing a sgRNA targeting the beta-2 microglobulin (B2M) gene. The transduction-competent Cas9-VLP titer (TU/mL) was quantified for each Cas9-VLP preparation (FIG. 1E), and was used to calculate the multiplicity of infection (MOI, TU/cell) required to achieve 50% editing (effective concentration 50, EC50 MOI) in the Jurkat cells (FIG. 1F). It was confirmed that as increasing amounts of Gag-Cas9 are packaged per particle, a lower EC50 MOI is needed to achieve genome editing (FIG. 1G) with an approximate MOI=2.6 required to achieve 50% indels using Cas9-VLP formulation B and MOI=0.9 using Cas9-VLP formulation D.

FIG. 1A-1G. Production and characterization of Cas9-VLPs. (A) Schematic of plasmids for Cas9-VLP production. GP=glycoprotein. LV=lentiviral transfer plasmid. LTR=long terminal repeat. (B) Schematic of an immature Cas9-VLP produced through transient transfection. An HIV-1 protease cleavable linker (SQNYPIVQ; SEQ ID NO: 155) was inserted between Gag and Cas9. (C) Western blot of Cas9-VLP content with various ratios of Gag-pol and Gag-Cas9 plasmids used for production. Anti-FLAG (Cas9) and anti-p24 (capsid, CA) antibodies were used for detection. (D) Quantification of Cas9-VLPs produced per transfected p100 dish by CA ELISA, n=2 technical replicates. (E) Jurkat cell (“Jurkats”) were treated with B2M-targeting Cas9-VLP and the transducing units (TU) per mL titer was calculated. (F) Percent B2M indels plotted against the multiplicity of infection (MOI) using a sigmoidal four parameter logistic fit. Indels were quantified using Synthego's ICE analysis tool. (G) The predicted MOI for each Cas9-VLP formulation to achieve 50% indels, (interpolated from F). EC50=effective concentration at which a drug gives a half-maximal response. n=3 technical replicates (E, F), except for formulation A (n=2) (F). Error bars indicate standard error of the mean (D, E, F) and 95% confidence interval (G). ND=not detected.

### Characterization of Cas9-VLPs for Genome Editing

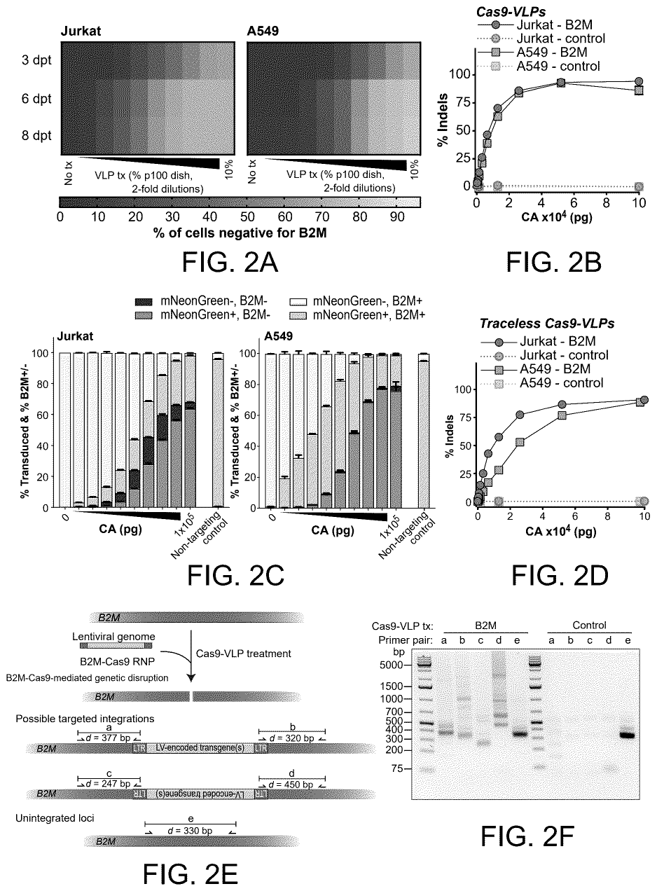

The kinetics of genome editing following Cas9-VLP treatment was assessed. Jurkat and A549 cells were treated with formulation D B2M-targeting Cas9-VLPs and cell-surface expressed B2M protein was assessed by flow cytometry at 3, 6, and 8 days post treatment. Dose-dependent knockout of B2M protein was observed by day 3 (FIG. 2A), with a maximum loss of protein expression achieved by day 6. Genetic knockout was further confirmed by next-generation sequencing and observed B2M-guide specific indels at the B2M locus (FIG. 2B). Similar to what has been observed for Cas9-packaging MLV VLPs (Mangeot et al., 2019), mixing Cas9-VLPs with a DNA template was sufficient to mediate homology-directed repair (HDR) in a fluorescence reporter assay (Richardson et al., 2016). It was found that Cas9-VLP-directed HDR activity could be further enhanced by electroporating Cas9-VLPs with the DNA template prior to the treatment of target cells, which may promote complexing of Cas9-VLPs with the HDR template (FIG. 5A-5E).

Current RNP-based genome editing approaches have not permitted the quantification of cells edited as a function of the number of cells receiving RNPs. It was reasoned that Cas9-VLPs co-delivering Cas9 RNPs and a lentiviral genome may enable tracking cells that receive Cas9 RNPs. To assess whether transduction is a marker of RNP-edited cells, A549s and Jurkats were treated with serial dilutions of B2M-Cas9-VLPs delivering the mNeonGreen transgene and quantified B2M expression at day 6 post treatment (FIG. 2C, FIG. 6A-6F). For Jurkats, successfully-edited cells largely correlated with the transduction marker mNeonGreen; however, a population of B2M-knockout cells that did not express mNeonGreen was observed. It was hypothesized that this discordance could be explained by a proportion of Cas9-VLPs not co-packaging both the lentiviral genome and Cas9 RNPs. However, in A549 cells treated with the same Cas9-VLP preparation, cells lacking B2M overwhelmingly expressed the transduction marker. This suggests that in the A549 cell line, transduction is a reliable marker for identifying the cell population edited by Cas9 RNPs and that Jurkats may employ a cell-intrinsic mechanism restricting reverse transcription of the lentiviral genome, nuclear import, or integration independent of Cas9-mediated genome editing.

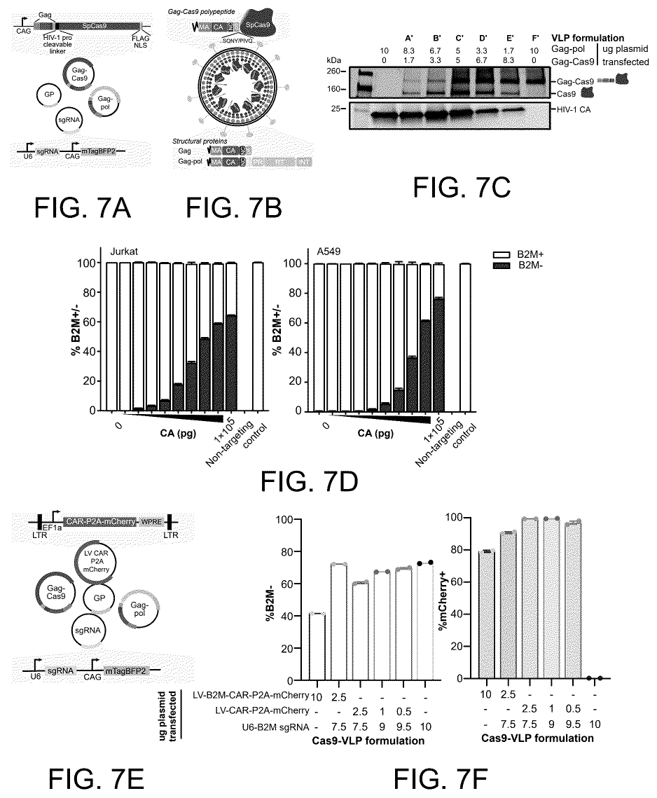

As the sgRNA expression cassette is embedded within the lentiviral genome, sgRNA transcription could occur both in the packaging cell line during Cas9-VLP production and in transduced cells. To assess whether Cas9 RNP formation occurs predominantly in the packaging cells or in the treated cells, Cas9-VLPs lacking a lentiviral genome were produced; the B2M sgRNA was expressed instead from an orthogonal expression plasmid. It was found that treatment with “traceless” Cas9-VLPs mediated high levels of editing (FIG. 2D, FIG. 7A-7D) suggesting the majority of Cas9 RNPs are formed within the packaging cell line. A slight increase in editing efficiency was noted when a lentiviral genome including the sgRNA expression cassette was co-packaged within the Cas9-VLP (FIG. 2B vs. FIG. 2D) suggesting, at this concentration, a fraction of VLP-packaged Cas9 may remain in the guideless apo-Cas9 state until sgRNA transcription occurs in treated cells. It was also possible to generate hybrid Cas9-VLPs that co-package a lentiviral genome but do not require a lentiviral-encoded guide RNA expression cassette (FIG. 7E-7F). Together, the ability of Cas9-VLPs to deliver Cas9 RNPs without co-packaging a lentiviral genome enables Cas9-VLPs to mediate genome editing in the absence of transgene integration, which may be advantageous for clinical applications.

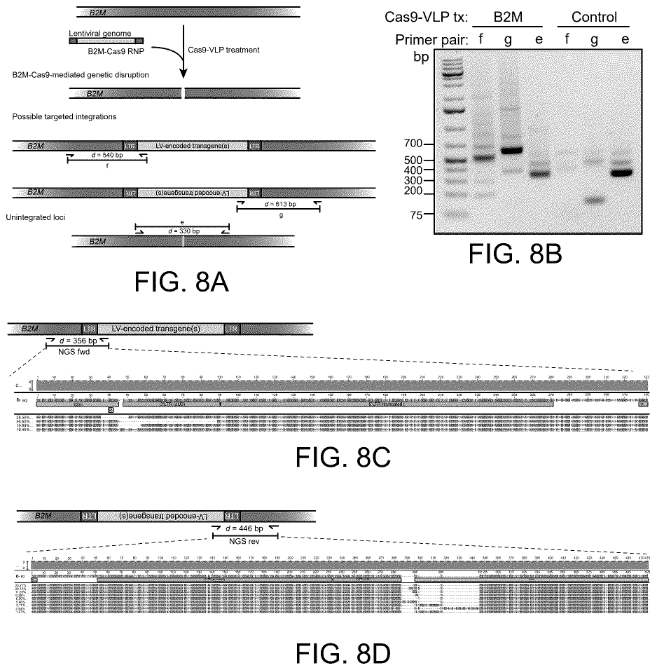

As Cas9-VLPs deliver the reverse-transcribed viral genome concomitantly with dsDNA-break inducing Cas9 RNPs, it was reasoned that targeted lentiviral insertion may occur at the genomic site targeted for genome editing. To investigate this possibility, DNA was isolated from cells treated with either B2M-targeting or non-targeting Cas9-VLPs co-packaging a lentiviral genome. Amplification of cellular genomic DNA with primers specific to the B2M locus and the lentiviral LTR resulted in detectable viral insertion at the Cas9-targeted region (FIG. 2E-F). This was further validated using primers specific to the B2M locus and the lentiviral Psi sequence, and next-generation sequencing confirmed targeted lentiviral integration (FIG. 8A-8D).

FIG. 2A-2F. Cas9-VLPs efficiently mediate genome editing. B2M-targeting Cas9-VLP treatment results in genome editing of Jurkat and A549 cells. (A) Flow cytometry quantification of B2M expression at 3, 6, and 8 days post treatment (dpt). Heat maps represent the mean of technical replicates, n=3, except for A549 at 8 dpt (n=2). The highest treatment dose=10% of Cas9-VLPs produced in a p100 dish. (B) Amplicon sequencing quantification of indels, 3 dpt. Control=tdTom298 sgRNA. n=3 technical replicates, except for A549 treated with 10×10⁴ pg CA (n=2). (C) Flow cytometry quantification of B2M expression and transduction (mNeonGreen+), 6 dpt. Non-targeting control=guideless Cas9-VLP. n=3 technical replicates. (D) Treatment with Cas9-VLPs that target B2M but do not co-package a lentiviral genome. Amplicon sequencing quantification of indels, 3 dpt. Control=tdTom298 sgRNA. n=3 technical replicates, except for Jurkats treated with 10×10⁴ pg CA traceless B2M Cas9-VLPs (n=2). (E) Schematic of hypothetical lentiviral insertion at the Cas9 RNP-induced DNA break. (F) PCR assessment of targeted lentiviral integration. DNA was isolated from 293T cells 3 dpt with B2M-targeting or non-targeting Cas9-VLPs and indicated primer pairs were used for analysis. Error bars indicate standard error of the mean.

FIG. 5A-5E. Cas9-VLPs mediate homology-directed repair (HDR). (A) Schematic of nucleofection of Cas9-VLPs and single-stranded DNA homology-directed repair templates (HDRT, purple). (B) Assessment of different Lonza nucleofection buffers and pulse codes, 5 days post treatment. Cas9-VLPs packaging BFP-targeting RNPs were mixed with 80 pmol HDRT and nucleofected using the indicated nucleofection buffers and pulse codes. Nucleofected HDRT/Cas9-VLPs were subsequently used to treat a BFP-to-GFP HDR reporter HEK293 cell line (Richardson et al., 2016) where BFP knockout is indicative of non-homologous end joining and GFP expression is representative of HDR. (C) HDR-mediated GFP expression induced treatment with Cas9-VLPs nucleofected (Lonza, CM-150) with 500 pmol HDRT in different buffers, 7 days post treatment. (D) HDR-mediated GFP expression with varying concentrations of HDRT nucleofected (Lonza, CM-150) with Cas9-VLPs in SE buffer (Lonza), 7 days post treatment. (E) Pre-nucleofection of Cas9-VLPs and HDRT enhances HDR activity. Cas9-VLPs (2.59×10⁶ pg CA) and 500 pmol HDRT were mixed in SE buffer and either directly added to BFP-to-GFP reporter cells or subjected to nucleofection (Lonza, CM-150) prior to cell treatment. BFP-positive and GFP-positive cells were quantitated by flow cytometry at 7 days post treatment. All error bars represent standard error of the mean.

FIG. 6A-6F. All Cas9-VLP formulations mediate genome editing, Jurkat or A549 cells were treated with B2M-Cas9-VLP formulations A-E and transduction (mNeonGreen+) and B2M expression were assessed by flow cytometry 6 days post treatment. Of note, cells transfected to produce B2M-targeted Cas9-VLPs themselves undergo genome editing (DNA isolated 3 days post transfection). n=3 technical replicates were performed at each Cas9-VLP treatment dose and error bars represent standard error of the mean.

FIG. 7A-7F. Traceless Cas9-VLPs mediate genome editing without viral transgene insertion and hybrid Cas9-VLPs do not require a lentiviral-encoded guide RNA expression cassette. (A) Schematic of plasmids used for the production of traceless Cas9-VLPs. GP=glycoprotein. (B) Schematic of an immature, pre-proteolytically processed Cas9-VLP, produced through transient transfection and lacking a lentiviral genome. An HIV-1 protease cleavable linker containing SQNY/PIVQ was inserted between the c-termini of Gag and the n-termini of Cas9 to promote the separation during proteolytic virion maturation. (C) Western blot of Cas9-VLP content when various ratios of Gag-pol to Gag-Cas9 plasmids are used for production. An anti-Flag antibody was used for Cas9 detection and an anti-HIV-1 capsid (CA) antibody was used to detect Cas9-VLP production. A′ is used to indicate VLP formulation “A” lacking a packaged lentiviral genome. (D) Flow cytometry quantification of B2M expression in A549 and Jurkats 6 days post treatment with traceless Cas9-VLPs. Non-targeting control=Cas9-VLPs packaging the tdTom298 sgRNA. n=3 technical replicates were performed at each Cas9-VLP treatment dose and error bars indicate standard error of the mean. (E) Schematic of plasmids used for the production of Cas9-VLPs that co-package Cas9 RNPs and a lentiviral genome that lacks a guide RNA expression cassette (“hybrid Cas9-VLPs”). (F) Optimization of hybrid Cas9-VLPs. Cas9-VLPs were produced as indicated and used to treat Jurkat cells. Targeted protein disruption (% of cells negative for B2M expression) and transduction (% of cells mCherry positive) was quantified at day 7. LV-B2M-CAR-P2A-mCherry=lentiviral transfer plasmid that encodes the U6-promoter driven expression of a B2M-targeting guide RNA and the EF1a-promoter driven expression of a CAR-P2A-mCherry transgene. LV-CAR-P2A-mCherry=lentiviral transfer plasmid that encodes the CAR-P2A-mCherry expression cassette alone. U6-B2M=a transient guide RNA expression plasmid.

FIG. 8A-8D. Targeted integration of the lentiviral genome into the Cas9 RNP target site. (A) Schematic of hypothetical lentiviral insertion at the Cas9 RNP-induced double-stranded DNA break. (B) PCR to assess targeted lentiviral integration. DNA was isolated from 293T cells 3 days post treatment with B2M-targeting or non-targeting Cas9-VLPs and the indicated primer pairs were used for analysis. (C) MiSeq analysis of the targeted “forward” lentiviral integration in cells treated with B2M Cas9-VLPs. Reads mapped to the hypothetical B2M-lentiviral junction are shown. (D) MiSeq analysis of the targeted “reverse” lentiviral integration in cells treated with B2M Cas9-VLPs. Reads mapped to the hypothetical B2M-lentiviral junction are shown. Amplicon sizes include Illumina adaptor sequences, see Table S2 (FIG. 15).

### Example 2: Cas9-VLPs Efficiently Edit Primary Human T Cells

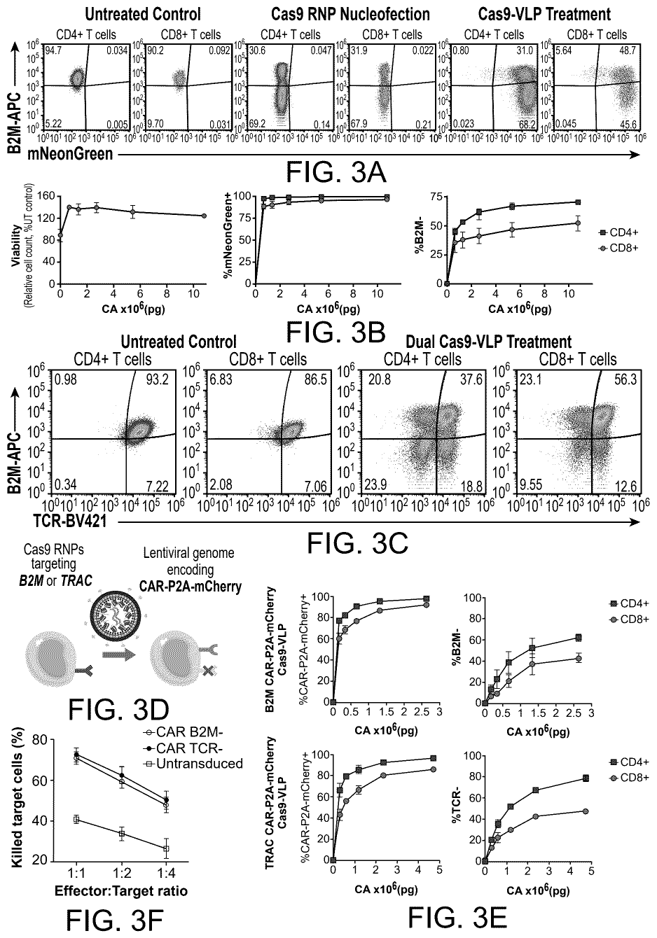

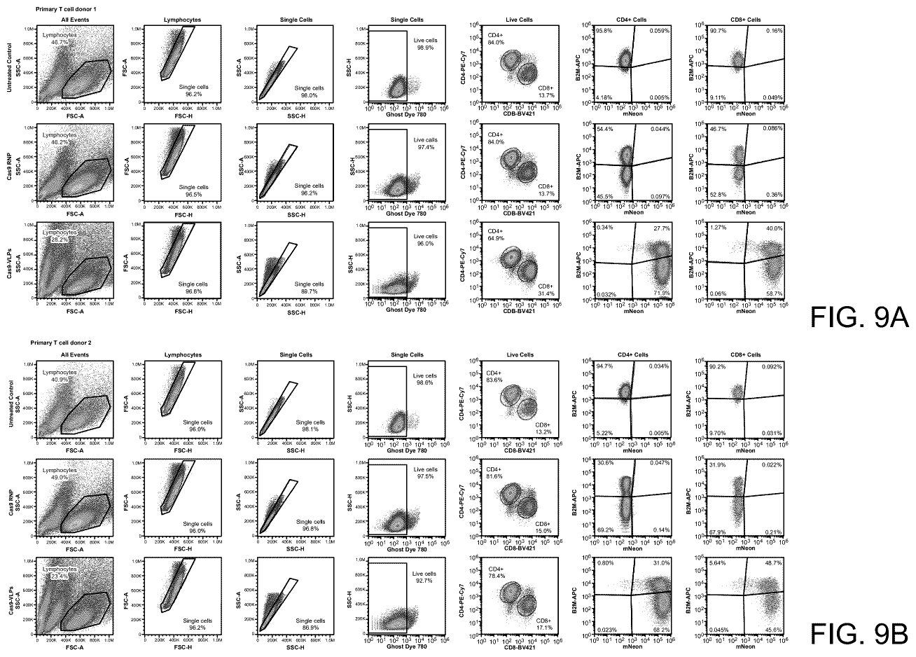

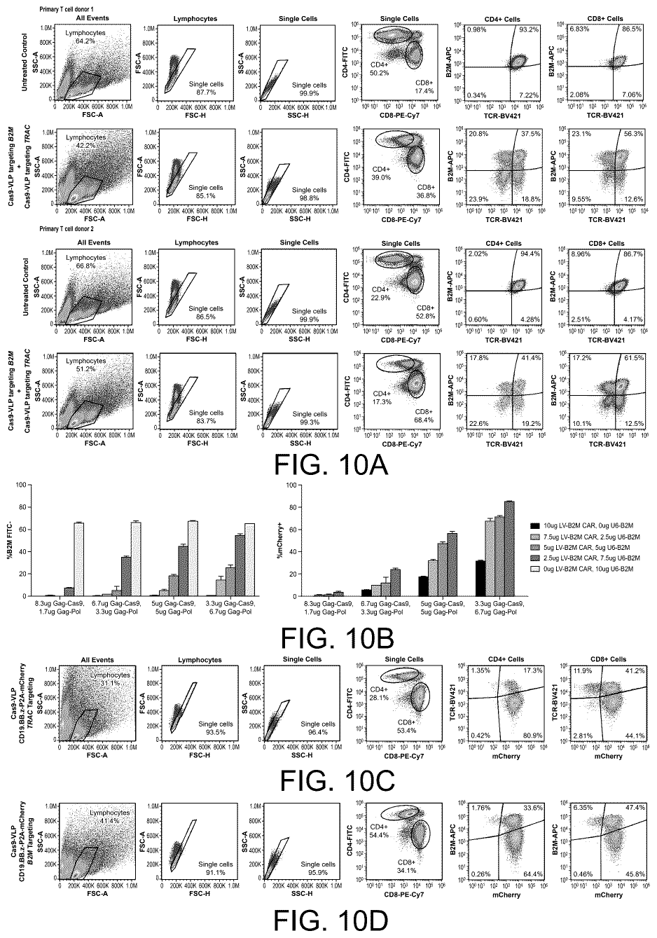

Engineered T cell therapies are transforming the treatment of certain cancers by retargeting T cell activity through introduction of antigen-specific receptors such as chimeric antigen receptors (CARs) (Fesnak et al., 2016; Sadelain et al., 2017). It was tested if Cas9-VLPs could mediate genome editing in primary human T cells. Transducing bulk CD4+ and CD8+ primary human T cells with Cas9-VLPs resulted in B2M knockout levels comparable to Cas9 RNP electroporation, the current clinical standard (FIG. 3A, FIG. 3B, FIG. 9A-9B). Cas9-VLP-mediated transduction and B2M knockout was dose-dependent and cellular viability (as measured by relative cell count) was improved compared to previous reports employing Cas9 RNP nucleofection (Roth et al., 2018) (FIG. 3B).

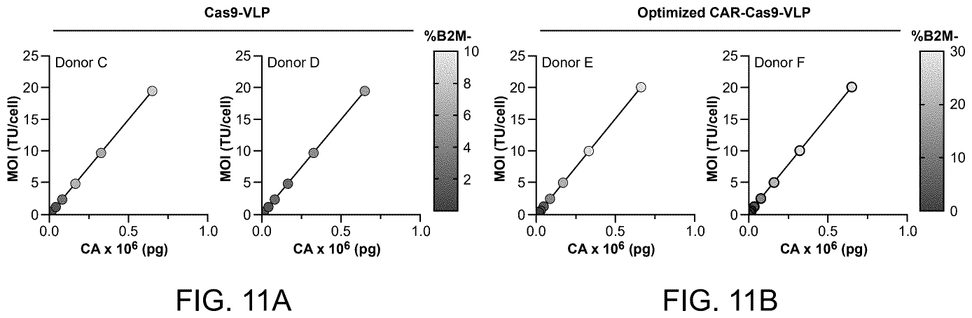

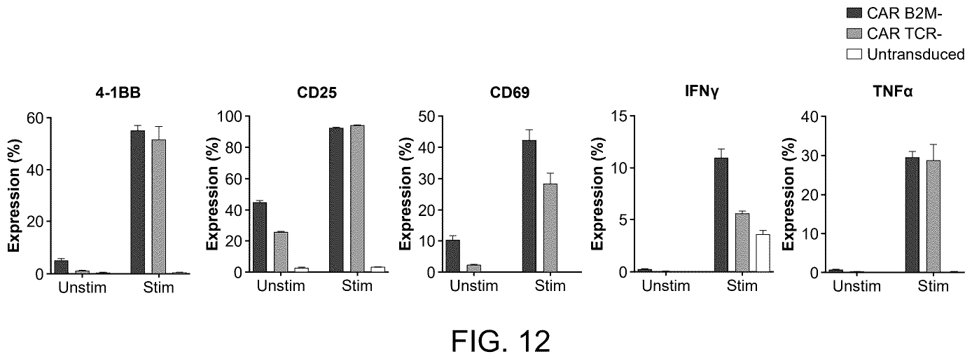

Recently, transgenic T cell receptor (TCR) T cells modified by CRISPR-Cas9 were tested in the first phase I clinical trial (Stadtmauer et al., 2020). The engineered T cell product was produced by electroporation of Cas9 RNPs to first disrupt expression of PD-1 and the endogenous TCR (by targeting PDCD1 and TRAC, respectively) followed by subsequent lentiviral transduction to integrate an exogenous TCR for retargeting antigen specificity. It was hypothesized that Cas9-VLPs could simplify the production of multiply-edited engineered T cells by simultaneously delivering Cas9 RNPs and a lentiviral genome encoding a transgenic TCR or CAR (FIG. 3D). To test this, it was assessed whether it was possible to multiplex genetic knockout by treating primary human T cells with Cas9-VLPs targeting two genetic loci for disruption. Treatment of primary human T cells with separate Cas9-VLPs targeting B2M or TRAC resulted in 23.9% CD4+ and 9.55% CD8+ double-knockout cells by 13 days post treatment (FIG. 3C, FIG. 10A). The production of Cas9-VLPs was optimized to maximize the simultaneous integration of a lentiviral-encoded CAR and knockout of B2M expression in Jurkats (“CAR-Cas9-VLPs”) (FIG. 10B). To determine how capsid quantity correlates to MOI in primary T cells, genome editing levels generated using both mNeonGreen and CAR Cas9-VLPs were assessed. An approximate MOI=20 for mNeonGreen Cas9-VLPs resulted in ˜7% of cells lacking B2M protein while the equivalent MOI for CAR-Cas9-VLPs resulted in ˜28% B2M-negative cells (FIG. 11A-11B). The enhanced editing efficiency of CAR-Cas9-VLPs may be explained by a higher proportion of VLP-packaged Cas9 being associated with guide RNA, as the optimized CAR-Cas9-VLP production involves over-expression of guide RNA in VLP producer cells (FIG. 10B). CAR-Cas9-VLPs packaging Cas9 RNPs targeting either B2M or TRAC for disruption were generated. Primary T cells treated with such RNPs exhibited dose-dependent CAR-P2A-mCherry expression and reduction in surface-expressed B2M or TCR (FIG. 3E, FIG. 10D-10D). Additionally, Cas9-VLP-engineered CAR-T cells were functionalized to kill CD19+ Nalm-6 target cells (FIG. 3F) and stimulation resulted in effector profiles for cytokine production and activation marker expression (FIG. 12). Together, Cas9-VLPs provide a simplified workflow for manufacturing complex CRISPR-modified CAR-T cells in a single step which compares favorably to current clinical manufacturing methods.

FIG. 3A-3F. Generation of highly engineered CAR-expressing primary human T cells using Cas9-VLPs. (A) Cas9 RNP nucleofection and Cas9-VLP treatment of primary human T cells. Flow cytometry quantification of the mNeonGreen transduction marker and B2M expression, 7dpt. (B) Viability, transduction, and B2M expression in primary human T cells treated with Cas9-VLPs. B2M expression is plotted for CD4+ (red squares) and CD8+ (blue circles) subpopulations. (C) Simultaneous treatment with two Cas9-VLPs results in multiplexed genome editing. Cas9-VLPs targeting B2M and Cas9-VLPs targeting TRAC were used to co-treat primary human T cells. Surface expression of B2M and TCR was assessed by flow cytometry, 13 dpt. n=2 biological replicates from independent donors were used (A, B, C) and representative flow cytometry plots are shown for one donor (A, C). (D) Schematic of a single-step method to generate highly engineered CAR-T cells. (E) Primary human T cells were treated with CAR-Cas9-VLPs targeting B2M (top panels) or TRAC (bottom panels). Knockout was assessed for both CD4+ (red squares) and CD8+ (blue circles) subpopulations, 12 dpt. (F) CAR-T cells generated by Cas9-VLP treatment, or untreated primary human T cells, were co-cultured with CD19+ Nalm-6 cells and cytotoxic killing activity was measured at 24 h. Error bars indicate standard error of the mean.

FIG. 9A-9B. Representative flow cytometry gating strategy for quantifying genome editing in primary human T cells. (A) Flow cytometry gating strategy to assess surface-expressed B2M in primary human T cells after no treatment, nucleofection of Cas9 RNPs, and treatment with Cas9-VLPs from donor 1. (B) Flow cytometry gating strategy to assess surface-expressed B2M in primary human T cells after no treatment, nucleofection of Cas9 RNPs, and treatment with Cas9-VLPs from donor 2.

FIG. 10A-10D. Optimization of CAR-Cas9-VLP production & representative flow cytometry gating strategy for Cas9-VLP-mediated multiplexed genome engineering of primary human CAR-T cells. (A) Flow cytometry gating strategy to assess the dual knockout of surface-expressed TCR and B2M by simultaneous treatment with Cas9-VLPs targeting TRAC and Cas9-VLPs targeting B2M in two independent T cell donors. Cas9-VLPs optimized for simultaneous CAR transgene insertion and B2M knockout were used (FIG. 9B). (B) Optimization of Cas9-VLP production to maximize simultaneous CAR transgene integration and genome editing. Cas9-VLPs were produced with various ratios of plasmids encoding the Gag-Cas9 and Gag-pol structural proteins, and with various ratios of plasmids encoding a lentiviral transfer plasmid (encoding expression cassettes for U6-B2M CAR-P2A-mCherry) and a U6-B2M guide RNA expression plasmid. Jurkats were treated, passed at day 4 post treatment to maintain subconfluent culture conditions and flow cytometry was performed at 6 days post treatment to quantify B2M expression (B, left) and CAR-P2A-mCherry expression (B, right). Cas9-VLPs produced through transient transfection with the following plasmids were most efficient at mediating simultaneous knockout of B2M and CAR-P2A-mCherry transgene expression: 1 μg VSV-G, 3.3 μg Gag-Cas9, 6.7 μg Gag-pol plasmid, 2.5 μg LV-B2M, and 7.5 μg U6-B2M. n=2 replicates per treatment, error bars represent standard error of the mean. (C) Flow cytometry gating strategy to assess the knockout of surface-expressed TCR and expression of CAR-P2A-mCherry in primary human T cells by treatment with Cas9-VLPs. (D) Flow cytometry gating strategy to assess the knockout of surface-expressed B2M and expression of CAR-P2A-mCherry in primary human T cells by treatment with Cas9-VLPs.

FIG. 11A-11B. Cas9-VLP genome editing as a function of MOI and quantity of CA. (A) Cas9-VLPs co-packaging B2M-targeting Cas9 RNPs and a lentiviral genome encoding mNeonGreen were generated (as used in FIG. 3) and (B) Cas9-VLPs optimized to co-package B2M-targeting Cas9 RNPs and a lentiviral genome encoding CAR-P2A-mCherry were produced. The transducing units/mL (TU/mL) titer and capsid (CA) content were quantified for each Cas9-VLP preparation. Primary T cells from two human donors were treated with indicated multiplicity of infection (MOI) and picogram (pg) CA and cells negative for B2M protein were quantified by flow cytometry at day 7.

FIG. 12. Functional cytokine production and surface receptor expression in Cas9-VLP generated CAR-T cells. Cytokine and surface receptor expression were quantified in stimulated and unstimulated CAR-T cells generated from Cas9-VLPs at 24 h. For all, n=2 biological replicates from independent donors were used and error bars indicate standard error of the mean.

### Example 3: Cell-Type Specific Editing Via Pseudotyping of Cas9-VLPs

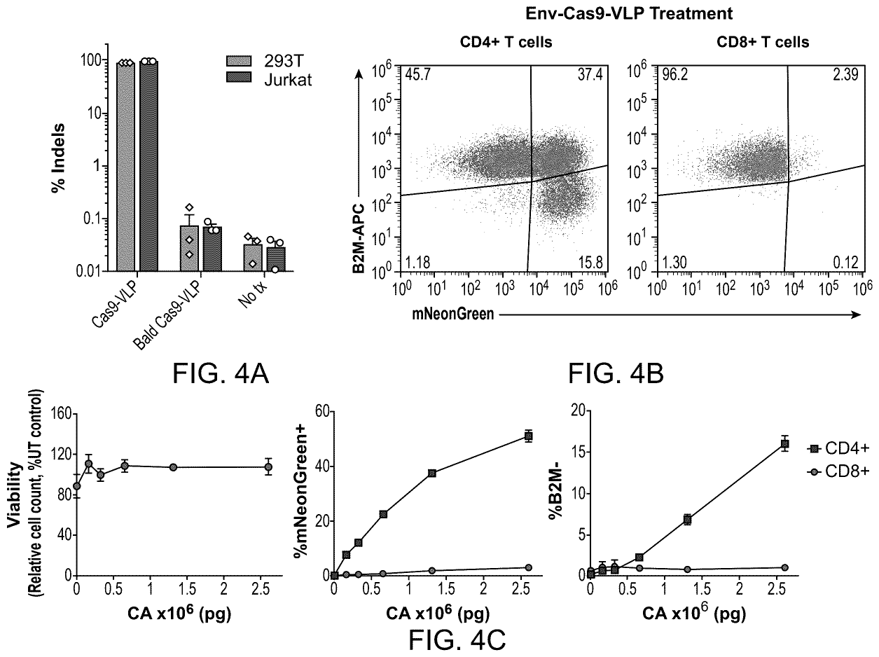

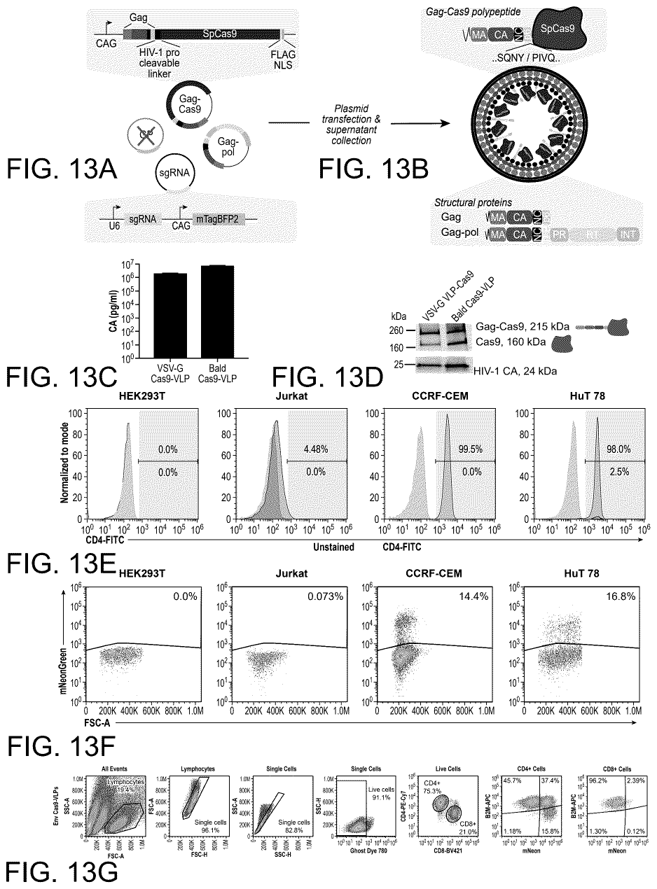

Virus and VLP cell-type specificity may be altered through pseudotyping with varied surface glycoproteins (Cronin et al., 2005). To test whether the Cas9-VLP glycoproteins were essential for the genome editing of mammalian cells, Cas9-VLPs lacking viral glycoproteins (“bald” Cas9-VLPs) were produced and their ability to mediate genome editing was assessed. “Bald” Cas9-VLPs were effectively produced (FIG. 13A-13D) but cellular treatment resulted in <0.1% of reads containing indels by deep sequencing, a 3-log reduction in genome editing compared to treatment with VSV-G pseudotyped Cas9-VLPs (FIG. 4A). Efficient delivery of VLP-packaged Cas9 RNPs is therefore dependent upon the expression of viral glycoproteins. To test whether Cas9-VLPs could be engineered to target a specific cell type for genome editing, Cas9-VLPs pseudotyped with the HIV-1 envelope glycoprotein (Env), the viral determinant for HIV-1's CD4+ T cell tropism (Clapham and McKnight, 2001), were produced (FIG. 13E-13F). Env-Cas9-VLPs were produced packaging Cas9 RNPs targeting human B2M locus and an mNeonGreen-expressing lentiviral genome. A mixture of CD4+ and CD8+ T cells were treated with Env-Cas9-VLPs and transduction and B2M protein expression were assessed. At the highest treatment dose, Env-Cas9-VLPs preferentially transduced CD4+ cells over CD8+ cells (53.20% vs. 2.51%, respectively) (FIG. 4B-C, FIG. 13G). Concomitantly, Env-Cas9-VLP treatment resulted in knockout of B2M in CD4+ T cells while co-cultured CD8+ T cells remained unmodified. This establishes Cas9-VLP pseudotyping as a promising approach to specifically retarget Cas9 RNP-mediated genome editing to predetermined cell types within a mixed cell population without unintended modification of bystander cells.

FIG. 4A-4C. HIV-1 Envelope pseudotyping targets Cas9-VLP genome editing to CD4+ T cells. (A) A viral glycoprotein is essential for Cas9-VLP-mediated genome editing. 293T and Jurkat cells were treated with B2M-targeting Cas9-VLPs pseudotyped with VSV-G (Cas9-VLP), without VSV-G (Bald Cas9-VLP) or were left untreated (No tx). Indels were quantified by amplicon sequencing, 3 dpt, n=3. (B) B2M-targeting Cas9-VLPs pseudotyped with the HIV-1 envelope glycoprotein (Env-Cas9-VLPs) were used to treat primary human T cells (a mixture of CD4+ and CD8+ cells). (C) Viability, transduction (mNeonGreen) and B2M expression were assessed for CD4+(red squares) and CD8+ (blue circles) subpopulations 7 dpt. n=2 biological replicates from independent donors were used (B, C) and representative flow cytometry plots are shown for one donor (B). Error bars indicate standard error of the mean.

FIG. 13A-13G. Characterization of bald and HIV-1 Env pseudotyped Cas9-VLPs. (A) Production of “bald” Cas-VLPs. Schematic of plasmids used for the production of bald Cas9-VLPs that lack a glycoprotein. (B) Schematic of an immature, pre-proteolytically processed Cas9-VLP produced through transient transfection. (C) Quantification of Cas9-VLP production by CA ELISA. Amount of CA produced per transfected p100 dish is shown. (D) Western blot of Cas9-VLP content. An anti-Flag antibody was used for Cas9 detection and an anti-HIV-1 capsid (CA) antibody was used to detect Cas9-VLP production. (E) Env-Cas9-VLPs are specific for CD4+ cells. Cell surface expression of CD4 in HEK293T, Jurkat, CCRF-CEM, and HuT 78 cell lines. (F) Transduction of Cas9-VLPs pseudotyped with the HIV-1 envelope correlates with cellular CD4 expression. (G) Representative flow cytometry gating strategy to assess the cell-type specificity of B2M knockout by Env-Cas9-VLPs within a mixed population of primary human T cells.

### REFERENCES

Aoki, T., Miyauchi, K., Urano, E., Ichikawa, R., and Komano, J. (2011). Protein transduction by pseudotyped lentivirus-like nanoparticles. Gene Ther. 18, 936-941.
Bailey, S. R., and Maus, M. V. (2019). Gene editing for immune cell therapies. Nature Biotechnology 37, 1425-1434.
Cai, Y., Bak, R. O., Krogh, L. B., Staunstrup, N. H., Moldt, B., Corydon, T. J., Schroder, L. D., and Mikkelsen, J. G. (2014a). DNA transposition by protein transduction of the piggyBac transposase from lentiviral Gag precursors. Nucleic Acids Res. 42, e28.
Cai, Y., Bak, R. O., and Mikkelsen, J. G. (2014b). Targeted genome editing by lentiviral protein transduction of zinc-finger and TAL-effector nucleases. Elife 3, e01911.
Chen, G., Abdeen, A. A., Wang, Y., Shahi, P. K., Robertson, S., Xie, R., Suzuki, M., Pattnaik, B. R., Saha, K., and Gong, S. (2019). A biodegradable nanocapsule delivers a Cas9 ribonucleoprotein complex for in vivo genome editing. Nat. Nanotechnol. 14, 974-980.
Choi, J. G., Dang, Y., Abraham, S., Ma, H., Zhang, J., Guo, H., Cai, Y., Mikkelsen, J. G., Wu, H., Shankar, P., et al. (2016). Lentivirus pre-packed with Cas9 protein for safer gene editing. Gene Ther. 23, 627-633.
Clapham, P. R., and McKnight, A. (2001). HIV-1 receptors and cell tropism. British Medical Bulletin 58, 43-59.
Cronin, J., Zhang, X.-Y., and Reiser, J. (2005). Altering the tropism of lentiviral vectors through pseudotyping. Curr. Gene Ther. 5, 387-398.
Del'Guidice, T., Lepetit-Stoffaes, J.-P., Bordeleau, L.-J., Roberge, J., Théberge, V., Lauvaux, C., Barbeau, X., Trottier, J., Dave, V., Roy, D.-C., et al. (2018). Membrane permeabilizing amphiphilic peptide delivers recombinant transcription factor and CRISPR-Cas9/Cpf1 ribonucleoproteins in hard-to-modify cells. PLoS One 13, e0195558.
Doudna, J. A. (2020). The promise and challenge of therapeutic genome editing. Nature 578, 229-236.
Fesnak, A. D., June, C. H., and Levine, B. L. (2016). Engineered T cells: the promise and challenges of cancer immunotherapy. Nat. Rev. Cancer 16, 566-581.
van Haasteren, J., Li, J., Scheideler, O. J., Murthy, N., and Schaffer, D. V. (2020). The delivery challenge: fulfilling the promise of therapeutic genome editing. Nat. Biotechnol. 38, 845-855.
Hill, Z. B., Martinko, A. J., Nguyen, D. P., and Wells, J. A. (2018). Human antibody-based chemically induced dimerizers for cell therapeutic applications. Nat. Chem. Biol. 14, 112-117.
Hu, W., Zi, Z., Jin, Y., Li, G., Shao, K., Cai, Q., Ma, X., and Wei, F. (2019). CRISPR/Cas9-mediated PD-1 disruption enhances human mesothelin-targeted CAR T cell effector functions. Cancer Immunology, Immunotherapy 68, 365-377.
Indikova, I., and Indik, S. (2020). Highly efficient “hit-and-run” genome editing with unconcentrated lentivectors carrying Vpr.Prot.Cas9 protein produced from RRE-containing transcripts. Nucleic Acids Research 48, 8178-8187.
Izmiryan, A., Basmaciogullari, S., Henry, A., Paques, F., and Danos, O. (2011). Efficient gene targeting mediated by a lentiviral vector-associated meganuclease. Nucleic Acids Res. 39, 7610-7619.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-821.
Lim, W. A., and June, C. H. (2017). The Principles of Engineering Immune Cells to Treat Cancer. Cell 168, 724-740.
Lyu, P., Javidi-Parsijani, P., Atala, A., and Lu, B. (2019). Delivering Cas9/sgRNA ribonucleoprotein (RNP) by lentiviral capsid-based bionanoparticles for efficient “hit-and-run” genome editing. Nucleic Acids Research 47, e99-e99.
Mangeot, P. E., Risson, V., Fusil, F., Mamef, A., Laurent, E., Blin, J., Mournetas, V., Massouridès, E., Sohier, T. J. M., Corbin, A., et al. (2019). Genome editing in primary cells and in vivo using viral-derived Nanoblades loaded with Cas9-sgRNA ribonucleoproteins. Nat. Commun. 10, 45.
Michel, G., Yu, Y., Chang, T., and Yee, J.-K. (2010). Site-specific gene insertion mediated by a Cre-loxP-carrying lentiviral vector. Mol. Ther. 18, 1814-1821.
Miyauchi, K., Urano, E., Takizawa, M., Ichikawa, R., and Komano, J. (2012). Therapeutic potential of HIV protease-activable CASP3. Sci. Rep. 2, 359.
Muller, Y. D., Nguyen, D. P., Ferreira, L. M. R., Ho, P., and Raffin, C. (2020). The CD28-transmembrane domain mediates chimeric antigen receptor heterodimerization with CD28. bioRxiv.
Newick, K., O'Brien, S., Moon, E., and Albelda, S. M. (2017). CAR T Cell Therapy for Solid Tumors. Annu. Rev. Med. 68, 139-152.
Nguyen, D. N., Roth, T. L., Li, P. J., Chen, P. A., Apathy, R., Mamedov, M. R., Vo, L. T., Tobin, V. R., Goodman, D., Shifrut, E., et al. (2020). Polymer-stabilized Cas9 nanoparticles and modified repair templates increase genome editing efficiency. Nat. Biotechnol. 38, 44-49.
Porteus, M. H. (2019). A New Class of Medicines through DNA Editing. N. Engl. J. Med. 380, 947-959.
Ramakrishna, S., -B. Kwaku Dad, A., Beloor, J., Gopalappa, R., Lee, S.-K., and Kim, H. (2014). Gene disruption by cell-penetrating peptide-mediated delivery of Cas9 protein and guide RNA. Genome Research 24, 1020-1027.
Richardson, C. D., Ray, G. J., DeWitt, M. A., Curie, G. L., and Corn, J. E. (2016). Enhancing homology-directed genome editing by catalytically active and inactive CRISPR-Cas9 using asymmetric donor DNA. Nat. Biotechnol. 34, 339-344.
Roth, T. L., Puig-Saus, C., Yu, R., Shifrut, E., Carnevale, J., Li, P. J., Hiatt, J., Saco, J., Krystofinski, P., Li, H., et al. (2018). Reprogramming human T cell function and specificity with non-viral genome targeting. Nature 559, 405-409.
Rouet, R., Thuma, B. A., Roy, M. D., Lintner, N. G., Rubitski, D. M., Finley, J. E., Wisniewska, H. M., Mendonsa, R., Hirsh, A., de Onate, L., et al. (2018). Receptor-Mediated Delivery of CRISPR-Cas9 Endonuclease for Cell-Type-Specific Gene Editing. J. Am. Chem. Soc. 140, 6596-6603.
Rupp, L. J., Schumann, K., Roybal, K. T., Gate, R. E., Ye, C. J., Lim, W. A., and Marson, A. (2017). CRISPR/Cas9-mediated PD-1 disruption enhances anti-tumor efficacy of human chimeric antigen receptor T cells. Sci. Rep. 7, 737.
Sadelain, M., Rivibre, I., and Riddell, S. (2017). Therapeutic T cell engineering. Nature 545, 423-431.
Schenkwein, D., Turkki, V., Karkkainen, H.-R., Airenne, K., and Yla-Herttuala, S. (2010). Production of HIV-1 integrase fusion protein-carrying lentiviral vectors for gene therapy and protein transduction. Hum. Gene Ther. 21, 589-602.
Staahl, B. T., Benekareddy, M., Coulon-Bainier, C., Banfal, A. A., Floor, S. N., Sabo, J. K., Urnes, C., Munares, G. A., Ghosh, A., and Doudna, J. A. (2017). Efficient genome editing in the mouse brain by local delivery of engineered Cas9 ribonucleoprotein complexes. Nat. Biotechnol. 35, 431-434.
Stadtmauer, E. A., Fraietta, J. A., Davis, M. M., Cohen, A. D., Weber, K. L., Lancaster, E., Mangan, P. A., Kulikovskaya, I., Gupta, M., Chen, F., et al. (2020). CRISPR-engineered T cells in patients with refractory cancer. Science 367.
Tang, N., Cheng, C., Zhang, X., Qiao, M., Li, N., Mu, W., Wei, X.-F., Han, W., and Wang, H. (2020). TGF-β inhibition via CRISPR promotes the long-term efficacy of CAR T cells against solid tumors. JCI Insight 5.
Wagner, J., Wickman, E., DeRenzo, C., and Gottschalk, S. (2020). CAR T Cell Therapy for Solid Tumors: Bright Future or Dark Reality? Mol. Ther. 28, 2320-2339.
Wang, H.-X., Song, Z., Lao, Y.-H., Xu, X., Gong, J., Cheng, D., Chakraborty, S., Park, J. S., Li, M., Huang, D., et al. (2018). Nonviral gene editing via CRISPR/Cas9 delivery by membrane-disruptive and endosomolytic helical polypeptide. Proc. Natl. Acad. Sci. U.S.A 115, 4903-4908.
Weber, E. W., Maus, M. V., and Mackall, C. L. (2020). The Emerging Landscape of Immune Cell Therapies. Cell 181, 46-62.
Wei, T., Cheng, Q., Min, Y.-L., Olson, E. N., and Siegwart, D. J. (2020). Systemic nanoparticle delivery of CRISPR-Cas9 ribonucleoproteins for effective tissue specific genome editing. Nat. Commun. 11, 3232.
Wilson, R. C., and Gilbert, L. A. (2018). The Promise and Challenge of In Vivo Delivery for Genome Therapeutics. ACS Chem. Biol. 13, 376-382.
Wu, X., Liu, H., Xiao, H., Kim, J., Seshaiah, P., Natsoulis, G., Boeke, J. D., Hahn, B. H., and Kappes, J. C. (1995). Targeting foreign proteins to human immunodeficiency virus particles via fusion with Vpr and Vpx. J. Virol. 69, 3389-3398.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A system comprising:
a) a first nucleic acid comprising a nucleotide sequence encoding a fusion polypeptide comprising:
i) a lentiviral gag polyprotein comprising a matrix (MA) polypeptide, a capsid (CA) polypeptide, and a nucleocapsid (NC) polypeptide; and
ii) a CRISPR-Cas effector polypeptide;
wherein the fusion polypeptide comprises proteolytically cleavable linker between the gag polyprotein and the CRISPR-Cas effector polypeptide;
b) a second nucleic acid comprising a nucleotide sequence encoding a therapeutic polypeptide, wherein the second nucleic acid comprises a recombinant lentiviral nucleic acid;
c) a third nucleic acid comprising a nucleotide sequence encoding a pseudotyping viral envelope protein and/or a polypeptide that provides for binding to a target cell; and
d) a fourth nucleic acid comprising a nucleotide sequence encoding a lentiviral pol polyprotein comprising a reverse transcriptase and an integrase;
wherein the system comprises a nucleic acid comprising a nucleotide sequence encoding a CRISPR-Cas guide RNA, wherein the CRISPR-Cas guide RNA-encoding nucleic acid is either:
i) part of the second nucleic acid; or
ii) a fifth nucleic acid.

2. The system of claim 1, wherein the therapeutic polypeptide has a length of from about 250 amino acids to about 3,000 amino acids.

3. The system of claim 2, wherein the therapeutic polypeptide has a length of from about 500 amino acids to about 2,500 amino acids.

4. The system of any one of claims 1-3, wherein the therapeutic polypeptide is a chimeric antigen receptor (CAR), a T-cell receptor, a synNotch polypeptide, a natural killer cell receptor, or an antibody.

5. The system of claim 4, wherein the CAR comprises an antigen-binding domain specific for a cancer-associated antigen.

6. The system of any one of claims 1-5, wherein the recombinant lentiviral nucleic acid is a recombinant human immunodeficiency virus-1 nucleic acid.

7. The system of any one of claims 1-6, wherein the lentiviral gag polyprotein is a human immunodeficiency virus (HIV) gag polyprotein comprising a MA polypeptide, a CA polypeptide, a p2 polypeptide, an NC polypeptide, a p1 polypeptide, and a p6 polypeptide.

8. The system of any one of claims 1-7, wherein the CRISPR-Cas effector polypeptide is a type II CRISPR-Cas effector polypeptide, a type V CRISPR-Cas effector polypeptide, or a type VI CRISPR-Cas effector polypeptide.

9. The system of claim 8, wherein the type II CRISPR/Cas effector polypeptide is a Cas9 polypeptide.

10. The system of any one of claims 1-9, wherein the CRISPR/Cas effector polypeptide is a fusion polypeptide comprising one or more nuclear localization signals.

11. The system of any one of claims 1-10, further comprising a donor template nucleic acid, or a nucleotide sequence encoding the donor template nucleic acid.

12. A eukaryotic cell comprising the system of any one of claims 1-11.

13. The eukaryotic cell of claim 12, wherein the cell is a packaging cell.

14. A virus-like particle (VLP) comprising:
a) a CRISPR-Cas effector polypeptide; and
b) a recombinant lentivirus comprising a nucleotide sequence encoding a therapeutic polypeptide having a length of from about 250 amino acids to about 3,000 amino acids,
wherein the VLP comprises a pseudotyping viral glycoprotein and/or a polypeptide that provides for binding to a target cell.

15. The VLP of claim 14, wherein the therapeutic polypeptide has a length of from about 500 amino acids to about 2,500 amino acids.

16. The VLP of claim 14 or 15, wherein the therapeutic polypeptide is a chimeric antigen receptor (CAR), a T-cell receptor, a synNotch polypeptide, a natural killer cell receptor, or an antibody.

17. The VLP of claim 16, wherein the CAR comprises an antigen-binding domain specific for a cancer-associated antigen.

18. The VLP of any one of claims 14-17, wherein the pseudotyping viral glycoprotein is a human immunodeficiency virus-1 envelope protein, a measles virus hemagglutinin, an HTLV-1 glycoprotein, or a VSV-G glycoprotein.

19. The VLP of any one of claims 14-17, wherein the polypeptide that provides for binding to a target cell is an antibody, optionally wherein the antibody is a single-chain Fv or a nanobody.

20. The VLP of any one of claims 14-17, wherein the polypeptide that provides for binding to a target cell is a DARPin.

21. The VLP of any one of claims 14-20, wherein the CRISPR-Cas effector polypeptide is a type II CRISPR-Cas effector polypeptide, a type V CRISPR-Cas effector polypeptide, or a type VI CRISPR-Cas effector polypeptide.

22. The VLP of claim 21, wherein the type II CRISPR/Cas effector polypeptide is a Cas9 polypeptide.

23. The VLP of any one of claims 14-22, wherein the CRISPR/Cas effector polypeptide is a fusion polypeptide comprising one or more nuclear localization signals.

24. The VLP of any one of claims 14-23, comprising a CRISPR/Cas guide RNA.

25. The VLP of any one of claims 14-24, comprising a donor template nucleic acid.

26. A composition comprising the VLP of any one of claims 14-25.

27. The composition of claim 26, further comprising a donor template nucleic acid.

28. A method of delivering a therapeutic polypeptide to a eukaryotic cell, the method comprising contacting the cell with the VLP of any one of claims 14-25, or the composition of claim 26, wherein said contacting results in synthesis of the therapeutic polypeptide by the cell.

29. The method of claim 28, wherein the eukaryotic cell is in vivo.

30. The method of claim 24, wherein the eukaryotic cell is in vitro.

31. The method of any one of claims 28-30, wherein the eukaryotic cell is a T cell.

32. The method of any one of claims 28-30, wherein the eukaryotic cell is a B cell.

33. The method of any one of claims 28-30, wherein the eukaryotic cell is a stem cell.

34. A method of making a virus-like particle (VLP) comprising a therapeutic polypeptide, the method comprising:
a) introducing the system of any one of claims 1-11 into a packaging cell; and
b) harvesting VLPs produced by the packaging cell.

35. A method of delivering a therapeutic polypeptide to a eukaryotic cell, and carrying out homology-directed repair (HDR) in the cell, the method comprising:
a) electroporating a solution comprising: i) a VLP of any one of claims 14-24; and ii) a donor template nucleic acid, forming an electroporated VLP/donor template solution; and
b) contacting the cell with the electroporated VLP/donor template solution, wherein said contacting results in HDR and in synthesis of the therapeutic polypeptide in the cell.

36. The method of claim 35, wherein the eukaryotic cell is a T cell, a B cell, or a stem cell.

37. The method of claim 35, wherein the eukaryotic cell is a T cell, wherein the therapeutic polypeptide is a chimeric antigen receptor, and wherein the guide RNA present in the VLP provides for knockout, via HDR, of endogenous 3-2 microglobulin such that, following HDR, the cell does not substantially display MHC class I polypeptides on its surface.
